(19) United States
(12) Patent Application Publication
(10) Pub. No.: US 2026/0216330 A1
(43) Pub. Date: Jul. 30, 2026

(54) USE OF T-CELL ENGAGING PROTEINS IN THE TREATMENT OF CANCER

(71) Applicant: Boehringer Ingelheim International GmbH, Ingelheim am Rhein (DE)

(72) Inventors: Susanne HIPP, Biberach an der Riß (DE); Matus STUDENY, Bratislava (SK); Tillmann TAUBE, Biberach an der Riss (DE)

(21) Appl. No.: 19/395,141

(22) Filed: Nov. 20, 2025

(30) Foreign Application Priority Data
Nov. 20, 2024 (EP) 24214347.7

(51) Int. Cl. A61K 40/11; A61K 40/32; A61K 40/42; A61P 35/00; C07K 16/28

(52) U.S. Cl. CPC A61K 40/11; A61K 40/32; A61K 40/4202; A61P 35/00; C07K 16/2818; C07K 16/2827; A61K 2239/55

(57) ABSTRACT

The present invention relates to T-cell engaging protein comprising a first antigen binding unit specifically binding to DLL3 and a second antigen binding unit specifically binding to CD3 for use in a method for treating a DLL3-positive cancer or a cancer selected from a Neuroendocrine Neo-plasm (NEN), Merkel cell carcinoma (MCC), medullary thyroidal carcinoma and glioma in a subject exhibiting at least a threshold of DLL3-positivity, expressed in percents, the method comprising administering to the subject a dose of said T-cell engaging protein, wherein
said T-cell engaging protein comprises a first antigen binding unit specifically binding to DLL3, comprising light chain CDRs comprising the amino acid sequences of SEQ ID NO:13 (CDR1), SEQ ID NO:14 (CDR2) and SEQ ID NO:15 (CDR3) and heavy chain CDRs comprising the amino acid sequences of SEQ ID NO:16 (CDR1), SEQ ID NO:17 (CDR2) and SEQ ID NO:18 (CDR3) and a second antigen binding unit specifically binding to CD3 comprising light chain CDRs comprising the amino acid sequences of SEQ ID NO:55 (CDR1), SEQ ID NO:56 (CDR2) and SEQ ID NO:57 (CDR3) and heavy chain CDRs comprising the amino acid sequences of SEQ ID NO:58 (CDR1), SEQ ID NO:59 (CDR2) and SEQ ID NO:60 (CDR3).

### INCORPORATION OF SEQUENCE LISTING

A sequence listing containing the file named “SequenceListing.xml” which is 67,348 bytes (measured in MS-Windows®) and created on Nov. 20, 2025, and comprises 90 sequences, is incorporated herein by reference in its entirety.

### TECHNICAL FIELD

The invention generally relates to the treatment of cancer by using T cell engaging protein that specifically binds to CD3 and DLL3. In particular, the present invention relates to selecting a subject suitable for the treatment. Furthermore, the present invention particularly relates to uses and methods of using DLL3/CD3 binding T cell engaging proteins in a dosage and administration regimen for the treatment of cancer.

### BACKGROUND INFORMATION

Extrapulmonary neuroendocrine carcinoma (epNEC) refers to neuroendocrine carcinomas (NECs) occurring in any organ type outside of the lung or with an unknown primary site and comprises around 9% of all NECs [Dasari A, Mehta K, Byers L A, Sorbye H & Yao J C. Comparative study of lung and extrapulmonary poorly differentiated neuroendocrine carcinomas: A SEER database analysis of 162,983 cases. Cancer. 2018; 124(4):807-815]. Clinically, epNECs present with an aggressive course and are characterized by early distant metastatic spread, commonly to liver, lung, and bone. Consequently, most cases are diagnosed late as advanced or metastatic disease and are thus not amenable to surgical treatment [Kawasaki K, Rekhtman N, Quintanal-Villalonga A and Rudin C M. Neuroendocrine neoplasms of the lung and gastrointestinal system: convergent biology and a path to better therapies. Nat Rev Clin Oncol. 2023; 20(1):16-32.]. Even though NECs are chemo- and radiosensitive with a good objective response rate (ORR) to the initial therapy, the prognosis remains dismal as nearly all patients experience relapse and succumb to the disease [Kawasaki K, Rekhtman N, Quintanal-Villalonga A and Rudin C M. Neuroendocrine neoplasms of the lung and gastrointestinal system: convergent biology and a path to better therapies. Nat Rev Clin Oncol. 2023; 20(1):16-32., Alese O B, Jian R, Shaib W, et al. High-Grade Gastrointestinal Neuroendocrine Carcinoma Management and Outcomes: A National Cancer Database Study. Oncologist. 2019; 24(7):911-902.].

Small cell lung carcinoma (SCLC) is a subtype of NEC and represents both the most common pulmonary NEC and NEC overall. Most patients (around 70%) present with extensive stage/metastatic disease at diagnosis [SEER*Explorer: An interactive website for SEER cancer statistics [Internet]. Surveillance Research Program, National Cancer Institute. [Cited 2022 Oct. 19]. Available from https://seer.cancer.gov/statistics-network/explorer/.]. The clinical course of SCLC is highly aggressive, and the disease usually progresses very rapidly [Pietanza M C, Byers L A, Minna J D, Rudin C M. Small cell lung cancer: will recent progress lead to improved outcomes?Clin Cancer Res. 2015; 21(10):2244-2255.]. While tumors are generally sensitive to chemo- and radiotherapy initially, tumors will usually relapse quickly. Treatment outcomes have improved in the past years, but remain comparatively poor, especially in the setting of ES-SCLC [Bogart J A, Waqar S N, Mix MD. Radiation and Systemic Therapy for Limited-Stage Small-Cell Lung Cancer. J Clin Oncol. 2022; 40(6):661-670.]. For patients experiencing relapse, outcomes are even worse.

A rare variant of pulmonary NEC is large-cell neuroendocrine carcinoma of the lung (LCNEC of the lung or LCNEC-L/LCNEC-Lung). LCNEC of the lung represents 2.1 to 3.5% of all lung cancers in a series of surgically resected cases [Fasano M, Della Corte C M, Papaccio F, Ciardiello F, Morgillo F. Pulmonary large-cell neuroendocrine carcinoma: from epidemiology to therapy. J Thorac Oncol 2015; 10:1133-41.]. The disease is characterized by aggressive clinical behavior, with a tendency towards early metastatic spread. Consequently, diagnosis is typically made in advanced/metastatic stages of disease. As a result, prognosis is poor, and the overall survival of advanced/metastatic stage LCNEC-Lung is very limited, ranging from 8 to 12 months [Andrini, E et al. Large Cell Neuroendocrine Carcinoma of the Lung: Current Understanding and Challenges. J. Clin. Med. 2022, 11, 1461. https://doi.org/10.3390/jcm11051461].

Despite the advancements in the treatment of such cancers, improved therapies are still being sought.

### BRIEF SUMMARY OF THE INVENTION

The present invention is based on a treatment using a T-cell engaging protein comprising a first antigen binding unit specifically binding to DLL3 and a second antigen binding unit specifically binding to CD3, wherein said T-cell engaging protein comprises a first antigen binding unit specifically binding to DLL3, comprising light chain CDRs comprising the amino acid sequences of SEQ ID NO:13 (CDR1), SEQ ID NO:14 (CDR2) and SEQ ID NO:15 (CDR3) and heavy chain CDRs comprising the amino acid sequences of SEQ ID NO:16 (CDR1), SEQ ID NO:17 (CDR2) and SEQ ID NO:18 (CDR3) and a second antigen binding unit specifically binding to CD3 comprising light chain CDRs comprising the amino acid sequences of SEQ ID NO:55 (CDR1), SEQ ID NO:56 (CDR2) and SEQ ID NO:57 (CDR3) and heavy chain CDRs comprising the amino acid sequences of SEQ ID NO:58 (CDR1), SEQ ID NO:59 (CDR2) and SEQ ID NO:60 (CDR3) of a subject exhibiting at least a threshold of DLL-3 positivity in order to be selected for said treatment. Due to treating a subject exhibiting at least said threshold with said T-cell engaging protein, significantly improved objective responses in treatments are achieved compared to overall responses in treatments of subjects below said threshold of T-cell positivity. Hence, the present invention in particular involves the selection of a patient for treatment in view of the DLL-3 positivity of the subject in view of the threshold.

In a first aspect, the present invention provides a T-cell engaging protein comprising a first antigen binding unit specifically binding to DLL3 and a second antigen binding unit specifically binding to CD3 for use in a method for treating a DLL3-positive cancer or a cancer selected from a Neuroendocrine Neoplasm (NEN), Merkel cell carcinoma (MCC), medullary thyroidal carcinoma and glioma in a subject exhibiting at least a threshold of DLL3-positivity, the method comprising administering to the subject a dose of said T-cell engaging protein, wherein
said T-cell engaging protein comprises a first antigen binding unit specifically binding to DLL3, comprising light chain CDRs comprising the amino acid sequences of SEQ ID NO:13 (CDR1), SEQ ID NO:14 (CDR2) and SEQ ID NO:15 (CDR3) and heavy chain CDRs comprising the amino acid sequences of SEQ ID NO:16 (CDR1), SEQ ID NO:17 (CDR2) and SEQ ID NO:18 (CDR3) and a second antigen binding unit specifically binding to CD3 comprising light chain CDRs comprising the amino acid sequences of SEQ ID NO:55 (CDR1), SEQ ID NO:56 (CDR2) and SEQ ID NO:57 (CDR3) and heavy chain CDRs comprising the amino acid sequences of SEQ ID NO:58 (CDR1), SEQ ID NO:59 (CDR2) and SEQ ID NO:60 (CDR3).

In a second aspect, the present invention provides a method for selecting a subject suffering from a DLL3-positive cancer or a cancer selected from a Neuroendocrine Neoplasm (NEN), Merkel cell carcinoma (MCC), medullary thyroidal carcinoma and glioma for treatment with a T-cell engaging protein comprising a first antigen binding unit specifically binding to DLL3 and a second antigen binding unit specifically binding to CD3, the method comprising:
(a) determining or having determined in a sample that the subject exhibits DLL3 positivity, wherein the subject is selected for treatment with said T-cell engaging protein when the DLL3-positivity exhibits at least a threshold, and
wherein said T-cell engaging protein comprises a first antigen binding unit specifically binding to DLL3, comprising light chain CDRs comprising the amino acid sequences of SEQ ID NO:13 (CDRT), SEQ ID NO:14 (CDR2) and SEQ ID NO:15 (CDR3) and heavy chain CDRs comprising the amino acid sequences of SEQ ID NO:16 (CDR1), SEQ ID NO:17 (CDR2) and SEQ ID NO:18 (CDR3) and a second antigen binding unit specifically binding to CD3 comprising light chain CDRs comprising the amino acid sequences of SEQ ID NO:55 (CDRT), SEQ ID NO:56 (CDR2) and SEQ ID NO:57 (CDR3) and heavy chain CDRs comprising the amino acid sequences of SEQ ID NO:58 (CDR1), SEQ ID NO:59 (CDR2) and SEQ ID NO:60 (CDR3).

In a third aspect, the present invention provides a T-cell engaging protein comprising a first antigen binding unit that specifically binds to DLL3 and a second antigen binding unit that specifically binds to CD3 for use in a method of treatment of a DLL3-positive cancer or a cancer selected from a Neuroendocrine Neoplasm (NEN), Merkel cell carcinoma (MCC), medullary thyroidal carcinoma and glioma,
the method comprising a step-in cycle, said step-in cycle comprising the steps of:
   administering to a subject an initial dose of said T-cell engaging protein, said initial dose ranging from about 20 μg/kg of the subject's body weight to about 40 μg/kg of the subject's body weight,
   administering to the subject an elevated dose, which is elevated as compared to said initial dose, of said T-cell engaging protein, the elevated dose ranging from about 60 μg/kg of the subject's body weight to about 100 μg/kg of the subject's body weight,
   administering to the subject a further dose of the T-cell engaging protein, said further dose ranging from about 5 mg to about 70 mg.

In a fourth aspect, the present invention provides a T-cell engaging protein comprising a first antigen binding unit that specifically binds to DLL3 and a second antigen binding unit that specifically binds to CD3 for use in a method of treatment of a DLL3-positive cancer or a cancer selected from a Neuroendocrine Neoplasm (NEN), Merkel cell carcinoma (MCC), medullary thyroidal carcinoma and glioma,
the method comprising a step-in cycle, said step-in cycle comprising the steps of: said step-in cycle comprising the steps of:
   administering to a subject an initial dose of said T-cell engaging protein, said initial dose ranging from about 5 μg/kg of the subject's body weight to about 15 μg/kg of the subject's body weight,
   administering to the subject an elevated dose, which is elevated as compared to said initial dose, of said T-cell engaging protein, the elevated dose ranging from about 20 μg/kg of the subject's body weight to about 40 μg/kg of the subject's body weight,
   administering to the subject a further dose of said T-cell engaging protein, said further dose ranging from about 80 μg/kg of the subject's body weight to about 1600 μg/kg of the subject's bodyweight; or
the method comprising a step-in cycle, the step-in cycle comprising the steps of:
   administering to the subject an initial dose, an elevated dose, which is elevated as compared to said initial dose, and a further dose of said T-cell engaging protein, wherein a ratio between said initial, elevated and further dose per kilogram of the subject's body weight is about 1:about 3:about 9.

Embodiments of the first aspect of the invention define mutatis mutandis preferred embodiments of said second, third and fourth aspects.

For each aspect of the present invention, a premedication and/or a post-medication may be administered prior to a respective dose of the T-cell engaging protein (premedication) and/or after respective dose of the T-cell engaging protein (post-medication). Premedications and/or post-medications may comprise a non-steroidal anti-inflammatory drug, such as acetaminophen, an anti-histamine and/or a steroid, preferably a corticosteroid such as prednisolone or dexamethasone, respectively. In certain embodiments, the premedication and post-medication are used during the step-in doses of the T-cell engaging protein, i.e. initial, elevated and/or further doses. The use of premedication and post-medication leads to reduction of adverse events and, therefore, increases the safety of the treatment.

Further aspects, embodiments, uses and methods involving the binding proteins of the invention will become clear from the following detailed description of the invention and from the appended claims.

### BRIEF DESCRIPTION OF THE DRAWINGS

FIG. 1: Classification of Neuroendocrine Neoplasms adapted from Rindi et al., Endocr Pathol; 2022; 33; 115-154.

FIG. 2: Safety Results from regimens A, B1 and B2 (data output Q1/2023): Frequency of patients with investigator defined treatment related AEs occurring with incidence in preferred term >=5% by highest CTCAE grade, on treatment period.

FIGS. 3A1-A3: Frequency [N (%)] of patients with cytokine release syndrome by dose level and highest CTCAE grade—Treated set—Regimen A (data output Q1/2024).

FIG. 3B: Frequency [N (%)] of patients with cytokine release syndrome by dose level and highest CTCAE grade—Treated set—Regimen B1 (data output Q1/2024).

FIGS. 3C1-C3: Frequency [N (%)] of patients with cytokine release syndrome by treatment period, target dose and highest CTCAE grade—Treated set—Regimen B2 (data output Q1/2024).

FIGS. 3D1-D3: Frequency [N (%)] of patients with cytokine release syndrome by treatment period, target dose and highest CTCAE grade—Treated set—Regimen B3 (data output Q1/2024).

FIG. 3E: Frequency [N (%)] of patients with cytokine release syndrome by treatment period, highest CTCAE grade (grouped) on treatment period, Treated set—Regimen B2 and B3 (data output Q1/2024).

FIG. 3F: Frequency [N (%)] of patients with drug-related potential neurologic toxicity including ICANS (narrow) by user-defined categories by regimen, highest CTCAE grade (grouped), and preferred term on-treatment period treated set—Regimen B2 and B3 (data output Q1/2024).

FIG. 4A Best overall tumor response and disease control for regimens A, B1, B2, B3 (data output Q1/2024)

FIGS. 4B1-B3: Best overall tumor response and disease control by dose level—Efficacy Set—Regimen B2 (data output Q1/2024)

FIGS. 4C1-C2: Best overall tumor response and disease control by dose level—Efficacy Set—Regimen B3 (data output Q1/2024)

FIG. 4D: Best overall tumor response and disease control by tumor type—Efficacy Set—Regimen B2 (data output Q1/2024)

FIG. 4E: Best overall tumor response and disease control by tumor type—Efficacy Set—Regimen B3 (data output Q1/2024)

FIG. 4F: Waterfall plot of best percentage change from baseline in target lesions by dose level—Efficacy Set—Regimen B2 (data output Q1/2024)

FIG. 5A: Cytokine levels in peripheral blood at different timepoints after administration of BI 764532 in a Q3W regimen (Regimen A). Each line represents the mean of the patients as indicated at the bottom of the Figure. Arrows at the top of the figure indicated timing of BI 764532 administration (data output Q1/2023).

FIG. 5B: Cytokine levels in peripheral blood at different timepoints after administration of BI 764532 in a QW regimen (Regimen B1). Each line represents the mean of the number of patients as indicated at the bottom of the Figure. Arrows at the top of the figure indicated timing of BI 764532 administration (data output Q1/2023).

FIG. 5C: Cytokine levels in peripheral blood at different timepoints after administration of BI 764532 in a QW regimen with a first dose of 30 μg/kg, a second dose of 90 μg/kg, and a target dose of either 90, 180, 270, 360, or 540 μg/kg (Regimen B2). Each line represents the mean of the number of patients as indicated at the bottom of the Figure. Arrows at the top of the figure indicated timing of BI 764532 administration (data output Q1/2023).

FIG. 5D: Cytokine levels in peripheral blood at different timepoints after administration of BI 764532 in a QW regimen with a first dose of 30 μg/kg, a second dose of 90 μg/kg, and a target dose of either 90, 180, 270, 360, 540, 720, 1080, or 1530 μg/kg (Regimen B2). Each line represents the mean of the number of patients as indicated at the bottom of the Figure. Arrows at the top of the figure indicated timing of BI 764532 administration (data output Q1/2024).

FIG. 5E: Cytokine levels in peripheral blood at different timepoints after administration of BI 764532 in a QW regimen with a first dose of 10 μg/kg, a second dose of 30 μg/kg, a third dose of 90 μg/kg, and a target dose of either 90, 180, 270, 360, 540, 720, or 1080 μg/kg (Regimen B3). Each line represents the mean of the number of patients as indicated at the bottom of the Figure. Arrows at the top of the figure indicated timing of BI 764532 administration (data output Q1/2024).

FIG. 6A: Dot plot of correlation of DLL3% with best overall response—Patients with DLL3% positivity in Efficacy Set (Partial Response (PR), Stable Disease (SD) or Progressive Disease (PD))—B2 regimen.

FIG. 6B-E: Best overall tumor response and disease control by DLL3 expression (75%, 50%, 25% cutoff or all)—Patients with DLL3% positive in Efficacy Set—Regimen B2.

FIG. 7A: Dot plot of correlation of DLL3% with best overall response—Patients with DLL3% positivity in Efficacy Set (Partial Response (PR), Stable Disease (SD) or Progressive Disease (PD))—B3 regimen

FIG. 7B-E: Best overall tumor response and disease control by DLL3 expression (75%, 50%, 25% cutoff or all)—Patients with DLL3% positive in Efficacy Set—Regimen B3

FIG. 8A: Dot plot of correlation of DLL3% with best overall response—Patients with DLL3% positivity in Efficacy Set (Complete Response (CR), Partial Response (PR), Stable Disease (SD) or Progressive Disease (PD))—B2+B3 regimen

FIG. 8B-E: Best overall tumor response and disease control by DLL3 expression (75%, 50%, 25% cutoff or all)—Patients with DLL3% positive in Efficacy Set—Regimen B2+B3

FIG. 9A: Dot plot of correlation of DLL3% with best overall response—epNEC Patients with DLL3% positivity in Efficacy Set—B2+B3 regimen

FIG. 9B-E: Best overall tumor response and disease control by DLL3 expression (75%, 50%, 25% cutoff or all)—epNEC Patients with DLL3% positive in Efficacy Set—Regimen B2+B3

FIG. 10A: Dot plot of correlation of DLL3% with best overall response—SCLC Patients with DLL3% positivity in Efficacy Set—B2+B3 regimen

FIG. 10B-E: Best overall tumor response and disease control by DLL3 expression (75%, 50%, 25% cutoff or all)—SCLC Patients with DLL3% positive in Efficacy Set—Regimen B2+B3

FIG. 11A: Dot plot of correlation of DLL3% with best overall response—LCNEC Patients with DLL3% positivity in Efficacy Set—B2+B3 regimen

FIG. 11B-E: Best overall tumor response and disease control by DLL3 expression (75%, 50%, 25% cutoff or all)—LCNEC Patients with DLL3% positive in Efficacy Set—Regimen B2+B3

FIG. 12A: Frequency (N (%)) of patients with the most frequent (>5%) obrixtamig related AEs by treatment, maximum CTCAE grade and preferred term (Dareon™-9).

FIG. 12B: Frequency (N (%)) of patients with potential neurologic toxicity including ICANS (narrow) by user-defined categories (Dareon™-9).

FIG. 12C: Frequency (N (%)) of patients with topotecan related AEs by treatment, maximum CTCAE grade and preferred term. Only AEs occurring in >5% of patients are displayed (Dareon™-9).

FIG. 12D: Best overall tumor response (unconfirmed) and disease control according to RECIST V1.1 (investigator assessment) by treatment (Dareon™-9).

FIG. 12E: Best overall tumor response (confirmed) and disease control according to RECIST V1.1. (investigator assessment) by treatment (Dareon™-9).

FIG. 12F: Duration of response (DoR) (confirmed) by treatment (Dareon™-9).

FIG. 12G: Waterfall plot of best percentage change from baseline in target lesions by treatment (Dareon™-9).

FIG. 13A: Disposition of patients—screened set (Dareon™-7).

FIG. 13B: Frequency (N (%)) of patients with obrixtamig related AEs by treatment, maximum CTCAE grade and preferred term—obrixtamig-treated set. Only AEs occurring in >5% of the patients are displayed (Dareon™-7).

FIG. 13C: Frequency (N (%)) of patients with potential neurologic toxicity including ICANS (narrow) by user-defined categories—obrixtamig related, by treatment, maximum CTCAE grade and preferred term—obrixtamig-treated set (Dareon™-7).

FIG. 13D: Frequency (N (%)) of patients with chemotherapy-related AEs by treatment, maximum CTCAE grade and preferred term—obrixtamig-related set. Only AEs occurring in >5% of patients are displayed (Dareon™-7).

FIG. 13E: Best overall tumor response (unconfirmed) and disease control according to RECIST V1.1 (investigator assessment) by treatment (Dareon™-7).

FIG. 13F: Best overall tumor response (confirmed) and disease control according to RECIST V1.1 (investigator assessment) by treatment (Dareon™-7).

FIG. 13G: Duration of objective response (DoR) (confirmed) by treatment (Dareon™-7).

FIG. 14A: Disposition of patients—screened set (Dareon™-8).

FIG. 14B: Frequency (N (%)) of patients with obrixtamig-related AEs by treatment, maximum CTCAE grade and preferred term—obrixtamig-treated set. Only AEs occurring in >5% of patients are displayed (Dareon™-8).

FIG. 14C: Frequency (N (%)) of patients with potential neurologic toxicity including ICANS (narrow) by user-defined categories—obrixtamig-related, by treatment, maximum CTCAE grade and preferred term—obrixtamig-treated set (Dareon™-8).

FIG. 14D: Frequency (N (%)) of patients with atezolizumab/chemotherapy-related AEs by treatment, maximum CTCAE grade and preferred term—Obrixtamig-treated set. Only AEs occurring in >10% of patients are displayed (Dareon™-8).

FIG. 14E: Best overall tumor response (unconfirmed) and disease control according to RECIST V1.1 (investigator assessment) by treatment (Dareon™-8).

FIG. 14F: Best overall tumor response (unconfirmed) and disease control according to RECIST V1.1 (investigator assessment) by treatment (Dareon™-8).

FIG. 14G: Duration of objective response (DoR) (confirmed) by treatment (Dareon™-8).

FIG. 14H: Waterfall plot of best percentage change from baseline in target lesions by treatment (Dareon™-8).

FIG. 14I: PPS by treatment (Dareon™-8).

FIG. 14J: Kaplan-Meier plot of progression-free survival (Dareon™-8).

FIG. 15: Main Analysis Set: Best Objective Response (Confirmed), Disease Control, median DoR, median PFS, According to RECIST v1.1 (Investigator Assessment), median OS, by DLL3 Expression.

FIG. 16: Summary of Objective Response (Confirmed), Disease Control, and Duration of Objective Response According to RECIST v1.1 (Investigator Assessment) and Progression Free Survival by DLL3 Expression—1L epNEC from DAREON-7 (EFS1)

FIG. 17: Efficacy by DLL3 expression—2L+SCLC, in Efficacy Set 1 (1438-0001 B2/B3) and Efficacy Set 1 (Dareon™-5, Dose Pooling) Combined

FIG. 18: Objective Response (Confirmed), Disease Control, and Duration of Objective Response According to RECIST v1.1 (Investigator Assessment) by DLL3 Expression—1L ES-SCLC, in Efficacy Set 1 (Dareon™-8, Dose Pooling).

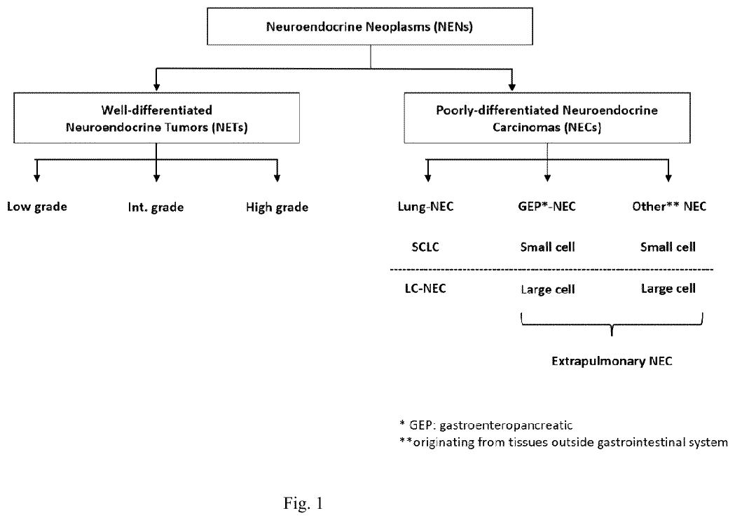

### DETAILED DESCRIPTION OF THE INVENTION

### Used Terms and Definitions

The above and other aspects and embodiments of the invention will become clear from the further description herein.

Any definition given below serves at least two purposes: establish the meaning of terms used in this disclosure and provide a description of preferred or exemplary embodiments.

Unless indicated or defined otherwise, all terms used have their usual meaning in the art, which will be clear to the skilled person. Reference is for example made to the standard handbooks, such as Sambrook et al, “Molecular Cloning: A Laboratory Manual” (2nd Ed.), Vols. 1-3, Cold Spring Harbor Laboratory Press (1989); Lewin, “Genes IV”, Oxford University Press, New York, (1990), and Roitt et al, “Immunology” (2nd Ed.), Gower Medical Publishing, London, New York (1989), as well as to the general background art cited herein. Furthermore, unless indicated otherwise, all methods, steps, techniques and manipulations that are not specifically described in detail can be performed and have been performed in a manner known per se, as will be clear to the skilled person. Reference is for example again made to the standard handbooks, to the general background art referred to above and to the further references cited therein.

When used herein the term “comprising” and variations thereof such as “comprises” and “comprise” can be substituted with the term “containing” or “including” or “having.”

The T cell engagers of the present invention are generally referred to as “T-cell engaging proteins” or, with equivalent meaning, “T-cell engaging molecules”, or “TCEs”. The term “protein” has its art-established meaning and refers to a biomolecule comprising or consisting of one or a plurality of polypeptide chains, wherein in case of plurality of polypeptide chains, said chains are bound to each other non-covalently or covalently such as via one or more disulfide bonds. A “polypeptide” or “polypeptide chain” is a polycondensate of amino acids, preferably of the naturally occurring proteinogenic amino acids. Preferred polypeptide sequences comprised in T-cell engaging proteins of the invention are reproduced further below as well as in the attached sequence listing.

TCEs to be used in accordance with the invention comprise the two binding units defined in the first and other aspects of the invention. As such, further binding units may be, but not have to be present. In other words, the TCEs may be tri-specific or multi-specific, while preference is given to bispecific TCEs which are subject of preferred embodiments or disclosed in WO 2019/234220.

Said T-cell engaging proteins, in particular said bispecific TCEs, preferably share structural and functional features with antibodies, more specifically Immunoglobulins of the G type (IgGs). Such shared features preferably include the presence of complementarity determining regions (CDRs) conferring specificity as well as the molecular architecture made of immunoglobulin domains. This is why sometimes in the context of characterizing said bispecific T-cell engaging proteins, terminology from the field of antibodies is used. Having said that, at least certain preferred bispecific T-cell engaging proteins are not antibodies in a strict sense. Key differences include the bi-specificity as well as linkers (as defined further below) connecting the constant domain of a given light chain with the variable domain of the corresponding heavy chain (“corresponding” in this context meaning that the two mentioned chains comprise a particular antigen binding unit). As a consequence, preferred molecular architectures of said bispecific T-cell engaging proteins comprise or consist of two polypeptide chains (as opposed to four polypeptide chains generally present in an IgG molecule).

The term “sequence” as used herein (for example in terms like “heavy/light chain sequence”, “antibody sequence”, “variable domain sequence”, “constant domain sequence” or “protein sequence”), should generally be understood to include both the relevant amino acid sequence as well as nucleic acid sequences or nucleotide sequences encoding the same, unless the context requires a more limited interpretation.

An “antigen binding unit” as used herein refers to a polypeptide capable of binding to its specific target or antigen and comprising the minimal structural requirements derived from an antibody (typically present in an antibody) which allow for target binding. Thus, an antigen binding unit comprises at least the presence of three light chain and three heavy chain CDR sequences, preferably at least a light chain variable domain and a heavy chain variable domain.

The generalized structure of an antibody or immunoglobulin is well known to those of skill in the art. These molecules are heterotetrameric glycoproteins, typically of about 150,000 daltons, composed of two identical light (L) chains and two identical heavy (H) chains and are typically referred to as full length antibodies. Each light chain is covalently linked to a heavy chain by one disulfide bond to form a heterodimer, and the heterotetrameric molecule is formed through a covalent disulfide linkage between the two identical heavy chains of the heterodimers. Although the light and heavy chains are linked together by one disulfide bond, the number of disulfide linkages between the two heavy chains varies by immunoglobulin isotype. Each heavy and light chain also has regularly spaced intrachain disulfide bridges. Each heavy chain has at the N-terminus a variable domain (VH), followed by three or four (in case of IgE) constant domains (CH1, CH2, CH3, and CH4), as well as a hinge region between CH1 and CH2. Each light chain has two domains, an N-terminal variable domain (VL) and a C-terminal constant domain (CL). The VL domain associates non-covalently with the VH domain, whereas the CL domain is commonly covalently linked to the CH1 domain via a disulfide bond. Particular amino acid residues are believed to form an interface between the light and heavy chain variable domains (Chothia et al, 1985, J. Mol. Biol. 186:651-663). Variable domains are also referred to herein as variable regions or Fv and denote the part that confers specificity to an antibody for the antigen by carrying the antigen-binding site.

The “light chain variable domain” (or “light chain variable region”) and “heavy chain variable domain” (or “heavy chain variable region”) as used herein have the same general structure and each domain essentially consists of four framework (FR) regions whose sequences are widely conserved, which are referred to in the art and hereinbelow as “framework region 1” or “FR1”; as “framework region 2” or “FR2”; as “framework region 3” or “FR3”; and as “framework region 4” or “FR4”, respectively; which framework regions are interrupted by three hypervariable regions, HVRs (or CDRs), which are referred to in the art and herein below as “complementarity determining region for “CDR1”; as “complementarity determining region 2” or “CDR2”; and as “complementarity determining region 3” or “CDR3”, respectively. Thus, the general structure or sequence of an immunoglobulin variable domain can be indicated as follows: FR1-CDR1-FR2-CDR2-FR3-CDR3 FR4. The framework regions adopt a beta-sheet conformation and the CDRs may form loops connecting the beta-sheet structure. The CDRs in each chain are held in their three-dimensional structure by the framework regions and form together with the CDRs from the other chain the antigen binding site.

Within the context of this invention, reference to CDR's is based on the definition of CCG, also referred to as IMGT (Lefranc M P, Pommie C, Ruiz M, Giudicelli V, Foulquier E, Truong L, Thouvenin-Contet V, Lefranc G. “IMGT unique numbering for immunoglobulin and T cell receptor variable domains and Ig superfamily V-like domains.” Dev Comp Immunol. 2003 January; 27(1):55-77; Giudicelli V, Brochet X, Lefranc M P. “IMGT/V-QUEST: IMGT standardized analysis of the immunoglobulin (IG) and T cell receptor (TR) nucleotide sequences”. Cold Spring Harb Protoc. 2011; 2011 (6):695-715. An alternative definition of CDRs known in the art is based on Chothia (Chothia and Lesk, J. Mol. Biol. 1987, 196: 901-917), together with Kabat (E. A. Kabat, T. T. Wu, H. Bilofsky, M. Reid-Miller and H. Perry, Sequence of Proteins of Immunological Interest, National Institutes of Health, Bethesda (1983)).

The term “constant domains” or “constant region” as used within the current application denotes the sum of the domains of an antibody other than the variable region. Such constant domains and regions are well known in the state of the art and e.g. described by Kabat et al. (“Sequence of proteins of immunological interest”, US Public Health Services, NIH Bethesda, MD, Publication No. 91).

The “Fc part” or “Fc domain” of an antibody is not involved directly in binding of an antis body to an antigen, but exhibits various effector functions. An “Fc part/domain of an antibody” is a term well known to the skilled artisan and defined on the basis of papain cleavage of antibodies. Depending on the amino acid sequence of the constant region of their heavy chains, antibodies or immunoglobulins are divided in the classes: IgA, IgD, IgE, IgG and IgM. According to the heavy chain constant regions the different classes of immunoglobulins are called a, d, e, g, and m respectively. Several of these may be further divided into subclasses (isotypes), e.g. IgG1, IgG2, IgG3, and IgG4, IgA1, and IgA2. The Fc part of an antibody is directly involved in ADCC (antibody dependent cell-mediated cytotoxicity) and CDC (complement-dependent cytotoxicity) based on complement activation, Clq binding and Fc receptor binding. Complement activation (CDC) is initiated by binding of complement factor Clq to the Fc part of most IgG antibody subclasses. While the influence of an antibody on the complement system is dependent on certain conditions, binding to Clq is caused by defined binding sites in the Fc part. Such binding sites are e.g. L234, L235, D270, N297, E318, K320, K322, P331 and P329 (numbering according to EU numbering (Edelman et al, Proc Natl Acad Sci USA. 1969 May; 63(1):78-85)). Most crucial among these residues in mediating Clq and Fcgamma receptor binding in IgG1 are L234 and L235 (Hezareh et al, J. Virology 75 (2001) 12161-12168, Shields et al (2001) JBC, 276 (9): 6591-6604). Antibodies of subclass IgG1 and IgG3 usually show complement activation and Clq and C3 binding, whereas IgG2 and IgG4 do not activate the complement system and do not bind Clq and C3.

In the context of the present invention, an Fc domain is for example derived from the heavy chain of an IgG, for example an IgG₁, IgG₂ or IgG₄. For example, an Fc domain of the present invention is a Fc domain of a heavy chain of an IgG₁ or IgG₄ and comprises a hinge region and two constant domains (C_{H2} and C_{H3}). Examples of Fc domains (including a hinge region) are shown in SEQ ID NOs:81 and 84.

The numbering of the amino acids in the amino acid chains of a protein of the present invention is herein according to the EU numbering system (Edelman, Cunningham et al. 1969), unless otherwise specified. This means that the amino acid numbers indicated herein correspond to the positions in a heavy chain of the corresponding sub-type (e.g. IgG₁ or IgG₄), according to the EU numbering system, unless otherwise specified.

The term “antibody” or “antibody molecule” (used synonymously herein) encompasses a monoclonal antibody, a polyclonal antibody, a human antibody, a humanized antibody, a chimeric antibody, multispecific antibodies (e.g., bispecific antibodies), a fragment of an antibody, in particular a Fv, Fab, Fab′, or F(ab′)2 fragment. The antibody may have an effector function, such as ADCC or CDC, that is usually mediated by the Fc part (antibody constant region) of the antibody, or it may have no effector function, e.g. by lacking a Fc part or having a blocked, masked Fc part, in essence a Fc part that is not or insufficiently recognized by immune cells or immune system components, like the complement system.

Monoclonal antibodies (mAb) are monospecific antibodies that are identical in amino acid sequence. They may be produced by hybridoma technology from a hybrid cell line (called hybridoma) representing a clone of a fusion of a specific antibody-producing B cell with a myeloma (B cell cancer) cell (Kohler G, Milstein C. Continuous cultures of fused cells secreting antibody of predefined specificity. Nature 1975; 256:495-7.). Alternatively, monoclonal antibodies may be produced by recombinant expression in host cells (Norderhaug L, Olafsen T, Michaelsen T E, Sandlie I. (May 1997). “Versatile vectors for transient and stable expression of recombinant antibody molecules in mammalian cells.” J Immunol Methods 204 (1): 77-87; see also below). A “recombinant antibody” or “recombinant binding protein” is an antibody or binding protein which has been produced by a recombinantly engineered host cell. It is optionally isolated or purified.

Full length antibodies can be treated with enzymes such as papain or pepsin to generate useful antibody fragments. Papain digestion is used to produce two identical antigen-binding antibody fragments called “Fab” fragments, each with a single antigen-binding site, and a residual “Fc” fragment. The Fab fragment also contains the constant domain of the light chain and the CH1 domain of the heavy chain. Pepsin treatment yields a F(ab′)2 fragment that has two antigen-binding sites and is still capable of cross-linking antigen. Fab′ fragments differ from Fab fragments by the presence of additional residues including one or more cysteines from the antibody hinge region at the C-terminus of the CH1 domain. F(ab′)2 antibody fragments are pairs of Fab′ fragments linked by cysteine residues in the hinge region. Other chemical couplings of antibody fragments are also known.

“Fv” fragment contains a complete antigen-recognition and binding site consisting of a dimer of one heavy and one light chain variable domain in tight, non-covalent association. In this configuration, the three CDRs of each variable domain interact to define an antigen-biding site on the surface of the VH-VL dimer. Collectively, the six CDRs confer antigen binding specificity to the antibody.

A “single-chain Fv” or “scFv” antibody fragment is a single chain Fv variant comprising the VH and VL domains of an antibody where the domains are present in a single polypeptide chain. The single chain Fv is capable of recognizing and binding an antigen. The scFv polypeptide may optionally also contain a polypeptide linker positioned between the VH and VL domains in order to facilitate formation of a desired three-dimensional structure for antigen binding by the scFv (see, e.g., Pluckthun, 1994, In The Pharmacology of monoclonal Antibodies, Vol. 113, Rosenburg and Moore eds., Springer-Verlag, New York, pp. 269-315).

A “single-chain Fab” or “scFab” antibody fragment is a single chain Fab variant comprising the VL, CL, VH and CH1 domains of an antibody where the domains are present in a single polypeptide chain. Said polypeptide chain may consist of said domains, consist essentially thereof or comprise them. The single chain Fab is capable of recognizing and binding an antigen. The scFab polypeptide may optionally also contain a polypeptide linker positioned between the CL and VH domains (Hust et al (2007) BMC Biotechnology). To the extent said polypeptide chain comprises said VL, CL, VH and CH1 domains, yet further domains, preferably Ig domains, more preferably CH2 and/or CH3 domains may be present.

For application in man, it is often desirable to reduce immunogenicity of therapeutic molecules, such as antibodies or binding proteins comprising an antigen binding unit as described herein, originally derived from other species, like mouse. This can be done by construction of chimeric antibodies/binding proteins, or by a process called “humanization”. In this context, a “chimeric antibody”; or “chimeric antigen binding unit” is understood to be an antibody or an antigen binding unit comprising a sequence part (e.g. a variable domain) derived from one species (e.g. mouse) fused to a sequence part (e.g. the constant domains) derived from a different species (e.g. human). In this context, a “humanized antibody”, “a humanized binding protein” or a “humanized antigen binding unit” is an antibody, a protein or antigen binding unit comprising a variable domain originally derived from a non-human species, wherein certain amino acids have been mutated to make the overall sequence of that variable domain more closely resemble a sequence of a human variable domain. Methods of humanization of antibodies are well-known in the art (Billetta R, Lobuglio A F. “Chimeric antibodies”. Int Rev Immunol 1993; 10(2-3): 165-76; Riechmann L, Clark M, Waldmann H, Winter G (1988). “Reshaping human antibodies for therapy”. Nature: 332:323).

An “optimized antibody” or an “optimized antigen binding unit or protein” is a specific type of humanized antibody or humanized antigen binding unit/protein which includes an immunoglobulin amino acid sequence variant, or fragment thereof, which is capable of binding to a predetermined antigen and which comprises one or more FRs having substantially the amino acid sequence of a human immunoglobulin and one or more CDRs having substantially the amino acid sequence of a non-human immunoglobulin. This non-human amino acid sequence often referred to as an “import” sequence is typically taken from an “import” antibody domain, particularly a variable domain. In general, an optimized antibody includes at least the CDRs (or HVLs) of a non-human antibody or derived from a non-human antibody, inserted between the FRs of a human heavy or light chain variable domain. It will be understood that certain mouse FR residues may be important to the function of the optimized antibodies and therefore certain of the human germline sequence heavy and light chain variable domains residues are modified to be the same as those of the corresponding mouse sequence. During this process undesired amino acids may also be removed or changed, for example to avoid deamidation, undesirable charges or lipophilicity or non-specific binding. An “optimized antibody”, an “optimized antibody fragment” or “optimized” may sometimes be referred to as “humanized antibody”, “humanized antibody fragment” or “humanized”, or as “sequence-optimized”.

Furthermore, technologies have been developed for creating antibodies or VH/VL domains based on sequences derived from the human genome, for example by phage display or use of transgenic animals (WWW. Ablexis.com/technology-alivamab.php; WO 90/05144; D. Marks, H. R. Hoogenboom, T. P. Bonnert, J. McCafferty, A. D. Griffiths and G. Winter (1991) “By-passing immunisation. Human antibodies from V-gene libraries displayed on phage.” J. Mol. Biol, 222, 581-597; Knappik et al, J. Mol. Biol. 296: 57-86, 2000; S. Carmen and L. Jermutus, “Concepts in antibody phage display”. Briefings in Functional Genomics and Proteomics 2002 1(2): 189-203; Lonberg N, Huszar D. “Human antibodies from transgenic mice”. Int Rev Immunol. 1995; 13(1):65-93.; Bruggemann M, Taussig M J. “Production of human antibody repertoires in transgenic mice”. Curr Opin Biotechnol. 1997 August; 8(4):455-8.). Such antibodies or antigen binding units or VH/VL domains are “human antibodies,” “human antigen binding units,” or “human VH/VL domains” in the context of the present invention.

The term “human antibody”, “human antigen binding unit”, or “human VH/VL domain” as used herein, is intended to include antibodies, antigen binding units or VH/VL domains having variable (and constant, if applicable) regions derived from human germline immunoglobulin sequences. The human antibodies, antigen binding units, proteins or VH/VL domains of the present technology may include amino acid residues not encoded by human germline immunoglobulin sequences (e.g., mutations introduced by random or site-specific mutagenesis in vitro or by somatic mutation in vivo). However, the term “human antibody”, “human antigen binding unit”, or “human VH/VL domain” as used herein, is not intended to include antibodies in which CDR sequences derived from the germline of another (mammalian species), such as a mouse, rat or rabbit, have been grafted onto human framework sequences. Thus, as used herein, the term “human antibody”, “human antigen binding unit”, or “human VH/VL domain” refer to an antibody, antigen binding unit or VH/VL domain in which substantially every part of the protein (e.g., CDR, framework, CL, CH domains (e.g., CH1, CH2, CH3), hinge, VL, VH) is substantially non-immunogenic in humans, with only minor sequence changes or variations. Such changes or variations optionally and preferably retain or reduce the immunogenicity in humans or other species relative to non-modified antibodies or antigen binding units.

Thus, a human antibody, human antigen binding unit or human VH/VL domain is distinct from e.g., a chimeric or humanized antibody. It is pointed out that a human antibody, human antigen binding unit or human VH/VL domain can be produced by a non-human animal or prokaryotic or eukaryotic cell that is capable of expressing functionally rearranged human immunoglobulin (e.g., heavy chain and/or light chain) genes. The term “monomer” refers to a homogenous form of an antibody or a multi-specific protein as described herein.

For example, for a full-length antibody, monomer means a monomeric antibody having two identical heavy chains and two identical light chains. In the context of the present invention, a monomer means a protein of the present invention having a single antigen binding unit specific for DLL3, and a single antigen binding unit specific for CD3 as described herein. For example, a monomer of a binding protein described herein may have two chains, a first chain comprising a single chain Fab with a first antigen binding unit and optionally a first Fc domain and a second chain comprising a single chain Fab with a second antigen binding unit and optionally a second Fc domain. An epitope is a region of an antigen that is bound by an antibody or antigen binding moiety (e.g. the antigen binding unit of the proteins described herein). The term “epitope” includes any polypeptide determinant capable of specific binding to an antibody or antigen binding moiety. In certain embodiments, epitope determinants include chemically active surface groupings of molecules such as amino acids, glycan side chains, phosphoryl, or sulfonyl, and, in certain embodiments, may have specific three-dimensional structural characteristics, and/or specific charge characteristics. Conformational and non-conformational epitopes are distinguished in that the binding to the former but not the latter is lost in the presence of denaturing solvents.

An antigen binding molecule/protein (such as an immunoglobulin, an antibody, an antigen binding unit, or a fragment of such antigen binding molecule/protein) that can“bind”, “bind to”, “specifically bind”, or “specifically bind to”, that “has affinity for”, “is specific for” and/or that “has specificity for” a certain epitope, antigen or protein (or for at least one part, fragment or epitope thereof) is said to be “against” or “directed against” said epitope, antigen or protein or is a “binding” molecule/protein with respect to such epitope, antigen or protein.

As used herein, the terms “binding” and “specific binding” refer to the binding of the antibody or antigen binding moiety (such as an immunoglobulin, an antibody, an antigen binding unit, or a fragment of such antigen binding molecule/protein) to an epitope of the antigen in an in vitro assay, preferably in a plasmon resonance assay ((Malmqvist M., “Surface plasmon resonance for detection and measurement of antibody-antigen affinity and kinetics.”, Curr Opin Immunol. 1993 April; 5(2):282-6.)) with purified wild-type antigen. Antibody affinity can also be measured using kinetic exclusion assay (KinExA) technology (Darling, R. J., and Brault P-A., “Kinetic exclusion assay technology: Characterization of Molecular Interactions.” ASSAY and Drug Development Technologies. 2004, December 2(6): 647-657).

Generally, the term “specificity” refers to the number of different types of antigens or epitopes to which a particular antigen binding molecule/protein (such as an immunoglobulin, an antibody, an antigen binding unit, or a fragment of such antigen binding molecule/protein) can bind. The specificity of an antigen-binding molecule/protein can be determined based on its affinity and/or avidity. The affinity, represented by the equilibrium constant for the dissociation of an antigen with an antigen-binding protein (KD), is a measure for the binding strength between an epitope and an antigen-binding site on the antigen-binding molecule/protein: the lesser the value of the KD, the stronger the binding strength between an epitope and the antigen-binding molecule/protein (alternatively, the affinity can also be expressed as the affinity constant (KA), which is 1/KD). As will be clear to the skilled person (for example on the basis of the further disclosure herein), affinity can be determined in a manner known per se, depending on the specific antigen of interest. Avidity is the measure of the strength of binding between an antigen-binding molecule/protein (such as an immunoglobulin, an antibody, an antigen binding unit, or fragment of such antigen binding molecule/protein) and the pertinent antigen. Avidity is related to both the affinity between an epitope and its antigen binding site on the antigen-binding molecule/protein and the number of pertinent binding sites present on the antigen-binding molecule/protein.

The term “isolated,” as used herein, refers to material that is removed from its original or native environment (e.g. the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature. For example, a nucleic acid, protein/polypeptide molecule is considered to be “(in) essentially isolated (form)”—when compared to its native biological source and/or the reaction medium or cultivation medium from which it has been obtained—when it has been separated from at least one other component with which it is usually associated in said source or medium, such as another nucleic acid, another protein/polypeptide, another biological component or macromolecule or at least one contaminant, impurity or minor component. In particular, a nucleic acid or protein/polypeptide molecule is considered “essentially isolated” when it has been purified at least 2-fold, in particular at least 10-fold, more in particular at least 100-fold, and up to 1000-fold or more. A nucleic acid or protein/polypeptide molecule that is “in essentially isolated form” is preferably essentially homogeneous, as determined using a suitable technique, such as a suitable chromatographical technique, e.g., polyacrylamide-gel electrophoresis.

The term “covalently linked” as used herein means either a direct covalent bond between residues, or an indirect association where two residues are not directly bonded but are both covalently bonded to an intermediate molecule or domain, e.g. an intermediate domain of an immunoglobulin.

It is to be inferred without explicit recitation and unless otherwise intended, that when the present technology relates to a polypeptide, protein, polynucleotide or antibody, an equivalent or a biologically equivalent of such is intended within the scope of the present technology.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in an eukaryotic cell. The expression level of a gene may be determined by measuring the amount of mRNA or protein in a cell or tissue sample.

As used herein the term “biological sample” means a sample material derived from or contacted by living cells. The term is intended to include tissues, cells and biological fluids isolated from a subject. As used herein, the term “tissue sample” shall refer to a cellular sample that preserves the cross-sectional spatial relationship between the cells as they existed within the subject from which the sample was obtained. Biological samples can also be obtained from biopsies of internal organs or from cancers.

“Histochemistry” and cytochemistry” are techniques used to identify a molecule within the context of intact cells by labeling the samples with an agent that binds specifically to the molecule in a manner than can be visualized on a microscope. “Immunohistochemistry” and “immunocytochemistry” are types of histochemistry and cytochemistry that use antibodies to label the molecules.

“DLL3” as used herein refers to human. Delta-like ligand 3 (DLL3), also known as delta-like protein 3, which is a developmental protein which plays a key role in somitogenesis during embryonic development (Geffers et al., J Cell Biol; 2007; 178(3); 465-476). The protein sequence of human DLL3 can be retrieved, for example, from the UniProt database (Uniprot: Q9NYJ7). DLL3 is a member of the Notch signaling pathway, more specifically a Notch ligand.

“CD3” as used herein refers to human CD3 (cluster of differentiation 3). CD3 is a composed of four polypeptide chains, i.e., two CD3 epsilon chains (UniProt: P07766), a CD3 gamma chain (Uniprot: P09693) and a CD3 delta chain (UniProt: P04234). Since T-cells express CD3 on their surface, it is the specificity of proteins of the invention for this antigen which gives rise to the designation of said molecules as “T-cell engagers” or “T-cell engaging protein”. Preferably, TCEs bind to CD3 epsilon.

As used herein, the term “about” may encompass deviations of ±10%, optionally ±5% of the numerical value denoted with this term.

The terms “patient” and “subject” are used interchangeably herein. All subjects, patients or similar denotations refer to a subject, especially a human being, who receives an administration of the T-cell engaging protein and is in need of a treatment with that substance.

As used herein a “a method of treatment of a cancer selected from Neuroendocrine Neoplasms (NEN), glioma, glioblastoma, medullary thyroidal carcinoma, and Merkel cell carcinoma” means that a medical indication for administering the T-cell engaging molecule is present in these indications.

As used herein, the term “week” is defined as a time period encompassing 7 days. However, if the last day of the week is a holiday, the end of the 7 days period may be the day before or after the holiday or even after said holiday. Therefore, the term “week” in general encompasses 7 days but may be altered within the criteria mentioned.

The term “cycle” as used herein refers to one or more than one such as three administrations. A cycle may optionally be repeated. Conceptually, this disclosure distinguishes between a “step-in cycle” and a “treatment cycle”. The step-in cycle, as e.g., subject of the first aspect and other aspects of this invention, involves at least three administrations. Apart from exceptional cases discussed herein, said step-in cycle is not repeated. A treatment cycle on the other hand may be, and preferably is, repeated. Repetitions are preferably performed to secure tumor control or eradication. Preferred numbers of repetitions of treatments cycles are disclosed herein. As regards the number of doses/administrations to be given within a single treatment cycle, preference is given to three for first treatment cycles, and to one for second treatment cycles.

An administration on a certain day of a step-in or a treatment cycle, such as “day 1”, of a three weeks' time period may also admit a tolerance of two, or optionally one day, before or after that day, because a patient may not be able to appear at the exact appointment for various reasons. In addition, depending on the nomenclature, “day 1” of a step-in or treatment cycle can also be denoted as “day 0” and all following days are adapted accordingly.

The term “directly follows” as used to define the succession of cycles (first treatment cycle following step-in cycle, second treatment cycle following first treatment cycle) means that there is no interruption between cycles. To explain further, where a last dose within a given cycle is to be administered in a given week, the first dose of the respective subsequent cycle is to be given in the week directly following said given week. A preferred time to elapse is a week or seven days. To give an example: the 1st administration (cycle1 day1) occurs on a Tuesday; the 2nd administration (cycle1 day8) will occur on Tuesday the following week; the 3rd administration (cycle1 day15) will occur on Tuesday one week after; and the next administration (cycle2 day1) will occur on Tuesday one week (7 days) after the 3rd administration in cycle1. In this example, “cycle1” may refer to the step-in cycle, and “cycle2” may refer to the first treatment cycle; or “cycle1” may refer to the first treatment cycle, and “cycle2” may refer to the second treatment cycle.

As used herein, a “regimen” is a totality of all cycles, i.e., step-in cycle and first and following treatment cycles, when treating a patient.

As used herein, an “administration” of the T-cell engaging protein is usually carried out within one treatment day and not over several days. A preferred administration route is intravenous (i.v.). A preferred duration of an i.v. administration is between about 0.5 and about 5 hours, more preferably between about 1 and about 3 hours such as about 2 hours.

A cytokine release syndrome (CRS) may be classified by a Common Toxicity Criteria (NCI CTC) grade throughout this disclosure. A preferred grading scheme for both CRS and ICANS is the ASTCT consensus grading as described in Lee et al., Biol Blood Marrow Transplant 25, 625-638 (2019). Response is evaluated according to Response Evaluation Criteria In Solid Tumours (RECIST, version 1.1; Eisenhauer E A et al., New response evaluation criteria in solid tumours: revised RECIST guideline (version 1.1). Eur J Cancer 45, 228-247 (2009).

As used herein, objective response (OR) is defined as the best overall response of confirmed complete response (CR) or confirmed partial response (PR) according to RECIST v1.1 by investigator assessment from the date of treatment start until the earliest date of disease progression, death, or last evaluable tumor assessment before start of subsequent anticancer therapy, loss to follow-up, or withdrawal of consent.

For clarification, whenever “carboplatin or cisplatin and etoposide” is mentioned, the alternative of both combinations, i.e. carboplatin and etoposide or cisplatin and etoposide” is meant.

The terms BI 764532 and obrixtamig to denote the T-cell engaging protein will be used interchangeably herein.

As used herein, the abbreviation “i.v.” or (IV) denotes an intravenous administration.

As used herein, the abbreviation “p.o.” denotes an oral administration.

As used herein, the term “equivalent” in the context with a drug denotes medications that are considered interchangeable because they meet specific criteria for similarity in composition and therapeutic effect. While the active ingredient might be different “or equivalent” refers to therapeutic equivalence, i.e. drugs that are pharmaceutically equivalent and are expected to have the similar clinical effect and safety profile when administered under the same conditions. For example, a steroid, such as dexamethasone, may have an equivalent.

As used herein, a “premedication”, which is specified hereinbelow, typically refers to a medicament administered directly prior to a respective administration of the T-cell engaging protein as defined herein for safety reasons, e.g. for reducing CRS. In this context, administration “directly prior” usually means that the premedication is administered about 0.25 to about 2 hours, such as about 0.5 hours to about 1 hour, prior to the respective point in time of the start of the administration of a respective dose of the T-cell engaging protein. Unless indicated otherwise, “directly prior” and “prior” are used interchangeably in this context of administration of the premedication.

As used herein, a “post-medication”, which is specified hereinbelow, typically refers to a medicament administered directly after a respective administration of the T-cell engaging protein as defined herein for safety reasons, e.g. for reducing CRS. In this context, “directly after” usually means that the post-medication is administered at least 6 hours up to about 12 hours, such as 8±2 hours, following point in time of the start of the administration of a respective dose of the T-cell engaging protein. In particular, whenever the administration of the post-medication is disclosed herein, the point in time for starting the post-medication, such as 8±2 hours after, is typically calculated from the point in time of the start of the administration of the respective dose of the T-cell engaging protein. Unless indicated otherwise “directly after” and “after” are used interchangeably in this context of administration of the post-medication.

The terms “step-up dosing” and “step-in dosing” dosing are used interchangeably herein, unless indicated otherwise.

Whenever used herein for describing trial results, the abbreviations have the following meanings: ORR=objective response rates, DC=disease control, DoR=duration of response, SD=stable disease.

### Embodiments

Of note, this disclosure makes use of short-hand identifiers such as B2F, B2, B3F and B3 for preferred dosage regimens. The actual doses to be administered and intervals to be observed are defined in the general description and the claims and exemplified in the Examples.

For the avoidance of doubt, the disclosure of the first aspect is combinable which each of the following preferred embodiments of the following sub-sections of the detailed description.

The first aspect of the present invention is directed to a T-cell engaging protein comprising a first antigen binding unit specifically binding to DLL3 and a second antigen binding unit specifically binding to CD3 for use in a method for treating a DLL3-positive cancer or a cancer selected from a Neuroendocrine Neo-plasm (NEN), Merkel cell carcinoma (MCC), medullary thyroidal carcinoma and glioma in a subject exhibiting at least a threshold of DLL3-positivity, the method comprising administering to the subject a dose of said T-cell engaging protein, wherein
said T-cell engaging protein comprises a first antigen binding unit specifically binding to DLL3, comprising light chain CDRs comprising the amino acid sequences of SEQ ID NO:13 (CDR1), SEQ ID NO:14 (CDR2) and SEQ ID NO:15 (CDR3) and heavy chain CDRs comprising the amino acid sequences of SEQ ID NO:16 (CDR1), SEQ ID NO:17 (CDR2) and SEQ ID NO:18 (CDR3) and a second antigen binding unit specifically binding to CD3 comprising light chain CDRs comprising the amino acid sequences of SEQ ID NO:55 (CDR1), SEQ ID NO:56 (CDR2) and SEQ ID NO:57 (CDR3) and heavy chain CDRs comprising the amino acid sequences of SEQ ID NO:58 (CDR1), SEQ ID NO:59 (CDR2) and SEQ ID NO:60 (CDR3).

In particular, said threshold may be >0%. Alternatively, the threshold may be ≥1%.

In certain embodiments, the subject has been determined to exhibit at least a threshold of DLL3-positivity.

In certain embodiments, the method is comprising the step of determining or having determined that the subject exhibits at least a threshold of DLL3-positivity.

In other words, the determination of the threshold does not necessarily be conducted by the same person, laboratory, institute or entity carrying out the administration.

In certain embodiments, the threshold of DLL3-positivity is at least about 10%, at least about 20% at least 25%, at least about 30%, at least about 40%, at least about 50% or at least about 75%. In preferred embodiments, the threshold is at least about 25%, at least about 50%, or at least about 75%. In specific embodiment if the T-cell engaging protein is the only therapeutic agent within the method of treatment, the threshold may be at least 50%. However, in case of a co-administration with another cancer therapeutic agent, such as carboplatin/cisplatin+etoposide±an anti-PD-L1 antibody, topotecan, lurbinectedin, or amrubicin, the threshold may for example be at least about 10% or at least about 20% or at least about 25%.

Most preferably, the DLL3-positivity threshold may be at least about 50% or about 50%.

Alternatively, the DLL3-positivity threshold may be at least about 25% or about 25%.

In certain embodiments, the DLL3 positivity in a sample is calculated according to the following formula:

$$DLL 3 - positivity [ \% ] = \frac{DLL 3 - positive tumor cells in the sample}{\begin{matrix}( DLL 3 - positive tumors cells in the sample ) + \\ ( DLL 3 - negative tumor cells in the sample )\end{matrix}} .$$

Further details about the DLL3-positivity will be provided herein below.

In certain embodiments, said Neuroendocrine Neoplasm (NEN) is a Neuroendocrine Carcinoma (NEC), preferably selected from the group consisting of Lung-NEC such as LCNEC of the lung, Extrapulmonary (ep) NEC including NEC with unknown primary site; small cell lung cancer (SCLC) including extensive-stage (ES)-SCLC, and limited-stage (LS) SCLC; or said NEN is a Neuroendocrine Tumor (NET) such as grade 3 NET or, in the alternative, epNEC excluding Neuroendocrine prostate cancer (NEPC).

In preferred embodiments, the threshold is at least about 50%, more preferably about 50%, and the cancer is epNEC or epNEC excluding NEPC.

In other preferred embodiments, the threshold is at least about 25%, more preferably about 25%.

In further preferred embodiments, the threshold is at least about 75%, more preferably about 75%.

In certain embodiments, at least one further anti-cancer agent is to be administered, said further anti-cancer agent preferably being a chemotherapeutic agent and/or an immune-checkpoint inhibitor such as an anti-PD-1 antibody or an anti-PD-L1 antibody.

### Method of Selecting a Subject

In a second aspect, the present invention provides method for selecting a subject suffering from a DLL3-positive cancer or a cancer selected from a Neuroendocrine Neoplasm (NEN), Merkel cell carcinoma (MCC), medullary thyroidal carcinoma and glioma for treatment with a T-cell engaging protein comprising a first antigen binding unit specifically binding to DLL3 and a second antigen binding unit specifically binding to CD3, the method comprising:
(a) determining or having determined in a sample that the subject exhibits DLL3 positivity, wherein the subject is selected for treatment with said T-cell engaging protein when the DLL3-positivity exhibits at least a threshold, and
   wherein said T-cell engaging protein comprises a first antigen binding unit specifically binding to DLL3, comprising light chain CDRs comprising the amino acid sequences of SEQ ID NO:13 (CDR1), SEQ ID NO:14 (CDR2) and SEQ ID NO:15 (CDR3) and heavy chain CDRs comprising the amino acid sequences of SEQ ID NO:16 (CDR1), SEQ ID NO:17 (CDR2) and SEQ ID NO:18 (CDR3) and a second antigen binding unit specifically binding to CD3 comprising light chain CDRs comprising the amino acid sequences of SEQ ID NO:55 (CDR1), SEQ ID NO:56 (CDR2) and SEQ ID NO:57 (CDR3) and heavy chain CDRs comprising the amino acid sequences of SEQ ID NO:58 (CDR1), SEQ ID NO:59 (CDR2) and SEQ ID NO:60 (CDR3).

Determining in this context means that the determination step is actively carried out. However, “having determined” means that the step may be carried out somewhere else, e.g. by another entity or another company.

Furthermore, determining or having determined the DLL3-positivity can also mean that a method which has a different measurement protocol, such as those disclosed in the section “DLL3-positivity”, from which a DLL3-positivity can be derived, either qualitatively or quantitatively may be measured. Preferably, the measurement protocol, based on which DLL-3 positivity is measured, is carried out quantitatively.

All embodiments, definitions and explanations of the section “DLL3-positivity” equally apply here and will not be repeated for the sake of conciseness.

In certain embodiments, DLL3-positivity is calculated according to the following formula:

$$DLL 3 - positivity [ \% ] = \frac{DLL 3 - positive tumor cells in the sample}{\begin{matrix}( DLL 3 - positive tumors cells in the sample ) + \\ ( DLL 3 - negative tumor cells in the sample )\end{matrix}} .$$

In certain embodiments, the threshold is at least about 10%, at least about 20% at least 25%, at about least 30%, at least about 40%, at least about 50% or at least about 75%.

In preferred embodiments, the threshold is at least about 25%, at least about 50%, or at least about 75%. In more preferred embodiments, the threshold is at least about 50%. In other preferred embodiments, the threshold is at least about 25%.

In certain embodiments, the number of DLL3-positive tumor cells in the sample is determined by counting immunostained tumor cells in the sample.

In particular, the method of selecting a subject may be performed ex vivo or in vitro using a sample originating from the subject.

### DLL3-Positivity

Provided herein are methods of treating a DLL3-positive cancer in a subject and methods for selecting a subject suffering from a DLL3-positive cancer for treatment. Preferably, the DLL3-positive cancer expresses DLL3 on the cell surface. All DLL3-positive cancers or cancers described in combination with the aspects of the present invention may be considered for determining expression in terms of DLL3-positivity as described herein. In some embodiments, the DLL3-positive cancer expresses DLL3 on the cell surface and in the cytoplasm. For any of the methods described herein, DLL3 expression may be determined by any suitable method in the art of which some are disclosed hereinbelow.

Preferably, DLL3 protein expression—at the cell surface and/or in the cytoplasm—is determined by immunohistochemistry (IHC). Any IHC assay kit suitable to detect DLL3 expression in tumor cells can be used as a basis for a determination of DLL3 positivity in a tissue sample. In preferred embodiments, the IHC assay is authorized or approved by a regulatory agency. Typically an anti DLL3 antibody intended for detection of DLL3 in human samples may be part of such assay kit. In particular, an anti-DLL3-specific antibody kit (e.g. VENTANA® DLL3 (SP347) Assay, Roche Diagnostics) intended for detection of DLL3 in sections of human formalin-fixed and paraffin-embedded (FFPE) tissues by immunohistochemistry (IHC) may be used.

In certain embodiments, DLL3 positivity is calculated based on the following formula:

$$DLL 3 positivity [ \% ] = \frac{DLL 3 positive tumor cells}{( DLL 3 positive tumors cells ) + ( DLL 3 negative tumor cells )}$$

In some embodiments, the threshold for DLL3 positivity is at least any one of about 10%, about 20%, about 25%, about 30%, about 40%, about 50%, about 60% or about 75%.

In preferred embodiments, DLL3 positive and negative tumor cells are determined using an IHC assay (e.g., VENTANA® DLL3 (SP347), Assay described above and in example 1 below). In some embodiments, DLL3-specific tumor cell staining is expressed as the percent of all tumor cells showing membranous and/or cytoplasmic staining at moderate to strong intensity in evaluable tumor cells. Moderate and strong intensity are well established, known and characterized terms for the skilled person in the field of pathology.

In particular, strong membranous and/or cytoplasmic staining of tumor cells is scored as “3” or “3+,”, moderate membranous and/or cytoplasmic staining as “2” or “2+,” and weak membranous and/or cytoplasmic staining as “1” or “1+.” Absence of staining was given a score of “0” based on the methodology disclosed in Huang et al., Arch Pathol Lab Med, 143 (11): 1373-137 (2019). In some embodiments, “DLL3 positivity” of the tumor samples is defined as greater than 0% DLL3-positive tumor cells at moderate to strong intensity. Determination of DLL3 positivity is based only on tissue sections that are considered evaluable by a qualified pathologist, uses at least 100 evaluable tumor cells and any magnification may be used. In some embodiments, DLL3 positivity is compared to a control tissue or cell pellet with a known DLL3 expression, which is analyzed using the same IHC assay.

DLL3 positivity may also be determined by calculating an H-score using the following formula: H-score=1×percentage of cells staining at weak intensity+2×percentage of cells staining at moderate intensity+3×percentage of cells staining at strong intensity.

In some embodiments, methods known in the art based on quantifying DLL3 positive tumor cells in cancer samples such as SCLC or epNEC (e.g. mRNA levels) may be used for determining DLL3-positivity. Preferably such detection method may encompass flow cytometry-based methods, polymerase chain reaction-based methods, in-situ hybridisation-based methods (such as RNAscope), RNA sequencing analysis, Northern blot analysis, reverse-transcriptase polymerase chain reaction (RT-PCR analysis), or any other methods known to the skilled person.

### Dosing Regimen B2F

In a third aspect, the present invention provides a T-cell engaging protein comprising a first antigen binding unit that specifically binds to DLL3 and a second antigen binding unit that specifically binds to CD3 for use in a method of treatment of a DLL3-positive cancer or a cancer selected from a Neuroendocrine Neoplasm (NEN), Merkel cell carcinoma (MCC), medullary thyroidal carcinoma and glioma,
the method comprising a step-in cycle, said step-in cycle comprising the steps of:
   administering to a subject an initial dose of said T-cell engaging protein, said initial dose ranging from about 20 μg/kg of the subject's body weight to about 40 μg/kg of the subject's body weight,
   administering to the subject an elevated dose, which is elevated as compared to said initial dose, of said T-cell engaging protein, the elevated dose ranging from about 60 μg/kg of the subject's body weight to about 100 μg/kg of the subject's body weight,
   administering to the subject a further dose of the T-cell engaging protein, said further dose ranging from about 5 mg to about 70 mg.

In certain embodiments, the T-cell engaging protein comprising a first antigen binding unit specifically binding to DLL3 and a second antigen binding unit specifically binding to CD3 is intended for use in a method for treating a DLL3-positive cancer or a cancer selected from a Neuroendocrine Neo-plasm (NEN), Merkel cell carcinoma (MCC), medullary thyroidal carcinoma and glioma in a subject exhibiting at least a threshold of DLL3-positivity, the method comprising administering to the subject a dose of said T-cell engaging protein, wherein
said T-cell engaging protein comprises a first antigen binding unit specifically binding to DLL3, comprising light chain CDRs comprising the amino acid sequences of SEQ ID NO:13 (CDR1), SEQ ID NO:14 (CDR2) and SEQ ID NO:15 (CDR3) and heavy chain CDRs comprising the amino acid sequences of SEQ ID NO:16 (CDR1), SEQ ID NO:17 (CDR2) and SEQ ID NO:18 (CDR3) and a second antigen binding unit specifically binding to CD3 comprising light chain CDRs comprising the amino acid sequences of SEQ ID NO:55 (CDR1), SEQ ID NO:56 (CDR2) and SEQ ID NO:57 (CDR3) and heavy chain CDRs comprising the amino acid sequences of SEQ ID NO:58 (CDR1), SEQ ID NO:59 (CDR2) and SEQ ID NO:60 (CDR3), the method comprising a step-in cycle, said step-in cycle comprising the steps of:
   administering to a subject an initial dose of said T-cell engaging protein, said initial dose ranging from about 20 μg/kg of the subject's body weight to about 40 μg/kg of the subject's body weight,
   administering to the subject an elevated dose, which is elevated as compared to said initial dose, of said T-cell engaging protein, the elevated dose ranging from about 60 μg/kg of the subject's body weight to about 100 μg/kg of the subject's body weight,
   administering to the subject a further dose of the T-cell engaging protein, said further dose ranging from about 5 mg to about 70 mg.

The third aspect of the present invention may also be an embodiment of the first aspect of the present invention as defined in the section “Brief Summary of the Invention” or in the section “Embodiments”

Of note, said further dose to be administered within said step-in cycle may already be the dose to be administered within the preferably ensuing treatment cycle. There is no requirement in that respect, though, in case the clinical symptoms of the patient permit such administration, the final dose, i.e. further dose, to be administered within said step-in cycle may be viewed as a first treatment dose—although formally subsumed, in adherence to the nomenclature used herein, under the term “step-in cycle”.

A preferred initial dose is between about 25 and about 35 μg/kg such as about 30 μg/kg.

A preferred elevated dose is between about 80 and 100 μg/kg such as about 90 μg/kg. In that respect it is of note that beneficial effects in terms of treatment may already occur in response to said elevated dose, noting that about 10 mg are considered as the minimal effective dose in terms of treatment.

In other preferred embodiments, said further dose is about 10 mg, about 30 mg and about 60 mg. To the extent these values are lower than preferred target doses to be administered in the first treatment cycle as specified below, these lower values may be applied dependent on the clinical symptoms of the patient and/or serve to avoid the onset of severe side effects such as CRS and ICANS, severity preferably being defined in terms of the respective scoring schemes as defined further below.

In a more preferred embodiment, the initial dose is about 30 μg/kg, the elevated dose is about 90 μg/kg of the subject's bodyweight and the further dose is about 10 mg.

In an alternative more preferred embodiment, the initial dose is about 30 μg/kg, the elevated dose is about 90 μg/kg of the subject's bodyweight and the further dose is about 30 mg.

In an alternative more preferred embodiment, the initial dose is about 30 μg/kg, the elevated dose is about 90 μg/kg of the subject's bodyweight and the further dose is about 60 mg.

As such, said further dose is preferably between about 10 and about 60 mg. In particular, the further dose may be about 10 mg, about 30 mg or about 60 mg.

In those cases where treatment tolerability is good, said initial dose is given once, said elevated dose is also given once, and said further dose, also to be given once, is a preferred target dose as defined in the context of the first treatment cycle.

Accordingly, in further embodiments, said initial dose, said elevated dose and/or said further dose is/are administered once; or
said initial dose, said elevated dose and/or said further dose is/are administered more than once, optionally 2 to 10 times,
wherein optionally the number of administrations is determined based on presence and/or degree of a cytokine release syndrome (CRS) and/or of an immune effector cell-associated neurotoxicity syndrome (ICANS),
and wherein further optionally said elevated dose follows the initial dose only if said CRS, to the extent determined, is rated grade 1 or less in terms of NCI CTC grade and/or if said ICANS, to the extend determined, is rated grade 1 or less, and/or wherein further optionally said further dose follows the elevated dose only if said CRS, to the extent determined, is rated grade 1 or less in terms of NCI CTC grade and/or if said ICANS, to the extend determined, is rated grade 1 or less. In other words, said initial dose may be administered more than once, optionally 2 to 10 times, in view of the above NCI CTC or ICANS criteria. This means that the initial dose may be repeated before an elevated dose can be administered. Preferably, such repetition is done once in a week. The administrations and repetitions may be spaced apart a week. More preferably, said initial dose may be repeated 2, 3, 4, 5, 6, 7, 8, 9 or 10 times. In addition or alternatively, the same also applies for the elevated dose before the further dose is administered within the step-in cycle. Yet further, also said further dose may be repeated, e.g., 2, 3, 4, 5, 6, 7, 8, 9 or 10 times where deemed necessary in view of adverse responses.

Moreover, in case adverse responses occur, alternatively or in addition to repeating a dose (initial dose or elevated dose), a given dose may be reduced as compared to the preceding initial or elevated dose, e.g., to about 10% to about 90% of the preceding dose such about ⅔ or about ⅓, preferably about 50% of the preceding dose. Of note, the option of dose reduction does not only apply to the step-in cycle, but also to any of the treatment cycles disclosed herein.

In preferred embodiments, said initial dose is administered three times to ten times within the step-in cycle, preferably 3, 4, 5, 6, 7, 8, 9 or 10 times. Alternatively or in addition, the same also applies to the elevated dose.

In preferred embodiments, the administration of the doses may be spaced one week apart.

In preferred embodiments, said initial dose is administered three times within three weeks, optionally on day 1, day 8 and day 15 of the step-in cycle, if it is administered for three times. For example, the elevated dose may then be administered on day 21 of the step-in cycle, if the above NCI CTC and/or ICANS criteria are fulfilled. If the initial dose is administered 2 times, it may be administered on day 1 and day 8 in the step-in cycle, wherein the elevated dose follows in day 15. The same considerations apply to the elevated dose. If, after administration of the elevated dose, the NCI CTC and/or ICANS criteria are fulfilled, the further dose is administered next.

In some embodiments, said step-in cycle has a duration of at least three weeks. In preferred embodiments, said step-in cycle may have a duration of three weeks, wherein optionally the initial dose may be administered on day 1, the elevated dose may be administered on day 8 and the further dose may be administered on day 15. Depending on how many times the initial and/or the first dose may be administered, the step-in cycle may have a longer duration than three weeks.

In some embodiments, said initial, elevated and further doses are administered within three weeks, said initial dose being administered once, wherein optionally said initial dose is administered on day 1, said elevated dose is administered on day 8 and said further dose is administered on day 15 of said step-in cycle.

To the extent reference is made to specific days such as day 1, day 8, day 15 etc. it is to be understood that these are particularly preferred days which, however, allow for certain deviations depending on the patient's compliance or availability as well as the attending clinician's capacities. In other words, administrations on, say, days 1, 9 and 14 are within the scope of the present invention. Generally speaking, it will be sufficient to administer once per week regardless of the specific day within a given week. More preferably, deviations from the most preferred scheme (days 1, 8, 15 etc.) should not exceed three, two or one days in either direction.

The above considerations relating to the intervals between subsequent administrations apply generally, i.e., not only to the step-in cycle but also to treatment cycles as disclosed further below.

In certain embodiments, the method further comprises a first treatment cycle following said step-in cycle, said first treatment cycle comprising the steps of:
administering to a subject a first target dose of the T-cell engaging protein, said first target dose ranging from about 5 mg to about 70 mg,
administering to the subject a second dose of the T-cell engaging protein, said second dose ranging from about 5 mg to about 70 mg, and
administering to the subject a third dose of the T-cell engaging protein, said third dose ranging from about 5 mg to about 70 mg,
   wherein optionally the amount of all three target doses is identical.

In preferred embodiments, the amount of all three target doses is identical and the amount ranges from about 5 mg to about 10 mg, preferably being about 10 mg, for the first, second and third target dose, respectively.

In other preferred embodiments, the amount of all target doses is identical, and the amount is about 60 mg for the first, second and third target dose, respectively.

In further preferred embodiments, the amount of all target doses is identical, and the amount is about 30 mg for the first, second and third target dose, respectively.

In preferred embodiments, the amount of all three target doses are identical and about 10 mg. In case of safety concerns, said amount may be further reduced and be in the range of about 5 mg to about 10 mg, and is in particular about 5 mg.

In alternative preferred embodiments, the amount of all three target doses are identical and about 60 mg. In case of safety concerns, said amount may be further reduced and be in the range of about 20 mg to about 60 mg, and is in particular about 30 mg.

Of note, the dependence of a given dose on the patient's body weight during the step-in cycle confers distinct advantages. In particular, such dosing provides improved tolerability including reduced frequency and/or severity of CRS, and furthermore allows for higher target doses in the course of treatment cycles. Without wishing to be bound by a specific theory, it is considered that these advantageous effects results from a controlled cytokine depletion during the step-in cycle.

In preferred embodiments, said first treatment cycle is not repeated or said first treatment cycle is repeated at least once. Preferred numbers of repetitions are disclosed herein and exemplified in the Examples.

In preferred embodiments, said first, second and third target doses are administered within three weeks of said first treatment cycle, wherein optionally said first target dose is administered on day 1, the second target dose is administered on day 8 and the third target dose is administered on day 15 of said first treatment cycle, wherein optionally said first treatment cycle has a duration of three weeks. Reference is made to a certain flexibility regarding intervals or specific treatment days as explained further above in the context of the description of the step-in cycle.

In preferred embodiments, said the first treatment cycle is followed by a second treatment cycle, said second treatment cycle comprising the step of or consisting of the step:
administering to the subject a target dose of the T-cell engaging protein, said target dose ranging from about 5 mg to about 70 mg;
   wherein said second treatment cycle is optionally repeated at least once. Preferably, said target dose is about 10 mg, about 30 mg or about 60 mg.

In preferred embodiments, said second treatment cycle has a total duration of three weeks and/or said target dose is administered on day 1 of said three weeks. Reference is made to optional or preferred repetitions of treatments cycle as disclosed herein.

In other words, if a patient is considered to be in need of further treatment after the first treatment cycle, the burden on the patient can be reduced by not requiring any more that the patient presents itself once a week in the clinic, but in larger intervals, a preferred larger interval including, but not being confined to three weeks as is subject of the preferred embodiment above. Alternatively, also intervals of about two weeks or every ten or every 20 days may be considered.

In preferred embodiments, said first treatment cycle directly follows said step-in cycle; and/or said second treatment cycle directly follows said first treatment cycle. To explain further, a dosage regimen comprising step-in cycle as well as first and second treatment cycle may involve administrations on days 1, 8, 15, 22, 29, 36, 43, 64, 85 etc., wherein days 1, 8 and 15 would correspond to the step-in cycle (step-in doses being tolerable in this example such that no repetitions are required), days 22, 29, 36 (counting continued from the day of administration of the first dose of the step-in cycle) would correspond to one application of the first treatment cycle (weekly target dose), and days 43, 64, 85 etc. would correspond to repeated (here three) applications of the second treatment cycle (3 week administration intervals in this example).

In some embodiments, essentially no cytokine release syndrome occurs after said elevated dose of said step-in cycle in the subject. In particular, if no CRS is observed after the step in dose, the hospitalization is only about 6 hours, especially instead of 24 hours. Preferably, a dexamethasone premedication may be about 8 mg, especially instead of about 16 mg.

This would generally be scenario where the patient under consideration would qualify for said further dose of said step-in cycle being the same as a preferred target dose of said first treatment cycle. Optionally, premedication may be adjusted under such circumstances as disclosed herein.

In some embodiments, said T-cell engaging protein is the only anti-cancer agent to be administered.

In some embodiments, at least one further anti-cancer agent is to be administered, said further anti-cancer agent preferably being a chemotherapeutic agent. Preferred chemotherapeutic agents are those disclosed in therein below, specifically in the section entitled “Co-administration” as well as those referred to in the Examples.

In certain embodiments, the subject has been treated with a chemotherapy (e.g. one or two cycles) involving administration of inhibitors of cell growth and/or division prior to the step-in cycle.

In certain embodiments, an occurrence of cytokine release syndrome is to be treated with an anti-cytokine release syndrome agent, preferably selected from the group consisting of a corticosteroid, an IL-6 antagonist and an IL-6R antagonist.

Overall, preference is given to treatment cycles of 3 weeks duration.

In specific embodiments, said initial dose is about 30 μg/kg;
said elevated dose is about 90 μg/kg; and
said further dose is about 10 mg.

In specific embodiments, said initial dose is about 30 μg/kg;
said elevated dose is about 90 μg/kg; and
said further dose is about 30 mg.

In specific embodiments, said initial dose is about 30 μg/kg;
said elevated dose is about 90 μg/kg; and
said further dose is about 60 mg.

In particular, dosing scheme B2F, i.e. the third aspect of the present invention, or any dosing scheme falling within the first or second aspect of the present invention can be co-administered, e.g. with the following cancer treatment agents.

In certain embodiments, the method further comprises a step of co-administering topotecan to the subject, preferably for use in the treatment of SCLC (any treatment line), including ES-SCLC, preferably second line (2L) treatment of ES-SCLC that progressed or recurred following Standard of Care (SoC) treatment and LS-SCLC, preferably first line (1L) treatment.

In certain embodiments, the method further comprises a step of co-administering lurbinectedin to the subject, preferably for use in the treatment of SCLC (any treatment line) including ES-SCLC, preferably 2L treatment of ES-SCLC that progressed or recurred following SoC, and LS-SCLC, preferably 1L treatment.

In certain embodiment, the method further comprises a step of co-administering amrubicin to the subject, preferably for use in the treatment of SCLC (any treatment line) including ES-SCLC, preferably 2L treatment of ES-SCLC that progressed or recurred following SoC, and LS-SCLC, preferably 1L treatment.

In certain embodiments, the method further comprises a step of co-administering etoposide and carboplatin or cisplatin, and optionally also anti-PD-L1 antibody e.g. atezolizumab or durvalumab, to the subject, preferably for use in the treatment of SCLC including ES-SCLC, especially for 1L, 2L and/or 3L treatment, and LS-SCLC (any treatment line).

In certain embodiments, the method further comprises a step of co-administering etoposide and carboplatin or cisplatin, to the subject, preferably for use in the treatment of epNEC or LCNEC of the lung (any treatment line), preferably first line treatment of epNEC or LCNEC of the lung.

In some embodiments, preferred is a regimen within a method of treatment as illustrated in Table 1A below.

TABLE 1A
Embodiment of an aspect of the present invention.
Regimen B2F:
One cycle duration = 3 weeks
Step-in cycle as defined below
One first treatment cycle: Target doses administered weekly
Second treatment cycles: Target doses administered every three weeks (q3w)
until progression or toxicity, preferably for a maximum of 36 months

| | | | | | | | Second |
|---|---|---|---|---|---|---|---|
| | | | | | | | treatment |
| | | | | First treatment cycles | | | cycle |
| | Step-in cycle | | | (optionally repeated) | | | (repeated) |
| | d 1 | d 8 | d 15 | d 1 | d 8 | d 15 | d 1 |
| dose | 30 | 90 | Target dose | Target | Target | Target | Target |
| | μg/kg | μg/kg | (same for all | dose: | dose: | dose: | dose: |
| | | | following | 10 mg, 30 | 10 mg, 30 | 10 mg, 30 | 10 mg. 30 |
| | | | treatment steps): | mg or 60 | mg, 60 mg | mg, 60 mg | mg, 60 mg |
| | | | 10 mg, 30 mg | mg | | | |
| | | | or 60 mg | | | | |

In particular, as can be seen from the data in the examples below, the step in dosing of Table 1A above, in particular with relation to d1 and d8 has been found out to be safe and, therefore, flat doses, such as 10 mg, 30 mg or 60 mg can be used in the next steps.

### Dosing Regimen B3F

In a fourth aspect, the present invention provides a T-cell engaging protein comprising a first antigen binding unit that specifically binds to DLL3 and a second antigen binding unit that specifically binds to CD3 for use in a method of treatment of a DLL3-positive cancer or a cancer selected from a Neuroendocrine Neoplasm (NEN), Merkel cell carcinoma (MCC), medullary thyroidal carcinoma and glioma,
the method comprising a step-in cycle, said step-in cycle comprising the steps of:
   administering to a subject an initial dose of said T-cell engaging protein, said initial dose ranging from about 5 μg/kg of the subject's body weight to about 15 μg/kg of the subject's body weight,
   administering to the subject an elevated dose, which is elevated as compared to said initial dose, of said T-cell engaging protein, the elevated dose ranging from about 20 μg/kg of the subject's body weight to about 40 μg/kg of the subject's body weight,
   administering to the subject a further dose of said T-cell engaging protein, said further dose ranging from about 80 μg/kg of the subject's body weight to about 1600 μg/kg of the subject's bodyweight; or the method comprising a step-in cycle, the step-in cycle comprising the steps of:
   administering to the subject an initial dose, an elevated dose, which is elevated as compared to said initial dose, and a further dose of said T-cell engaging protein, wherein a ratio between said initial, elevated and further dose per kilogram of the subject's body weight is about 1:about 3:about 9.

In certain embodiments, the T-cell engaging protein comprising a first antigen binding unit specifically binding to DLL3 and a second antigen binding unit specifically binding to CD3 is intended for use in a method for treating a DLL3-positive cancer or a cancer selected from a Neuroendocrine Neo-plasm (NEN), Merkel cell carcinoma (MCC), medullary thyroidal carcinoma and glioma in a subject exhibiting at least a threshold of DLL3-positivity, the method comprising administering to the subject a dose of said T-cell engaging protein, wherein
said T-cell engaging protein comprises a first antigen binding unit specifically binding to DLL3, comprising light chain CDRs comprising the amino acid sequences of SEQ ID NO:13 (CDR1), SEQ ID NO:14 (CDR2) and SEQ ID NO:15 (CDR3) and heavy chain CDRs comprising the amino acid sequences of SEQ ID NO:16 (CDR1), SEQ ID NO:17 (CDR2) and SEQ ID NO:18 (CDR3) and a second antigen binding unit specifically binding to CD3 comprising light chain CDRs comprising the amino acid sequences of SEQ ID NO:55 (CDR1), SEQ ID NO:56 (CDR2) and SEQ ID NO:57 (CDR3) and heavy chain CDRs comprising the amino acid sequences of SEQ ID NO:58 (CDR1), SEQ ID NO:59 (CDR2) and SEQ ID NO:60 (CDR3), the method comprising a step-in cycle, said step-in cycle comprising the steps of:
administering to a subject an initial dose of said T-cell engaging protein, said initial dose ranging from about 5 μg/kg of the subject's body weight to about 15 μg/kg of the subject's body weight,
   administering to the subject an elevated dose, which is elevated as compared to said initial dose, of said T-cell engaging protein, the elevated dose ranging from about 20 μg/kg of the subject's body weight to about 40 μg/kg of the subject's body weight,
   administering to the subject a further dose of said T-cell engaging protein, said further dose ranging from about 80 μg/kg of the subject's body weight to about 1600 μg/kg of the subject's bodyweight; or the method comprising a step-in cycle, the step-in cycle comprising the steps of:
   administering to the subject an initial dose, an elevated dose, which is elevated as compared to said initial dose, and a further dose of said T-cell engaging protein, wherein a ratio between said initial, elevated and further dose per kilogram of the subject's body weight is about 1:about 3:about 9.

The fourth aspect of the present invention may also be an embodiment of the first aspect or second aspect of the present invention as defined in the section “Brief Summary of the Invention”.

In a preferred embodiment, said initial dose is about 10 μg/kg, said elevated dose is about 30 μg/kg, and said further dose is about 90 μg/kg.

Other than the step-in dosing, the preferred embodiments of the third aspect of the invention, set forth under sub-section “Dosing Regimen B2F” apply mutatis mutandis.

In particular, a duration of the step-in cycle of three weeks is preferred, in particular in those cases where there is good treatment tolerability. In that context, administration at days 1, 8 and 15 is preferred.

In certain embodiments, the present invention provides a T-cell engaging protein comprising a first antigen binding unit specifically binding to DLL3 and a second antigen binding unit specifically binding to CD3 for use in a method of treatment of a DLL3-positive cancer or a cancer selected from a Neuroendocrine Neoplasm (NEN), Merkel cell carcinoma (MCC), medullary thyroidal carcinoma and glioma,
the method comprising a step-in cycle, the step-in cycle comprising the steps of:
   administering to a subject an initial dose of said T-cell engaging protein, said initial dose ranging from about 5 μg/kg of the subject's body weight to about 15 μg/kg of the subject's body weight,
   administering to the subject an elevated dose, which is elevated as compared to said initial dose, of said T-cell engaging protein, the elevated dose ranging from about 20 μg/kg of the subject's body weight to about 40 μg/kg of the subject's body weight,
   administering to the subject a further dose of said T-cell engaging protein, said further dose ranging from about 80 μg/kg of the subject's body weight to about 1600 μg/kg of the subject's bodyweight.

In the following, preferred embodiments of the above aspects of the invention are given. Of note, additional preferred embodiments as well as explanation as given further above in the context of the first aspect of the invention and preferred embodiments thereof apply mutatis mutandis also to the second, third and any further aspects of the invention.

In some embodiments, said initial dose, said elevated dose and/or said further dose is/are administered once; or
said initial dose, said elevated dose and/or said further dose is/are administered more than once, optionally 2 to 10 times,
wherein optionally the number of administrations is determined based on presence and/or degree of a cytokine release syndrome (CRS) and/or of an immune effector cell-associated neurotoxicity syndrome (ICANS),
and wherein further optionally said elevated dose follows the initial dose only if said CRS, to the extent determined, is rated grade 1 or less in terms of NCI CTC grade and/or if said ICANS, to the extend determined, is rated grade 1 or less, and/or wherein
further optionally said further dose follows the elevated dose only if said CRS, to the extent determined, is rated grade 1 or less in terms of NCI CTC grade and/or if said ICANS, to the extend determined, is rated grade 1 or less.

In some embodiments, said initial dose, said elevated dose and/or said further dose is/are administered three times to ten times within the step-in cycle.

In some embodiments, said initial dose is administered three times within three weeks, optionally on day 1, day 8 and day 15 of the three weeks.

In some embodiments, said step-in cycle has a duration of at least three weeks.

In some embodiments, said initial, elevated and further doses are administered within three weeks, said initial dose being administered once, wherein optionally said initial dose is administered on day 1, said elevated dose is administered on day 8 and the said further dose is administered on day 15 of the step-in cycle.

In preferred embodiments, said initial dose is about 10 μg/kg of the subject's body weight; said elevated dose is about 30 μg/kg of the subject's body weight; and said further dose is about is 90 μg/kg of the subject's body weight.

In certain embodiments, the method further comprises a first treatment cycle following the step-in cycle, the first treatment cycle comprising the steps of:
administering to a subject a first target dose of the T-cell engaging protein, said first target dose ranging from about 5 mg to about 70 mg,
administering to the subject a second target dose of the T-cell engaging protein, said second target dose ranging from about 5 mg to about 70 mg, and
administering to the subject a third target dose of the T-cell engaging protein, said third dose ranging from about 5 mg to about 70 mg,
   wherein optionally the amount of all three target doses is identical.

Preferably, at least one of the first, second or third target doses of the first treatment cycle comprises more than 30 mg.

Preferably, at least one of said first, second or third target doses of the first treatment cycle is about 5 mg, about 10 mg, about 20 mg, about 30 mg, about 40 mg, about 50 mg, about 60 mg, or about 70 mg, optionally about 10 mg or about 60 mg or about 30 mg.

In a more preferred embodiment, the amounts of all three target doses are identical and about 10 mg. In case of safety concerns, said amount may be further reduced and be in the range of about 5 mg to about 10 mg, and is in particular about 5 mg.

In another more preferred embodiment, the amounts of all three target doses are identical and about 60 mg. In case of safety concerns, said amount may be further reduced and be in the range of about 20 mg to about 60 mg, and is in particular about 30 mg.

In more preferred embodiments, said initial dose is about 10 μg/kg of the subject's body weight; said elevated dose is about 30 μg/kg of the subject's body weight; and
said further dose is about is 90 μg/kg of the subject's body weight, and
the amounts of all three target doses are identical and about 10 mg, about 30 mg or about 60 mg.

In preferred embodiments, said first, second and third target doses are administered within three weeks of the first treatment cycle, wherein optionally said first target dose is administered on day 1, said second target dose is administered on day 8 and said third target dose is administered on day 15 of said first treatment cycle, wherein optionally said first treatment cycle has a duration of three weeks.

In preferred embodiments, said first treatment cycle is not repeated or is repeated at least once.

In preferred embodiments, said first treatment cycle is followed by a second treatment cycle, said second treatment cycle comprising the step of or consisting of the step:
administering to the subject a target dose of said T-cell engaging protein, said target dose ranging from about 5 mg to about 70 mg;
   wherein said second treatment cycle is optionally repeated at least once.

Preferably, said second treatment cycle has a total duration of three weeks and/or said target dose is administered on day 1 of the three weeks. Preferably, said target dose is about 10 mg or about 60 mg. In case of safety concerns, said dose of about 60 mg may be further reduced and be in the range of about 20 mg to about 60 mg, and is in particular about 30 mg. Alternatively, in said dose of about 10 mg may be further reduced and be in the range of about 5 mg to about 10 mg, and is in particular about 5 mg.

In certain embodiments, all target doses may be about 30 mg. Alternatively, all target doses may be about 60 mg.

In certain embodiments, the first treatment cycle directly follows said step-in cycle; and/or the second treatment cycle directly follows said first treatment cycle.

In some embodiments, essentially no cytokine release syndrome occurs after said elevated dose of said step-in cycle in the subject.

In some embodiments, said T-cell engaging protein is the only anti-cancer agent to be administered.

In some embodiments, at least one further anti-cancer agent is to be administered, said further anti-cancer agent preferably being a chemotherapeutic agent and/or an anti-PD-L1 antibody.

In certain embodiments, the subject has been treated with a chemotherapy involving administration of inhibitors of cell growth and/or division prior to the step-in cycle, optionally the subject has been treated with one or two chemotherapy cycles (e.g. etoposide and platinum-based therapy, topotecan, lurbinectedin or amrubicin)

In certain embodiments, an occurrence of cytokine release syndrome is to be treated with an anti-cytokine release syndrome agent, preferably selected from the group consisting of a corticosteroid, an IL-6 antagonist and an IL-6R antagonist.

In particular, dosing scheme B3F, i.e. the fourth aspect of the present invention, or any dosing scheme falling within the first or second aspect of the present invention can be co-administered, e.g. with the following cancer treatment agents.

In certain embodiments, the method further comprises a step of co-administering topotecan to the subject, preferably for use in the treatment of SCLC (any treatment line) including ES-SCLC, preferably second line (2L) treatment of ES-SCLC that progressed or recurred following Standard of Care (SoC) treatment and LS-SCLC, preferably first line (1L) treatment.

In certain embodiments, the method further comprises a step of co-administering lurbinectedin to the subject, preferably for use in the treatment of SCLC (any treatment line) including ES-SCLC, preferably 2L treatment of ES-SCLC that progressed or recurred following SoC, and LS-SCLC, preferably 1L treatment.

In certain embodiment, the method further comprises a step of co-administering amrubicin to the subject, preferably for use in the treatment of SCLC (any treatment line) including ES-SCLC, preferably 2L treatment of ES-SCLC that progressed or recurred following SoC, and LS-SCLC, preferably 1L treatment.

In certain embodiments, the method further comprises a step of co-administering etoposide and carboplatin or cisplatin, and optionally also anti-PD-L1 antibody e.g. atezolizumab or durvalumab, to the subject, preferably for use in the treatment of SCLC including ES-SCLC, especially for 1L, 2L and/or 3L treatment, and LS-SCLC (any treatment line).

In certain embodiments, the method further comprises a step of co-administering etoposide and carboplatin or cisplatin, to the subject, preferably for use in the treatment of epNEC or LCNEC of the lung (any treatment line), preferably first line treatment of epNEC or LCNEC of the lung.

Whenever disclosed herein, lurbinectedin, if co-administered may optionally be administered at the approved regimen of 3.2 mg/m² on day 1 of each 21 day cycle as an intravenous infusion, especially for 60 minutes.

Whenever disclosed herein, amrubicin, if co-administered may optionally be administered at the approved regimen of 40 mg/m² on days 1 to 3 of each 21 day cycle. Especially, amrubicin may be administered as an intravenous infusion.

Whenever disclosed herein, topotecan, if co-administered may optionally be administered at the approved regimen of 2.3 mg/m² orally (p.o.) or 1.5 mg/m² by intravenous infusion daily on day 1 to day 5 during each 21 day cycle.

Whenever disclosed herein, carboplatin and etoposide, if co-administered may optionally be administered at the approved regimen of AUC 5 mg/mL min (carboplatin) and 100 mg/m² (etoposide), wherein carboplatin is administered at day 1 during each 21 day cycle and etoposide is administered on days 1 to 3 during each 21 day cycle.

Whenever disclosed herein, cisplatin and etoposide, if co-administered may optionally be administered at the approved regimen of 75 mg/m² (cisplatin) and 100 mg/m² (etoposide), wherein cisplatin is administered at day 1 during each 21 day cycle and etoposide is administered on days 1 to 3 during each 21 day cycle.

In certain embodiments, a treatment regimen may be as follows according to dosing regimen B3F may be as follows:

TABLE 1B
Embodiment of the method of treatment
according to the present invention.
Regimen B3F:
one cycle = 3 weeks duration
Step-in cycle as defined below
One first treatment cycle: Target doses administered weekly
Second treatment cycles: Target doses administered every
three weeks until progression or toxicity, preferably
for a maximum of 36 months

| | | | | | | | second |
|---|---|---|---|---|---|---|---|
| | | | | | | | treatment |
| | | | | first treatment cycle | | | cycles |
| | step-in cycle | | | (optionally repeated) | | | (repeated) |
| | d 1 | d 8 | d 15 | d 1 | d 8 | d 15 | d 1 |
| dose | 10 | 30 | 90 | Target | Target | Target | Target |
| | μg/kg | μg/kg | μg/kg | dose | dose: | dose: | dose: |
| | | | | (same | 10 mg, | 10 mg, | 10 mg, |
| | | | | for every | 30 mg, | 30 mg, | 30 mg, |
| | | | | treatment | 60 mg | 60 mg | 60 mg |
| | | | | step): | | | |
| | | | | 10 mg, | | | |
| | | | | 30 mg, | | | |
| | | | | 60 mg | | | |

### Indications/Patient Population/Monotherapy

Preferred medical indications amenable to treatment with the T-cell engaging proteins of the aspects of the invention are disclosed herein above and below. Corresponding treatment regimens are subject of the various aspects of the invention as disclosed herein above and below.

Of note, this and further aspects of the invention, in one alternative refers to a cancer which is DLL3 positive. While recognizing that “DLL3 positive” is a characterization in molecular terms, it should not go unnoticed that with the advent of molecular medicine, the characterization of a disorder in molecular terms (markers, causally involved molecules as well as molecules qualifying as a target for therapeutic intervention) has become common. Referring to the term “DLL3 positive” it is of note that there is a significant body of evidence that certain tumors, in particular those which are defined in clinical terms herein, do express DLL3 and are thus considered DLL3-positive. As such, terms “DLL3 positive” and “known to express DLL3” are considered to be equivalent.

As regards the notion of “DLL3 positive” on the one hand and the clinical indications (such as NEN, glioma etc.) on the other hand being presented alternatives, it is understood that in scientific terms these are not considered to be alternatives, let alone mutually exclusive.

For the avoidance of doubt, it is understood that said subject is a subject in need of said treatment (also referred to as “patient” herein).

Indications amenable to treatment with the T cell engaging proteins of the invention include any Neuroendocrine Neoplasm (NENs), in particular any Neuroendocrine Carcinoma (NEC), including but not limited to
ES-SCLC, initial treatment (first line (1L) or after failure of at least one prior line of SoC (second line (2L) or higher; e.g. after failure of first line (1L) platinum based chemotherapy and/or anti-PD-L1 therapy),
LS-SCLC initial treatment (1L) or 2L after failure of SoC
LCNEC of the lung, 1L or 2L or higher after failure of SoC (e.g. platinum based therapy)
epNEC, 1L or 2L or higher after failure of SoC (e.g. platinum based therapy)
Grade 3 NET, 1L or 2L.

In some preferred embodiments of these indications, the cancer is locally advanced, unresectable and/or metastatic.

In some embodiments, the cancer is epNEC, a monotherapy with the T-cell engaging protein, such as obrixtamig, may be applied in accordance with the dosing schedules disclosed herein, the line of treatment is ≥2L (patients must have progressed or recurred after at least one platinum-based regimen) and optionally the DLL3-positivity is about 50%.

Further indications include medullary thyroid carcinoma (any treatment line), Merkel Cell Carcinoma (MCC, any treatment line), Neuroendocrine Prostate Cancer (NEPC, any treatment line), glioma/glioblastoma (1L or 2L, e.g. after prior temozolamide (TMZ) treatment).

Particularly preferred indications amenable to treatment with the T cell engaging proteins of the invention are relapsed/refractory extensive-stage small cell lung cancer and other relapsed/refractory neuroendocrine carcinomas, e.g., ES-SCLC which progressed during or following at least two prior lines of treatment including platinum-based chemotherapy, ES-SCLC which progressed during or following at least one prior line of treatment, including platinum-based chemotherapy or anti PD-1/anti PD-L1 therapy, and (metastatic) epNEC which progressed during or following at least one prior line of treatment including platinum-based chemotherapy, and LCNEC of the lung, which progressed during or following at least one prior line of treatment (e.g. platinum-based regimen). In some preferred embodiments of these preferred indications, epNEC or LCNEC are locally advanced, unresectable and/or metastatic.

As becoming apparent from the list of disorders from the first aspect, epNEC in particular excludes MCC and medullary thyroidal carcinoma, as the latter two are listed as separate items. Furthermore, in alternative embodiments, epNEC may exclude Neuroendocrine prostate cancer (NEPC).

In certain embodiments, the cancer of the subject to be treated is Neuroendocrine prostate cancer (NEPC).

In the following various embodiments about a monotherapy are discussed.

In certain embodiments, monotherapy, i.e. only administering the T-cell engaging protein to a patient as defined herein are intended using the dosing schemes disclosed herein. In such cases the DLL3-positivity threshold for the patient selection according to the first and second aspect may be at least about 50% or about 50%. As can be seen from the examples, selecting a threshold of at least about 50% has proven to be effective. Alternatively, the DLL3-positivity threshold may be at least about 25%, especially about 25%.

In any of the preferred embodiments above, the target dose may range from about 5 mg to about 60 mg. In particular, the target dose may be any one of 10 mg, 30 mg or 60 mg. Preferably, the target dose is administered weekly during a first 21 day treatment cycle, followed by a q3w administration (once every three weeks).

In a preferred embodiment, the cancer is epNEC (e.g. epNEC that progressed or recurred after SoC treatment), optionally epNEC excluding NEPC defined above, said threshold may be at least about 25%, such as about 25%, at least about 50% or about 50%. More preferably, in this case the method involves a step in cycle before the first treatment cycle, wherein said initial dose is about 10 μg/kg; 10 μg/kg of the subject's bodyweight;
said elevated dose is about 30 μg/kg of the subject's bodyweight; and
said further dose is about 90 μg/kg of the subject's bodyweight.

More preferably, the amount of said target dose for a treatment cycle is about 10 mg. Alternatively, with a step-in dosing of 10, 30, 90 μg/kg, the target dose for a treatment cycle is about 30 mg.

Further alternatively, with a step-in dosing of 10, 30, 90 μg/kg, the target dose for a treatment cycle is about 60 mg.

In another preferred embodiment, the cancer is SCLC (e.g., ES-SCLC that progressed or recurred after SoC). As defined above, said threshold may be at least about 25%, such as about 25%, at least about 50% or about 50%. More preferably, in this case the method involves a step in cycle,
wherein said initial dose is about 10 μg/kg of the subject's bodyweight;
said elevated dose is about 30 μg/kg of the subject's bodyweight; and
said further dose is about 90 μg/kg of the subject's bodyweight.

More preferably, the amount of said target dose for a treatment cycle is about 10 mg. Alternatively, the target dose for a treatment cycle is about 30 mg.

Further alternatively, the target dose for a treatment cycle is about 60 mg.

In another preferred embodiment, the cancer is NEPC (e.g. NEPC that progressed or recurred after SoC treatment). As defined above, said threshold may be at least about 25%, or about 25%, at least about 50%, or about 50%. More preferably, in this case the method involves a step in cycle,
wherein said initial dose is about 10 μg/kg of the subject's bodyweight;
said elevated dose is about 30 μg/kg of the subject's bodyweight; and
said further dose is about 90 μg/kg of the subject's bodyweight.

More preferably, the amount of said target dose for a treatment cycle is about 10 mg. Alternatively, the target dose for a treatment cycle is about 30 mg.

Further alternatively, the target dose for a treatment cycle is about 60 mg.

In a preferred embodiment, the cancer is LCNEC of the lung (e.g. LCNEC of the lung that progressed or recurred after SoC treatment). As defined above, said threshold may be at least 25%, or about 25%, at least about 50% or about 50%. More preferably, in this case the method involves a step in cycle,
wherein said initial dose is about 10 μg/kg of the subject's bodyweight;
said elevated dose is about 30 μg/kg of the subject's bodyweight; and
said further dose is about 90 μg/kg of the subject's bodyweight.

More preferably, the amount of said target dose for a treatment cycle is about 10 mg. Alternatively, the target dose for a treatment cycle is about 30 mg.

Further alternatively, the target dose for a treatment cycle is about 60 mg.

In a preferred embodiment, the cancer is NEC, such as epNEC. As defined above, said threshold may be at least about 25% or about 25%, at least about 50% or about 50%. More preferably, in this case the method involves a step in cycle,
wherein said initial dose is about 10 μg/kg of the subject's bodyweight;
said elevated dose is about 30 μg/kg of the subject's bodyweight; and
said further dose is about 90 μg/kg of the subject's bodyweight.

More preferably, the amount of said target dose for a treatment cycle is about 10 mg. Alternatively, the target dose for a treatment cycle is about 30 mg.

Further alternatively, target dose for a treatment cycle is about 60 mg.

In certain embodiments, the T cell engaging protein (e.g. BI 764532) is administered as maintenance treatment following 1 to 6 treatment cycles of chemotherapy in 1L or 2L or higher treatment of any NEC, including SCLC (ES-SCLC or LS-SCLC), epNEC, LCNEC, or 1L or 2L treatment of glioma/glioblastoma.

### Method of Treatment and Uses for the Manufacture of a Medicament

The aspects of the present invention and the corresponding embodiments can also be understood in terms of a method of treatment.

Accordingly, one aspect of the present invention provides a method of treatment of a DLL3-positive cancer or a cancer selected from a Neuroendocrine Neoplasm (NEN), Merkel cell carcinoma (MCC), medullary thyroidal carcinoma and glioma in a subject exhibiting at least a threshold of DLL3-positivity, the method comprising administering to the subject a dose of said T-cell engaging protein, wherein
said T-cell engaging protein comprises a first antigen binding unit specifically binding to DLL3, comprising light chain CDRs comprising the amino acid sequences of SEQ ID NO:13 (CDR1), SEQ ID NO:14 (CDR2) and SEQ ID NO:15 (CDR3) and heavy chain CDRs comprising the amino acid sequences of SEQ ID NO:16 (CDR1), SEQ ID NO:17 (CDR2) and SEQ ID NO:18 (CDR3) and a second antigen binding unit specifically binding to CD3 comprising light chain CDRs comprising the amino acid sequences of SEQ ID NO:55 (CDR1), SEQ ID NO:56 (CDR2) and SEQ ID NO:57 (CDR3) and heavy chain CDRs comprising the amino acid sequences of SEQ ID NO:58 (CDR1), SEQ ID NO:59 (CDR2) and SEQ ID NO:60 (CDR3).

In a further aspect, the present invention a method of treatment of a DLL3-positive cancer or a cancer selected from a Neuroendocrine Neoplasm (NEN), Merkel cell carcinoma (MCC), medullary thyroidal carcinoma and glioma,
the method comprising a step-in cycle, said step-in cycle comprising the steps of: said step-in cycle comprising the steps of:
   administering to a subject an initial dose of said T-cell engaging protein, said initial dose ranging from about 20 μg/kg of the subject's body weight to about 40 μg/kg of the subject's body weight,
   administering to the subject an elevated dose, which is elevated as compared to said initial dose, of said T-cell engaging protein, the elevated dose ranging from about 60 μg/kg of the subject's body weight to about 100 μg/kg of the subject's body weight,
   administering to the subject a further dose of the T-cell engaging protein, said further dose ranging from about 5 mg to about 70 mg.

In a further aspect, the present invention provides a method of treatment of a DLL3-positive cancer or a cancer selected from a Neuroendocrine Neoplasm (NEN), Merkel cell carcinoma (MCC), medullary thyroidal carcinoma and glioma,
the method comprising a step-in cycle, said step-in cycle comprising the steps of:
said step-in cycle comprising the steps of:
   administering to a subject an initial dose of said T-cell engaging protein, said initial dose ranging from about 5 μg/kg of the subject's body weight to about 15 μg/kg of the subject's body weight,
   administering to the subject an elevated dose, which is elevated as compared to said initial dose, of said T-cell engaging protein, the elevated dose ranging from about 20 μg/kg of the subject's body weight to about 40 μg/kg of the subject's body weight,
   administering to the subject a further dose of said T-cell engaging protein, said further dose ranging from about 80 μg/kg of the subject's body weight to about 1600 μg/kg of the subject's bodyweight; or
the method comprising a step-in cycle, the step-in cycle comprising the steps of:
   administering to the subject an initial dose, an elevated dose, which is elevated as compared to said initial dose, and a further dose of said T-cell engaging protein, wherein a ratio between said initial, elevated and further dose per kilogram of the subject's body weight is about 1:about 3:about 9.

In a further aspect, the present invention provides a use of T-cell engaging protein comprising a first antigen binding unit that specifically binds to DLL3 and a second antigen binding unit that specifically binds to CD3 for the manufacture of a medicament for use in a method of treatment of a DLL3-positive cancer or a cancer selected from a Neuroendocrine Neoplasm (NEN), Merkel cell carcinoma (MCC), medullary thyroidal carcinoma and glioma in a subject exhibiting at least a threshold of DLL3-positivity, the method comprising administering to the subject a dose of said T-cell engaging protein, wherein
said T-cell engaging protein comprises a first antigen binding unit specifically binding to DLL3, comprising light chain CDRs comprising the amino acid sequences of SEQ ID NO:13 (CDR1), SEQ ID NO:14 (CDR2) and SEQ ID NO:15 (CDR3) and heavy chain CDRs comprising the amino acid sequences of SEQ ID NO:16 (CDR1), SEQ ID NO:17 (CDR2) and SEQ ID NO:18 (CDR3) and a second antigen binding unit specifically binding to CD3 comprising light chain CDRs comprising the amino acid sequences of SEQ ID NO:55 (CDR1), SEQ ID NO:56 (CDR2) and SEQ ID NO:57 (CDR3) and heavy chain CDRs comprising the amino acid sequences of SEQ ID NO:58 (CDR1), SEQ ID NO:59 (CDR2) and SEQ ID NO:60 (CDR3).

In a further aspect, the present invention provides a use of a T-cell engaging protein comprising a first antigen binding unit specifically binding to DLL3 and a second antigen binding unit specifically binding to CD3 for the manufacture of a medicament for use in a treatment of a DLL3-positive cancer or a cancer selected from a Neuroendocrine Neoplasm (NEN), Merkel cell carcinoma (MCC), medullary thyroidal carcinoma and glioma, T-cell engaging protein comprising a first antigen binding unit that specifically binds to DLL3 and a second antigen binding unit that specifically binds to CD3 for use in a method of treatment of a DLL3-positive cancer or a cancer selected from a Neuroendocrine Neoplasm (NEN), Merkel cell carcinoma (MCC), medullary thyroidal carcinoma and glioma, the method comprising a step-in cycle, said step-in cycle comprising the steps of:
said step-in cycle comprising the steps of:
   administering to a subject an initial dose of said T-cell engaging protein, said initial dose ranging from about 20 μg/kg of the subject's body weight to about 40 μg/kg of the subject's body weight,
   administering to the subject an elevated dose, which is elevated as compared to said initial dose, of said T-cell engaging protein, the elevated dose ranging from about 60 μg/kg of the subject's body weight to about 100 μg/kg of the subject's body weight,
   administering to the subject a further dose of the T-cell engaging protein, said further dose ranging from about 5 mg to about 70 mg.

In a further aspect, the present invention provides a T-cell engaging protein comprising a first antigen binding unit specifically binding to DLL3 and a second antigen binding unit specifically binding to CD3 for the manufacture of a medicament for use in a treatment of a DLL3-positive cancer or a cancer selected from a Neuroendocrine Neoplasm (NEN), Merkel cell carcinoma (MCC), medullary thyroidal carcinoma and glioma,
the method comprising a step-in cycle, the step-in cycle comprising the steps of:
   administering to a subject an initial dose of said T-cell engaging protein, said initial dose ranging from about 5 μg/kg of the subject's body weight to about 15 μg/kg of the subject's body weight,
   administering to the subject an elevated dose, which is elevated as compared to said initial dose, of said T-cell engaging protein, the elevated dose ranging from about 20 μg/kg of the subject's body weight to about 40 μg/kg of the subject's body weight,
   administering to the subject a further dose of said T-cell engaging protein, said further dose ranging from about 80 μg/kg of the subject's body weight to about 1600 μg/kg of the subject's bodyweight; or
the method comprising a step-in cycle, the step-in cycle comprising the steps of:
   administering to the subject an initial dose, an elevated dose, which is elevated as compared to said initial dose, and a further dose of said T-cell engaging protein, wherein a ratio between said initial, elevated and further dose per kilogram of the subject's body weight is about 1:about 3:about 9.

For brevity, the embodiments corresponding to these aspects as disclosed above, are referred to but not repeated. In particular, embodiments of the first, second and third aspect are also embodiments of the further aspects disclosed above.

### Co-Administration

The T-cell engaging protein as disclosed herein, and in particular in context with the aspects according to the present invention disclosed herein, can be co-administered with certain other cancer treatment agents. Therefore, the disclosures and features relating to the four aspects of the present invention are suitably combinable with the following disclosure.

In particular, it can be stated that the threshold of DLL3-positivity for co-administrations could be assumed smaller than for a monotherapy in certain embodiments, as without wishing to be bound by a particular theory, it is assumed that the T-cell engaging protein and the other cancer treatment agent used for co-administration might provide a combined effect in a way that the DLL3-positivity threshold can be lower than for monotherapy. For example such threshold then may be at least about 10%, such as about 10%, at least about 20%, such as about 20%, or at least about 25%, such as about 25%, or at least about 30%, such as about 30%, or at least about 40%, such as about 40%. Preferably, the threshold may range from 10% to about 40%. In a most preferred embodiment, the DLL3-positivity threshold may be at least 50% or about 50%. In alternative embodiments, the DLL3-positivity threshold may be at least about 25% or about 25%.

However, this does not exclude other embodiments, where the threshold may be higher as disclosed elsewhere herein for certain applications.

The above thresholds may apply to the following embodiments.

In certain embodiments, the method further comprises a step of co-administering topotecan to the subject, preferably for use in the treatment of SCLC (any treatment line) including ES-SCLC, preferably second line (2L) treatment of ES-SCLC that progressed or recurred following Standard of Care (SoC) treatment and LS-SCLC, preferably first line (1L) treatment.

In certain embodiments, the method further comprises a step of co-administering lurbinectedin to the subject, preferably for use in the treatment of SCLC (any treatment line) including ES-SCLC, preferably 2L treatment of ES-SCLC that progressed or recurred following SoC, and LS-SCLC, preferably 1L treatment

In certain embodiment, the method further comprises a step of co-administering amrubicin to the subject, preferably for use in the treatment of SCLC (any treatment line) including ES-SCLC, preferably 2L treatment of ES-SCLC that progressed or recurred following SoC, and LS-SCLC, preferably 1L treatment.

In certain embodiments, the method further comprises a step of co-administering etoposide and carboplatin or cisplatin, and optionally also anti-PD-L1 antibody e.g. atezolizumab or durvalumab, to the subject, preferably for use in the treatment of SCLC including ES-SCLC, especially for 1L, 2L and/or 3L treatment, and LS-SCLC (any treatment line).

In certain embodiments, the method further comprises a step of co-administering etoposide and carboplatin or cisplatin (optionally also anti-PD-L1 antibody), to the subject, preferably for use in the treatment of epNEC or LCNEC of the lung (any treatment line), preferably first line treatment of epNEC or LCNEC of the lung.

In preferred embodiments, the T-cell engaging protein, carboplatin and cisplatin and etoposide, optionally also anti-PD-L1 antibody, are co-administered for 3 to 5 treatment cycles, in-volving a step in cycle. In more preferred embodiments, only the T-cell engaging protein, optionally together with anti-PD-L1 antibody, will be administered after said 3-5 treatment cycles, in particular for a maximum of 36 months. In other words, a monotherapy or combination therapy with only PD-L1 antibody will continue from the end of the 3-5 cycles. These cycles may involve the step-in cycle for each regimen as disclosed above.

In addition or alternatively, in any of the preferred embodiments above or below, one or more treatment cycles (e.g. one or two treatment cycles) including only chemotherapy may take place prior to any of the combination treatments with the T cell engaging protein described herein above and below.

Whenever disclosed herein, lurbinectedin, if co-administered may optionally be administered at the approved regimen of 3.2 mg/m² on day 1 of each 21 day cycle as an intravenous infusion, especially for 60 minutes.

Whenever disclosed herein, amrubicin, if co-administered may optionally be administered at the approved regimen of 40 mg/m² on days 1 to 3 of each 21 day cycle. Especially, amrubicin may be administered as an intravenous infusion.

Whenever disclosed herein, topotecan, if co-administered may optionally be administered at the approved regimen of 2.3 mg/m² orally (p.o.) or 1.5 mg/m² by intravenous infusion daily on day 1 to day 5 during each 21 day cycle.

Whenever disclosed herein, carboplatin and etoposide, if co-administered may optionally be administered at the approved regimen of AUC 5 mg/mL min (carboplatin) and 100 mg/m² (etoposide), wherein carboplatin is administered at day 1 during each 21 day cycle and etoposide is administered on days 1 to 3 during each 21 day cycle.

Whenever disclosed herein, cisplatin and etoposide, if co-administered may optionally be administered at the approved regimen of 75 mg/m² (cisplatin) and 100 mg/m² (etoposide), wherein cisplatin is administered at day 1 during each 21 day cycle and etoposide is administered on days 1 to 3 during each 21 day cycle.

In more preferred embodiments, disclosed herein is a co-administration of BI 764532 according to any one of the aspects of the present invention with cisplatin or carboplatin and etoposide with either atezolizumab or durvalumab (Dareon™-8) as first line treatment of ES-SCLC, optionally followed by maintenance treatment with BI 764532 alone or in combination with anti-PD-L1 (atezolizumab or durvalumab)

In other more preferred embodiments, disclosed herein is a co-administration of BI 764532 according to any one of the aspects described herein with cisplatin or carboplatin and etoposide with either atezolizumab or durvalumab as second line or above (≥2L) treatment (e.g. after failure of Standard of Care (SoC), such as platinum based chemotherapy) of ES-SCLC, optionally followed by maintenance treatment with BI 764532 alone or in combination with anti-PD-L1 (atezolizumab or durvalumab).

In other more preferred embodiments, disclosed herein is a co-administration of BI 764532 according to any one of the aspects described and topotecan for ES-SCLC that progressed or recurred following platinum-based treatment, or following anti-PD-1 or PD-L1 as applicable, and is eligible for topotecan treatment (Dareon™-9), optionally followed by maintenance treatment with BI 764532 alone or in combination with anti PD-L1.

In other more preferred embodiments, disclosed herein is a co-administration of BI764532 according to any one of the aspects described and lurbinectedin for ES-SCLC that progressed or recurred following platinum-based treatment, or following anti-PD-1 or PD-L1 as applicable, and is eligible for lurbinectedin treatment (Dareon™-9), optionally followed by maintenance treatment with BI 764532 alone or in combination with anti PD-L1.

In other more preferred embodiments, disclosed herein is a co-administration of BI764532 according to any one of the aspects described (e.g. Dareon™-5) and amrubicin for ES-SCLC that progressed or recurred following platinum-based treatment, or following anti-PD-1 or PD-L1 as applicable, and is eligible for amrubicin treatment (Dareon™-9), optionally followed by maintenance treatment with BI 764532 alone or in combination with anti PD-L1.

In other more preferred embodiments, disclosed herein is a chemotherapy (e.g., platinum-based plus etoposide) and/or anti-PDL1 and/or radiotherapy with BI 764532 according to any one of the aspects described in 1L LS-SCLC; optionally followed by maintenance treatment with BI 764532 alone or in combination with anti PD-L1.

In other more preferred embodiments, disclosed herein is a co-administration of BI 764532 according to any one of the aspects described with platinum-based therapy (e.g., carboplatin or cisplatin+etoposide (Dareon™-7) as first-line treatment for locally advanced, unresectable and/or metastatic NEC (extra pulmonary or unknown primary) or LCNEC of the lung, optionally followed by maintenance treatment with BI 764532 alone or in combination with anto-PD-L1.

In other more preferred embodiments, disclosed herein is a co-administration of BI 764532 according to any one of the aspects described with platinum-based therapy (e.g., carboplatin or cisplatin+etoposide as second line (after failure of SoC such as platinum based chemotherapy) treatment for locally advanced or metastatic or unresectable NEC (extra pulmonary or unknown primary) or LCNEC of the lung, optionally followed by maintenance treatment with BI 764532 alone or in combination with anto-PD-L1.

In other more preferred embodiments, disclosed herein is a co-administration of BI 764532 according to any one of the aspects described with SoC, e.g. temozolamide (TMZ), or radiotherapy in first line glioma/glioblastoma.

In other more preferred embodiments, disclosed herein is a co-administration of BI 764532 according to any one of the aspects described with SoC, e.g bevacizumab in second line glioma/glioblastoma (e.g. recurrent or progressive glioma/glioblastoma after prior TMZ treatment).

In other more preferred embodiments, disclosed herein is first line maintenance treatment after completion of chemotherapy in ES-SCLC, LS-SCLC, epNEC or LCNEC with BI764532 according to any one of the aspects described combined with either atezolizumab or durvalumab.

In other more preferred embodiments, disclosed herein is a second line maintenance treatment after completion of chemotherapy (e.g. topotecan, lurbinectedin, amrubicin) in ES-SCLC, epNEC, LCNEC of the lung with BI 764532, optionally in combination with anti-PD-L1.

In other more preferred embodiments, disclosed herein is a co-administration of an PD-1 inhibitor (e.g. ezabenlimab) with BI 764542 according to any of the aspects described, wherein said PD-1 inhibitor may be administered on day 1 of the step-in cycle and each subsequent treatment cycle.

In other preferred embodiments, disclosed herein is co-administration of BI 764532 according to any of the aspects described with SoC, e.g. anti-PD1 (avelumab, nivolumab, pembrolizumab) in 1L Merkel Cell Carcinoma (MCC, disseminated disease), In other preferred embodiments, disclosed herein is first or second line maintenance treatment of MCC after completion of chemotherapy with BI 764532 either alone or in combination with anti-PD1.

In other preferred embodiments, disclosed herein is co-administration of BI 764532 according to any of the aspects described with SoC, e.g. platin plus etoposide, or BI 764532 monotherapy in 2L Merkel Cell Carcinoma (MCC, disseminated disease).

In other preferred embodiments, disclosed herein is co-administration of BI 764532 according to any of the aspects described with SoC, e.g. platin plus etoposide, in 1L Grade 3NET (previously untreated, advanced, unresectable or metastatic.

In other preferred embodiments, disclosed herein maintenance treatment of 1L Grade 3NET (previously untreated, advanced, unresectable or metastatic) after completion of chemotherapy with BI 764532 and anti-PD1 or BI 764532 alone.

### Formulation

Pharmaceutical preparations for parenteral administration, may for example be sterile solutions, suspensions, dispersions, emulsions, or powders which comprise the active ingredients and which are suitable, optionally after a further dissolution or dilution step, for infusion or injection. Suitable carriers or diluents for such preparations for example include, without limitation, sterile water and pharmaceutically acceptable aqueous buffers and solutions such as physiological phosphate-buffered saline, Ringer's solutions, dextrose solution, and Hank's solution; water oils; glycerol; ethanol; glycols such as propylene glycol, as well as mineral oils, animal oils and vegetable oils, for example peanut oil, soybean oil, as well as suitable mixtures thereof.

Solutions of the T cell engaging proteins disclosed herein may also contain a preservative to prevent the growth of microorganisms, such as antibacterial and antifungal agents, for example, p-hydroxybenzoates, parabens, chlorobutanol, phenol, sorbic acid, thiomersal, (alkali metal salts of) ethylenediamine tetraacetic acid, and the like. In many cases, it will be preferable to include isotonic agents, for example, sugars, buffers or sodium chloride. Optionally, emulsifiers and/or dispersants may be used. The proper fluidity can be maintained, for example, by the formation of liposomes, by the maintenance of the required particle size in the case of dispersions or by the use of surfactants. Other agents delaying absorption, for example, aluminum monostearate and gelatin, may also be added. The solutions may be filled into injection vials, ampoules, infusion bottles, and the like.

In all cases, the ultimate dosage form must be sterile, fluid and stable under the conditions of manufacture and storage. Sterile injectable solutions are prepared by incorporating the active compound in the required amount (e.g. 10 mg/ml to 50 mg/ml, preferably any one of 10, 20, 30, 40 or 50 mg/ml) in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Generally, suitable formulations for therapeutic proteins such as the T cell engaging proteins disclosed herein are buffered protein solutions, such as solutions including the protein in a suitable concentration and an aqueous buffer such as:
phosphate buffered saline, pH 7.4,
other phosphate buffers, pH 6.2 to 8.2,
acetate buffers, pH 3.2 to 7.5, preferably pH 4.8 to 5.5
histidine buffers, pH 5.5 to 7.0,
succinate buffers, pH 3.2 to 6.6, and
citrate buffers, pH 2.1 to 6.2,
   and, optionally, salts (e.g. NaCl) and/or sugars (such as e.g. sucrose and trehalose) and/or other polyalcohols (such as e.g. mannitol and glycerol) for providing isotonicity of the solution.

In addition, other agents such as a detergent, e.g. 0.02% Tween-20 or Tween-80, may be included in such solutions.

The above described formulations can optionally be provided as lyophilized formulation that is to be reconstituted in a solution, e.g. in water for injection (WFI).

It will be clear to the person skilled in the art that the ingredients and the amounts thereof as given above do only represent one, preferred option. Alternatives and variations thereof will be immediately apparent to the skilled person, or can easily be conceived starting from the above disclosure.

The T-cell engaging protein is preferably formulated in an aqueous solution or as lyophilisate, preferably buffered, and optionally comprising one, more or all of the following: a mono- or disaccharide, methionine, and a surfactant or detergent. A preferred pH value is between about 5 and about 7 such as about 6. Preferred buffer substances include histidine, citrate and acetate. Preferred mono- and disaccharides include trehalose and sucrose (e.g. between 160 to 260 mM). A preferred surfactant is polysorbate 20 (PS20).

Particularly preferred formulations for the T cell engaging proteins (e.g. at a concentration of any one of 10, 20, 30, 40 or 50 mg/ml) are:
10 mM histidine, 240 mM trehalose, optionally 10 mM methionine, and 0.4 mg/ml PS20; or
10 mM histidine, 240 mM sucrose, and 0.4 mg/ml PS20;
in either case adjusted to a pH of about 6.0.

### Kit

In a fifth aspect, the present invention provides a kit comprising:
a container including the T-cell engaging protein disclosed herein (such as BI 764532); and
written instructions for a method of treating a DLL3-positive cancer or a cancer selected from a Neuroendocrine Neo-plasm (NEN), Merkel cell carcinoma (MCC), medullary thyroidal carcinoma and glioma in a subject exhibiting at least a threshold of DLL3-positivity, the method comprising administering to the subject a dose of said T-cell engaging protein, wherein
said T-cell engaging protein comprises a first antigen binding unit specifically binding to DLL3, comprising light chain CDRs comprising the amino acid sequences of SEQ ID NO:13 (CDR1), SEQ ID NO:14 (CDR2) and SEQ ID NO:15 (CDR3) and heavy chain CDRs comprising the amino acid sequences of SEQ ID NO:16 (CDR1), SEQ ID NO:17 (CDR2) and SEQ ID NO:18 (CDR3) and a second antigen binding unit specifically binding to CD3 comprising light chain CDRs comprising the amino acid sequences of SEQ ID NO:55 (CDR1), SEQ ID NO:56 (CDR2) and SEQ ID NO:57 (CDR3) and heavy chain CDRs comprising the amino acid sequences of SEQ ID NO:58 (CDR1), SEQ ID NO:59 (CDR2) and SEQ ID NO:60 (CDR3).

The T-cell engaging protein is typically contained in the container in its lyophilized form for reconstitution. The container may include further components suitable for the lyophilization process such as shown in section “FORMULATION” herein. Furthermore, the T-cell engaging protein may be present at an amount of 10 mg, 30 mg or 60 mg in the container.

The features and embodiments of the first aspect and second aspect, especially shown in item list 1, are combinable with the fifth aspect as written instructions. The features and embodiments will not be repeated for the sake of brevity.

In a sixth aspect, the present invention provides a kit comprising:
a container including the T-cell engaging protein disclosed herein (such as BI 764532); and
written instructions for a method of treating a DLL3-positive cancer or a cancer selected from a Neuroendocrine Neoplasm (NEN), Merkel cell carcinoma (MCC), medullary thyroidal carcinoma and glioma, the method comprising a step-in cycle, said step-in cycle comprising the steps of:
administering to a subject an initial dose of said T-cell engaging protein, said initial dose ranging from about 20 μg/kg of the subject's body weight to about 40 μg/kg of the subject's body weight,
administering to the subject an elevated dose, which is elevated as compared to said initial dose, of said T-cell engaging protein, the elevated dose ranging from about 60 μg/kg of the subject's body weight to about 100 μg/kg of the subject's body weight,
administering to the subject a further dose of the T-cell engaging protein, said further dose ranging from about 5 mg to about 70 mg.

The T-cell engaging protein is typically contained in the container in its lyophilized form for reconstitution. The container may include further components suitable for the lyophilization process such as shown in section “FORMULATION” herein. Furthermore, the T-cell engaging protein may be present at an amount of 10 mg, 30 mg or 60 mg in the container.

The features and embodiments of the third aspect, especially shown in item list 2 and section “Dosing Regimen B2F” are combinable with the sixth aspect as written instructions. The features and embodiments will not be repeated for the sake of brevity.

In a seventh aspect, the present invention provides a kit comprising:
a container including the T-cell engaging protein disclosed herein (such as BI 764532); and
written instructions for a method of treating of a DLL3-positive cancer or a cancer selected from a Neuroendocrine Neo-plasm (NEN), Merkel cell carcinoma (MCC), medullary thyroidal carcinoma and glioma,
the method comprising a step-in cycle, said step-in cycle comprising the steps of:
   administering to a subject an initial dose of said T-cell engaging protein, said initial dose ranging from about 5 μg/kg of the subject's body weight to about 15 μg/kg of the subject's body weight,
   administering to the subject an elevated dose, which is elevated as compared to said initial dose, of said T-cell engaging protein, the elevated dose ranging from about 20 μg/kg of the subject's body weight to about 40 μg/kg of the subject's body weight,
   administering to the subject a further dose of said T-cell engaging protein, said further dose ranging from about 80 μg/kg of the subject's body weight to about 1600 μg/kg of the subject's bodyweight; or
the method comprising a step-in cycle, the step-in cycle comprising the steps of:
   administering to the subject an initial dose, an elevated dose, which is elevated as compared to said initial dose, and a further dose of said T-cell engaging protein, wherein a ratio between said initial, elevated and further dose per kilogram of the subject's body weight is about 1:about 3:about 9.

The T-cell engaging protein is typically contained in the container in its lyophilized form for reconstitution. The container may include further components suitable for the lyophilization process such as shown in section “FORMULATION” herein. Furthermore, the T-cell engaging protein may be present at an amount of 10 mg, 30 mg or 60 mg in the container.

The features and embodiments of the fourth aspect, especially shown in item list 3 and in the section “Dosing regimen B3F” are combinable with the seventh aspect as written instructions. The features and embodiments will not be repeated for the sake of brevity.

In an eighth aspect, the present invention provides a kit comprising:
a container including the T-cell engaging protein disclosed herein (such as BI 764532); and
written instructions for method for treating a DLL3-positive cancer or a cancer selected from a Neuroendocrine Neo-plasm (NEN), Merkel cell carcinoma (MCC), medullary thyroidal carcinoma and glioma in a subject, the method comprising administering to the subject a dose of said T-cell engaging protein.

The T-cell engaging protein is typically contained in the container in its lyophilized form for reconstitution. The container may include further components suitable for the lyophilization process such as shown in section “FORMULATION” herein. Furthermore, the T-cell engaging protein may be present at an amount of 10 mg, 30 mg or 60 mg in the container.

The features and embodiments of the “further aspect on co-administration” disclosed herein, especially shown in the section “FURTHER ASPECT ON CO-ADMINISTRATION” are combinable with the eighth aspect as written instructions. The features and embodiments will not be repeated for the sake of brevity.

### Further Aspect on Co-Administrations

In a further aspect, the present invention provides a T-cell engaging protein comprising a first antigen binding unit specifically binding to DLL3 and a second antigen binding unit specifically binding to CD3 for use in a method for treating a DLL3-positive cancer or a cancer selected from a Neuroendocrine Neoplasm (NEN), Merkel cell carcinoma (MCC), medullary thyroidal carcinoma and glioma in a subject, the method comprising administering to the subject a dose of said T-cell engaging protein.

The further aspect above and their preferred embodiments below may be specifically combined with the features of the step in cycles and the treatment cycles shown herein above in the section “Embodiments”, “B2F” or B3F”, “co-administration” or item lists 1 to 3. Combination of these features will not be reproduced here for the sake of conciseness.

In certain embodiments, the method further comprises a step of co-administering topotecan to the subject, preferably for use in the treatment of SCLC (any treatment line) including ES-SCLC, preferably second line (2L) treatment of ES-SCLC that progressed or recurred following Standard of Care (SoC) treatment and LS-SCLC, preferably first line (1L) treatment.

In certain embodiments, the method further comprises a step of co-administering lurbinectedin to the subject, preferably for use in the treatment of SCLC (any treatment line) including ES-SCLC, preferably 2L treatment of ES-SCLC that progressed or recurred following SoC, and LS-SCLC, preferably 1L treatment.

In certain embodiment, the method further comprises a step of co-administering amrubicin to the subject, preferably for use in the treatment of SCLC (any treatment line) including ES-SCLC, preferably 2L treatment of ES-SCLC that progressed or recurred following SoC, and LS-SCLC, preferably 1L treatment.

In certain embodiments, the method further comprises a step of co-administering etoposide and carboplatin or cisplatin, and optionally also anti-PD-L1 antibody e.g. atezolizumab or durvalumab, to the subject, preferably for use in the treatment of SCLC including ES-SCLC, especially for 1L, 2L and/or 3L treatment, and LS-SCLC (any treatment line). Preferably, the treatment is 1L. In addition, it is preferred that, the co-administering—in addition to the T-cell engaging protein—involves administering atezolizumab, carboplatin and etoposide.

In certain embodiments, the method further comprises a step of co-administering etoposide and carboplatin or cisplatin (optionally also anti-PD-L1 antibody), to the subject, preferably for use in the treatment of epNEC or LCNEC of the lung (any treatment line), preferably first line treatment of epNEC or LCNEC of the lung.

Preferably, the line of treatment is 1L in DLL3 positive subjects. Optionally, those subjects have unresectable locally advanced or metastatic epNEC. Further preferably, the co-administration involves—in addition to the T-cell engaging protein—an administration of carboplatin and etoposide.

In preferred embodiments, the T-cell engaging protein, carboplatin and cisplatin and etoposide, optionally also anti-PD-L1 antibody, are co-administered for 3 to 5 treatment cycles, involving a step in cycle. In more preferred embodiments, only the T-cell engaging protein, optionally together with anti-PD-L1 antibody, will be administered after said 3-5 treatment cycles, in particular for a maximum of 36 months. In other words, a monotherapy or combination therapy with only PD-L1 antibody will continue from the end of the 3-5 cycles. These cycles may involve the step-in cycle for each regimen as disclosed above.

In addition or alternatively, in any of the preferred embodiments above or below, one or more treatment cycles (e.g. one or two treatment cycles) including only chemotherapy may take place prior to any of the combination treatments with the T cell engaging protein described herein above and below.

Whenever disclosed herein, lurbinectedin, if co-administered may optionally be administered at the approved regimen of 3.2 mg/m² on day 1 of each 21 day cycle as an intravenous infusion, especially for 60 minutes.

Whenever disclosed herein, amrubicin, if co-administered may optionally be administered at the approved regimen of 40 mg/m² on days 1 to 3 of each 21 day cycle. Especially, amrubicin may be administered as an intravenous infusion.

Whenever disclosed herein, topotecan, if co-administered may optionally be administered at the approved regimen of 2.3 mg/m² orally (p.o.) or 1.5 mg/m² by intravenous infusion daily on day 1 to day 5 during each 21 day cycle.

Whenever disclosed herein, carboplatin and etoposide, if co-administered may optionally be administered at the approved regimen of AUC 5 mg/mL min (carboplatin) and 100 mg/m² (etoposide), wherein carboplatin is administered at day 1 during each 21 day cycle and etoposide is administered on days 1 to 3 during each 21 day cycle.

Whenever disclosed herein, cisplatin and etoposide, if co-administered may optionally be administered at the approved regimen of 75 mg/m² (cisplatin) and 100 mg/m² (etoposide), wherein cisplatin is administered at day 1 during each 21 day cycle and etoposide is administered on days 1 to 3 during each 21 day cycle.

In more preferred embodiments, any of the preferred embodiments of the further aspects above may involve a step-in dosing wherein
an initial dose is about 10 μg/kg of the subject's body weight;
an elevated dose is about 30 μg/kg of the subject's body weight; and
a further dose is about is about 90 μg/kg of the subject's body weight,
wherein optionally lurbinectedin or amrubicin is co-administered.

In more preferred embodiments, any of the preferred embodiments of the further aspects may involve a treatment cycle wherein the target dose may range from about 5 mg to about 60 mg. In particular, the target dose may be any one of 10 mg, 30 mg or 60 mg. Preferably, the target dose is administered weekly during a first 21 day treatment cycle, followed by a q3w administration (once every three weeks).

In more preferred embodiments, any of the preferred embodiments of the further aspects above may involve a step-in dosing wherein
an initial dose is about 10 μg/kg of the subject's body weight;
an elevated dose is about 90 μg/kg of the subject's body weight; and
a further dose is about is 10 mg,
wherein optionally lurbinectedin or amrubicin is co-administered.

In additional more preferred embodiments, any of the preferred embodiments of the further aspect above may involve a step-in dosing wherein
an initial dose is about 10 μg/kg of the subject's body weight;
an elevated dose is about 10 mg; and
a further dose is about is 60 mg,
wherein optionally lurbinectedin or amrubicin is co-administered.

In alternative more preferred embodiments any of the preferred embodiments of the further aspect above may involve a step-in dosing, wherein
an initial dose is about 10 μg/kg of the subject's body weight;
an elevated dose is about 60 μg/kg of the subject's body weight; and
a further dose is about is 10 mg,
wherein optionally lurbinectedin or amrubicin is co-administered.

In additional alternative more preferred embodiments any of the preferred embodiments of the further aspect above may involve a step-in dosing, wherein
an initial dose is about 10 μg/kg of the subject's body weight;
an elevated dose is about 60 μg/kg of the subject's body weight; and
a further dose is about is 60 mg, wherein
optionally lurbinectedin or amrubicin is co-administered.

### Premedication, Post-Medication and Further Measures to Reduce Adverse Events (AES) and Ensure Patient Safety (Applies to all Aspects of the Invention)

In certain embodiments, a premedication comprising a non-steroidal anti-inflammatory drug, such as acetaminophen, an anti-histamine and/or a steroid, such as prednisolone or dexamethasone, prior to at least one of the doses of a T-cell engaging protein, optionally prior to each dose is administered.

Premedication as such is a measure established in the art. The attending clinician, upon examination of the patient's clinical status, will be in a position to decide when a premedication is be given, when the dose of the premedication can be lowered, and when premedication can be dispensed with altogether. As a rule, at least the initial dose of the step-in cycle will be preceded by premedication. In addition, any further dose of said step-in cycle may be preceded by premedication as well. To the extent necessary, premedication may also be extended to part or all of the treatment cycles. This is exemplified in the following.

The following preferred pre-medication scheme is tailored to the specific T-cell engaging protein BI 764532 but applies mutatis mutandis or can be adapted by the attending clinician without further ado to other T cell engaging proteins such as those disclosed herein or in WO 2019/234220. The same applies for the post-medication disclosed hereinbelow.

In addition to determining the optimal dosing/administration regimen (see above), the tolerability of BI 764532 administration can be further improved and frequency/severity of adverse events (e.g. CRS and ICANS) reduced by administering pre-medication (treatment given prior to BI 764532 administration to prevent and/or reduce side effects).

Premedication is given 30-60 min before the start of the BI 764532 infusion. The premedication before BI 764532 administration includes:
Acetaminophen/paracetamol 1000 mg p.o. or i.v., or equivalent
Antihistamine (diphenhydramine 50 mg p.o. or i.v., or equivalent) and/or
Steroids (dexamethasone 16 mg or 8 mg p.o. or i.v., or equivalent intermediate-acting corticosteroid).

If the individual patient has tolerated the T-cell engaging protein, e.g. BI 764532, with premedication including steroids without >Grade 1 IRR or CRS for at least 1 administration of target dose (i.e. through all step-in doses+target dose), steroids premedication may be reduced to 50% (e.g. steroids i.v., dexamethasone about 8 mg or about 4 mg) at start of subsequent administrations. If one BI 764532 infusion with reduced steroid premedication is tolerated without >Grade 1 IRR or CRS in an individual patient, steroid premedication should be skipped in repeated treatment cycles for the respective patient.

To further reduce the frequency and severity of adverse events (especially CRS) 1000 mL normal saline i.v. is administered within 1 hour after BI 764532 infusion at step-in doses and first target dose.

Hospitalization (24 h) for safety monitoring following step-in doses and first target dose and reduced safety monitoring at subsequent dose of the T-cell engaging protein, such as BI 764532, administrations can be applied if previously well tolerated.

Therefore, in certain embodiments, a hospitalization, such as for 24 hours, follows the initial, elevated and further doses, i.e. the step-in doses, and the first target dose of the T-cell engaging protein.

In other embodiments, a hospitalization, such as for 24 hours, follows the initial and elevated doses, i.e. the first two step-in doses, of the T-cell engaging protein.

In certain embodiments, the premedication is administered about 0.25 to about 2 hours, such as about 0.5 hours to about 1 hour, directly prior to the administration of the T-cell engaging protein. In this context, “directly prior” usually means that the premedication is administered about 0.25 to about 2 hours, such as about 0.5 hours to about 1 hour, prior to the respective point in time of the start of the administration of a respective dose of the T-cell engaging protein.

In certain embodiments, a post-medication is administered at least 6 hours up to 12 hours, such as 8±2 hours, directly after the respective administration of a dose of the T-cell engaging protein. In this context, “directly after” usually means that the post-medication is administered at least 6 hours up to about 12 hours, such as 8±2 hours, following the respective point in time of the start of the administration of a respective dose of the T-cell engaging protein. In particular, whenever the administration of the post-medication is disclosed herein, the point in time for starting the post-medication, such as 8±2 hours, is typically calculated from respective the point in time of the start of the administration of the respective dose of the T-cell engaging protein.

In other embodiments, no hospitalization follows after the initial, elevated and further dose, i.e. the step-in doses, and the target dose(s) of the T-cell engaging protein.

In certain embodiments, a premedication and a post-medication to improve the tolerability of the T-cell engaging protein, e.g. BI 764532, may be administered to the subject. Said premedication may comprise a non-steroidal anti-inflammatory drug, such as acetaminophen, an anti-histamine and/or a steroid, such as prednisolone or dexamethasone. Said premedication may be administered prior to at least one of the doses of the T-cell engaging protein, optionally prior to each dose of the Te-cell engaging is administered. Based on preliminary experience with post-medication, it was found that an implementation of a post-medication with any of those compounds can increase the safety for administrations of a T-cell engaging protein; in particular with respect to ICANS and/or CRS.

Said post-medication might comprise the same or a different substance as compared to the premedication. Said post-medication may comprise a non-steroidal anti-inflammatory drug, such as acetaminophen, an anti-histamine and/or a steroid, such as prednisolone or dexamethasone, administered directly after one or more doses of the T-cell engaging protein.

The administration route of the premedication may be p.o. (per os, i.e. oral administration) or i.v. (intravenously). Independently, the administration route of the post-medication may be p.o. or i.v.

Further, non-limiting compounds, of the premedication and/or the post-medication are given below.

In certain embodiments, the premedication and/or the post-medication is a steroid. In particular, the steroid is a corticosteroid. Preferably the corticosteroid may be selected from the group consisting of prednisolone and dexamethasone or a combination thereof. More preferably dexamethasone is used as the premedication and/or the post-medication in the aspects of the present invention.

In other embodiments, the steroid may be selected from the group consisting of cortisone, hydrocortisone, prednisone, methylprednisolone, dexamethasone or combinations thereof.

In certain embodiments, there might be one or more than one of the premedication prior to and/or the post-medication administered after a respective a dose of the T-cell engaging protein.

In certain embodiments, the premedication and/or the post-medication can also additionally or alternatively comprise different compounds. Those different compounds may be used in addition or instead of the non-steroidal anti-inflammatory drug, the anti-histamine and/or the steroid. Such different compounds may comprise an inhibitor/agonist of TNF-alpha (TNF) and/or TNF-alpha-Receptor (TNFR) reducing TNF/TFNR signaling and/or an inhibitor/agonist of IL6/IL6R that reduces IL6/IL6R signaling.

In one preferred embodiment, the inhibitor/agonist of TNF/TNFR that reduces TNF/TNFR signalling is selected from the group consisting of etanercept, infliximab, adalimumab, certolizumab pegol may be after any step-in dose of the T-cell engaging protein or after at least one dose of a following treatment cycle.

In one preferred embodiment, the premedication before an administration with a T-cell engaging protein, such as BI 764532, includes:
Acetaminophen/paracetamol 1000 mg p.o. or i.v., or equivalent
Antihistamine (diphenhydramine 50 mg p.o. or i.v., or equivalent) and/or
Steroids (dexamethasone 8 mg p.o. or i.v., or equivalent intermediate-acting corticosteroid);
   and a post-medication includes:
Dexamethasone 8 mg p.o. or i.v. or equivalent intermediate-acting corticosteroid administered 8 (±2) hours following the start of administration of the T-cell engaging protein, such as BI 764532.

In other embodiments, the steroid may be selected from the group consisting of cortisone, betamethasone, triamcinolone, hydrocortisone, hydrocortisone, prednisone, methylprednisolone, dexamethasone or combinations thereof.

Optionally, the premedication and post-medication within a treatment is homogenous. In other words, if a steroid, such as dexamethasone, is comprised in a premedication, it is usually also comprised in the post-medication for each dose of the T-cell engaging protein.

The definition of the premedication and/or post-medication comprising a non-steroidal anti-inflammatory drug, an antihistamine and/or a steroid can mean that one dose for administration is only containing a non-steroidal anti-inflammatory drug, an antihistamine, or a steroid. However, it can also mean that a dose can encompass a co-administration of e.g. a steroid and an antihistamine.

In certain embodiments, there might be more than one premedication administered prior to a respective initial and/or elevated dose of the T-cell engaging protein. Such premedications may be acetaminophen/paracetamol, antihistamine and/or steroids.

In certain embodiments, the pre- and/or post-medication can also additionally or alternatively comprise different compounds. Those different compounds may be used in addition to or instead of the non-steroidal anti-inflammatory drug, the antihistamine and/or the steroid. Such different compounds may comprise an inhibitor/agonist of TNF-alpha (TNF) and/or TNF-alpha-Receptor (TNFR) reducing TNF/TFNR signaling and/or an inhibitor/agonist of IL6/IL6R that reduces IL6/IL6R signaling.

In one preferred embodiment, the inhibitor/agonist of TNF/TNFR that reduces TNF/TNFR signaling is selected from the group consisting of etanercept, infliximab, adalimumab, certolizumab pegol and golimumab or a combination thereof.

In one preferred embodiment, inhibitor/agonist of IL6/IL6R that reduces IL6/IL6R signalling is tocilizumab.

In certain embodiments, pre and/or post-medication can comprise tocilizumab.

In certain embodiments, an amount of a dexamethasone dose comprised in the premedication ranges from about 10 mg to about 18 mg, such as about 16 mg. Any other possible steroid used for the premedication may be dosed equivalently as known by the skilled person who is aware of converting effectiveness among various corticosteroids.

In certain embodiments, an amount of a dexamethasone dose comprised in the premedication ranges from about 4 mg to about 10 mg, such as about 8 mg, and/or an amount of a dose comprised in a post-medication ranges from about 4 mg to about 10 mg, such as about 8 mg. Any other possible steroid used for the post-medication may be dosed equivalently as known by the skilled person who is aware of converting effectiveness among various corticosteroids. In preferred embodiments, the premedication and post-medication are or comprise dexamethasone. In particular, the amount of the premedication and/or post-medication dose, in particular with respect to a steroid, might be reduced to about 50%, if the subject has tolerated the T-cell engaging protein without >Grade 1 IRR or CRS for at least 1 administration of the target dose (i.e. through all step-in doses: initial dose, elevated dose and, if present further dose, plus one target dose). For example, if the subject has tolerated the T-cell engaging protein, such as BI 764532, with steroid premedication and post-medication without >Grade 1 IRR or CRS for at least 1 administration of target dose (i.e. through all step-in doses+target dose), steroids premedication and post-medication in this patient may be reduced to 50% (e.g. steroids i.v., dexamethasone about 4 mg) with subsequent administrations. If one T-cell engaging protein administration, such as an BI 764532 infusion, with reduced steroid premedication and post-medication is tolerated without >Grade 1 IRR or CRS in an individual subject, steroid premedication and post-medication may be skipped in repeated treatment cycles for the respective subject.

In certain embodiments, the premedication is administered directly prior the initial dose and a post-medication is administered directly after the initial dose. In particular, the premedication is administered about 0.25 to about 2 hours, such as about 0.5 hours to about 1 hour before the initial dose and/or the post-medication is administered at least 6 hours up to 12 hours, such as 8±2 hours, after the initial dose.

In certain embodiments, the premedication is administered directly prior the elevated dose and a post-medication is administered directly after the elevated dose. In particular, the pre-medication is administered about 0.25 to about 2 hours, such as 0.5 hours to about 1 hour before the elevated dose and/or the post-medication is administered at least 6 hours up to 12 hours, such as 8±2 hours, after the elevated dose.

In certain embodiments, the premedication is administered directly prior the further dose and a post-medication is administered directly after the further dose. In particular, the premedication is administered about 0.25 to about 2 hours, such as 0.5 hours to about 1 hour before the further dose and/or the post-medication is administered at least 6 hours up to 12 hours, such as 8±2 hours, after the further dose.

In certain embodiments, the premedication is administered directly prior the first treatment dose and a post-medication is administered directly after the first treatment dose. In particular, the pre-medication is administered about 0.25 to about 2 hours, such as 0.5 hours to about 1 hour before the first treatment dose and/or the post-medication is administered at least 6 hours up to 12 hours, such as 8±2 hours, after the first treatment dose.

In certain embodiments, the premedication comprises a non-steroidal anti-inflammatory drug, an antihistamine and a steroid. In preferred embodiments, the non-steroidal anti-inflammatory drug is acetaminophen/paracetamol, such as about 1000 mg p.o. or i.v., or equivalent, diphenhydramine, such as about 50 mg p.o. or i.v., or equivalent, dexamethasone, such as about 16 mg or about 8 mg p.o. or i.v., or equivalent intermediate-acting corticosteroid.

Depending on the duration of monitoring, as described hereinabove, patients may be discharged with an oral steroid and advised to take them at home.

Specific Dosing Schemes/Administrations for which the Premedication and the Post-MedicaTion are Applied

In certain embodiments, the dosing scheme for which premedication and/or post-medication is used is B3flat as described elsewhere herein.

In preferred embodiments, the initial dose amount of the T-cell engaging protein is about 10 μg/kg and the elevated dose amount of the T-cell engaging protein is about 30 μg/kg.

Before and after those initial and elevated doses, a dexamethasone premedication and post-medication of a dose amount of about 8 mg, respectively, is administered.

In certain embodiments, if no CRS/IRR/ICANS occurs in the subject, such as after the initial dose and the elevated dose, the further dose, and the first target dose a reduction of the amount of premedication and post-medication is considered. In alternative embodiments, if CRS/IRR/ICANS occurs, the dexamethasone doses with the next administration of the T-cell engaging protein is the same.

In certain embodiments, a premedication comprises a non-steroidal anti-inflammatory drug, such as acetaminophen/paracetamol, and an antihistamine, such as diphenhydramine. This premedication may be administered prior to the respective step-in doses of the T-cell engaging protein, such as the initial dose, e.g. being about 10 μg/kg, such as the elevated dose, e.g. being about 30 μg/kg, and such as the further dose, e.g. being 90 μg/kg. That premedication may also be administered prior to the first and optionally but not necessarily up to at least 6^{th} or more target doses of the T-cell engaging protein, e.g. being about 60 mg of the T-cell engaging protein. In addition, preferably a steroid, such as dexamethasone, is administered as premedication in addition to the non-steroidal anti-inflammatory drug and the antihistamine and as a post-medication. Prior to each of the initial dose, the elevated dose and the further dose and the first two target doses of the T-cell engaging protein, the steroid, such as dexamethasone, is administered as a premedication. In particular, about 8 mg of the steroid being dexamethasone is respectively administered prior to the initial dose, the elevated dose, and the further dose of the T-cell engaging protein as premedication and about 8 mg of the steroid being dexamethasone is respectively administered after the initial dose, the elevated dose and the further dose of the T-cell engaging protein as post-medication. Prior to the first target dose of the T-cell engaging protein about 4 mg of dexamethasone may be administered as a premedication and after the first target dose of the T-cell engaging protein, about 4 mg of dexamethasone may be administered as a post-medication, e.g. if the individual subject has tolerated the administration of the T-cell engaging protein, such as BI 764532, with steroid premedication and post-medication without >Grade 1 IRR or CRS for the step-in dosing. Furthermore, after the initial dose and the elevated dose and the further dose, of the T-cell engaging protein, the steroid being dexamethasone, may be administered, e.g. in an amount of about 8 mg with respect to dexamethasone, respectively. In particular, about 8 mg of dexamethasone may be administered after the initial dose, the elevated dose, the and further dose of the T-cell engaging protein. In particular, the premedication and/or post-medication steroid dose may be reduced after the first target dose of the T-cell engaging protein is well-tolerated i.e. if no CRS≥2 and/or ICANS (any grade) occurred following the prior target dose. In certain embodiments, the steroid, such as dexamethasone, can be omitted for the premedication and the post-medication starting from the second target dose of the T-cell engaging protein, if the individual subject has tolerated the administration of the T-cell engaging protein, such as BI 764532, with steroid premedication and post-medication without >Grade 1 IRR or CRS for at least 1 administration of target dose, i.e. the first target dose.

In addition, it is preferred that the co-administering—in addition to the T-cell engaging protein—involves administering atezolizumab, carboplatin and etoposide. In those preferred cases, the cancer is SCLC, such as ES-SCLC. Optionally, the threshold of the DLL3-positivity is at about 50% in this case.

In an alternative preferred embodiment, the line of treatment is 1L in DLL3 positive subjects. Optionally, those subjects have epNEC, for example unresectable locally advanced or metastatic epNEC. Further preferably, the co-administration involves—in addition to the T-cell engaging protein—an administration of carboplatin and etoposide. Optionally, the threshold of the DLL3-positivity is about 50% in this case.

### Bispecific T-Cell Engaging Protein

The bispecific T-cell engaging protein for use in the method of treatments according to the present invention comprises a first antigen binding unit specifically binding to DLL3 and a second antigen binding unit specifically binding to CD3. Such T-cell engaging molecules and methods of producing the same are disclosed in WO 2019/234220 A1, the contents of which is incorporated by reference in its entirety. Preferably, the T-cell engaging protein is obrixtamig.

In some embodiments, said bispecific T-cell engaging protein comprises immunoglobulin (Ig) domains, preferably 12 Ig domains. Optionally, said Ig domains are comprised in four polypeptide chains, wherein two light chains comprise two Ig domains each, and two heavy chains comprise four Ig domains each. Preferably, though, said Ig domains are comprised in two polypeptide chains. Advantages of only two polypeptide chains are discussed in the following as are preferred implementations of said Ig domains. For clarity, individual Ig domains are referred to as variable and constant domains of light and heavy chains, in alignment with the established nomenclature for antibodies.

In some embodiments, either antigen binding unit of said bispecific T-cell engaging protein comprises six complementarity determining regions (CDRs). Preferably, within either antigen binding unit, three CDRs are located on one Ig domain of a light chain and three CDRs are located on one Ig domain of a heavy chain. Said CDR-bearing domains are variable domains in accordance with the nomenclature used below.

In one preferred embodiment, the T-cell engaging protein of the invention comprises a first antigen binding unit specifically binding to DLL3, comprising light chain CDRs comprising the amino acid sequences of SEQ ID NO:1 (CDR1), SEQ ID NO:2 (CDR2) and SEQ ID NO:3 (CDR3) and heavy chain CDRs comprising the amino acid sequences of SEQ ID NO:4 (CDR1), SEQ ID NO:5 (CDR2) and SEQ ID NO:6 (CDR3) and a second antigen binding unit specifically binding to CD3, comprising light chain CDRs comprising the amino acid sequences of SEQ ID NO:55 (CDR1), SEQ ID NO:56 (CDR2) and SEQ ID NO:57 (CDR3) and heavy chain CDRs comprising the amino acid sequences of SEQ ID NO:58 (CDR1), SEQ ID NO:59 (CDR2) and SEQ ID NO:60 (CDR3).

In one preferred embodiment, the T-cell engaging protein of the invention comprises a first antigen binding unit specifically binding to DLL3, comprising light chain CDRs comprising the amino acid sequences of SEQ ID NO:7 (CDR1), SEQ ID NO:8 (CDR2) and SEQ ID NO:9 (CDR3) and heavy chain CDRs comprising the amino acid sequences of SEQ ID NO:10 (CDR1), SEQ ID NO:11 (CDR2) and SEQ ID NO:12 (CDR3) and a second antigen binding unit specifically binding to CD3, comprising light chain CDRs comprising the amino acid sequences of SEQ ID NO:55 (CDR1), SEQ ID NO:56 (CDR2) and SEQ ID NO:57 (CDR3) and heavy chain CDRs comprising the amino acid sequences of SEQ ID NO:58 (CDR1), SEQ ID NO:59 (CDR2) and SEQ ID NO:60 (CDR3).

In one preferred embodiment, the T-cell engaging protein of the invention comprises a first antigen binding unit specifically binding to DLL3, comprising light chain CDRs comprising the amino acid sequences of SEQ ID NO:13 (CDR1), SEQ ID NO:14 (CDR2) and SEQ ID NO:15 (CDR3) and heavy chain CDRs comprising the amino acid sequences of SEQ ID NO:16 (CDR1), SEQ ID NO:17 (CDR2) and SEQ ID NO:18 (CDR3) and a second antigen binding unit specifically binding to CD3, comprising light chain CDRs comprising the amino acid sequences of SEQ ID NO:55 (CDR1), SEQ ID NO:56 (CDR2) and SEQ ID NO:57 (CDR3) and heavy chain CDRs comprising the amino acid sequences of SEQ ID NO:58 (CDR1), SEQ ID NO:59 (CDR2) and SEQ ID NO:60 (CDR3).

In one preferred embodiment, the T-cell engaging protein of the invention comprises a first antigen binding unit specifically binding to DLL3, comprising light chain CDRs comprising the amino acid sequences of SEQ ID NO:1 (CDR1), SEQ ID NO:2 (CDR2) and SEQ ID NO:3 (CDR3) and heavy chain CDRs comprising the amino acid sequences of SEQ ID NO:4 (CDR1), SEQ ID NO:5 (CDR2) and SEQ ID NO:6 (CDR3) and a second antigen binding unit specifically binding to CD3 comprising light chain CDRs comprising the amino acid sequences of SEQ ID NO:61 (CDR1), SEQ ID NO:62 (CDR2) and SEQ ID NO:63 (CDR3) and heavy chain CDRs comprising the amino acid sequences of SEQ ID NO:64 (CDR1), SEQ ID NO:65 (CDR2) and SEQ ID NO:66 (CDR3).

In one preferred embodiment, the T-cell engaging protein of the invention comprises a first antigen binding unit specifically binding to DLL3, comprising light chain CDRs comprising the amino acid sequences of SEQ ID NO:7 (CDR1), SEQ ID NO:8 (CDR2) and SEQ ID NO:9 (CDR3) and heavy chain CDRs comprising the amino acid sequences of SEQ ID NO:10 (CDR1), SEQ ID NO:11 (CDR2) and SEQ ID NO:12 (CDR3) and a second antigen binding unit specifically binding to CD3, comprising light chain CDRs comprising the amino acid sequences of SEQ ID NO:61 (CDR1), SEQ ID NO:62 (CDR2) and SEQ ID NO:63 (CDR3) and heavy chain CDRs comprising the amino acid sequences of SEQ ID NO:64 (CDR1), SEQ ID NO:65 (CDR2) and SEQ ID NO:66 (CDR3).

In one preferred embodiment, the T-cell engaging protein of the invention comprises a first antigen binding unit specifically binding to DLL3, comprising light chain CDRs comprising the amino acid sequences of SEQ ID NO:13 (CDR1), SEQ ID NO:14 (CDR2) and SEQ ID NO:15 (CDR3) and heavy chain CDRs comprising the amino acid sequences of SEQ ID NO:16 (CDR1), SEQ ID NO:17 (CDR2) and SEQ ID NO:18 (CDR3) and a second antigen binding unit specifically binding to CD3, comprising light chain CDRs comprising the amino acid sequences of SEQ ID NO:61 (CDR1), SEQ ID NO:62 (CDR2) and SEQ ID NO:63 (CDR3) and heavy chain CDRs comprising the amino acid sequences of SEQ ID NO:64 (CDR1), SEQ ID NO:65 (CDR2) and SEQ ID NO:66 (CDR3).

In one preferred embodiment, the T-cell engaging protein of the invention comprises (i) a first antigen binding unit specifically binding to DLL3 comprising a light chain variable domain comprising the amino acid sequence of SEQ ID NO:37 and a heavy chain variable domain comprising the amino acid sequence of SEQ ID NO:38 and (ii) a second antigen binding unit specifically binding to CD3 comprising a light chain variable domain comprising the amino acid sequence of SEQ ID NO:67 and a heavy chain variable domain comprising the amino acid sequence of SEQ ID NO:68.

In one preferred embodiment, the T-cell engaging protein of the invention comprises (i) a first antigen binding unit specifically binding to DLL3 comprising a light chain variable domain comprising the amino acid sequence of SEQ ID NO:39 and a heavy chain variable domain comprising the amino acid sequence of SEQ ID NO:40 and (ii) a second antigen binding unit specifically binding to CD3 comprising a light chain variable domain comprising the amino acid sequence of SEQ ID NO:67 and a heavy chain variable domain comprising the amino acid sequence of SEQ ID NO:68.

In one preferred embodiment, the T-cell engaging protein of the invention comprises (i) a first antigen binding unit specifically binding to DLL3 comprising a light chain variable domain comprising the amino acid sequence of SEQ ID NO:41 and heavy chain variable domain comprising the amino acid sequence of SEQ ID NO:42 and (ii) a second antigen binding unit specifically binding to CD3 comprising a light chain variable domain comprising the amino acid sequence of SEQ ID NO:67 and a heavy chain variable domain comprising the amino acid sequence of SEQ ID NO:68.

In one preferred embodiment, the T-cell engaging protein of the invention comprises (i) a first antigen binding unit specifically binding to DLL3 comprising a light chain variable domain comprising the amino acid sequence of SEQ ID NO:37 and heavy chain variable domain comprising the amino acid sequence of SEQ ID NO:38 and (ii) a second antigen binding unit specifically binding to CD3 comprising a light chain variable domain comprising the amino acid sequence of SEQ ID NO:69 and a heavy chain variable domain comprising the amino acid sequence of SEQ ID NO:70.

In one preferred embodiment, the T-cell engaging protein of the invention comprises (i) a first antigen binding unit specifically binding to DLL3 comprising a light chain variable domain comprising the amino acid sequence of SEQ ID NO:39 and heavy chain variable domain comprising the amino acid sequence of SEQ ID NO:40 and (ii) a second antigen binding unit specifically binding to CD3 comprising a light chain variable domain comprising the amino acid sequence of SEQ ID NO:69 and a heavy chain variable domain comprising the amino acid sequence of SEQ ID NO:70.

In one preferred embodiment, the T-cell engaging protein of the invention comprises (i) a first antigen binding unit specifically binding to DLL3 comprising a light chain variable domain comprising the amino acid sequence of SEQ ID NO:41 and heavy chain variable domain comprising the amino acid sequence of SEQ ID NO:42 and a second antigen binding unit specifically binding to CD3 comprising a light chain variable domain comprising the amino acid sequence of SEQ ID NO:69 and a heavy chain variable domain comprising the amino acid sequence of SEQ ID NO:70.

In some embodiments of the T-cell engaging protein of the invention, the first and/or the second antigen binding unit further comprises a CL and a CH1 domain like in a light/heavy Fab domain of a conventional antibody molecule, thus said first binding unit comprises a) a VL domain (e.g., defined by the light chain CDR (LCCDR) or VL sequences of any one of DLL3 #1, DLL3 #2, DLL3 #3) covalently linked (directly or indirectly bound) to a first CL domain and b) a VH domain (e.g., defined by the heavy chain CDR (HCCDR) or VH sequences of any one of DLL3 #1, DLL3 #2, DLL3 #3,) covalently linked (directly or indirectly bound) to a first CH1 domain and/or said second antigen binding unit comprises a) a VL domain (e.g., defined by the LCCDR or VL sequences of CD3 #1 or CD3 #2) covalently linked (directly or indirectly bound) to a second CL domain and b) a VH domain (e.g., defined by HCCDR or VH sequences of CD3 #1 or CD3 #2) covalently linked (directly or indirectly bound) to a second CH1 domain.

For an explanation of the designations comprising a number sign (“#”), please see the table further below.

In the context of the present invention, a CL domain is the constant domain of an antibody light chain, for example a kappa (κ) or a lambda (λ) light chain. An example of a constant region of a kappa light chain is shown in SEQ ID NO:87. An example of a constant region of a lambda light chain is shown in SEQ ID NO:88. In some embodiments, the first and the second CL domain are the same, e.g. the first and the second CL domain are both a kappa light chain constant domain or the first and the second CL domain are both a lambda light chain constant domain. In some embodiments, the first and the second CL domain are different, e.g., the first CL domain is a constant kappa domain and the second CL domain is a constant lambda domain or vice versa.

In the context of the present invention, a CH1 domain is the first constant domain of an antibody heavy chain. An example of a constant CH1 domain is shown in SEQ ID NO:90.

In a preferred embodiment, the T-cell engaging protein of the invention comprises a first single chain Fab forming a first antigen binding unit comprising the sequence of SEQ ID NO:49 and a second single chain Fab comprising the sequence of SEQ ID NO:71 forming a second antigen binding unit. In a preferred embodiment, the T-cell engaging protein of the invention comprises a first single chain Fab forming a first antigen binding unit comprising the sequence of SEQ ID NO:50 and a second single chain Fab comprising the sequence of SEQ ID NO:71 forming a second antigen binding unit. In a preferred embodiment, the T-cell engaging protein of the invention comprises a first single chain Fab forming a first antigen binding unit comprising the sequence of SEQ ID NO:51 and a second single chain Fab comprising the sequence of SEQ ID NO:71 forming a second antigen binding unit.

In a preferred embodiment, the T-cell engaging protein of the invention comprises a first single chain Fab forming a first antigen binding unit comprising the sequence of SEQ ID NO:49 and a second single chain Fab comprising the sequence of SEQ ID NO:72 forming a second antigen binding unit. In a preferred embodiment, the T-cell engaging protein of the invention comprises a first single chain Fab forming a first antigen binding unit comprising the sequence of SEQ ID NO:50 and a second single chain Fab comprising the sequence of SEQ ID NO:72 forming a second antigen binding unit. In a preferred embodiment, the T-cell engaging protein of the invention comprises a first single chain Fab forming a first antigen binding unit comprising the sequence of SEQ ID NO:51 and a second single chain Fab comprising the sequence of SEQ ID NO:72 forming a second antigen binding unit.

In preferred embodiments, the T-cell engaging protein of the invention comprises a first polypeptide chain comprising a first single chain Fab specifically binding to DLL3 (e.g., any one of DLL3 #1, DLL3 #2, DLL3 #3 as defined by the respective CDR or VH/VL sequences above) and a first Fc domain (this polypeptide chain herein referred to also as “DLL3 chain”) and a second polypeptide chain comprising a second single chain Fab specifically binding to CD3 (e.g. CD3 #1 or CD3 #2 as defined by the respective CDR or VH/VL sequences above) and a second Fc domain (this polypeptide chain herein referred to herein also as “CD3 chain”). In some embodiments, the first and the second Fc domain are the same. In preferred embodiments, the first and the second Fc domains are different. The resulting T-cell engaging protein of the invention bear a full Fc and have two independent binding sites, a first binding unit for DLL3 and a second binding unit for CD3.

In another preferred embodiment, said bispecific T-cell engaging protein comprises a first polypeptide chain specifically binding to DLL3 and a second polypeptide chain specifically binding to CD3, wherein the first chain comprises a first light chain covalently linked (e.g. directly bound) to a first linker, which is itself covalently linked (e.g. directly bound) to a first heavy chain, and wherein the second chain specifically binding to CD3 comprises a second light chain covalently linked (e.g. directly bound) to a second linker, which is itself covalently linked (e.g. directly bound) to a second heavy chain.

In some embodiments, starting from its N-terminus, the first polypeptide chain comprises a first light chain variable region specifically binding to DLL3, a first light chain constant region, a first linker, a first heavy chain variable region specific for DLL3 and a first heavy chain constant region. In some embodiments, starting from its N-terminus, the second polypeptide chain comprises a second light chain variable region specifically binding to CD3, a second light chain constant region, a second linker, a second heavy chain variable region specific for CD3 and a second heavy chain constant region.

The resulting proteins bear a full Fc, which is marginally larger than an IgG (mostly owing to said first and second linker) and has two independent binding sites (e.g., each binding site being monovalent for the respective antigen), a first binding site for DLL3 and a second binding site for CD3. Preferably, the first and second polypeptide chain are linked via one or more disulfide bonds. As such, preferred bispecific-cell engaging protein of the invention are antibody-like structures, having the Y shaped structure of a conventional full length antibody. This bispecific format greatly reduces heterogeneity after expression and purification (e.g. by avoiding mispairing of light and heavy variable domains with different binding specificities), while maintaining the functional properties of the binding moieties within a structure less likely to generate unwanted immunogenic reactions. This also enables good expression of heterodimeric proteins, e.g. in mammalian cells.

In one preferred embodiment, the T-cell engaging protein comprises a first polypeptide chain specific for DLL3 comprising an amino acid sequence of SEQ ID NO:73 and a second polypeptide chain specific for CD3 comprising the amino acid sequence of SEQ ID NO:79.

In one preferred embodiment, the T-cell engaging protein comprises a first polypeptide chain specific for DLL3 comprising an amino acid sequence of SEQ ID NO:74 and a second polypeptide chain specific for CD3 comprising the amino acid sequence of SEQ ID NO:79.

In one preferred embodiment, the T-cell engaging protein comprises a first polypeptide chain specific for DLL3 comprising an amino acid sequence of SEQ ID NO:75 and a second polypeptide chain specific for CD3 comprising the amino acid sequence of SEQ ID NO:79, in the examples below also referred to as BI 764532.

In one preferred embodiment, the T-cell engaging protein comprises a first polypeptide chain specific for DLL3 comprising an amino acid sequence of SEQ ID NO:73 and a second polypeptide chain specific for CD3 comprising the amino acid sequence of SEQ ID NO:80.

In one preferred embodiment, the T-cell engaging protein comprises a first polypeptide chain specific for DLL3 comprising an amino acid sequence of SEQ ID NO:74 and a second polypeptide chain specific for CD3 comprising the amino acid sequence of SEQ ID NO:80.

In one preferred embodiment, the T-cell engaging protein comprises a first polypeptide chain specific for DLL3 comprising an amino acid sequence of SEQ ID NO:75 and a second polypeptide chain specific for CD3 comprising the amino acid sequence of SEQ ID NO:80.

Amino acid sequences defining preferred T-cell engaging proteins or parts thereof are set forth below. The SEQ ID NOs are those which are used in the sequence listing accompanying this application. In case of discrepancies, the sequences given in the table below or in WO 2019/234220 shall prevail.

TABLE 2

| | Brief | |
|---|---|---|
| | description | |
| SEQ ID | of | |
| Number | sequence | Sequence |
| SEQ ID | DLL3#1 | RASQSVSSNFLV |
| NO: 1 | LCCDR 1 | |
| SEQ ID | DLL3#1 | GASTRAS |
| NO: 2 | LCCDR 2 | |
| SEQ ID | DLL3#1 | QQYGDSPYT |
| NO: 3 | LCCDR | |
| | 3 | |
| SEQ ID | DLL3#1 | GNTFTNYYMH |
| NO: 4 | HCCDR 1 | |
| SEQ ID | DLL3#1 | IIDPSVGSKSYAQKFLG |
| NO: 5 | HCCDR 2 | |
| SEQ ID | DLL3#1 | AGKRFGESYFDY |
| NO: 6 | HCCDR 3 | |
| SEQ ID | DLL3#2 | RASQGISNYLA |
| NO: 7 | LCCDR 1 | |
| SEQ ID | DLL3#2 | AASSLQS |
| NO: 8 | LCCDR | |
| | 2 | |
| SEQ ID | DLL3#2 | LQHNSSPYT |
| NO: 9 | LCCDR 3 | |
| SEQ ID | DLL3#2 | GYTFTSYYMH |
| NO: 10 | HCCDR 1 | |
| SEQ ID | DLL3#2 | IINPSGGSTSYAQKFQG |
| NO: 11 | HCCDR 2 | |
| SEQ ID | DLL3#2 | GEAVGGNYYYYGMDV |
| NO: 12 | HCCDR 3 | |
| SEQ ID | DLL3#3 | RASQGISNYLV |
| NO: 13 | LCCDR 1 | |
| SEQ ID | DLL3#3 | AVSSLYS |
| NO: 14 | LCCDR 2 | |
| SEQ ID | DLL3#3 | LQHDSYPYT |
| NO: 15 | LCCDR 3 | |
| SEQ ID | DLL3#3 | GYTFTSYYVH |
| NO: 16 | HCCDR 1 | |
| SEQ ID | DLL3#3 | IINPGGGTTSYAQKFLG |
| NO: 17 | HCCDR 2 | |
| SEQ ID | DLL3#3 | GEAVTGNYFYYGMDV |
| NO: 18 | HCCDR 3 | |
| SEQ ID | DLL3#1 | EIVLTQSPGTLSLSPGERATLSCRASQSVSSNFLVWYQQKPGQAPR- |
| NO: 37 | VL | PLIYGASTRASGIP- |
| | | DRFSGSGSGADFTLTISRLEPEDFALYYCQQYGDSPYTFGQGTTLEIK |
| SEQ ID | DLL3#1 | QVQLVQSGAEVKKPGASVKVSCKASGNTFTNYYMHWVR- |
| NO: 38 | VH | QAPGPGLEWMGIIDPSVGSKSYA- |
| | | QKFLGRVTIARDTSTSTVFLDLYSLRSEDTAVYFCARAGKRFGESYFD |
| | | YWGQGTLVTVSS |
| SEQ ID | DLL3#2 | DIQMTQSPSAMSASVGDRVTITCRASQGISNYLAWFQQKPGKVPEP- |
| NO: 39 | VL | LIYAAS- |
| | | SLQSGVPSRFSGSGSVTEFTLTISSLQPEDFATYYCLQHNSSPYTFGQG |
| | | TKLEIK |
| SEQ ID | DLL3#2 | QVQLVQSGAEVKKPGASVKVSCKASGYTFTSYYMHWVR- |
| NO: 40 | VH | QAPGQGLEWMGIINPSGGSTSYA- |
| | | QKFQGRVTMTRDTSTSTVYMELSSLRSEDTAVYYCARGEAVGGNYY |
| | | YYGMDVWGQGTTVTVSS |
| SEQ ID | DLL3#3 | DIQMTQSPSAMSASVGDRVTITCRASQGISNYLVWFQQKPGKAP- |
| NO: 41 | VL | KRLIYAVSS- |
| | | LYSGVPSRFSGSGSGTEFTLTISSLQPEDFATYYCLQHDSYPYTFGQGT |
| | | KLEIK |
| SEQ ID | DLL3#3 | QVQLVQSGAEVKKPGASVKVSCKASGYTFTSYYVHWVR- |
| NO: 42 | VH | QAPGQGLEWMVIINPGGGTTSYA- |
| | | QKFLGRVTMTRDTSTNTVYMELKSLRSEDTAVYYCARGEAVTGNYF |
| | | YYGMDVWGQGTTVTVSS |
| SEQ ID | DLL3#1 | EIVLTQSPGTLSLSPGERATLSCRASQSVSSNFLVWYQQKPGQAPR- |
| NO: 49 | scFab | PLIYGASTRASGIP- |
| | | DRFSGSGSGADFTLTISRLEPEDFALYYCQQYGDSPYTFGQGTTLEIKR |
| | | TVAAPSVFIFPPSDEQLKSGTASVVCLLNNFYPREAK- |
| | | VQWKVDNALQSGNSQESVTE- |
| | | QDSKDSTYSLSSTLTLSKADYEKHKVYACEVTHQGLSSPVTKSFNRG |
| | | ECGGGGSEGKSSGSGSESKSTEGKSSGSGS- |
| | | ESKSTGGGGSQVQLVQSGAEVKKP- |
| | | GASVKVSCKASGNTFTNYYMHWVRQAPGPGLEWMGIIDPSVGSKSY |
| | | AQKFLGRVTIARDTSTSTVFLDLYS- |
| | | LRSEDTAVYFCARAGKRFGESYFDYWGQGTLVTVSSASTKGPSVFPL |
| | | APSSKSTSGGTAALGCLVKDYFPEPVTVSWNSGALTSGVHT- |
| | | FPAVLQSSGLYS- |
| | | LSSVVTVPSSSLGTQTYICNVNHKPSNTKVDKRVEPKSC |
| SEQ ID | DLL3#2 | DIQMTQSPSAMSASVGDRVTITCRASQGISNYLAWFQQKPGKVPEP- |
| NO: 50 | scFab | LIYAAS- |
| | | SLQSGVPSRFSGSGSVTEFTLTISSLQPEDFATYYCLQHNSSPYTFGQG |
| | | TKLEIKRTVAAPSVFIFPPSDEQLKSGTASVVCLLNNFYPREAK- |
| | | VQWKVDNALQSGNSQESVTEQDSKDSTYSLSSTLTLSKADYEKHKV |
| | | YACEVTHQGLSSPVTKSFNRGECGGGGSEGKSSGSGS- |
| | | ESKSTEGKSSGSGS- |
| | | ESKSTGGGGSQVQLVQSGAEVKKPGASVKVSCKASGYTFTSYYMHW |
| | | VRQAPGQGLEWMGIINPSGGSTSYA- |
| | | QKFQGRVTMTRDTSTSTVYMELSSLRSEDTAVYYCARGEAVGGNYY |
| | | YYGMDVWGQGTTVTVSSASTKGPSVFPLAPSSKSTSGG- |
| | | TAALGCLVKDYFPEPVTVSWNSGALTSGVHTFPAVLQSSGLYSLSSV |
| | | VTVPSSSLGTQTYICNVNHKPSNTKVDKRVEPKSC |
| SEQ ID | DLL3#3 | DIQMTQSPSAMSASVGDRVTITCRASQGISNYLVWFQQKPGKAP- |
| NO: 51 | scFab | KRLIYAVSS- |
| | | LYSGVPSRFSGSGSGTEFTLTISSLQPEDFATYYCLQHDSYPYTFGQGT |
| | | KLEIKRTVAAPSVFIFPPSDEQLKSGTASVVCLLNNFYPREAK- |
| | | VQWKVDNALQSGNSQESVTEQDSKDSTYSLSSTLTLSKADYEKHKV |
| | | YACEVTHQGLSSPVTKSFNRGECGGGGSEGKSSGSGS- |
| | | ESKSTEGKSSGSGS- |
| | | ESKSTGGGGSQVQLVQSGAEVKKPGASVKVSCKASGYTFTSYYVHW |
| | | VRQAPGQGLEWMVIINPGGGTTSYA- |
| | | QKFLGRVTMTRDTSTNTVYMELKSLRSEDTAVYYCARGEAVTGNYF |
| | | YYGMDVWGQGTTVTVSSASTKGPSVFPLAPSSKSTSGG- |
| | | TAALGCLVKDYFPEPVTVSWNSGALTSGVHTFPAVLQSSGLYSLSSV |
| | | VTVPSSSLGTQTYICNVNHKPSNTKVDKRVEPKSC |
| SEQ ID | CD3#1 | RSSTGAVTTSNYAN |
| NO: 55 | LCCDR | |
| | 1 | |
| SEQ ID | CD3#1 | GTNKRAP |
| NO: 56 | LCCDR | |
| | 2 | |
| SEQ ID | CD3#1 | ALWYSNLWV |
| NO: 57 | LCCDR | |
| | 3 | |
| SEQ ID | CD3#1 | GFTFNTYAMN |
| NO: 58 | HCCDR | |
| | 1 | |
| SEQ ID | CD3#1 | RIRSKYNNYATYYADSVKD |
| NO: 59 | HCCDR | |
| | 2 | |
| SEQ ID | CD3#1 | HGNFGNSYVSWFAY |
| NO: 60 | HCCDR | |
| | 3 | |
| SEQ ID | CD3#2 | RSSTGAVTTSNYAN |
| NO: 61 | LCCDR | |
| | 1 | |
| SEQ ID | CD3#2 | GTNKRAP |
| NO: 62 | LCCDR | |
| | 2 | |
| SEQ ID | CD3#2 | ALWYSNLWV |
| NO: 63 | LCCDR | |
| | 3 | |
| SEQ ID | CD3#2 | GFTFNTYAMN |
| NO: 64 | HCCDR | |
| | 1 | |
| SEQ ID | CD3#2 | RIRSKYINYATYYADSVKD |
| NO: 65 | HCCDR | |
| | 2 | |
| SEQ ID | CD3#2 | HGNFGNSYVSWFAY |
| NO: 66 | HCCDR | |
| | 3 | |
| SEQ ID | CD3#1 | EAVVTQEPSLTVSPGGTVTLTCRSSTGAVTTSNYANWVQEK- |
| NO: 67 | VL | PGQLPRGLIGGTNKRAP- |
| | | WVPARFSGSLLGGKAALTLSGAQPEDEAEYFCALWYSNLWVFGGGT |
| | | KLTVL |
| SEQ ID | CD3#1 | EVQLVESGGGLVQPGGSLKLSCAASGFTFNTYAMNWVR- |
| NO: 68 | VH | QAPGKGLEWVARIRSKYNNYATY- |
| | | YADSVKDRFTISRDDSKNTAYLQMNNLKTEDTAVYYCVRHGNFGNS |
| | | YVSWFAYWGQGTLVTVSA |
| SEQ ID | CD3#2 | EAVVTQEPSLTVSPGGTVTLTCRSSTGAVTTSNYANWVQEK- |
| NO: 69 | VL | PGQLPRGLIGGTNKRAP- |
| | | WVPARFSGSLLGGKAALTLSGAQPEDEAEYFCALWYSNLWVFGGGT |
| | | KLTVL |
| SEQ ID | CD3#2 | EVQLVESGGGLVQPGGSLKLSCAASGFTFNTYAMNWVR- |
| NO: 70 | VH | QAPGKGLEWVARIRSKYINYATY- |
| | | YADSVKDRFTISRDDSKNTAYLQMNNLKTEDTAVYYCVRHGNFGNS |
| | | YVSWFAYWGQGTLVTVSA |
| SEQ ID | CD3#1 | EAVVTQEPSLTVSPGGTVTLTCRSSTGAVTTSNYANWVQEK- |
| NO: 71 | scFab | PGQLPRGLIGGTNKRAP- |
| | | WVPARFSGSLLGGKAALTLSGAQPEDEAEYFCALWYSNLWVFGGGT |
| | | KLTVLGQPKAAPSVTLFPPSSEELQANKATLVCLISDFYPGAVKVAW- |
| | | KADG- |
| | | SPVNTGVETTTPSKQSNNKYAASSYLSLTPEQWKSHRSYSCQVTHEG |
| | | STVEKTVAPAECSGGGGSEGKSSGSGSESKSTEGKSSGSGS- |
| | | ESKSTGGGGSEVQLVESGG- |
| | | GLVQPGGSLKLSCAASGFTFNTYAMNWVRQAPGKGLEWVARIRSKY |
| | | NNYATY- |
| | | YADSVKDRFTISRDDSKNTAYLQMNNLKTEDTAVYYCVRHGNFGN- |
| | | SYVSWFAY- |
| | | WGQGTLVTVSAASTKGPSVFPLAPSSKSTSGGTAALGCLVKDYFPEP |
| | | VTVSWNSGALTSGVHTFPAVLQSSGLYSLSSVVTVPSSSLGTQTYIC- |
| | | NVNHKPSNTKVDKRVEPKSC |
| SEQ ID | CD3#2 | EAVVTQEPSLTVSPGGTVTLTCRSSTGAVTTSNYANWVQEK- |
| NO: 72 | scFab | PGQLPRGLIGGTNKRAP- |
| | | WVPARFSGSLLGGKAALTLSGAQPEDEAEYFCALWYSNLWVFGGGT |
| | | KLTVLGQPKAAPSVTLFPPSSEELQANKATLVCLISDFYPGAVKVAW- |
| | | KADG- |
| | | SPVNTGVETTTPSKQSNNKYAASSYLSLTPEQWKSHRSYSCQVTHEG |
| | | STVEKTVAPAECSGGGGSEGKSSGSGSESKSTEGKSSGSGS- |
| | | ESKSTGGGGSEVQLVESGG- |
| | | GLVQPGGSLKLSCAASGFTFNTYAMNWVRQAPGKGLEWVARIRSKY |
| | | INYATY- |
| | | YADSVKDRFTISRDDSKNTAYLQMNNLKTEDTAVYYCVRHGNFGN- |
| | | SYVSWFAY- |
| | | WGQGTLVTVSAASTKGPSVFPLAPSSKSTSGGTAALGCLVKDYFPEP |
| | | VTVSWNSGALTSGVHTFPAVLQSSGLYSLSSVVTVPSSSLGTQTYIC- |
| | | NVNHKPSNTKVDKRVEPKSC |
| SEQ ID | DLL3#1 | EIVLTQSPGTLSLSPGERATLSCRASQSVSSNFLVWYQQKPGQAPR- |
| NO: 73 | chain | PLIYGASTRASGIP- |
| | | DRFSGSGSGADFTLTISRLEPEDFALYYCQQYGDSPYTFGQGTTLEIKR |
| | | TVAAPSVFIFPPSDEQLKSGTASVVCLLNNFYPREAK- |
| | | VQWKVDNALQSGNSQESVTE- |
| | | QDSKDSTYSLSSTLTLSKADYEKHKVYACEVTHQGLSSPVTKSFNRG |
| | | ECGGGGSEGKSSGSGSESKSTEGKSSGSGS- |
| | | ESKSTGGGGSQVQLVQSGAEVKKP- |
| | | GASVKVSCKASGNTFTNYYMHWVRQAPGPGLEWMGIIDPSVGSKSY |
| | | AQKFLGRVTIARDTSTSTVFLDLYS- |
| | | LRSEDTAVYFCARAGKRFGESYFDYWGQGTLVTVSSASTKGPSVFPL |
| | | APSSKSTSGGTAALGCLVKDYFPEPVTVSWNSGALTSGVHT- |
| | | FPAVLQSSGLYS- |
| | | LSSVVTVPSSSLGTQTYICNVNHKPSNTKVDKRVEPKSCDKTHTCPPC |
| | | PAPEAAGGPSVFLFPPKPKDTLMIS- |
| | | RTPEVTCVVVDVSHEDPEVKFNWYVDGVEVHNAK- |
| | | TKPREEQYNSTYRVVSVLTVLHQDWLNGKEYKCKVSNKALPAPIEK |
| | | TISKAKGQPREPQVYTLPPSREEMTKNQVSLWCLVKGFYPSDI- |
| | | AVEWESNGQPEN- |
| | | NYKTTPPVLDSDGSFFLYSKLTVDKSRWQQGNVFSCSVMHEALHNH |
| | | YTQKSLSLSPG |
| SEQ ID | DLL3#2 | DIQMTQSPSAMSASVGDRVTITCRASQGISNYLAWFQQKPGKVPEP- |
| NO: 74 | chain | LIYAAS- |
| | | SLQSGVPSRFSGSGSVTEFTLTISSLQPEDFATYYCLQHNSSPYTFGQG |
| | | TKLEIKRTVAAPSVFIFPPSDEQLKSGTASVVCLLNNFYPREAK- |
| | | VQWKVDNALQSGNSQESVTEQDSKDSTYSLSSTLTLSKADYEKHKV |
| | | YACEVTHQGLSSPVTKSFNRGECGGGGSEGKSSGSGS- |
| | | ESKSTEGKSSGSGS- |
| | | ESKSTGGGGSQVQLVQSGAEVKKPGASVKVSCKASGYTFTSYYMHW |
| | | VRQAPGQGLEWMGIINPSGGSTSYA- |
| | | QKFQGRVTMTRDTSTSTVYMELSSLRSEDTAVYYCARGEAVGGNYY |
| | | YYGMDVWGQGTTVTVSSASTKGPSVFPLAPSSKSTSGG- |
| | | TAALGCLVKDYFPEPVTVSWNSGALTSGVHTFPAVLQSSGLYSLSSV |
| | | VTVPSSSLGTQTYICNVNHKPSNTKVDKRVE- |
| | | PKSCDKTHTCPPCPAPEAAGGPSVFLF- |
| | | PPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEK- |
| | | TISKAKGQPREPQVYTLPPSREEMTKNQVSLWCLVKGFYPSDIAVEW |
| | | ESNGQPENNYKTTPPVLDSDGSFFLYSKLTVDKSRWQQGNVF- |
| | | SCSVMHEALHNHYTQKSLSLSPG |
| SEQ ID | DLL3#3 | DIQMTQSPSAMSASVGDRVTITCRASQGISNYLVWFQQKPGKAP- |
| NO: 75 | chain | KRLIYAVSS- |
| | | LYSGVPSRFSGSGSGTEFTLTISSLQPEDFATYYCLQHDSYPYTFGQGT |
| | | KLEIKRTVAAPSVFIFPPSDEQLKSGTASVVCLLNNFYPREAK- |
| | | VQWKVDNALQSGNSQESVTEQDSKDSTYSLSSTLTLSKADYEKHKV |
| | | YACEVTHQGLSSPVTKSFNRGECGGGGSEGKSSGSGS- |
| | | ESKSTEGKSSGSGS- |
| | | ESKSTGGGGSQVQLVQSGAEVKKPGASVKVSCKASGYTFTSYYVHW |
| | | VRQAPGQGLEWMVIINPGGGTTSYA- |
| | | QKFLGRVTMTRDTSTNTVYMELKSLRSEDTAVYYCARGEAVTGNYF |
| | | YYGMDVWGQGTTVTVSSASTKGPSVFPLAPSSKSTSGG- |
| | | TAALGCLVKDYFPEPVTVSWNSGALTSGVHTFPAVLQSSGLYSLSSV |
| | | VTVPSSSLGTQTYICNVNHKPSNTKVDKRVE- |
| | | PKSCDKTHTCPPCPAPEAAGGPSVFLF- |
| | | PPKPKDTLMISRTPEVTCVVVDVSHEDPEVKFNWYVDGVEVHNAKT |
| | | KPREEQYNSTYRVVSVLTVLHQDWLNGKEYKCKVSNKALPAPIEK- |
| | | TISKAKGQPREPQVYTLPPSREEMTKNQVSLWCLVKGFYPSDIAVEW |
| | | ESNGQPENNYKTTPPVLDSDGSFFLYSKLTVDKSRWQQGNVF- |
| | | SCSVMHEALHNHYTQKSLSLSPG |
| SEQ ID | CD3#1 | EAVVTQEPSLTVSPGGTVTLTCRSSTGAVTTSNYANWVQEK- |
| NO: 79 | chain | PGQLPRGLIGGTNKRAP- |
| | | WVPARFSGSLLGGKAALTLSGAQPEDEAEYFCALWYSNLWVFGGGT |
| | | KLTVLGQPKAAPSVTLFPPSSEELQANKATLVCLISDFYPGAVKVAW- |
| | | KADG- |
| | | SPVNTGVETTTPSKQSNNKYAASSYLSLTPEQWKSHRSYSCQVTHEG |
| | | STVEKTVAPAECSGGGGSEGKSSGSGSESKSTEGKSSGSGS- |
| | | ESKSTGGGGSEVQLVESGG- |
| | | GLVQPGGSLKLSCAASGFTFNTYAMNWVRQAPGKGLEWVARIRSKY |
| | | NNYATY- |
| | | YADSVKDRFTISRDDSKNTAYLQMNNLKTEDTAVYYCVRHGNFGN- |
| | | SYVSWFAY- |
| | | WGQGTLVTVSAASTKGPSVFPLAPSSKSTSGGTAALGCLVKDYFPEP |
| | | VTVSWNSGALTSGVHTFPAVLQSSGLYSLSSVVTVPSSSLGTQTYIC- |
| | | NVNHKP- |
| | | SNTKVDKRVEPKSCDKTHTCPPCPAPEAAGGPSVFLFPPKPKDTLMIS |
| | | RTPEVTCVVVDVSHEDPEVKFNWYVDGVEVHNAK- |
| | | TKPREEQYNSTYRVVSVLTVLHQDWLNGKEYKCKVSNKALPAPIEK |
| | | TISKAKGQPREPQVYTLPPSREEMTKNQVSLSCAVKGFYPSDI- |
| | | AVEWESNGQPEN- |
| | | NYKTTPPVLDSDGSFFLVSKLTVDKSRWQQGNVFSCSVMHEALHNR |
| | | FTQKSLSLSPG |
| SEQ ID | CD3#2 | EAVVTQEPSLTVSPGGTVTLTCRSSTGAVTTSNYANWVQEK- |
| NO: 80 | chain | PGQLPRGLIGGTNKRAP- |
| | | WVPARFSGSLLGGKAALTLSGAQPEDEAEYFCALWYSNLWVFGGGT |
| | | KLTVLGQPKAAPSVTLFPPSSEELQANKATLVCLISDFYPGAVKVAW- |
| | | KADG- |
| | | SPVNTGVETTTPSKQSNNKYAASSYLSLTPEQWKSHRSYSCQVTHEG |
| | | STVEKTVAPAECSGGGGSEGKSSGSGSESKSTEGKSSGSGS- |
| | | ESKSTGGGGSEVQLVESGG- |
| | | GLVQPGGSLKLSCAASGFTFNTYAMNWVRQAPGKGLEWVARIRSKY |
| | | INYATY- |
| | | YADSVKDRFTISRDDSKNTAYLQMNNLKTEDTAVYYCVRHGNFGN- |
| | | SYVSWFAY- |
| | | WGQGTLVTVSAASTKGPSVFPLAPSSKSTSGGTAALGCLVKDYFPEP |
| | | VTVSWNSGALTSGVHTFPAVLQSSGLYSLSSVVTVPSSSLGTQTYIC- |
| | | NVNHKP- |
| | | SNTKVDKRVEPKSCDKTHTCPPCPAPEAAGGPSVFLFPPKPKDTLMIS |
| | | RTPEVTCVVVDVSHEDPEVKFNWYVDGVEVHNAK- |
| | | TKPREEQYNSTYRVVSVLTVLHQDWLNGKEYKCKVSNKALPAPIEK |
| | | TISKAKGQPREPQVYTLPPSREEMTKNQVSLSCAVKGFYPSDI- |
| | | AVEWESNGQPEN- |
| | | NYKTTPPVLDSDGSFFLVSKLTVDKSRWQQGNVFSCSVMHEALHNR |
| | | FTQKSLSLSPG |
| SEQ ID | Fc domain* | DKTHTCPPCPAPELLGGPSVFLFPPKPKDTLMIS- |
| NO: 81 | (IgG1) | RTPEVTCVVVDVSHEDPEVKFNWY- |
| | | VDGVEVHNAKTKPREEQYNSTYRVVSVLTVLHQDWLNGKEYKCKV |
| | | SNKALPAPIEK- |
| | | TISKAKGQPREPQVYTLPPSREEMTKNQVSLTCLVKGFYPSDI- |
| | | AVEWESNGQPENNYKTTPPVLDSDGSFFLYSKLTVDKSRWQQGNVF |
| | | SCSVMHEALHNHYTQKSLSLSPG |
| SEQ ID | Fc W | DKTHTCPPCPAPEAAGGPSVFLFPPKPKDTLMIS- |
| NO: 82 | domain | RTPEVTCVVVDVSHEDPEVKFNWY- |
| | (IgG1, | VDGVEVHNAKTKPREEQYNSTYRVVSVLTVLHQDWLNGKEYKCKV |
| | LALA) | SNKALPAPIEK- |
| | | TISKAKGQPREPQVYTLPPSREEMTKNQVSLWCLVKGFYPSDI- |
| | | AVEWESNGQPENNYKTTPPVLDSDGSFFLYSKLTVDKSRWQQGNVF |
| | | SCSVMHEALHNHYTQKSLSLSPG |
| SEQ ID | Fc SAV | DKTHTCPPCPAPEAAGGPSVFLFPPKPKDTLMIS- |
| NO: 83 | domain | RTPEVTCVVVDVSHEDPEVKFNWY- |
| | (IgG1, | VDGVEVHNAKTKPREEQYNSTYRVVSVLTVLHQDWLNGKEYKCKV |
| | RF/LALA) | SNKALPAPIEKTISKAKGQPREPQVYTLPPSREEMTKNQVSLSCAV- |
| | | KGFYPSDI- |
| | | AVEWESNGQPENNYKTTPPVLDSDGSFFLVSKLTVDKSRWQQGNVF |
| | | SCSVMHEALHNRFTQKSLSLSPG |
| SEQ ID | Fc | ESKYGPPCPPCPAPEFLGGPSVFLFPPKPKDTLMIS- |
| NO: 84 | domain | RTPEVTCVVVDVSQEDPEVQFNWY- |
| | (IgG4Pro) | VDGVEVHNAKTKPREEQFNSTYRVVSVLTVLHQDWLNGKEYKCKV |
| | | SNKGLPSSIEKTISKAKGQPREPQVYTLPP- |
| | | SQEEMTKNQVSLTCLVKGFYPSDI- |
| | | AVEWESNGQPENNYKTTPPVLDSDGSFFLYSRLTVDKSRWQEGNVFS |
| | | CSVMHEALHNHYTQKSLSLSLG |
| SEQ ID | Fc W | ESKYGPPCPPCPAPEFLGGPSVFLFPPKPKDTLMIS- |
| NO: 85 | domain | RTPEVTCVVVDVSQEDPEVQFNWY- |
| | (IgG4Pro) | VDGVEVHNAKTKPREEQFNSTYRVVSVLTVLHQDWLNGKEYKCKV |
| | | SNKGLPSSIEKTISKAKGQPREPQVYTLPP- |
| | | SQEEMTKNQVSLWCLVKGFYPSDI- |
| | | AVEWESNGQPENNYKTTPPVLDSDGSFFLYSRLTVDKSRWQEGNVFS |
| | | CSVMHEALHNHYTQKSLSLSLG |
| SEQ ID | Fc SAV | ESKYGPPCPPCPAPEFLGGPSVFLFPPKPKDTLMIS- |
| NO: 86 | domain | RTPEVTCVVVDVSQEDPEVQFNWY- |
| | (IgG4Pro, | VDGVEVHNAKTKPREEQFNSTYRVVSVLTVLHQDWLNGKEYKCKV |
| | RF) | SNKGLPSSIEKTISKAKGQPREPQVYTLPPSQEEMTKNQVSLSCAV- |
| | | KGFYPSDI- |
| | | AVEWESNGQPENNYKTTPPVLDSDGSFFLVSRLTVDKSRWQEGNVFS |
| | | CSVMHEALHNRFTQKSLSLSLG |
| SEQ ID | constant | RTVAAPSVFIFPPSDEQLKSGTASVVCLLNNFYPREAK- |
| NO: 87 | region of | VQWKVDNALQSGNSQESVTE- |
| | a kappa | QDSKDSTYSLSSTLTLSKADYEKHKVYACEVTHQGLSSPVTKSFNRG |
| | light | EC |
| | chain | |
| SEQ ID | constant | GQPKAAPSVTLFPPSSEELQANKATLVCLISDFYPGAVKVAWKADG- |
| NO: 88 | region of | SPVNTGVETTTPSKQSNNKYAASSYLSLTPEQWKSHRSYSCQVTHEG |
| | a lambda | STVEKTVAPAECS |
| | light | |
| | chain | |
| SEQ ID | Linker | GGGGSEGKSSGSGSESKSTEGKSSGSGSESKSTGGGGS |
| NO: 89 | | |
| SEQ ID | Constant | ASTKGPSVFPLAPSSKSTSGG- |
| NO: 90 | region | TAALGCLVKDYFPEPVTVSWNSGALTSGVHT- |
| | of heavy | FPAVLQSSGLYSLSSVVTVPSSSLGTQTYICNVNHKPSNTKVDKRVEP |
| | chain CH1 | KSC |

### The Present Invention Furthermore Relates to the Following Items According to List 1

1. A T-cell engaging protein comprising a first antigen binding unit specifically binding to DLL3 and a second antigen binding unit specifically binding to CD3 for use in a method for treating a DLL3-positive cancer or a cancer selected from a Neuroendocrine Neo-plasm (NEN), Merkel cell carcinoma (MCC), medullary thyroidal carcinoma and glioma in a subject exhibiting at least a threshold of DLL3-positivity the method comprising administering to the subject a dose of said T-cell engaging protein, wherein said T-cell engaging protein comprises a first antigen binding unit specifically binding to DLL3, comprising light chain CDRs comprising the amino acid sequences of SEQ ID NO:13 (CDR1), SEQ ID NO:14 (CDR2) and SEQ ID NO:15 (CDR3) and heavy chain CDRs comprising the amino acid sequences of SEQ ID NO:16 (CDR1), SEQ ID NO:17 (CDR2) and SEQ ID NO:18 (CDR3) and a second antigen binding unit specifically binding to CD3 comprising light chain CDRs comprising the amino acid sequences of SEQ ID NO:55 (CDR1), SEQ ID NO:56 (CDR2) and SEQ ID NO:57 (CDR3) and heavy chain CDRs comprising the amino acid sequences of SEQ ID NO:58 (CDR1), SEQ ID NO:59 (CDR2) and SEQ ID NO:60 (CDR3).

2. The T-cell engaging protein for use according to item 1, the method comprising a first treatment cycle, said first treatment cycle comprising the steps of:
administering to a subject a first target dose of the T-cell engaging protein, said first target dose ranging from about 5 mg to about 70 mg,
administering to the subject a second dose of the T-cell engaging protein, said second dose ranging from about 5 mg to about 70 mg, and
administering to the subject a third dose of the T-cell engaging protein, said third dose ranging from about 5 mg to about 70 mg,
   wherein optionally the amount of all three target doses is identical.

3. The T-cell engaging protein for use according to item 2, wherein the amount of all three target doses is identical and the amount ranges from about 5 mg to about 10 mg, preferably being about 10 mg, for the first, second and third target dose, respectively.

4. The T-cell engaging protein for use according to item 2, wherein the amount of all target doses is identical, and the amount is about 30 mg for the first, second and third target dose, respectively.

5. The T-cell engaging protein for use according to item 2, wherein the amount of all target doses is identical, and the amount is about 60 mg for the first, second and third target dose, respectively.

6. The T-cell engaging protein for use according to any one of the preceding items; the method comprising a first treatment cycle, said first treatment cycle comprising the steps of:
administering to a subject a first target dose of said T-cell engaging protein, said first target dose ranging from about 80 μg/kg of the subject's body weight to about 1600 μg/kg of the subject's body weight,
administering to the subject a second target dose of said T-cell engaging protein, said second dose ranging from about 80 μg/kg of the subject's body weight to about 1600 μg/kg of the subject's body weight, and
administering to the subject a third target dose of said T-cell engaging protein, said third dose ranging from about 80 μg/kg of the subject's body weight to about 1600 μg/kg of the subject's bodyweight,
   wherein optionally the amount of all three target doses is identical.

7. The T-cell engaging protein for use according to any one of items 2 to 6, wherein said first treatment cycle is not repeated or said first treatment cycle is repeated at least once.

8. The T-cell engaging protein for use according to any one of items 2 to 7, wherein said first, second and third target doses are administered within three weeks of said first treatment cycle, wherein optionally said first target dose is administered on day 1, the second target dose is administered on day 8 and the third target dose is administered on day 15 of said first treatment cycle, wherein optionally said first treatment cycle has a duration of three weeks.

9. The T-cell engaging protein for use according to any one of items 2 to 8, wherein said first treatment cycle is followed by a second treatment cycle, said second treatment cycle comprising the step of or consisting of the step:
administering to the subject a target dose of said T-cell engaging protein, said target dose ranging from about 80 μg/kg of the subject's body weight to about 1600 μg/kg of the subject's body weight;
   wherein said second treatment cycle is optionally repeated at least once.

10. The T-cell engaging protein for use according to any one of items 2 to 9, wherein said the first treatment cycle is followed by a second treatment cycle, said second treatment cycle comprising the step of or consisting of the step:
administering to the subject a target dose of the T-cell engaging protein, said target dose ranging from about 5 mg to about 70 mg;
   wherein said second treatment cycle is optionally repeated at least once.

11. The T-cell engaging protein according to item 10, wherein the amount of said target dose is about 10 mg.

12. The T-cell engaging protein according to item 10, wherein the amount of said target dose is about 30 mg.

13. The T-cell engaging protein according to item 10, wherein the amount said target dose is about 60 mg.

14. The T-cell engaging protein for use according to item 2 to 13, wherein said second treatment cycle has a total duration of three weeks and/or said target dose is administered on day 1 of said three weeks.

15. The T-cell engaging protein for use according to any one of items 2 to 14 wherein said first treatment cycle directly follows a step-in cycle; and/or said second treatment cycle directly follows said first treatment cycle.

16. The T-cell engaging protein for use according to any one of the preceding items, wherein said Neuroendocrine Neoplasm (NEN) is a Neuroendocrine Carcinoma (NEC), preferably selected from the group consisting of Lung-NEC such as LCNEC of the lung, Extrapulmonary (ep) NEC including NEC with unknown primary site; small cell lung cancer (SCLC) including extensive-stage (ES)-SCLC, and limited-stage (LS) SCLC; or said NEN is a Neuroendocrine Tumor (NET) such as grade 3 NET, or, in the alternative epNEC excluding Neuroendocrine prostate cancer (NEPC).

17. The T-cell engaging protein for use according to any one of the preceding items, the administering comprising a step-in cycle preceding the first treatment cycle, said step-in cycle comprising the steps of:
administering to a subject an initial dose of said T-cell engaging protein, said initial dose ranging from about 5 μg/kg of the subject's body weight to about 40 μg/kg of the subject's body weight,
administering to the subject an elevated dose, which is elevated as compared to said initial dose, of said T-cell engaging protein, the elevated dose ranging from about 15 μg/kg of the subject's body weight to about 100 μg/kg of the subject's body weight, or the elevated dose ranging from about 5 mg to about 20 mg, and
administering to the subject a further dose of the T-cell engaging protein, said further dose ranging from about 80 μg/kg of the subject's body weight to about 1600 μg/kg of the subject's bodyweight, wherein said further dose is equal to or higher than said elevated dose or said further dose ranging from about 5 mg to about 70 mg.

18. The T-cell engaging protein for use according to item 17, the administering comprising a step-in cycle, said step-in cycle comprising the steps of:
administering to a subject an initial dose of said T-cell engaging protein, said initial dose ranging from about 5 μg/kg of the subject's body weight to about 15 μg/kg of the subject's body weight,
administering to the subject an elevated dose, which is elevated as compared to said initial dose, of said T-cell engaging protein, the elevated dose ranging from about 15 μg/kg of the subject's body weight to about 100 μg/kg of the subject's body weight, or the elevated dose ranging from about 5 mg to about 20 mg, and
administering to the subject a further dose of the T-cell engaging protein, said further dose ranging from about 5 mg to about 70 mg, preferably from about 5 mg to about 10 mg or from about 50 mg to about 70 mg.

19. The T-cell engaging protein for use according to item 17, the administering comprising a step-in cycle, said step-in cycle comprising the steps of:
administering to a subject an initial dose of said T-cell engaging protein, said initial dose ranging from about 20 μg/kg of the subject's body weight to about 40 μg/kg of the subject's body weight,
administering to the subject an elevated dose, which is elevated as compared to said initial dose, of said T-cell engaging protein, the elevated dose ranging from about 60 μg/kg of the subject's body weight to about 100 μg/kg of the subject's body weight,
said further dose ranging from about 80 μg/kg of the subject's body weight to about 1600 μg/kg of the subject's bodyweight, wherein said further dose is equal to or higher than said elevated dose.

20. The T-cell engaging protein for use according to item 17, the administering comprising a step-in cycle, said step-in cycle comprising the steps of:
administering to a subject an initial dose of said T-cell engaging protein, said initial dose ranging from about 5 μg/kg of the subject's body weight to about 15 μg/kg of the subject's body weight,
administering to the subject an elevated dose, which is elevated as compared to said initial dose, of said T-cell engaging protein, the elevated dose ranging from about 20 μg/kg of the subject's body weight to about 40 μg/kg of the subject's body weight,
administering to the subject a further dose of the T-cell engaging protein, said further dose ranging from about 80 μg/kg of the subject's body weight to about 1600 μg/kg of the subject's bodyweight, wherein said further dose is equal to or higher than said elevated dose.

21. The T-cell engaging protein for use according to item 17, the administering comprising a step-in cycle, said step-in cycle comprising the steps of:
administering to a subject an initial dose of said T-cell engaging protein, said initial dose ranging from about 20 μg/kg of the subject's body weight to about 40 μg/kg of the subject's body weight,
administering to the subject an elevated dose, which is elevated as compared to said initial dose, of said T-cell engaging protein, the elevated dose ranging from about 60 μg/kg of the subject's body weight to about 100 μg/kg of the subject's body weight,
administering to the subject a further dose of the T-cell engaging protein, said further dose ranging from about 5 mg to about 70 mg.

22. The T-cell engaging protein for use according to item 17, the administering comprising a step-in cycle, said step-in cycle comprising the steps of:
administering to a subject an initial dose of said T-cell engaging protein, said initial dose ranging from about 5 μg/kg of the subject's body weight to about 15 μg/kg of the subject's body weight,
administering to the subject an elevated dose, which is elevated as compared to said initial dose, of said T-cell engaging protein, the elevated dose ranging from about 20 μg/kg of the subject's body weight to about 40 μg/kg of the subject's body weight,
administering to the subject a further dose of the T-cell engaging protein, said further dose ranging from about 5 mg to about 70 mg.

23. The T-cell engaging protein for use according to item 21, wherein said initial dose ranges from about 5 μg/kg of the subject's body weight to about 15 μg/kg of the subject's body weight;
said elevated dose ranges from about 55 μg/kg of the subject's body weight to about 95 μg/kg of the subject's body weight, and optionally said elevated dose is about 90 μg/kg of the subject's body weight; and
said further dose ranges from about 5 mg to about 15 mg, or about 20 mg to about 40 mg, about 50 mg to about 70 mg.

24. The T-cell engaging protein for use according to item 21,
wherein said initial dose is about 30 μg/kg;
said elevated dose is about 90 μg/kg; and
said further dose is about 10 mg.

25. The T-cell engaging protein for use according to item 21,
wherein said initial dose is about 30 μg/kg;
said elevated dose is about 90 μg/kg; and
said further dose is about 30 mg.

26. The T-cell engaging protein for use according to item 21,
wherein said initial dose is about 30 μg/kg;
said elevated dose is about 90 μg/kg; and
said further dose is about 60 mg.

27. The T-cell engaging protein for use according to item 20,
wherein said initial dose is about 10 μg/kg;
said elevated dose is about 30 μg/kg; and
said further dose is about 90 μg/kg.

28. The T-cell engaging protein for use according to item 22,
wherein said initial dose is about 10 μg/kg;
said elevated dose is about 30 μg/kg; and
said further dose is about 30 mg.

29. The T-cell engaging protein for use according to item 22,
wherein said initial dose is about 10 μg/kg;
said elevated dose is about 30 μg/kg; and
said further dose is about 60 mg.

30. The T-cell engaging protein for use according to item 22,
wherein said initial dose is about 10 μg/kg;
said elevated dose is about 60 μg/kg; and
said further dose is about 10 mg.

31. The T-cell engaging protein for use according to item 22,
wherein said initial dose is about 10 μg/kg;
said elevated dose is about 60 μg/kg; and
said further dose is about 60 mg.

32. The T-cell engaging protein for use according to item 22,
wherein said initial dose is about 10 μg/kg;
said elevated dose is about 10 mg; and
said further dose is about 60 mg.

33. The T-cell engaging protein for use according to item 17, wherein said initial dose
is about 10 μg/kg of the subject's body weight;
said elevated dose is about 90 μg/kg of the subject's body weight; and
said further dose is about is 10 mg.

34. The T-cell engaging protein for use according to item 19, wherein said initial dose ranges from about 25 μg/kg of the subject's body weight to about 35 μg/kg of the subject's body weight, and optionally said initial dose is about 30 μg/kg of the subject's body weight;
said elevated dose ranges from about 85 μg/kg of the subject's body weight to about 95 μg/kg of the subject's body weight, and optionally said elevated dose is about 90 μg/kg of the subject's body weight; and
said further dose ranges from about 85 μg/kg of the subject's body weight to about 1550 μg/kg of the subject's body weight, and optionally the further dose is about 90 μg/kg, about 180 μg/kg, about 270 μg/kg, about 360 μg/kg, about 540 μg/kg, about 720 μg/kg, about 1080 μg/kg or about 1530 μg/kg of the subject's body weight.

35. The T-cell engaging protein for use according to item 17, said initial dose ranging from about 5 μg/kg of the subject's body weight to about 15 μg/kg of the subject's body weight,
the elevated dose ranging from about 20 μg/kg of the subject's body weight to about 40 μg/kg of the subject's body weight; and
said further dose ranges from about 85 μg/kg of the subject's body weight to about 1550 μg/kg of the subject's body weight, and optionally the further dose is about 90 μg/kg, about 180 μg/kg, about 270 μg/kg, about 360 μg/kg, about 540 μg/kg, about 720 μg/kg, about 1080 μg/kg or about 1530 μg/kg, preferably 90 μg/kg, of the subject's body weight.

36. The T-cell engaging protein for use according to any preceding item,
the administering comprising a step-in cycle, the step-in cycle comprising the steps of:
   administering to the subject an initial dose, an elevated dose, which is elevated as compared to said initial dose, and a further dose of said T-cell engaging protein, wherein a ratio between said initial, elevated and further dose per kilogram of the subject's body weight is about 1:about 3:about 9.

37. The T-cell engaging protein for use according to item 17, wherein at least one of said first, second or third target doses of the first treatment cycle is about 5 mg, about 10 mg, about 20 mg, about 30 mg, about 40 mg, about 50 mg, about 60 mg, or about 70 mg, optionally about 10 mg or about 60 mg.

38. The T-cell engaging protein for use according to any preceding item, wherein said T-cell engaging protein comprises immunoglobulin (Ig) domains, preferably 12 Ig domains.

39. The T-cell engaging protein for use according to item 38, wherein said Ig domains are comprised in two polypeptide chains, wherein each polypeptide chain comprises six Ig domains.

40. The T-cell engaging protein for use according any preceding item, wherein the subject has been determined to exhibit at least a threshold of DLL3-positivity.

41. The T-cell engaging protein for use according to any of the preceding claims, comprising the step of determining or having determined that the subject exhibits at least a threshold of DLL3-positivity.

42. The T-cell engaging protein for use according to any of the preceding claims, wherein the threshold is at least about 10%, at least about 20% at least about 25%, at least about 30%, at least about 40%, at least about 50% or at least about 75%, most optionally at least 50% or at about 50%.

43. The T-cell engaging protein for use according to any of the preceding claims, wherein the threshold is at least about 25%, at least about 50%, or at least about 75%.

44. The T-cell engaging protein for use according any one of the preceding items, wherein the DLL3 positivity in a sample is calculated according to the following formula:

$$DLL 3 - positivity [ \% ] = \frac{DLL 3 - positive tumor cells in the sample}{\begin{matrix}( DLL 3 - positive tumors cells in the sample ) + \\ ( DLL 3 - negative tumor cells in the sample )\end{matrix}} .$$

45. The T-cell engaging protein for use according to item 44, wherein the DLL3-positive tumor cell exhibits moderate to strong membranous and/or cytoplasmic staining.

46. The T-cell engaging protein for use according to any one of the preceding items, wherein DLL3 positivity is determined using an immunohistochemical (IHC) assay.

47. The T-cell engaging protein for use according to any one of the preceding items, wherein DLL3 positivity is determined in a tissue sample from the subject, preferably in a formalin fixed paraffin embedded (FFPE) tissue sample.

48. The T-cell engaging protein for use according to any one of the preceding items, wherein at least one further anti-cancer agent is to be administered, said further anti-cancer agent preferably being a chemotherapeutic agent and/or an immune-checkpoint inhibitor such as an anti-PD-1 antibody or an anti-PD-L1 antibody.

49. The T-cell engaging protein for use according to any one the preceding items, wherein a premedication comprising a non-steroidal anti-inflammatory drug, such as acetaminophen, an anti-histamine and/or a steroid, preferably a corticosteroid such as prednisolone or dexamethasone, prior to at least one of the doses, optionally prior to each dose, such as any target dose or any step-in dose, wherein prior to optionally means that the premedication is administered about 0.25 to about 2 hours, such as about 0.5 hours to about 1 hour, prior to the respective point in time of the start of the administration of a respective dose of the T-cell engaging protein.

50. The T-cell engaging protein for use according to one of the preceding items, wherein the subject has been treated with a chemotherapy involving administration of a cytostatic agent prior to the treatment with the T-cell engaging protein, optionally for 1 or 2 cycles prior to the treatment with the T cell engaging protein.

51. The T-cell engaging protein for use according to any one the preceding items, wherein an occurrence of cytokine release syndrome is to be treated with an anti-cytokine release syndrome agent, preferably selected from the group consisting of a corticosteroid, an IL-6 agonist and an IL-6R antagonist.

52. The T-cell engaging protein for use according to item 39, wherein said T-cell engaging protein comprises (i) a first antigen binding unit specifically binding to DLL3 comprising a light chain variable domain comprising the amino acid sequence of SEQ ID NO:41 and heavy chain variable domain comprising the amino acid sequence of SEQ ID NO:42 and (ii) a second antigen binding unit specifically binding to CD3 comprising a light chain variable domain comprising the amino acid sequence of SEQ ID NO:67 and a heavy chain variable domain comprising the amino acid sequence of SEQ ID NO:68.

53. The T-cell engaging protein for use according to item 52, wherein said T-cell engaging protein comprises a first single chain Fab forming a first antigen binding unit comprising the sequence of SEQ ID NO:51 and a second single chain Fab comprising the sequence of SEQ ID NO:71 forming a second antigen binding unit.

54. The T-cell engaging protein for use according to any one of the preceding items, wherein said T-cell engaging protein comprises of a first polypeptide chain specific for DLL3 comprising an amino acid sequence of SEQ ID NO:75 and a second polypeptide chain specific for CD3 comprising the amino acid sequence of SEQ ID NO:79.

55. The T-cell engaging protein for use according to any one the preceding items, wherein essentially no cytokine release syndrome occurs after said initial and/or said elevated dose of a step-in cycle in the subject.

56. The T-cell engaging protein for use according to any one of the preceding items, wherein said T-cell engaging protein is the only anti-cancer agent to be administered.

57. The T-cell engaging protein for use according to any one of the preceding items, wherein at least one further anti-cancer agent is to be administered, said further anti-cancer agent preferably being carboplatin or cisplatin and etoposide and/or an anti-PD-L1 antibody, more preferably the anti-PD-L1 antibody is atezolizumab or durvalumab.

58. The T-cell engaging protein for use according to any one of the preceding items, the method further comprising administering to the subject a dose of lurbinectedin.

59. The T-cell engaging protein for use according to any one of the preceding items, the method further comprising administering to the subject a dose of amrubicin.

60. The T-cell engaging protein for use according to any one of the preceding items, comprising a step of co-administering topotecan to the subject, preferably for use in the treatment of SCLC (any treatment line) including ES-SCLC, preferably second line (2L) treatment of ES-SCLC that progressed or recurred following Standard of Care (SoC) treatment and LS-SCLC, preferably first line (1L) treatment.

61. The T-cell engaging protein for use according to any one of the preceding items, comprising a step of co-administering lurbinectedin to the subject, preferably for use in the treatment of SCLC (any treatment line) including ES-SCLC, preferably 2L treatment of ES-SCLC that progressed or recurred following SoC, and LS-SCLC, preferably 1L treatment.

62. The T-cell engaging protein for use according to any one of the preceding items, comprising a step of co-administering amrubicin to the subject, preferably for use in the treatment of SCLC (any treatment line) including ES-SCLC, preferably 2L treatment of ES-SCLC that progressed or recurred following SoC, and LS-SCLC, preferably 1L treatment.

63. The T-cell engaging protein for use according to any one of the preceding items, comprising a step of co-administering etoposide and carboplatin or cisplatin, and optionally also anti-PD-L1 antibody e.g. atezolizumab or durvalumab, to the subject, preferably for use in the treatment of SCLC including ES-SCLC, especially for 1L, 2L and/or 3L treatment, and LS-SCLC (any treatment line).

64. The T-cell engaging protein for use according to any one of the preceding items, comprising a step of co-administering etoposide and carboplatin or cisplatin (optionally also anti-PD-L1 antibody), to the subject, preferably for use in the treatment of epNEC or LCNEC of the lung (any treatment line), preferably first line treatment of epNEC or LCNEC of the lung.

65. A method for selecting a subject suffering from a DLL3-positive cancer or a cancer selected from a Neuroendocrine Neoplasm (NEN), Merkel cell carcinoma (MCC), medullary thyroidal carcinoma and glioma for treatment with a T-cell engaging protein comprising a first antigen binding unit specifically binding to DLL3 and a second antigen binding unit specifically binding to CD3, the method comprising:
(a) determining or having determined in a sample that the subject exhibits a DLL3-positivity,
wherein the subject is selected for treatment with said T-cell engaging protein when the DLL3-positivity exhibits at least a threshold, and
wherein said T-cell engaging protein comprises a first antigen binding unit specifically binding to DLL3, comprising light chain CDRs comprising the amino acid sequences of SEQ ID NO:13 (CDR1), SEQ ID NO:14 (CDR2) and SEQ ID NO:15 (CDR3) and heavy chain CDRs comprising the amino acid sequences of SEQ ID NO:16 (CDR1), SEQ ID NO:17 (CDR2) and SEQ ID NO:18 (CDR3) and a second antigen binding unit specifically binding to CD3 comprising light chain CDRs comprising the amino acid sequences of SEQ ID NO:55 (CDR1), SEQ ID NO:56 (CDR2) and SEQ ID NO:57 (CDR3) and heavy chain CDRs comprising the amino acid sequences of SEQ ID NO:58 (CDR1), SEQ ID NO:59 (CDR2) and SEQ ID NO:60 (CDR3).

66. The method according to item 65, wherein the DLL-3 positivity is calculated according to the following formula:

$$DLL 3 - positivity [ \% ] = \frac{DLL 3 - positive tumor cells in the sample}{\begin{matrix}( DLL 3 - positive tumors cells in the sample ) + \\ ( DLL 3 - negative tumor cells in the sample )\end{matrix}} .$$

67. The method according to 65 or 66, wherein the threshold is at least 10%, at least about 20% at least about 25%, at least about 30%, at least about 40%, at least about 50% or at least 75%, most optionally at least about 50%.

68. The method according to any one of 65 to 67, wherein the threshold is at least about 25%, at least about 50%, or at least about 75%, most optionally at least about 50%.

69 The method according to any items 65 to 68, wherein the number of DLL3-positive tumor cells in the sample is determined by counting immunostained tumor cells in the sample.

70. The T-cell engaging protein for use according to any one of items 1 to 64 or the method according to any one of items 65 to 69, wherein the subject is a human.

71. The T-cell engaging protein for use according to any one of items 1 to 64, or the method according to any one of items 65 to 69, wherein said T-cell engaging protein is administered by intravenous (IV) infusion.

72. The T-cell engaging protein for use according to item 63 or 64, wherein the T-cell engaging protein, carboplatin or cisplatin and etoposide are co-administered for 3 to 5 treatment cycles, involving a step in cycle.

73. The T-cell engaging protein for use according to item 72, wherein only the T-cell engaging protein will be administered after said 3-5 treatment cycles, in particular for a maximum of 36 months.

74. The T-cell engaging protein for use according to any one of items 1 to 64 or 70 to 73, wherein the cancer is epNEC, optionally epNEC excluding NEPC.

75. The T-cell engaging protein for use according to any one of items 1 to 64 or 70 to 73, wherein the cancer is SCLC.

76. The T-cell engaging protein for use according to any one of items 1 to 64 or 70 to 73, wherein the cancer is LCNEC of the lung.

77. The T-cell engaging protein for use according to any one of items 1 to 64 or 70 to 73, wherein the cancer is glioma.

78. The T-cell engaging protein for use according to any one of items 1 to 64 or 70 to 73, wherein the cancer is NEC.

79. The T-cell engaging protein for use according to any one of items 1 to 64 or 70 to 73, wherein the cancer is NEPC.

80. The T-cell engaging protein for use according to any one of items 1 to 64 or 70 to 79, wherein carboplatin and cisplatin and etoposide, optionally also anti-PD-L1 antibody, are co-administered for 3 to 5 treatment cycles, in-volving a step in cycle, wherein more preferably only the T-cell engaging protein, optionally together with anti-PD-L1 antibody, is be administered after said 3-5 treatment cycles, in particular for a maximum of 36 months, wherein further optionally, these cycles may involve the step-in cycle for each regimen as disclosed above.

81. The T-cell engaging protein for use according to any one of items 1 to 64 or 70 to 80, wherein one or more treatment cycles (e.g. one or two treatment cycles) include only chemotherapy taking place prior to any of the co-administration treatments with the T-cell engaging protein.

82. The T-cell engaging protein for use according to any one of items 1 to 64 or 70 to 81, wherein lurbinectedin, if co-administered is administered at the approved regimen of 3.2 mg/m² on day 1 of each 21 day cycle as an intravenous infusion, especially for 60 minutes.

83. The T-cell engaging protein for use according to any one of items 1 to 64 or 70 to 82, wherein amrubicin, if co-administered may optionally be administered at the approved regimen of 40 mg/m² on days 1 to 3 of each 21 day cycle, wherein optionally amrubicin may be administered as an intravenous infusion.

84. The T-cell engaging protein for use according to any one of items 1 to 64 or 70 to 83, wherein topotecan, if co-administered may optionally be administered at the approved regimen of 2.3 mg/m² orally (p.o.) or 1.5 mg/m² by intravenous infusion daily on day 1 to day 5 during each 21 day cycle.

85. The T-cell engaging protein for use according to any one of items 1 to 64 or 70 to 84, wherein carboplatin and etoposide, if co-administered may optionally be administered at the approved regimen of AUC 5 mg/mL min (carboplatin) and 100 mg/m² (etoposide), wherein carboplatin is administered at day 1 during each 21 day cycle and etoposide is administered on days 1 to 3 during each 21 day cycle.

86. The T-cell engaging protein for use according to any one of items 1 to 64 or 70 to 85, wherein cisplatin and etoposide, if co-administered may optionally be administered at the approved regimen of 75 mg/m² (cisplatin) and 100 mg/m2 (etoposide), wherein cisplatin is administered at day 1 during each 21 day cycle and etoposide is administered on days 1 to 3 during each 21 day cycle.

87. The T-cell engaging protein for use according to any one of the preceding items, wherein a post-medication is administered directly after the administration of the T-cell engaging protein, wherein optionally “directly after” means that the post-medication is administered following the respective point in time of the start of the administration of a respective dose of the T-cell engaging protein.

88. The T-cell engaging protein for use according to item 87, wherein the post-medication comprises a non-steroidal anti-inflammatory drug, such as acetaminophen, an anti-histamine and/or a steroid, such as prednisolone or dexamethasone.

89. The T-cell engaging protein for use according to any one of item 87 to 88, wherein the post-medication comprises the same or a different substance(s) as compared to the premedication according to item 49.

90. The T-cell engaging protein for use according to any one of items 87 to 89, wherein the post-medication is administered orally (p.o.) or intravenously (i.v).

91. The T-cell engaging protein for use according to any one of items 49 and 87 to 90, wherein the pre-medication is administered orally (p.o.) or intravenously (i.v.).

92. The T-Cell engaging protein for use according to any one of items 49 and 87 to 91, wherein the post-medication is administered at least 6 hours up to 12 hours, such as 8±2 hours, after the respective administration of a dose of the T-cell engaging protein, wherein optionally “after” means that the post-medication is administered at least 6 hours up to about 12 hours, such as 8±2 hours, following the respective point in time of the start of the administration of a respective dose of the T-cell engaging protein.

93. The T-cell engaging protein for use according to any one of items 49 and 87 to 92, wherein the premedication is administered 0.25 to 2 hours, such as 0.5 hours to about 1 hour, prior to the respective administration of the T-cell engaging protein, wherein optionally “prior” means that the premedication is administered 0.25 to 2 hours, such as about 0.5 hours to about 1 hour, prior to the respective point in time of the start of the administration of a respective dose of the T-cell engaging protein.

94. The T-cell engaging protein for use according to any one of items 49 and 87 to 92, wherein the pre- and/or or post-medication comprises or is a steroid 95. The T-cell engaging protein according to item 94, wherein the steroid is a corticosteroid.

96. The T-cell engaging protein for use according to item 95, wherein the corticosteroid is selected from the group consisting of prednisolone and dexamethasone or a combination thereof.

97. The T-cell engaging protein for use according to item 96, wherein dexamethasone is comprised in the premedication and/or post-medication.

98. The T-cell engaging protein for use according to any one of items 49 and 87 to 92, wherein the steroid is selected from the group consisting of cortisone, betamethasone, triamcinolone, hydrocortisone, hydrocortisone, prednisone, methylprednisolone, dexamethasone or combinations thereof.

99. The T-cell engaging protein for use according to any of items 49 and 87 to 98, wherein an amount of a dexamethasone dose comprised in a premedication ranges from about 10 mg to about 18 mg, such as about 16 mg.

100. The T-cell engaging protein for use according to any of items 49 and 87 to 99, wherein an amount of a dexamethasone dose comprised in a premedication ranges from about 4 mg to about 10 mg, such as about 8 mg.

101. The T-cell engaging protein for use according to one any of items 49 and 87 to 100, wherein an amount of a dexamethasone dose comprised in a post-medication ranges from about 4 mg to about 10 mg, such as about 8 mg.

102. The T-cell engaging protein for use according to any one of items 17 to 101, wherein a premedication is administered directly prior the initial dose and a post-medication is administered directly after the initial dose, wherein the pre-medication is preferably administered about 0.25 to about 2 hours, such as 0.5 hours to about 1 hour before the initial dose and/or the post-medication is preferably administered at least 6 hours up to 12 hours, such as 8±2 hours, after the initial dose, wherein optionally “directly prior” means that the premedication is administered 0.25 to 2 hours, such as about 0.5 hours to about 1 hour, prior to the respective point in time of the start of the administration of the initial dose of the T-cell engaging protein and optionally “directly after” means that the post-medication is administered at least 6 hours up to about 12 hours, such as 8±2 hours, following the respective point in time of the start of the administration of the initial dose of the T-cell engaging protein.

103. The T-cell engaging protein for use according to any one of items 17 to 102, wherein a premedication is administered directly prior the elevated dose and a post-medication is administered directly after the elevated dose, wherein the pre-medication is preferably administered about 0.25 to about 2 hours, such as 0.5 hours to about 1 hour before the elevated dose and/or the post-medication is preferably administered at least 6 hours up to 12 hours, such as 8±2 hours, after the elevated dose, wherein optionally “directly prior” means that the premedication is administered 0.25 to 2 hours, such as about 0.5 hours to about 1 hour, prior to the respective point in time of the start of the administration of the elevated dose of the T-cell engaging protein and optionally directly after” means that the post-medication is administered at least 6 hours up to about 12 hours, such as 8±2 hours, following the respective point in time of the start of the administration of the elevated dose of the T-cell engaging protein.

104. The T-cell engaging protein for use according to any one of items 17 to 103, wherein a premedication is administered directly prior the further dose and a post-medication is administered directly after the further dose, wherein the pre-medication is preferably administered about 0.25 to about 2 hours, such as 0.5 hours to about 1 hour before the further dose and/or the post-medication is preferably administered at least 6 hours up to 12 hours, such as 8±2 hours, after the further dose, wherein optionally “directly prior” means that the premedication is administered 0.25 to 2 hours, such as about 0.5 hours to about 1 hour, prior to the respective point in time of the start of the administration of the further dose of the T-cell engaging protein and optionally “directly after” means that the post-medication is administered at least 6 hours up to about 12 hours, such as 8±2 hours, following the respective point in time of the start of the administration of the further dose of the T-cell engaging protein.

105. The T-cell engaging protein for use according to any one of items 49 and 87 to 104, wherein the antihistamine of the premedication and/or the post-medication is diphenylhydramine, such as in an amount of about 50 mg.

106. The T-cell engaging protein for use according to any one of items 49 and 87 to 105, wherein the non-steroidal anti-inflammatory drug of the premedication and/or the post-medication is acetaminophen/paracetamol, such as in an amount of about 1000 mg.

107. The T-cell engaging protein for use according to any one of items 17 to 106, wherein a hospitalization, such as for 24 hours, follows the initial, elevated and/or further doses, i.e. the step-in doses, and/or the first target dose of the T-cell engaging protein.

108. The T-cell engaging protein for use according to any one of items 17 to 107, wherein a hospitalization, such as for 24 hours, follows the initial and elevated doses, i.e. the first two step-in doses, of the T-cell engaging protein.

109. The T-cell engaging protein for use according to any one of items 17 to 108, wherein no hospitalization follows after the initial, elevated and further dose, i.e. the step-in doses, and the target dose(s) of the T-cell engaging protein.

110. The T-cell engaging protein for use according to any one of items 49 and 87 to 109, wherein the premedication and/or the post-medication comprise different compounds.

111. The T-cell engaging protein for use according to any one of items 49 and 87 to 110, wherein the premedication and/or the post-medication comprise an inhibitor/agonist of TNF-alpha (TNF) and/or TNF-alpha-Receptor (TNFR) reducing TNF/TFNR signalling and/or an inhibitor/agonist of IL6/IL6R that reduces IL6/IL6R signalling.

112. The T-cell engaging protein for use according to item 111, wherein the inhibitor/agonist of IL6/IL6R that reduces IL6/IL6R signaling is tocilizumab.

113. The T-cell engaging protein for use according to any one of items 49 and 87 to 112, wherein the premedication and/or post-medication comprise a steroid, wherein the steroid is selected from the group consisting of cortisone, betamethasone, triamcinolone, hydrocortisone, hydrocortisone, prednisone, methylprednisolone, dexamethasone or combinations thereof.

114. The T-cell engaging protein for use according to any one of items 49 and 87 to 113, wherein the premedication and/or the post-medication comprise an inhibitor/agonist of TNF/TNFR that reduces TNF/TNFR signaling being selected from the group consisting of etanercept, infliximab, adalimumab, certolizumab pegol and golimumab or a combination thereof.

115. The T-cell engaging protein for use according to item 114, wherein inhibitor/agonist of IL6/IL6R that reduces IL6/IL6R signalling is tocilizumab.

116. The T-cell engaging protein for use according to any one of items 49 and 77 to 115, wherein the premedication and/or the post-medication comprises a non-steroidal anti-inflammatory drug, an antihistamine and a steroid.

117. The T-cell engaging protein for use according to item 116, wherein the non-steroidal anti-inflammatory drug is acetaminophen/paracetamol, such as about 1000 mg p.o. or i.v., or equivalent, diphenhydramine, such as about 50 mg p.o. or i.v., or equivalent, dexamethasone, such as about 16 mg or about 8 mg p.o. or i.v., or equivalent intermediate-acting corticosteroid.

118. The T-cell engaging protein for use according to any one of items 17 to 117, wherein the initial dose amount of the T-cell engaging protein is about 10 μg/kg and the elevated dose amount of the T-cell engaging protein is about 30 μg/kg, and wherein prior to and after those initial and elevated doses, a dexamethasone premedication and post-medication of a dose amount of about 8 mg is administered, respectively.

119. The T-cell engaging protein for use according to any one of items 49 and 17 to 118, wherein the premedication comprises a non-steroidal anti-inflammatory drug, such as acetaminophen/paracetamol, and an antihistamine, such as diphenhydramine, and optionally a steroid.

120. The T-cell engaging protein for use according to item 119, wherein the premedication is administered prior to the respective step-in doses of the T-cell engaging protein, such as the initial dose, e.g. being about 10 μg/kg, such as the elevated dose, e.g. being about 30 μg/kg, and such as the further dose, e.g. being about 90 μg/kg.

121. The T-cell engaging protein for use according to item 119 or 120, wherein premedication may also be administered prior to the first to at least 6th or more target doses of the T-cell engaging protein, e.g. being about 60 mg of the T-cell engaging protein.

122. The T-cell engaging protein for use according to any one of items 119 to 121, wherein a steroid, such as dexamethasone, is administered as premedication in addition to the non-steroidal anti-inflammatory drug and the antihistamine and as a post-medication.

123. The T-cell engaging protein for use according to any one of items 119 to 122, wherein prior to each of the initial dose, the elevated dose and the further dose and the first two target doses of the T-cell engaging protein, the steroid, such as dexamethasone, is administered as a premedication.

124. The T-cell engaging protein for use according to item 123, wherein about 8 mg of the steroid being dexamethasone is respectively administered prior to the initial dose, the elevated dose, and the further dose of the T-cell engaging protein as premedication and about 8 mg of the steroid being dexamethasone is respectively administered after the initial dose, the elevated dose and the further dose of the T-cell engaging protein as post-medication.

125. The T-cell engaging protein according to item 123 or 124, wherein prior to the first target dose of the T-cell engaging protein about 4 mg of dexamethasone is administered as a premedication and after the first target dose of the T-cell engaging protein about 4 mg of dexamethasone is administered as a post-medication.

126. The T-cell engaging protein according to any one of items 119 to 125, wherein after the initial dose, the elevated dose, the further dose and the first target dose of the T-cell engaging protein, the steroid, such as dexamethasone, is administered, e.g. in an amount of about 8 mg, respectively.

127. The T-cell engaging protein according to any one of items 119 to 126, wherein the amount of the steroid, such as dexamethasone, is reduced at the first target dose of the T-cell engaging protein for the premedication and/or post-medication, if the T-cell engaging protein is well-tolerated, i.e. if no CRS≥2 and/or ICANS (any grade) occurred following the prior dose.

128. The T-cell engaging protein according to any one of items 119 to 127, wherein the steroid, such as dexamethasone, is omitted for the premedication and the post-medication starting from the second target dose of the T-cell engaging protein.

129. The T-cell engaging protein according to any one of items 119 to 128, wherein no more steroid, such as dexamethasone, is administered as a premedication from the third target dose of the T-cell engaging protein, if it is well tolerated.

130. The T-cell engaging protein for use according to any one of items 119 to 129, wherein if CRS/IRR/ICANS occur, the steroid, e.g. dexamethasone, dose for at least premedication at the next drug administration must remain the same as for the current dose and must not be reduced.

131. The T-cell engaging protein for use according to any one of items 119 to 130, wherein the line of treatment is 1L in DLL3 positive subjects and optionally, those subjects have unresectable locally advanced or metastatic epNEC, wherein, the co-administration involves—in addition to the T-cell engaging protein—an administration of carboplatin and etoposide.

132. The T-cell engaging protein for use according to any one of items 119 to 130, wherein the treatment is 1L and the co-administering—in addition to the T-cell engaging protein—involves administering atezolizumab, carboplatin and etoposide, and wherein the cancer is SCLC, such as ES-SCLC.

133. The T-cell engaging protein for use according to any one of items 49 and 87 to 132, wherein a premedication is administered directly prior the first target dose and a post-medication is administered directly after the first target dose, wherein the pre-medication is preferably administered about 0.25 to about 2 hours, such as 0.5 hours to about 1 hour before the first target dose and/or the post-medication is preferably administered at least 6 hours up to 12 hours, such as 8±2 hours, after the first target dose, wherein optionally “directly prior” means that the premedication is administered 0.25 to 2 hours, such as about 0.5 hours to about 1 hour, prior to the respective point in time of the start of the administration of the first target dose of the T-cell engaging protein and optionally “directly after” means that the post-medication is administered at least 6 hours up to about 12 hours, such as 8±2 hours, following the respective point in time of the start of the administration of the first target dose of the T-cell engaging protein.

### The Present Invention Furthermore Relates to the Following Items According to List 2

1. A T-cell engaging protein comprising a first antigen binding unit that specifically binds to DLL3 and a second antigen binding unit that specifically binds to CD3 for use in a method of treatment of a DLL3-positive cancer or a cancer selected from a Neuroendocrine Neoplasm (NEN), Merkel cell carcinoma (MCC), medullary thyroidal carcinoma and glioma,
the method comprising a step-in cycle, said step-in cycle comprising the steps of:
   administering to a subject an initial dose of said T-cell engaging protein, said initial dose ranging from about 20 μg/kg of the subject's body weight to about 40 μg/kg of the subject's body weight,
   administering to the subject an elevated dose, which is elevated as compared to said initial dose, of said T-cell engaging protein, the elevated dose ranging from about 60 μg/kg of the subject's body weight to about 100 μg/kg of the subject's body weight,
   administering to the subject a further dose of the T-cell engaging protein, said further dose ranging from about 5 mg to about 70 mg.

2. The T-cell engaging protein for use according to item, wherein said further dose is equal to a target dose, said target dose being the dose to be administered during a first treatment cycle which optionally directly follows said step-in cycle.

3. The T-cell engaging protein for use according to items 1 or 2, wherein
said initial dose, said elevated dose and/or said further is/are administered once; or
said initial dose, said elevated dose and/or said further is/are administered more than once, optionally 2 to 10 times,
wherein optionally the number of administrations is determined based on treatment tolerability including presence and/or degree of a cytokine release syndrome (CRS) and/or of an immune effector cell-associated neurotoxicity syndrome (ICANS),
and wherein further optionally said elevated dose follows the initial dose only if said CRS, to the extent determined, is rated grade 1 or less in terms of NCI CTC grade and/or if said ICANS, to the extend determined, is rated grade 1 or less, and/or wherein further optionally said further dose follows the elevated dose only if said CRS, to the extent determined, is rated grade 1 or less in terms of NCI CTC grade and/or if said ICANS, to the extend determined, is rated grade 1 or less.

4. The T-cell engaging protein for use according to any one of the preceding items, wherein said initial dose is administered three times to ten times within the step-in cycle.

5. The T-cell engaging protein for use according to any one of the preceding items, wherein said initial dose is administered three times within three weeks, optionally on day 1, day 8 and day 15 of the three weeks.

6. The T-cell engaging protein for use according to any one of the preceding items, wherein said step-in cycle has a duration of at least three weeks.

7. The T-cell engaging protein for use according to any one of the preceding items, wherein said initial, elevated and further doses are administered within three weeks, said initial dose being administered once, wherein optionally said initial dose is administered on day 1, said elevated dose is administered on day 8 and said further dose is administered on day 15 of said step-in cycle.

8. The T-cell engaging protein for use according to any one of the preceding items, wherein said initial dose ranges from about 25 μg/kg of the subject's body weight to about 35 μg/kg of the subject's body weight, and optionally said initial dose is about 30 μg/kg of the subject's body weight;
said elevated dose ranges from about 85 μg/kg of the subject's body weight to about 95 μg/kg of the subject's body weight, and optionally said elevated dose is about 90 μg/kg of the subject's body weight; and
said further dose ranges from about 5 mg to about 70 mg, and optionally said further dose is about 10 mg, about 30 mg or about 60 mg.

9. The T-cell engaging protein for use according to any one of the preceding items; the method further comprising a first treatment cycle following the step-in cycle, said first treatment cycle comprising the steps of:
administering to a subject a first target dose of said T-cell engaging protein, said first target dose ranging from about 80 μg/kg of the subject's body weight to about 1600 μg/kg of the subject's body weight,
administering to the subject a second target dose of said T-cell engaging protein, said second dose ranging from about 80 μg/kg of the subject's body weight to about 1600 μg/kg of the subject's body weight, and
administering to the subject a third target dose of said T-cell engaging protein, said third dose ranging from about 80 μg/kg of the subject's body weight to about 1600 μg/kg of the subject's bodyweight,
   wherein optionally the amount of all three target doses is identical.

10. The T-cell engaging protein for use according to any one of the preceding items, the method further comprising a first treatment cycle following said step-in cycle, said first treatment cycle comprising the steps of:
administering to a subject a first target dose of the T-cell engaging protein, said first target dose ranging from about 5 mg to about 70 mg,
administering to the subject a second dose of the T-cell engaging protein, said second dose ranging from about 5 mg to about 70 mg, and
administering to the subject a third dose of the T-cell engaging protein, said third dose ranging from about 5 mg to about 70 mg,
   wherein optionally the amount of all three target doses is identical.

11. The T-cell engaging protein for use according to item 10, wherein the amount of all three target doses is identical and the amount ranges from about 5 mg to about 10 mg, preferably being about 10 mg, for the first, second and third target dose, respectively.

12. The T-cell engaging protein for use according to item 10, wherein the amount of all target doses is identical, and the amount is about 60 mg for the first, second and third target dose, respectively.

13. The T-cell engaging protein for use according to item 10, wherein the amount of all target doses is identical, and the amount is about 30 mg for the first, second and third target dose, respectively.

14. The T-cell engaging protein for use according to item 13, wherein at least one of said first, second or third target doses of the first treatment cycle is about 5 mg, about 10 mg, about 20 mg, about 30 mg, about 40 mg, about 50 mg, about 60 mg, or about 70 mg, optionally about 10 mg or about 60 mg.

15. The T-cell engaging protein for use according to item 14, wherein at least one of said first, second or third target doses of the first treatment cycle is about 10 mg.

16. The T-cell engaging protein for use according to item 16, wherein at least one of said first, second or third target doses of the first treatment cycle is about 60 mg.

17. The T-cell engaging protein for use according to any one of items 2 to 16, wherein said first treatment cycle is not repeated or said first treatment cycle is repeated at least once.

18. The T-cell engaging protein for use according to any one of items 2 to 17, wherein said first, second and third target doses are administered within three weeks of said first treatment cycle, wherein optionally said first target dose is administered on day 1, the second target dose is administered on day 8 and the third target dose is administered on day 15 of said first treatment cycle, wherein optionally said first treatment cycle has a duration of three weeks.

19. The T-cell engaging protein for use according to any one of items 2 to 17, wherein said first treatment cycle is followed by a second treatment cycle, said second treatment cycle comprising the step of or consisting of the step:
administering to the subject a target dose of said T-cell engaging protein, said target dose ranging from about 80 μg/kg of the subject's body weight to about 1600 μg/kg of the subject's body weight;
   wherein said second treatment cycle is optionally repeated at least once.

20 The T-cell engaging protein according to item 19, wherein the amount of said target dose is about 90 μg/kg.

21. The T-cell engaging protein according to item 19, wherein the amount of said target dose is about 180 μg/kg.

22. The T-cell engaging protein according to item 19, wherein the amount of said target dose is about 270 μg/kg.

23. The T-cell engaging protein according to item 19, wherein the amount of said target dose is about 360 μg/kg.

24. The T-cell engaging protein according to item 19, wherein the amount of said target dose is about 540 μg/kg.

25. The T-cell engaging protein according to item 19, wherein the amount of said target dose is about 720 μg/kg.

26. The T-cell engaging protein according to item 19, wherein the amount of said target dose is about 1080 μg/kg.

27. The T-cell engaging protein according to item 19, wherein the amount of said target dose is about 1530 μg/kg.

28. The T-cell engaging protein for use according to any one of items 2 to 27, wherein said first treatment cycle is followed by a second treatment cycle, said second treatment cycle comprising the step of or consisting of the step:
administering to the subject a target dose of the T-cell engaging protein, said target dose ranging from about 5 mg to about 70 mg;
   wherein said second treatment cycle is optionally repeated at least once.

29. The T-cell engaging protein according to item 28, wherein the amount of said target dose is about 10 mg.

30. The T-cell engaging protein according to item 28, wherein the amount said target dose is about 60 mg.

31. The T-cell engaging protein according to item 28, wherein the amount of said target dose is about 30 mg.

32. The T-cell engaging protein for use according to item 2 or 32, wherein said second treatment cycle has a total duration of three weeks and/or said target dose is administered on day 1 of said three weeks.

33. The T-cell engaging protein for use according to any one of items 2 to 32, wherein said first treatment cycle directly follows said step-in cycle; and/or said second treatment cycle directly follows said first treatment cycle.

34. The T-cell engaging protein for use according to any one of the preceding claims, wherein said Neuroendocrine Neoplasm (NEN) is a Neuroendocrine Carcinoma (NEC), preferably selected from the group consisting of Lung-NEC such as LCNEC of the lung, Extrapulmonary (ep) NEC including NEC with unknown primary site, small cell lung cancer (SCLC) including extensive-stage (ES)-SCLC, and limited-stage (LS) SCLC; or said NEN is a Neuroendocrine Tumor (NET) such as grade 3 NET or, in the alternative, epNEC excluding Neuroendocrine prostate cancer (NEPC).

35. The T-cell engaging protein for use according to any one of the preceding items, wherein a premedication comprising a non-steroidal anti-inflammatory drug, such as acetaminophen, an anti-histamine and/or a steroid, preferably a corticosteroid such as prednisolone or dexamethasone, prior to at least one of the doses, optionally prior to each dose, such as any target dose or any step-in dose, wherein prior to optionally means that the premedication is administered about 0.25 to about 2 hours, such as about 0.5 hours to about 1 hour, prior to the respective point in time of the start of the administration of a respective dose of the T-cell engaging protein.

36. The T-cell engaging protein for use according to any one of the preceding items, wherein the subject has been treated with a chemotherapy involving administration of a cytostatic agent prior to the step-in cycle.

37. The T-cell engaging protein for use according to any one of the preceding items, wherein an occurrence of cytokine release syndrome is to be treated with an anti-cytokine release syndrome agent, preferably selected from the group consisting of a corticosteroid, an IL-6 agonist and an IL-6R antagonist.

38. The T-cell engaging protein for use according to any one of the preceding items, said T-cell engaging protein comprises a first antigen binding unit specifically binding to DLL3, comprising light chain CDRs comprising the amino acid sequences of SEQ ID NO:13 (CDR1), SEQ ID NO:14 (CDR2) and SEQ ID NO:15 (CDR3) and heavy chain CDRs comprising the amino acid sequences of SEQ ID NO:16 (CDR1), SEQ ID NO:17 (CDR2) and SEQ ID NO:18 (CDR3) and a second antigen binding unit specifically binding to CD3 comprising light chain CDRs comprising the amino acid sequences of SEQ ID NO:55 (CDR1), SEQ ID NO:56 (CDR2) and SEQ ID NO:57 (CDR3) and heavy chain CDRs comprising the amino acid sequences of SEQ ID NO:58 (CDR1), SEQ ID NO:59 (CDR2) and SEQ ID NO:60 (CDR3).

39. The T-cell engaging protein for use according to item 38, wherein said T-cell engaging protein comprises immunoglobulin (Ig) domains, preferably 12 Ig domains.

40. The T-cell engaging protein for use according to item 39, wherein said Ig domains are comprised in two polypeptide chains, wherein each polypeptide chain comprises six Ig domains.

41. The T-cell engaging protein for use according to any one of items 38 to 40, wherein either antigen binding unit comprises six complementarity determining regions (CDRs).

42. The T-cell engaging protein for use according to item 41, wherein said T-cell engaging protein comprises a first antigen binding unit specifically binding to DLL3, comprising light chain CDRs comprising the amino acid sequences of SEQ ID NO:13 (CDR1), SEQ ID NO:14 (CDR2) and SEQ ID NO:15 (CDR3) and heavy chain CDRs comprising the amino acid sequences of SEQ ID NO:16 (CDR1), SEQ ID NO:17 (CDR2) and SEQ ID NO:18 (CDR3) and a second antigen binding unit specifically binding to CD3 comprising light chain CDRs comprising the amino acid sequences of SEQ ID NO:55 (CDR1), SEQ ID NO:56 (CDR2) and SEQ ID NO:57 (CDR3) and heavy chain CDRs comprising the amino acid sequences of SEQ ID NO:58 (CDR1), SEQ ID NO:59 (CDR2) and SEQ ID NO:60 (CDR3).

43. The T-cell engaging protein for use according to item 42, wherein said T-cell engaging protein comprises (i) a first antigen binding unit specifically binding to DLL3 comprising a light chain variable domain comprising the amino acid sequence of SEQ ID NO:41 and heavy chain variable domain comprising the amino acid sequence of SEQ ID NO:42 and (ii) a second antigen binding unit specifically binding to CD3 comprising a light chain variable domain comprising the amino acid sequence of SEQ ID NO:67 and a heavy chain variable domain comprising the amino acid sequence of SEQ ID NO:68.

44. The T-cell engaging protein for use according to item 43, wherein said T-cell engaging protein comprises a first single chain Fab forming a first antigen binding unit comprising the sequence of SEQ ID NO:51 and a second single chain Fab comprising the sequence of SEQ ID NO:71 forming a second antigen binding unit.

45. The T-cell engaging protein for use according to any one of items 38 to 44, wherein said T-cell engaging protein comprises of a first polypeptide chain specific for DLL3 comprising an amino acid sequence of SEQ ID NO:75 and a second polypeptide chain specific for CD3 comprising the amino acid sequence of SEQ ID NO:79.

46. The T-cell engaging protein for use according to any one of the preceding items, wherein essentially no cytokine release syndrome occurs after said initial and/or said elevated dose of said step-in cycle in the subject.

47. The T-cell engaging protein for use according to any one of the preceding items, wherein said T-cell engaging protein is the only anti-cancer agent to be administered.

48. The T-cell engaging protein for use according to any one of the preceding items, wherein at least one further anti-cancer agent is to be administered, said further anti-cancer agent preferably being a chemotherapeutic agent and/or an immune-checkpoint inhibitor such as an anti-PD-1 antibody or an anti-PD-L1 antibody.

49. The T-cell engaging protein for use according to any one the preceding items, wherein said initial dose ranges from about 20 μg/kg of the subject's body weight to about 40 μg/kg of the subject's body weight;
said elevated dose ranges from about 55 μg/kg of the subject's body weight to about 95 μg/kg of the subject's body weight, and optionally said elevated dose is about 90 μg/kg of the subject's body weight; and
said further dose ranges from about 5 mg to about 15 mg or about 50 mg to about 70 mg.

50. The T-cell engaging protein for use according to item 49,
wherein said initial dose is about 30 μg/kg;
said elevated dose is about 90 μg/kg; and
said further dose is about 10 mg.

51. The T-cell engaging protein for use according to item 49,
wherein said initial dose is about 30 μg/kg;
said elevated dose is about 90 μg/kg; and
said further dose is about 30 mg.

52. The T-cell engaging protein for use according to item 49,
wherein said initial dose is about 30 μg/kg;
said elevated dose is about 90 μg/kg; and
said further dose is about 60 mg.

53. The T-cell engaging protein for use according to any one of the preceding items, wherein said T-cell engaging protein is administered by intravenous (IV) infusion.

54. The T-cell engaging protein for use according to any one of the preceding claims, wherein the subject is a human.

55. The T-cell engaging protein for use according to any one of the preceding items, the method further comprising administering to the subject a dose of lurbinectedin.

56. The T-cell engaging protein for use according to any one of the preceding items, the method further comprising administering to the subject a dose of amrubicin.

57. The T-cell engaging protein for use according to any one of the preceding items, comprising a step of co-administering topotecan to the subject, preferably for use in the treatment of SCLC (any treatment line) including ES-SCLC, preferably second line (2L) treatment of ES-SCLC that progressed or recurred following Standard of Care (SoC) treatment and LS-SCLC, preferably first line (1L) treatment.

58. The T-cell engaging protein for use according to any one of the preceding items, comprising a step of co-administering lurbinectedin to the subject, preferably for use in the treatment of SCLC (any treatment line) including ES-SCLC, preferably 2L treatment of ES-SCLC that progressed or recurred following SoC, and LS-SCLC, preferably 1L treatment.

59. The T-cell engaging protein for use according to any one of the preceding items, comprising a step of co-administering amrubicin to the subject, preferably for use in the treatment of SCLC (any treatment line) including ES-SCLC, preferably 2L treatment of ES-SCLC that progressed or recurred following SoC, and LS-SCLC, preferably 1L treatment.

60. The T-cell engaging protein for use according to any one of the preceding items, comprising a step of co-administering etoposide and carboplatin or cisplatin, and optionally also anti-PD-L1 antibody e.g. atezolizumab or durvalumab, to the subject, preferably for use in the treatment of SCLC including ES-SCLC, especially for 1L, 2L and/or 3L treatment, and LS-SCLC (any treatment line).

61. The T-cell engaging protein for use according to item 60, wherein the T-cell engaging protein, carboplatin or cisplatin and etoposide are co-administered for 3 to 5 treatment cycles, in-volving a step in cycle.

62. The T-cell engaging protein for use according to item 61, wherein only the T-cell engaging protein will be administered after said 3-5 treatment cycles, in particular for a maximum of 36 months.

63. The T-cell engaging protein for use according to any one of the preceding items, comprising a step of co-administering etoposide and carboplatin or cisplatin (optionally also anti-PD-L1 antibody), to the subject, preferably for use in the treatment of epNEC or LCNEC of the lung (any treatment line), preferably first line treatment of epNEC or LCNEC of the lung.

64. The T-cell engaging protein for use according to any one of items 1 to 63, wherein the cancer is epNEC, optionally epNEC excluding NEPC.

65. The T-cell engaging protein for use according to any one of items 1 to 63, wherein the cancer is SCLC.

66. The T-cell engaging protein for use according to any one of items 1 to 63, wherein the cancer is LCNEC of the lung.

67. The T-cell engaging protein for use according to any one of items 1 to 63, wherein the cancer is glioma.

68. The T-cell engaging protein for use according to any one of items 1 to 63, wherein the cancer is NEC.

69. The T-cell engaging protein for use according to any one of items 1 to 63, wherein the cancer is NEPC.

70. The T-cell engaging protein for use according to any one of the preceding items, the T-cell engaging protein, carboplatin and cisplatin and etoposide, optionally also anti-PD-L1 antibody, are co-administered for 3 to 5 treatment cycles, involving a step in cycle, wherein more preferably, only the T-cell engaging protein, optionally together with anti-PD-L1 antibody, will be administered after said 3-5 treatment cycles, in particular for a maximum of 36 months, wherein more optionally these cycles may involve the step-in cycle for each regimen.

71. The T-cell engaging protein for use according to any one of the preceding items, wherein one or more treatment cycles (e.g. one or two treatment cycles) include only chemotherapy taking place prior to any of the co-administration treatments with the T-cell engaging protein.

72. The T-cell engaging protein according to any one of the preceding items, wherein lurbinectedin, if co-administered may optionally be administered at the approved regimen of 3.2 mg/m² on day 1 of each 21 day cycle as an intravenous infusion, especially for 60 minutes.

73. The T-cell engaging protein according to any one of the preceding items, wherein amrubicin, if co-administered may optionally be administered at the approved regimen of 40 mg/m² on days 1 to 3 of each 21 day cycle, wherein optionally amrubicin may be administered as an intravenous infusion.

74. The T-cell engaging protein according to any one of the preceding items, wherein topotecan, if co-administered may optionally be administered at the approved regimen of 2.3 mg/m² orally (p.o.) or 1.5 mg/m² by intravenous infusion daily on day 1 to day 5 during each 21 day cycle.

75. The T-cell engaging protein according to any one of the preceding items, wherein carboplatin and etoposide, if co-administered may optionally be administered at the approved regimen of AUC 5 mg/mL min (carboplatin) and 100 mg/m2 (etoposide), wherein carboplatin is administered at day 1 during each 21 day cycle and etoposide is administered on days 1 to 3 during each 21 day cycle.

76. The T-cell engaging protein according to any one of the preceding items, wherein cisplatin and etoposide, if co-administered may optionally be administered at the approved regimen of 75 mg/m2 (cisplatin) and 100 mg/m2 (etoposide), wherein cisplatin is administered at day 1 during each 21 day cycle and etoposide is administered on days 1 to 3 during each 21 day cycle.

77. The T-cell engaging protein for use according to any one of the preceding items, wherein a post-medication is administered directly after the administration of the T-cell engaging protein, wherein optionally “directly after” means that the post-medication is administered following the respective point in time of the start of the administration of a respective dose of the T-cell engaging protein.

78. The T-cell engaging protein for use according to item 77, wherein the post-medication comprises a non-steroidal anti-inflammatory drug, such as acetaminophen, an anti-histamine and/or a steroid, such as prednisolone or dexamethasone.

79. The T-cell engaging protein for use according to any one of item 77 to 78, wherein the post-medication comprises the same or a different substance(s) as compared to the premedication according to item 35.

80. The T-cell engaging protein for use according to any one of items 77 to 79, wherein the post-medication is administered orally (p.o.) or intravenously (i.v).

81. The T-cell engaging protein for use according to any one of items 35 and 77 to 80, wherein the pre-medication is administered orally (p.o.) or intravenously (i.v.).

82. The T-Cell engaging protein for use according to any one of items 35 and 77 to 81, wherein the post-medication is administered at least 6 hours up to 12 hours, such as 8±2 hours, after the respective administration of a dose of the T-cell engaging protein, wherein optionally “after” means that the post-medication is administered at least 6 hours up to about 12 hours, such as 8±2 hours, following the respective point in time of the start of the administration of a respective dose of the T-cell engaging protein.

83. The T-cell engaging protein for use according to any one of items 35 and 77 to 82, wherein the premedication is administered 0.25 to 2 hours, such as 0.5 hours to about 1 hour, prior to the respective administration of the T-cell engaging protein, wherein optionally “prior” means that the premedication is administered 0.25 to 2 hours, such as about 0.5 hours to about 1 hour, prior to the respective point in time of the start of the administration of a respective dose of the T-cell engaging protein.

84. The T-cell engaging protein for use according to any one of items 35 and 77 to 83, wherein the pre- and/or post-medication comprises or a steroid

85. The T-cell engaging protein according to item 84, wherein the steroid is a corticosteroid.

86. The T-cell engaging protein for use according to item 85, wherein the corticosteroid is selected from the group consisting of prednisolone and dexamethasone or a combination thereof.

87. The T-cell engaging protein for use according to item 86, wherein dexamethasone is used for the premedication and/or post-medication.

88. The T-cell engaging protein for use according to any one of items 35 and 77 to 87, wherein the steroid is selected from the group consisting of cortisone, betamethasone, triamcinolone, hydrocortisone, hydrocortisone, prednisone, methylprednisolone, dexamethasone or combinations thereof.

89. The T-cell engaging protein for use according to any of items 35 and 77 to 88, wherein an amount of a dexamethasone dose comprised in a premedication ranges from about 10 mg to about 18 mg, such as about 16 mg.

90. The T-cell engaging protein for use according to any of items 35 and 77 to 89, wherein an amount of a dexamethasone dose comprised in a premedication ranges from about 4 mg to about 10 mg, such as about 8 mg.

91. The T-cell engaging protein for use according to one any of items 35 and 77 to 90, wherein an amount of a dexamethasone dose comprised in a post-medication ranges from about 4 mg to about 10 mg, such as about 8 mg.

92. The T-cell engaging protein for use according to any one of the preceding items, where-in a premedication is administered directly prior the initial dose and a post-medication is administered directly after the initial dose, wherein the pre-medication is preferably administered about 0.25 to about 2 hours, such as 0.5 hours to about 1 hour before the initial dose and/or preferably the post-medication is preferably administered at least 6 hours up to 12 hours, such as 8±2 hours, after the initial dose, wherein optionally “directly prior” means that the premedication is administered 0.25 to 2 hours, such as about 0.5 hours to about 1 hour, prior to the respective point in time of the start of the administration of the initial dose of the T-cell engaging protein and optionally “directly after” means that the post-medication is administered at least 6 hours up to about 12 hours, such as 8±2 hours, following the respective point in time of the start of the administration of the initial dose of the T-cell engaging protein.

93. The T-cell engaging protein for use according to any one of the preceding items, where-in a premedication is administered directly prior the elevated dose and a post-medication is administered directly after the elevated dose, wherein the pre-medication is preferably administered about 0.25 to about 2 hours, such as 0.5 hours to about 1 hour before the elevated dose and/or t the post-medication is preferably administered at least 6 hours up to 12 hours, such as 8±2 hours, after the elevated dose, wherein optionally wherein optionally “directly prior” means that the premedication is administered 0.25 to 2 hours, such as about 0.5 hours to about 1 hour, prior to the respective point in time of the start of the administration of the elevated dose of the T-cell engaging protein and optionally “directly after” means that the post-medication is administered at least 6 hours up to about 12 hours, such as 8±2 hours, following the respective point in time of the start of the administration of the elevated dose of the T-cell engaging protein.

94. The T-cell engaging protein for use according to any one of the preceding items, where-in a premedication is administered directly prior the further dose and a post-medication is administered directly after the further dose, wherein the pre-medication is preferably administered about 0.25 to about 2 hours, such as 0.5 hours to about 1 hour before the further dose and/or the post-medication is preferably administered at least 6 hours up to 12 hours, such as 8±2 hours, after the further dose, wherein optionally “directly prior” means that the premedication is administered 0.25 to 2 hours, such as about 0.5 hours to about 1 hour, prior to the respective point in time of the start of the administration of the further dose of the T-cell engaging protein and optionally “directly after” means that the post-medication is administered at least 6 hours up to about 12 hours, such as 8±2 hours, following the respective point in time of the start of the administration of the further dose of the T-cell engaging protein.

95. The T-cell engaging protein for use according to any one of items 35 and 77 to 94, wherein the antihistamine of the premedication and/or the post-medication is diphenylhydramine, such as about 50 mg.

96. The T-cell engaging protein for use according to any one of items 35 and 77 to 95, wherein the non-steroidal anti-inflammatory drug of the premedication and/or the post-medication is acetaminophen/paracetamol, such as about 1000 mg.

97. The T-cell engaging protein for use according to any one of the preceding items, where-in a hospitalization, such as for 24 hours, follows the initial, elevated and/or further doses, i.e. the step-in doses, and/or the first target dose of the T-cell engaging protein.

98. The T-cell engaging protein for use according to any one of the preceding items, where-in a hospitalization, such as for 24 hours, follows the initial and elevated doses, i.e. the first two step-in doses, of the T-cell engaging protein.

99. The T-cell engaging protein for use according to any one of the preceding items, where-in no hospitalization follows after the initial, elevated and further dose, i.e. the step-in doses, and the target dose(s) of the T-cell engaging protein.

100. The T-cell engaging protein for use according to any one of items 35 and 77 to 99, wherein the premedication and/or the post-medication comprise different compounds.

101. The T-cell engaging protein for use according to any one of items 35 and 77 to 100, wherein the premedication and/or the post-medication comprise an inhibitor/agonist of TNF-alpha (TNF) and/or TNF-alpha-Receptor (TNFR) reducing TNF/TFNR signaling and/or an inhibitor/agonist of IL6/IL6R that reduces IL6/IL6R signaling.

102. The T-cell engaging protein for use according to item 101, wherein the inhibitor/agonist of IL6/IL6R that reduces IL6/IL6R signaling is tocilizumab.

103. The T-cell engaging protein for use according to any one of items 35 and 77 to 102, wherein the premedication and/or post-medication comprise a steroid, wherein the steroid is selected from the group consisting of cortisone, betamethasone, triamcinolone, hydrocortisone, hydrocortisone, prednisone, methylprednisolone, dexamethasone or combinations thereof.

104. The T-cell engaging protein for use according to any one of items 35 and 77 to 103, wherein the premedication and/or the post-medication comprise an inhibitor/agonist of TNF/TNFR that reduces TNF/TNFR signaling being selected from the group consisting of etanercept, infliximab, adalimumab, certolizumab pegol and golimumab or a combination thereof.

105. The T-cell engaging protein for use according to item 104, wherein inhibitor/agonist of IL6/IL6R that reduces IL6/IL6R signaling is tocilizumab.

106. The T-cell engaging protein for use according to any one of items 35 and 77 to 105, wherein the premedication and/or the post-medication comprises a non-steroidal anti-inflammatory drug, an antihistamine and a steroid.

107. The T-cell engaging protein for use according to item 106, wherein the non-steroidal anti-inflammatory drug is acetaminophen/paracetamol, such as about 1000 mg p.o. or i.v., or equivalent, diphenhydramine, such as about 50 mg p.o. or i.v., or equivalent, dexamethasone, such as about 16 mg or about 8 mg p.o. or i.v., or equivalent intermediate-acting corticosteroid.

108. The T-cell engaging protein for use according to any one the preceding items, where-in the initial dose amount of the T-cell engaging protein is about 10 μg/kg and the elevated dose amount of the T-cell engaging protein is about 30 μg/kg, and wherein prior to and after those initial and elevated doses, a dexamethasone premedication and post-medication of a dose amount of about 8 mg is administered, respectively.

109. The T-cell engaging protein for use according to any one of items 35 and 77 to 108, wherein the premedication comprises a non-steroidal anti-inflammatory drug, such as acetaminophen/paracetamol, and an antihistamine, such as diphenhydramine, and optionally a steroid.

110. The T-cell engaging protein for use according to item 109, wherein the premedication is administered prior to the respective step-in doses of the T-cell engaging protein, such as the initial dose, e.g. being about 10 μg/kg, such as the elevated dose, e.g. being about 30 μg/kg, and such as the further dose, e.g. being about 90 μg/kg.

111. The T-cell engaging protein for use according to item 109 or 110, wherein premedication may also be administered prior to the first to optionally at least 6th or more target doses of the T-cell engaging protein, e.g. being about 60 mg of the T-cell engaging protein.

112. The T-cell engaging protein for use according to any one of items 109 to 111, wherein a steroid, such as dexamethasone, is administered as premedication in addition to the non-steroidal anti-inflammatory drug and the antihistamine and as a post-medication.

113. The T-cell engaging protein for use according to any one of items 109 to 112, wherein prior to each of the initial dose, the elevated dose and the further dose and the first two target doses of the T-cell engaging protein, the steroid, such as dexamethasone, is administered as a premedication.

114. The T-cell engaging protein for use according to item 113, wherein about 8 mg of the steroid being dexamethasone is respectively administered prior to the initial dose, the elevated dose, and the further dose of the T-cell engaging protein as premedication and about 8 mg of the steroid being dexamethasone is respectively administered after the initial dose, the elevated dose and the further dose of the T-cell engaging protein as post-medication.

115. The T-cell engaging protein according to item 113 or 114, wherein prior to the first target dose of the T-cell engaging protein about 4 mg of dexamethasone is administered as a premedication and after the first target dose of the T-cell engaging protein about 4 mg of dexamethasone is administered as a post-medication.

116. The T-cell engaging protein according to any one of items 109 to 115, wherein after the initial dose, the elevated dose, the further dose and the first target dose of the T-cell engaging protein, the steroid, such as dexamethasone, is administered, e.g. in an amount of about 8 mg, respectively.

117. The T-cell engaging protein according to any one of items 109 to 116, wherein the amount of the steroid, such as dexamethasone, is reduced at the first target dose of the T-cell engaging protein for the premedication and/or post-medication, if the T-cell engaging protein is well-tolerated, i.e. if no CRS≥2 and/or ICANS (any grade) occurred following the prior dose.

118. The T-cell engaging protein according to any one of items 109 to 117, wherein the steroid, such as dexamethasone, is omitted for the premedication and the post-medication starting from the second target dose of the T-cell engaging protein.

119. The T-cell engaging protein according to any one of items 109 to 108, wherein no more steroid, such as dexamethasone, is administered as a premedication from the third target dose of the T-cell engaging protein, if it is well tolerated.

120. The T-cell engaging protein for use according to any one of items 109 to 119, wherein if CRS/IRR/ICANS occur, the steroid, e.g. dexamethasone, dose for at least premedication at the next drug administration must remain the same as for the current dose and must not be reduced.

121. The T-cell engaging protein for use according to any one of items 109 to 120, wherein the line of treatment is 1L in DLL3 positive subjects and those subjects have unresectable locally advanced or metastatic epNEC, wherein, the co-administration involves—in addition to the T-cell engaging protein—an administration of carboplatin and etoposide.

122. The T-cell engaging protein for use according to any one of items 109 to 120, wherein the treatment is 1L and the co-administering—in addition to the T-cell engaging protein—involves administering atezolizumab, carboplatin and etoposide, and wherein the cancer is ES-SCLC.

123. The T-cell engaging protein for use according to any one of items 35 and 77 to 122, wherein a premedication is administered directly prior the first target dose and a post-medication is administered directly after the first target dose, wherein the pre-medication is preferably administered about 0.25 to about 2 hours, such as 0.5 hours to about 1 hour before the first target dose and/or the post-medication is preferably administered at least 6 hours up to 12 hours, such as 8±2 hours, after the first target dose, wherein optionally “directly prior” means that the premedication is administered 0.25 to 2 hours, such as about 0.5 hours to about 1 hour, prior to the respective point in time of the start of the administration of the first target dose of the T-cell engaging protein and optionally “directly after” means that the post-medication is administered at least 6 hours up to about 12 hours, such as 8±2 hours, following the respective point in time of the start of the administration of the first target dose of the T-cell engaging protein.

### The Present Invention Furthermore Relates to the Following Item List 3

1. A T-cell engaging protein comprising a first antigen binding unit that specifically binds to DLL3 and a second antigen binding unit that specifically binds to CD3 for use in a method of treatment of a DLL3-positive cancer or a cancer selected from a Neuroendocrine Neoplasm (NEN), Merkel cell carcinoma (MCC), medullary thyroidal carcinoma and glioma,
the method comprising a step-in cycle, said step-in cycle comprising the steps of:
said step-in cycle comprising the steps of:
   administering to a subject an initial dose of said T-cell engaging protein, said initial dose ranging from about 5 μg/kg of the subject's body weight to about 15 μg/kg of the subject's body weight,
   administering to the subject an elevated dose, which is elevated as compared to said initial dose, of said T-cell engaging protein, the elevated dose ranging from about 20 μg/kg of the subject's body weight to about 40 μg/kg of the subject's body weight,
   administering to the subject a further dose of said T-cell engaging protein, said further dose ranging from about 80 μg/kg of the subject's body weight to about 1600 μg/kg of the subject's bodyweight; or
the method comprising a step-in cycle, the step-in cycle comprising the steps of:
   administering to the subject an initial dose, an elevated dose, which is elevated as compared to said initial dose, and a further dose of said T-cell engaging protein, wherein a ratio between said initial, elevated and further dose per kilogram of the subject's body weight is about 1:about 3:about 9.

2. The T-cell engaging protein for use according to item, wherein said further dose is equal to a target dose, said target dose being the dose to be administered during a first treatment cycle which optionally directly follows said step-in cycle.

3. The T-cell engaging protein for use according to items 1 or 2, wherein said initial dose, said elevated dose and/or said further is/are administered once; or said initial dose, said elevated dose and/or said further is/are administered more than once, optionally 2 to 10 times,
wherein optionally the number of administrations is determined based on treatment tolerability including presence and/or degree of a cytokine release syndrome (CRS) and/or of an immune effector cell-associated neurotoxicity syndrome (ICANS),
and wherein further optionally said elevated dose follows the initial dose only if said CRS, to the extent determined, is rated grade 1 or less in terms of NCI CTC grade and/or if said ICANS, to the extend determined, is rated grade 1 or less, and/or wherein further optionally
further optionally said further dose follows the elevated dose only if said CRS, to the extent determined, is rated grade 1 or less in terms of NCI CTC grade and/or if said ICANS, to the extend determined, is rated grade 1 or less.

4. The T-cell engaging protein for use according to any one of the preceding items, wherein said initial dose is administered three times to ten times within the step-in cycle.

5. The T-cell engaging protein for use according to any one of the preceding items, wherein said initial dose is administered three times within three weeks, optionally on day 1, day 8 and day 15 of the three weeks.

6. The T-cell engaging protein for use according to any one of the preceding items, wherein said step-in cycle has a duration of at least three weeks.

7. The T-cell engaging protein for use according to any one of the preceding items, wherein said initial, elevated and further doses are administered within three weeks, said initial dose being administered once, wherein optionally said initial dose is administered on day 1, said elevated dose is administered on day 8 and said further dose is administered on day 15 of said step-in cycle.

8. The T-cell engaging protein for use according to any one of the preceding items, wherein
said initial dose is about 10 μg/kg of the subject's body weight;
said elevated dose is about 30 μg/kg of the subject's body weight; and
said further dose is about is 90 μg/kg of the subject's body weight.

9. The T-cell engaging protein for use according to any one of the preceding items; the method further comprising a first treatment cycle following the step-in cycle, said first treatment cycle comprising the steps of:
administering to a subject a first target dose of said T-cell engaging protein, said first target dose ranging from about 80 μg/kg of the subject's body weight to about 1600 μg/kg of the subject's body weight,
administering to the subject a second target dose of said T-cell engaging protein, said second dose ranging from about 80 μg/kg of the subject's body weight to about 1600 μg/kg of the subject's body weight, and
administering to the subject a third target dose of said T-cell engaging protein, said third dose ranging from about 80 μg/kg of the subject's body weight to about 1600 μg/kg of the subject's bodyweight,
   wherein optionally the amount of all three target doses is identical.

10. The T-cell engaging protein for use according to any one of the preceding items, the method further comprising a first treatment cycle following said step-in cycle, said first treatment cycle comprising the steps of:
administering to a subject a first target dose of the T-cell engaging protein, said first target dose ranging from about 5 mg to about 70 mg,
administering to the subject a second dose of the T-cell engaging protein, said second dose ranging from about 5 mg to about 70 mg, and
administering to the subject a third dose of the T-cell engaging protein, said third dose ranging from about 5 mg to about 70 mg,
   wherein optionally the amount of all three target doses is identical.

11. The T-cell engaging protein for use according to item 10, wherein the amount of all three target doses is identical and the amount ranges from about 5 mg to about 10 mg, preferably being about 10 mg, for the first, second and third target dose, respectively.

12. The T-cell engaging protein for use according to item 10, wherein the amount of all target doses is identical, and the amount is about 60 mg for the first, second and third target dose, respectively.

13. The T-cell engaging protein for use according to item 10, wherein the amount of all target doses is identical, and the amount is about 30 mg for the first, second and third target dose, respectively.

14. The T-cell engaging protein for use according to item 13, wherein at least one of said first, second or third target doses of the first treatment cycle is about 5 mg, about 10 mg, about 20 mg, about 30 mg, about 40 mg, about 50 mg, about 60 mg, or about 70 mg, optionally about 10 mg or about 60 mg.

15. The T-cell engaging protein for use according to item 14, wherein at least one of said first, second or third target doses of the first treatment cycle is about 10 mg.

16. The T-cell engaging protein for use according to item 16, wherein at least one of said first, second or third target doses of the first treatment cycle is about 60 mg.

17. The T-cell engaging protein for use according to any one of items 2 to 16, wherein said first treatment cycle is not repeated or said first treatment cycle is repeated at least once.

18. The T-cell engaging protein for use according to any one of items 2 to 17, wherein said first, second and third target doses are administered within three weeks of said first treatment cycle, wherein optionally said first target dose is administered on day 1, the second target dose is administered on day 8 and the third target dose is administered on day 15 of said first treatment cycle, wherein optionally said first treatment cycle has a duration of three weeks.

19. The T-cell engaging protein for use according to any one of items 2 to 17, wherein said first treatment cycle is followed by a second treatment cycle, said second treatment cycle comprising the step of or consisting of the step:
administering to the subject a target dose of said T-cell engaging protein, said target dose ranging from about 80 μg/kg of the subject's body weight to about 1600 μg/kg of the subject's body weight;
   wherein said second treatment cycle is optionally repeated at least once.

20 The T-cell engaging protein according to item 19, wherein the amount of said target dose is about 90 μg/kg.

21. The T-cell engaging protein according to item 19, wherein the amount of said target dose is about 180 μg/kg.

22. The T-cell engaging protein according to item 19, wherein the amount of said target dose is about 270 μg/kg.

23. The T-cell engaging protein according to item 19, wherein the amount of said target dose is about 360 μg/kg.

24. The T-cell engaging protein according to item 19, wherein the amount of said target dose is about 540 μg/kg.

25. The T-cell engaging protein according to item 19, wherein the amount of said target dose is about 720 μg/kg.

26. The T-cell engaging protein according to item 19, wherein the amount of said target dose is about 1080 μg/kg.

27. The T-cell engaging protein according to item 19, wherein the amount of said target dose is about 1530 μg/kg.

28. The T-cell engaging protein for use according to any one of items 2 to 27, wherein said the first treatment cycle is followed by a second treatment cycle, said second treatment cycle comprising the step of or consisting of the step:
administering to the subject a target dose of the T-cell engaging protein, said target dose ranging from about 5 mg to about 70 mg;
   wherein said second treatment cycle is optionally repeated at least once.

29. The T-cell engaging protein according to item 28, wherein the amount of said target dose is about 10 mg.

30. The T-cell engaging protein according to item 28, wherein the amount said target dose is about 60 mg.

31. The T-cell engaging protein according to item 28, wherein the amount of said target dose is about 30 mg.

32. The T-cell engaging protein for use according to item 2 or 32, wherein said second treatment cycle has a total duration of three weeks and/or said target dose is administered on day 1 of said three weeks.

33. The T-cell engaging protein for use according to any one of items 2 to 32, wherein said first treatment cycle directly follows said step-in cycle; and/or said second treatment cycle directly follows said first treatment cycle.

34. The T-cell engaging protein for use according to any one of the preceding claims, wherein said Neuroendocrine Neoplasm (NEN) is a Neuroendocrine Carcinoma (NEC), preferably selected from the group consisting of Lung-NEC such as LCNEC of the lung, Extrapulmonary (ep) NEC including NEC with unknown primary site, small cell lung cancer (SCLC) including extensive-stage (ES)-SCLC, and limited-stage (LS) SCLC; or said NEN is a Neuroendocrine Tumor (NET) such as grade 3 NET or, in the alternative, epNEC excluding Neuroendocrine prostate cancer (NEPC).

35. The T-cell engaging protein for use according to any one of the preceding items, wherein a premedication comprising a non-steroidal anti-inflammatory drug, such as acetaminophen, an anti-histamine and/or a steroid, preferably a corticosteroid such as prednisolone or dexamethasone, prior to at least one of the doses, optionally prior to each dose, such as any target dose or any step-in dose, wherein prior to optionally means that the premedication is administered about 0.25 to about 2 hours, such as about 0.5 hours to about 1 hour, prior to the respective point in time of the start of the administration of a respective dose of the T-cell engaging protein.

36. The T-cell engaging protein for use according to any one of the preceding items, wherein the subject has been treated with a chemotherapy involving administration of a cytostatic agent prior to the step-in cycle.

37. The T-cell engaging protein for use according to any one of the preceding items, wherein an occurrence of cytokine release syndrome is to be treated with an anti-cytokine release syndrome agent, preferably selected from the group consisting of a corticosteroid, an IL-6 agonist and an IL-6R antagonist.

38. The T-cell engaging protein for use according to any one of the preceding items, said T-cell engaging protein comprises a first antigen binding unit specifically binding to DLL3, comprising light chain CDRs comprising the amino acid sequences of SEQ ID NO:13 (CDR1), SEQ ID NO:14 (CDR2) and SEQ ID NO:15 (CDR3) and heavy chain CDRs comprising the amino acid sequences of SEQ ID NO:16 (CDR1), SEQ ID NO:17 (CDR2) and SEQ ID NO:18 (CDR3) and a second antigen binding unit specifically binding to CD3 comprising light chain CDRs comprising the amino acid sequences of SEQ ID NO:55 (CDR1), SEQ ID NO:56 (CDR2) and SEQ ID NO:57 (CDR3) and heavy chain CDRs comprising the amino acid sequences of SEQ ID NO:58 (CDR1), SEQ ID NO:59 (CDR2) and SEQ ID NO:60 (CDR3).

39. The T-cell engaging protein for use according to item 38, wherein said T-cell engaging protein comprises immunoglobulin (Ig) domains, preferably 12 Ig domains.

40. The T-cell engaging protein for use according to item 39, wherein said Ig domains are comprised in two polypeptide chains, wherein each polypeptide chain comprises six Ig domains.

41. The T-cell engaging protein for use according to any one of items 38 to 40, wherein either antigen binding unit comprises six complementarity determining regions (CDRs).

42. The T-cell engaging protein for use according to item 41, wherein said T-cell engaging protein comprises a first antigen binding unit specifically binding to DLL3, comprising light chain CDRs comprising the amino acid sequences of SEQ ID NO:13 (CDR1), SEQ ID NO:14 (CDR2) and SEQ ID NO:15 (CDR3) and heavy chain CDRs comprising the amino acid sequences of SEQ ID NO:16 (CDR1), SEQ ID NO:17 (CDR2) and SEQ ID NO:18 (CDR3) and a second antigen binding unit specifically binding to CD3 comprising light chain CDRs comprising the amino acid sequences of SEQ ID NO:55 (CDR1), SEQ ID NO:56 (CDR2) and SEQ ID NO:57 (CDR3) and heavy chain CDRs comprising the amino acid sequences of SEQ ID NO:58 (CDR1), SEQ ID NO:59 (CDR2) and SEQ ID NO:60 (CDR3).

43. The T-cell engaging protein for use according to item 42, wherein said T-cell engaging protein comprises (i) a first antigen binding unit specifically binding to DLL3 comprising a light chain variable domain comprising the amino acid sequence of SEQ ID NO:41 and heavy chain variable domain comprising the amino acid sequence of SEQ ID NO:42 and (ii) a second antigen binding unit specifically binding to CD3 comprising a light chain variable domain comprising the amino acid sequence of SEQ ID NO:67 and a heavy chain variable domain comprising the amino acid sequence of SEQ ID NO:68.

44. The T-cell engaging protein for use according to item 43, wherein said T-cell engaging protein comprises a first single chain Fab forming a first antigen binding unit comprising the sequence of SEQ ID NO:51 and a second single chain Fab comprising the sequence of SEQ ID NO:71 forming a second antigen binding unit.

45. The T-cell engaging protein for use according to any one of items 38 to 44, wherein said T-cell engaging protein comprises of a first polypeptide chain specific for DLL3 comprising an amino acid sequence of SEQ ID NO:75 and a second polypeptide chain specific for CD3 comprising the amino acid sequence of SEQ ID NO:79.

46. The T-cell engaging protein for use according to any one of the preceding items, wherein essentially no cytokine release syndrome occurs after said initial and/or said elevated dose of said step-in cycle in the subject.

47. The T-cell engaging protein for use according to any one of the preceding items, wherein said T-cell engaging protein is the only anti-cancer agent to be administered.

48. The T-cell engaging protein for use according to any one of the preceding items, wherein at least one further anti-cancer agent is to be administered, said further anti-cancer agent preferably being a chemotherapeutic agent and/or an immune-checkpoint inhibitor such as an anti-PD-1 antibody or an anti-PD-L1 antibody.

49. The T-cell engaging protein for use according to any one of the preceding items, wherein said T-cell engaging protein is administered by intravenous (IV) infusion.

50. The T-cell engaging protein for use according to any one of the preceding claims, wherein the subject is a human.

51. The T-cell engaging protein for use according to any one of the preceding items, the method further comprising co-administering to the subject a dose of lurbinectedin.

52. The T-cell engaging protein for use according to any one of the preceding items, the method further comprising co-administering to the subject a dose of amrubicin.

53. The T-cell engaging protein for use according to any one of the preceding items, comprising a step of co-administering topotecan to the subject, preferably for use in the treatment of SCLC (any treatment line) including ES-SCLC, preferably second line (2L) treatment of ES-SCLC that progressed or recurred following Standard of Care (SoC) treatment and LS-SCLC, preferably first line (1L) treatment.

54. The T-cell engaging protein for use according to any one of the preceding items, comprising a step of co-administering lurbinectedin to the subject, preferably for use in the treatment of SCLC (any treatment line) including ES-SCLC, preferably 2L treatment of ES-SCLC that progressed or recurred following SoC, and LS-SCLC, preferably 1L treatment.

55. The T-cell engaging protein for use according to any one of the preceding items, comprising a step of co-administering amrubicin to the subject, preferably for use in the treatment of SCLC (any treatment line) including ES-SCLC, preferably 2L treatment of ES-SCLC that progressed or recurred following SoC, and LS-SCLC, preferably 1L treatment.

56. The T-cell engaging protein for use according to any one of the preceding items, comprising a step of co-administering etoposide and carboplatin or cisplatin, and optionally also anti-PD-L1 antibody e.g. atezolizumab or durvalumab, to the subject, preferably for use in the treatment of SCLC including ES-SCLC, especially for 1L, 2L and/or 3L treatment, and LS-SCLC (any treatment line).

57. The T-cell engaging protein for use according to item 56, wherein the T-cell engaging protein, carboplatin or cisplatin and etoposide are co-administered for 3 to 5 treatment cycles, in-volving a step in cycle.

58. The T-cell engaging protein for use according to item 57, wherein only the T-cell engaging protein will be administered after said 3-5 treatment cycles, in particular for a maximum of 36 months.

59. The T-cell engaging protein for use according to any one of the preceding items, comprising a step of co-administering etoposide and carboplatin or cisplatin, and optionally also anti-PDL1 e.g. atezolizumab, to the subject, preferably for use in the treatment of SCLC including ES-SCLC, especially for 1L, 2L and/or 3L treatment, and LS-SCLC.

60. The T-cell engaging protein for use according to any one of items 1 to 59, wherein the cancer is epNEC, optionally epNEC excluding NEPC.

61. The T-cell engaging protein for use according to any one of items 1 to 59, wherein the cancer is SCLC.

62. The T-cell engaging protein for use according to any one of items 1 to 59, wherein the cancer is LCNEC of the lung.

63. The T-cell engaging protein for use according to any one of items 1 to 59, wherein the cancer is glioma.

64. The T-cell engaging protein for use according to any one of items 1 to 59, wherein the cancer is NEC.

65. The T-cell engaging protein for use according to any one of items 1 to 59, wherein the cancer is NEPC.

66. The T-cell engaging protein for use according to any one of the preceding items, further comprising a step of co-administering etoposide and carboplatin or cisplatin (optionally also anti-PD-L1 antibody), to the subject, preferably for use in the treatment of eps NEC or LCNEC of the lung (any treatment line), preferably first line treatment of epNEC or LCNEC of the lung.

67. The T-cell engaging protein for use according to any one of the preceding items, wherein carboplatin and cisplatin and etoposide, optionally also anti-PD-L1 antibody, are co-administered for 3 to 5 treatment cycles, in-volving a step in cycle, wherein more preferably, only the T-cell engaging protein, optionally together with anti-PD-L1 antibody, are administered after said 3-5 treatment cycles, in particular for a maximum of 36 months, wherein more optionally these cycles may involve the step-in cycle for each regimen.

68. The T-cell engaging protein for use according to any one of the preceding items, wherein one or more treatment cycles (e.g. one or two treatment cycles) including only chemotherapy take place prior to any of the combination treatments with the T cell engaging protein.

69. The T-cell engaging protein for use according to any one of the preceding items, wherein lurbinectedin, if co-administered may optionally be administered at the approved regimen of 3.2 mg/m² on day 1 of each 21 day cycle as an intravenous infusion, especially for 60 minutes.

70. The T-cell engaging protein for use according to any one of the preceding items, wherein amrubicin, if co-administered may optionally be administered at the approved regimen of 40 mg/m² on days 1 to 3 of each 21 day cycle, wherein more optionally amrubicin may be administered as an intravenous infusion.

71. The T-cell engaging protein for use according to any one of the preceding items, wherein topotecan, if co-administered may optionally be administered at the approved regimen of 2.3 mg/m² orally (p.o.) or 1.5 mg/m² by intravenous infusion daily on day 1 to day during each 21 day cycle.

72. The T-cell engaging protein for use according to any one of the preceding items, wherein carboplatin and etoposide, if co-administered may optionally be administered at the approved regimen of AUC 5 mg/mL min (carboplatin) and 100 mg/m² (etoposide), wherein carboplatin is administered at day 1 during each 21 day cycle and etoposide is administered on days 1 to 3 during each 21 day cycle.

73. The T-cell engaging protein for use according to any one of the preceding items, wherein cisplatin and etoposide, if co-administered may optionally be administered at the approved regimen of 75 mg/m2 (cisplatin) and 100 mg/m2 (etoposide), wherein cisplatin is administered at day 1 during each 21 day cycle and etoposide is administered on days 1 to 3 during each 21 day cycle.

74. The T-cell engaging protein for use according to any one of the preceding items, wherein a post-medication is administered directly after the administration of the T-cell engaging protein, wherein optionally “directly after” means that the post-medication is administered following the respective point in time of the start of the administration of a respective dose of the T-cell engaging protein.

75. The T-cell engaging protein for use according to item 74, wherein the post-medication comprises a non-steroidal anti-inflammatory drug, such as acetaminophen, an anti-histamine and/or a steroid, such as prednisolone or dexamethasone.

76. The T-cell engaging protein for use according to any one of item 74 to 75, wherein the post-medication comprises the same or a different substance as compared to the premedication according to item 35.

77. The T-cell engaging protein for use according to any one of items 74 to 76, wherein the post-medication is administered orally (p.o.) or intravenously (i.v).

78. The T-cell engaging protein for use according to any one of items 35 and 74 to 77, wherein the pre-medication is administered orally (p.o.) or intravenously (i.v.).

79. The T-cell engaging protein for use according to any one of items 35 and 74 to 78, wherein the post-medication is administered at least 6 hours up to 12 hours, such as 8±2 hours, after the respective administration of a dose of the T-cell engaging protein, wherein optionally “after” means that the post-medication is administered at least 6 hours up to about 12 hours, such as 8±2 hours, following the respective point in time of the start of the administration of a respective dose of the T-cell engaging protein.

80. The T-cell engaging protein for use according to any one of items 35 and 74 to 79, wherein the premedication is administered 0.25 to 2 hours, such as 0.5 hours to about 1 hour, prior to the respective administration of the T-cell engaging protein, wherein optionally “prior” means that the premedication is administered 0.25 to 2 hours, such as about 0.5 hours to about 1 hour, prior to the respective point in time of the start of the administration of a respective dose of the T-cell engaging protein.

81. The T-cell engaging protein for use according to any one of items 35 and 74 to 80, wherein the pre- and/or or post-medication comprises or is a steroid

82. The T-cell engaging protein according to item 81, wherein the steroid is a corticosteroid.

83. The T-cell engaging protein for use according to item 82, wherein the corticosteroid is selected from the group consisting of prednisolone and dexamethasone or a combination thereof.

84. The T-cell engaging protein for use according to item 83, wherein dexamethasone is used for the premedication and/or post-medication.

85. The T-cell engaging protein for use according to any one of items 35 and 74 to 84, wherein the steroid is selected from the group consisting of cortisone, betamethasone, triamcinolone, hydrocortisone, hydrocortisone, prednisone, methylprednisolone, dexamethasone or combinations thereof.

86. The T-cell engaging protein for use according to any of items 35 and 74 to 85, wherein an amount of a dexamethasone dose comprised in the premedication ranges from about 10 mg to about 18 mg, such as about 16 mg.

87. The T-cell engaging protein for use according to any of items 35 and 74 to 86, wherein an amount of a dexamethasone dose comprised in the premedication ranges from about 4 mg to about 10 mg, such as about 8 mg.

88. The T-cell engaging protein for use according to one any of items 35 and 74 to 87, wherein an amount of a dexamethasone dose comprised in the post-medication ranges from about 4 mg to about 10 mg, such as about 8 mg.

89. The T-cell engaging protein for use according to any one of the preceding items, where-in a premedication is administered directly prior the initial dose and a post-medication is administered directly after the initial dose, wherein the pre-medication is preferably administered about 0.25 to about 2 hours, such as 0.5 hours to about 1 hour before the initial dose and/or the post-medication is preferably administered at least 6 hours up to 12 hours, such as 8±2 hours, after the initial dose, wherein optionally “directly prior” means that the premedication is administered 0.25 to 2 hours, such as about 0.5 hours to about 1 hour, prior to the respective point in time of the start of the administration of the initial dose of the T-cell engaging protein and optionally directly after” means that the post-medication is administered at least 6 hours up to about 12 hours, such as 8±2 hours, following the respective point in time of the start of the administration of the initial dose of the T-cell engaging protein.

90. The T-cell engaging protein for use according to any one of the preceding items, where-in a premedication is administered directly prior the elevated dose and a post-medication is administered directly after the elevated dose, wherein the pre-medication is preferably administered about 0.25 to about 2 hours, such as 0.5 hours to about 1 hour before the elevated dose and/or the post-medication is preferably administered at least 6 hours up to 12 hours, such as 8±2 hours, after the elevated dose, wherein optionally “directly prior” means that the premedication is administered 0.25 to 2 hours, such as about 0.5 hours to about 1 hour, prior to the respective point in time of the start of the administration of the elevated dose of the T-cell engaging protein and optionally “directly after” means that the post-medication is administered at least 6 hours up to about 12 hours, such as 8±2 hours, following the respective point in time of the start of the administration of the elevated dose of the T-cell engaging protein.

91. The T-cell engaging protein for use according to any one of the preceding items, where-in a premedication is administered directly prior the further dose and a post-medication is administered directly after the further dose, wherein the pre-medication is preferably administered about 0.25 to about 2 hours, such as 0.5 hours to about 1 hour before the further dose and/or wherein the post-medication is preferably administered at least 6 hours up to 12 hours, such as 8±2 hours, after the further dose, wherein the pre-medication is preferably administered about 0.25 to about 2 hours, such as 0.5 hours to about 1 hour before the elevated dose and/or the post-medication is preferably administered at least 6 hours up to 12 hours, such as 8±2 hours, after the further, wherein optionally “directly prior” means that the premedication is administered 0.25 to 2 hours, such as about 0.5 hours to about 1 hour, prior to the respective point in time of the start of the administration of the elevated dose of the T-cell engaging protein and optionally directly after” means that the post-medication is administered at least 6 hours up to about 12 hours, such as 8±2 hours, following the respective point in time of the start of the administration of the further dose of the T-cell engaging protein.

92. The T-cell engaging protein for use according to any one of items 35 and 74 to 91, wherein the antihistamine of the premedication and/or the post-medication is diphenylhydramine, such as about 50 mg.

93. The T-cell engaging protein for use according to any one of items 35 and 74 to 92, wherein the non-steroidal anti-inflammatory drug of the premedication and/or the post-medication is acetaminophen/paracetamol, such as about 1000 mg.

94. The T-cell engaging protein for use according to any one of the preceding items, where-in a hospitalization, such as for 24 hours, follows the initial, elevated and/or further doses, i.e. the step-in doses, and/or the first target dose of the T-cell engaging protein.

95. The T-cell engaging protein for use according to any one of the preceding items, where-in a hospitalization, such as for about 24 hours, follows the initial and elevated doses, i.e. the first two step-in doses, of the T-cell engaging protein.

96. The T-cell engaging protein for use according to any one of the preceding items, where-in no hospitalization follows after the initial, elevated and further dose, i.e. the step-in doses, and the target dose(s) of the T-cell engaging protein.

97. The T-cell engaging protein for use according to any one of items 35 and 74 to 96, wherein the premedication and/or the post-medication comprise different compounds.

98. The T-cell engaging protein for use according to any one of items 35 and 74 to 97, wherein the premedication and/or the post-medication comprise an inhibitor/agonist of TNF-alpha (TNF) and/or TNF-alpha-Receptor (TNFR) reducing TNF/TFNR signalling and/or an inhibitor/agonist of IL6/IL6R that reduces IL6/IL6R signalling.

99. The T-cell engaging protein for use according to item 98, wherein the inhibitor/agonist of IL6/IL6R that reduces IL6/IL6R signal-ling is tocilizumab.

100. The T-cell engaging protein for use according to any one of items 35 and 74 to 99, wherein the premedication and/or post-medication comprise a steroid, wherein the steroid is selected from the group consisting of cortisone, betamethasone, triamcinolone, hydrocortisone, hydrocortisone, prednisone, methylprednisolone, dexamethasone or combinations thereof.

101. The T-cell engaging protein for use according to any one of items 35 and 74 to 100, wherein the premedication and/or the post-medication comprise an inhibitor/agonist of TNF/TNFR that reduces TNF/TNFR signaling being selected from the group consisting of etanercept, infliximab, adalimumab, certolizumab pegol and golimumab or a combination thereof.

102. The T-cell engaging protein for use according to item 101, wherein inhibitor/agonist of IL6/IL6R that reduces IL6/IL6R signaling is tocilizumab.

103. The T-cell engaging protein for use according to any one of items 35 and 74 to 102, wherein the premedication and/or the post-medication comprises a non-steroidal anti-inflammatory drug, an antihistamine and a steroid.

104. The T-cell engaging protein for us according to item 103, wherein the non-steroidal anti-inflammatory drug is acetaminophen/paracetamol, such as about 1000 mg p.o. or i.v., or equivalent, diphenhydramine, such as about 50 mg p.o. or i.v., or equivalent, dexamethasone, such as about 16 mg or about 8 mg p.o. or i.v., or equivalent intermediate-acting corticosteroid.

105. The T-cell engaging protein for use according to any one the preceding items, where-in the initial dose amount of the T-cell engaging protein is about 10 μg/kg and the elevated dose amount of the T-cell engaging protein is about 30 μg/kg, and wherein prior to and after those initial and elevated doses, a dexamethasone premedication and post-medication of a dose amount of about 8 mg is administered, respectively.

106. The T-cell engaging protein for use according to any one of items 35 and 77 to 108, wherein the premedication comprises a non-steroidal anti-inflammatory drug, such as acetaminophen/paracetamol, and an antihistamine, such as diphenhydramine.

107. The T-cell engaging protein for use according to item 106, wherein the premedication is administered prior to the respective step-in doses of the T-cell engaging protein, such as the initial dose, e.g. being about 10 μg/kg, such as the elevated dose, e.g. being about 30 μg/kg, and such as the further dose, e.g. being about 90 μg/kg.

108. The T-cell engaging protein for use according to item 106 or 107, wherein premedication may also be administered prior to the first to at least 6th or more target doses of the T-cell engaging protein, e.g. being about 60 mg of the T-cell engaging protein.

109. The T-cell engaging protein for use according to any one of items 106 to 108, wherein a steroid, such as dexamethasone, is administered as premedication in addition to the non-steroidal anti-inflammatory drug and the antihistamine and as a post-medication.

110. The T-cell engaging protein for use according to any one of items 106 to 109, wherein prior to each of the initial dose, the elevated dose and the further dose and the first two target doses of the T-cell engaging protein, the steroid, such as dexamethasone, is administered as a premedication.

111. The T-cell engaging protein for use according to item 110, wherein about 8 mg of the steroid being dexamethasone is respectively administered prior to the initial dose, the elevated dose, and the further dose of the T-cell engaging protein as premedication and about 8 mg of the steroid being dexamethasone is respectively administered after the initial dose, the elevated dose and the further dose of the T-cell engaging protein as post-medication.

112. The T-cell engaging protein according to item 110 or 111, wherein prior to the first target dose of the T-cell engaging protein about 4 mg of dexamethasone is administered as a premedication and after the first target dose of the T-cell engaging protein about 4 mg of dexamethasone is administered as a post-medication.

113. The T-cell engaging protein according to any one of items 106 to 112, wherein after the initial dose, the elevated dose, the further dose and the first target dose of the T-cell engaging protein, the steroid being dexamethasone, is administered, e.g. in an amount of about 8 mg, respectively.

114. The T-cell engaging protein according to any one of items 106 to 113, wherein the amount of the steroid being dexamethasone, is reduced at the first target dose of the T-cell engaging protein for the premedication and/or post-medication, if the T-cell engaging protein is well-tolerated, i.e. if no CRS≥2 and/or ICANS (any grade) occurred following the prior dose.

115. The T-cell engaging protein according to any one of items 106 to 114, wherein the steroid, such as dexamethasone, is omitted for the premedication and the post-medication starting from the second target dose of the T-cell engaging protein.

116. The T-cell engaging protein according to any one of items 106 to 115, wherein no more steroid, such as dexamethasone, is administered as a premedication from the second target dose of the T-cell engaging protein, if it is well tolerated.

117. The T-cell engaging protein for use according to any one of items 106 to 116, wherein if CRS/IRR/ICANS occur, the steroid, e.g. dexamethasone, dose for at least premedication at the next drug administration must remain the same as for the current dose and must not be reduced.

118. The T-cell engaging protein for use according to any one of items 106 to 117, wherein the line of treatment is 1L in DLL3 positive subjects and optionally, those subjects have unresectable locally advanced or metastatic epNEC, wherein, the co-administration involves—in addition to the T-cell engaging protein—an administration of carboplatin and etoposide.

119. The T-cell engaging protein for use according to any one of items 106 to 117, wherein the treatment is 1L and the co-administering—in addition to the T-cell engaging protein—involves administering atezolizumab, carboplatin and etoposide and wherein the cancer is ES-SCLC.

120. The T-cell engaging protein for use according to any one of items 35 and 74 to 119, wherein a premedication is administered directly prior the first target dose and a post-medication is administered directly after the first target dose, wherein the pre-medication is preferably administered about 0.25 to about 2 hours, such as 0.5 hours to about 1 hour before the first target dose and/or the post-medication is preferably administered at least 6 hours up to 12 hours, such as 8±2 hours, after the first target dose, wherein optionally “directly prior” means that the premedication is administered 0.25 to 2 hours, such as about 0.5 hours to about 1 hour, prior to the respective point in time of the start of the administration of the first target dose of the T-cell engaging protein and optionally “directly after” means that the post-medication is administered at least 6 hours up to about 12 hours, such as 8±2 hours, following the respective point in time of the start of the administration of the first target dose of the T-cell engaging protein

### EXAMPLES

### Example 1: Phase I Study of BI 764532

The study described herein is a first-in-human, Phase I study of BI 764532 monotherapy in patients with DLL3 expressing SCLC, large cell NEC, NEC or small cell carcinomas of any other origin (NCT04429087). The objectives of this study are to determine the maximum tolerated dose(s) (MTD) or the Recommended Dose for Expansion (RDE) and dosing regimen for BI 764532 in patients with SCLC or other NECs, and to evaluate safety, tolerability, pharmacokinetics (PK) and preliminary efficacy.

### Methods and Analysis

Study Design This Phase I study is a multicenter, open-label, non-randomized, dose-escalation study. In the Phase Ia period, BI 764532 is administered using different dosing regimens:
Regimen A: target dose once every 3 weeks, i.e. on day 1 of a 3-week cycle,
Regimen B1: target dose once a week, i.e. on Days 1, 8 and 15 of a 3-week cycle,
Regimen B2: initial weekly step-in dose(s) followed by a target dose administered weekly i.e. on Days 1, 8 and 15 of a 3-week cycle
Regimen B3: initial weekly step-in dose(s) followed by a target dose administered weekly, i.e. on days 1, 8 and 15 of a 3-week cycle, wherein the step-in dosing is different from B2.

### Dosing Regimen A

Treatment cycles of 3 week (21 day) duration. BI 764532 monotherapy. Dosing regimen comprising once every three week (q3w) intravenous (i.v.) administration of the target (x μg/kg) dose in cycles 1−x (i.e. on day 1 of each 21 day cycle).

TABLE 3A
Regimen A:
One cycle duration = 3 weeks
Treatment cycle and following: target dose administered on day 1 of each
cycle (i.e. q3w) until progression or toxicity, maximum of 36 months

| | Treatment cycle and following |
|---|---|
| | d 1 |
| dose | Target dose: |
| | from 0.03 to 90 μg/kg: |
| | 0.03 μg/kg, 0.1 μg/kg, 0.3 μg/kg, 0.9 μg/kg, 2.5 μg/kg, 5 μg/kg, |
| | 10 μg/kg, 30 μg/kg, 90 μg/kg |

### Dosing Regimen B1

Treatment cycles of 3 week (21 day) duration. BI 764532 monotherapy. Dosing regimen comprising once every week (q1w) intravenous (i.v.) administration of the target (x g/kg) dose in cycles 1 and following (i.e. on day 1, 8 and 15 of each 21 day cycle).

TABLE 3B
Regimen B1:
One cycle duration = 3 weeks
Treatment cycle and following: target dose administered
on day 1, 8 and 15 of each cycle (i.e. q1w) until progression
or toxicity, maximum of 36 months

| | Treatment cycle and following | | |
|---|---|---|---|
| | d 1 | d 8 | d 15 |
| dose | Target dose: | Target dose: | Target dose: |
| | from 0.9 to 30 | from 0.9 to 30 | from 0.9 to 30 |
| | μg/kg: | μg/kg: | μg/kg: |
| | 0.9 μg/kg, 10 μg/kg, | 0.9 μg/kg, 10 μg/kg, | 0.9 μg/kg, 10 μg/kg, |
| | 30 μg/kg | 30 μg/kg | 30 μg/kg |

### Dosing Regimen B2

Cycles of 3 week (21 day) duration. BI 764532 monotherapy. Dosing regimen comprising intravenous (i.v.) administration of a first step in dose (x1 μg/kg) in step-in cycle on day 1, a second step-in dose (x2 μg/kg) in step-in cycle on day 8, and a target dose (x3 μg/kg) in step-in cycle on day 15, followed by weekly i.v. administration of the target (x ag/kg) dose in all treatment cycles (i.e. on day 1, 8 and 15 of each 21 day cycle). A switch to Regimen B2 could be decided if >grade 2 toxicity occurred in regimen A or B1 after the first administration and this toxicity was determined to be due to CRS or related symptoms.

TABLE 3C
Regimen B2:
One cycle duration = 3 weeks
Step-in cycle: initial 3 doses on weekly intervals including 2 “step-in”
doses (d 1, d 8) and first target dose (d 15); target doses tested: 90 μg/kg
180 μg/kg, 270 μg/kg, 360 μg/kg, 540 μg/kg, 720 μg/kg, 1080 μg/kg,
1530 μg/kg)
Treatment cycles: Target doses (90 μg/kg 180 μg/kg, 270 μg/kg, 360
μg/kg, 540 μg/kg, 720 μg/kg, 1080 μg/kg, 1530 μg/kg) administered
weekly until progression or toxicity, maximum of 36 months

| | Step-in cycle | | | Treatment cycle 1 (repeated) | | |
|---|---|---|---|---|---|---|
| | d 1 | d 8 | d 15 | d 1 | d 8 | d 15 |
| dose | 30 μg/kg | 90 μg/kg | Target | Target | Target | Target |
| | | | dose: | dose: | dose: | dose: |
| | | | from 90 | from 90 | from 90 | from 90 |
| | | | to 1530 | to 1530 | to 1530 | to 1530 |
| | | | μg/kg | μg/kg | μg/kg | μg/kg |

Regimens were assessed sequentially, starting with Regimen A. Patients were treated until disease worsening or for a maximum of 36 months.

These schedules of administration were selected according to pharmacokinetic modelling based on preclinical data.

Regimen A was selected to avoid clinically significant drug accumulation beyond the first cycle. Accordingly, it was considered appropriate to explore the safety of a single dose BI 764532 using a Bayesian Logistic Regression Model (BLRM) with an initial MTD period of 3 weeks.

Regimen B1 was initiated once data from Regimen A, including safety, pharmacokinetics, biomarker analysis, potential of drug accumulation and possible efficacy had been assessed.

Regimen B2 was initiated according to protocol when the observed adverse events (AEs), especially cytokine release syndrome (CRS) precluded further does escalation in Regimens A and B1. In regimens A and B1 the target dose was administered from the first administration and onwards (q3w or q1w); dose escalation in A and B1 was stopped at 90 and 30 μg/kg, respectively (safety findings at the initial 90 μg/kg administration did not allow further dose escalation despite premedication including steroids) and Regimen B2 (step-in dosing) was initiated with the aim to reduce the frequency and severity of adverse events at the initial administration(s), especially CRS and ICANS, and to reach higher therapeutic doses (target doses).

### Dosing Regimen B3

Treatment cycles of 3 week (21 day) duration. BI 764532 monotherapy. Dosing regimen comprising i.v. administration of a first step in dose (x1 μg/kg) in step-in cycle on day 1, a second step-in dose (x2 μg/kg) in step-in cycle on day 8, a third step-in dose (x3 μg/kg) in step-in cycle on day 15, followed by weekly i.v. administration of the target dose (x g/kg) in treatment cycle 1 (i.e. on day 1, 8 and 15 of the 21 day cycle) followed by once every three week (q3w) administration of the target dose in repeated treatment cycles 2 (i.e. on day 1).

TABLE 4
Regimen B3:
one cycle = 3 weeks duration
Step-in cycle: initial 3 doses on weekly intervals as “step-in”
Treatment cycle 1: Target doses (90 μg/kg, 270 μg/kg, 360 μg/kg,
540 μg/kg, 720 μg/kg, or 1080 μg/kg) administered weekly
Treatment cycles 2 (repeated): Target doses (90 μg/kg, 270 μg/kg,
360 μg/kg, 540 μg/kg, 720 μg/kg, or 1080 μg/kg) administered once
every 3 weeks (q3w) until progression or toxicity, maximum of 36 months

| | | | | | | | Treatment |
|---|---|---|---|---|---|---|---|
| | | | | | | | cycles 2 |
| | Step-in cycle | | | Treatment cycle 1 | | | (repeated) |
| | d 1 | d 8 | d 15 | d 1 | d 8 | d 15 | d 1 |
| dose | 10 | 30 | 90 | Target | Target | Target | Target |
| | μg/kg | μg/kg | μg/kg | dose: | dose: | dose: | dose: |
| | | | | 90- | 90- | 90- | from 90 |
| | | | | 1080 | 1080 | 1080 | to 1080 |
| | | | | μg/kg | μg/kg | μg/kg | μg/kg |

Regimen B3 was initiated to further improve tolerability by a modified step-in as well as reduce burden of long term weekly iv administration in Regimen B2. Regime B3 is a “hybrid regimen” starting with q1w dosing and continuing in treatment cycles 2 with q3w administration.

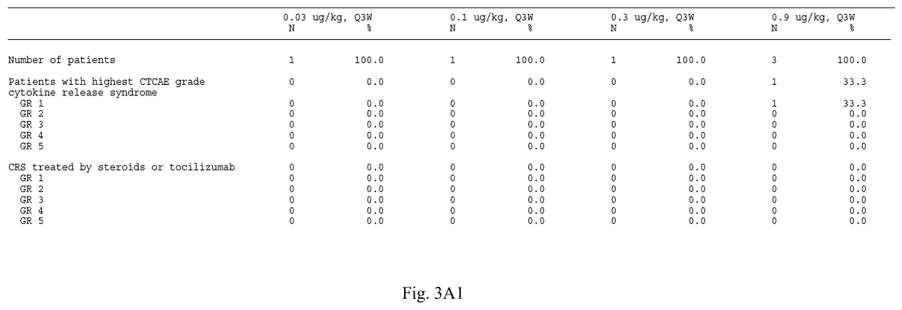

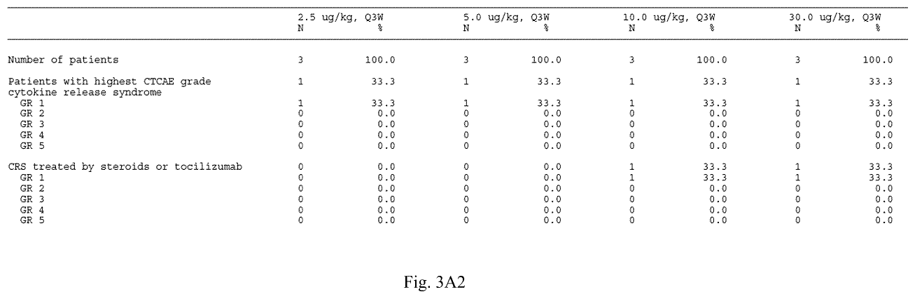

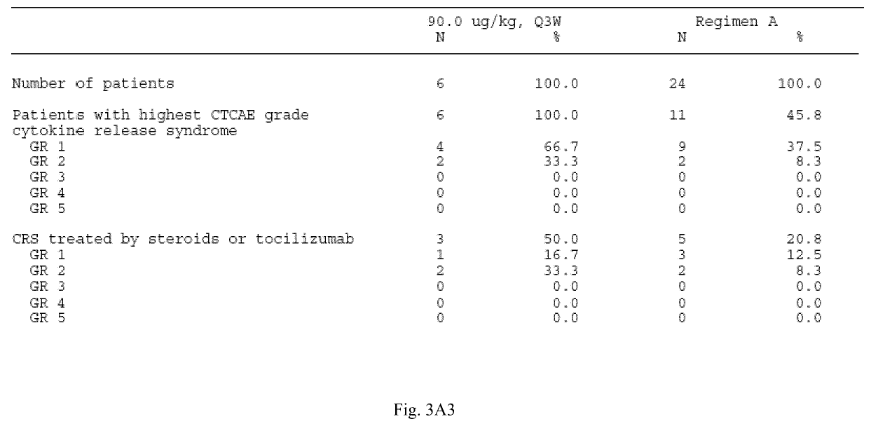

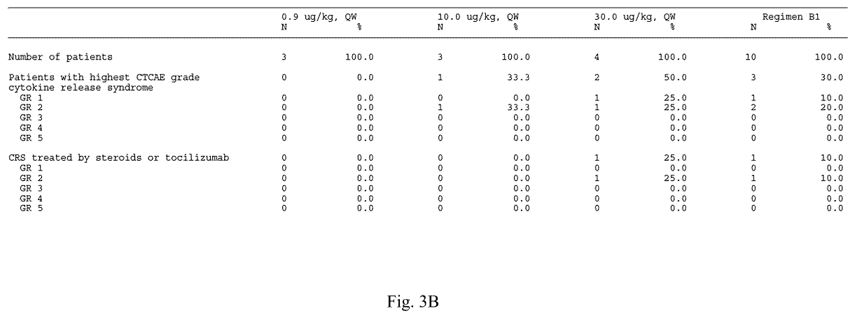

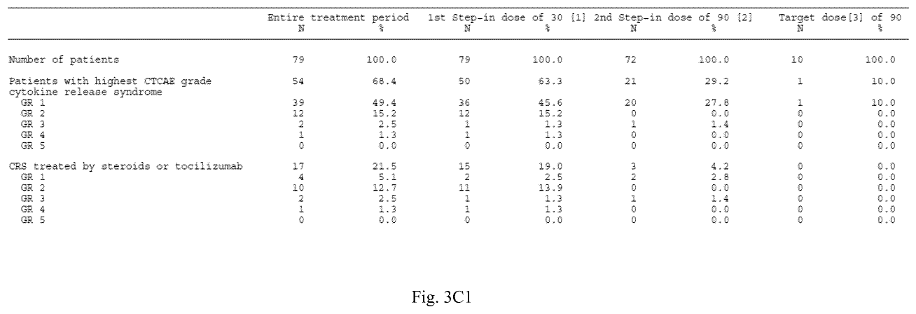

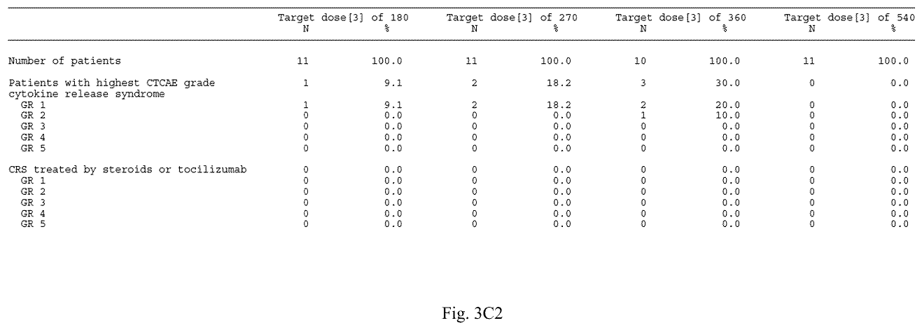

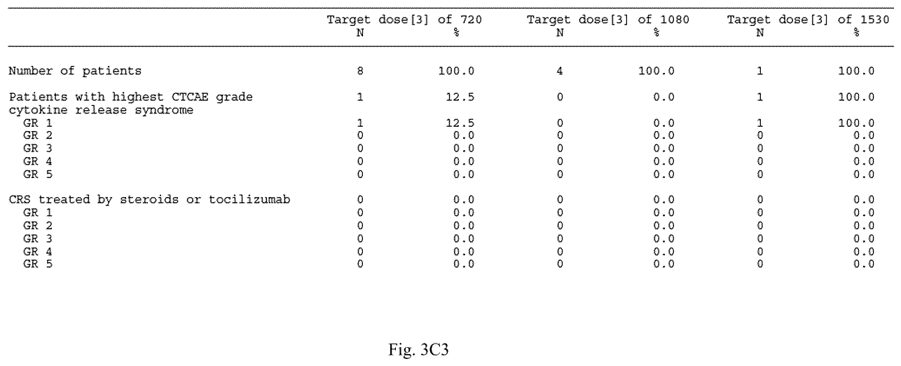

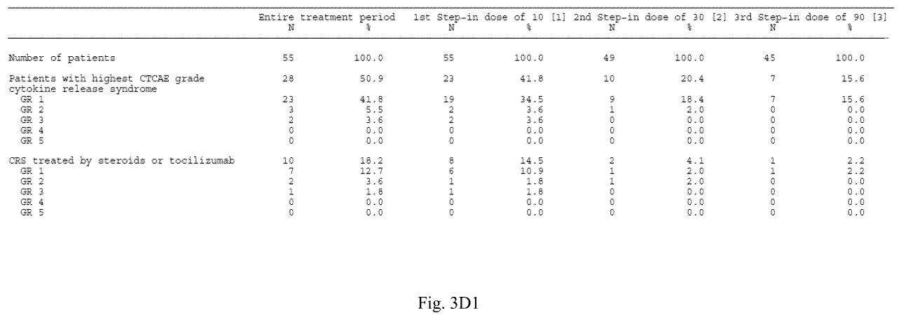

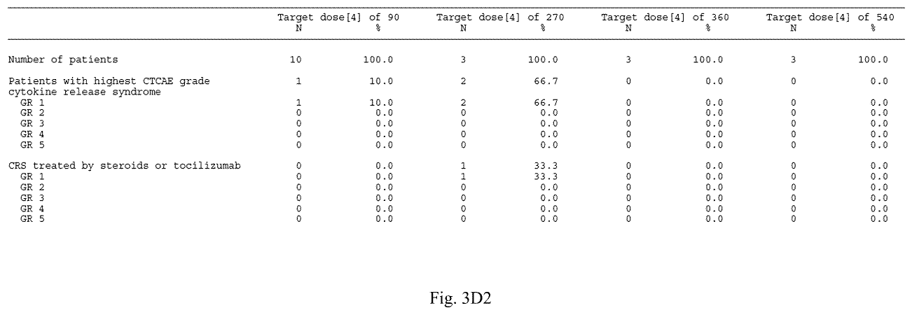

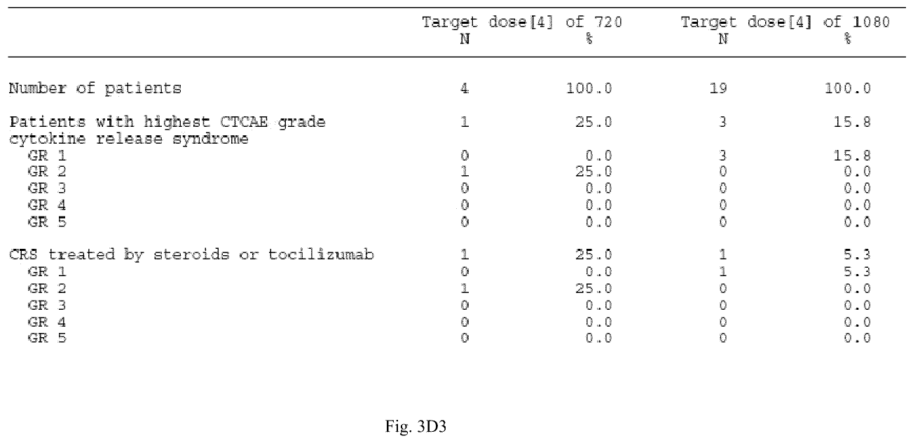

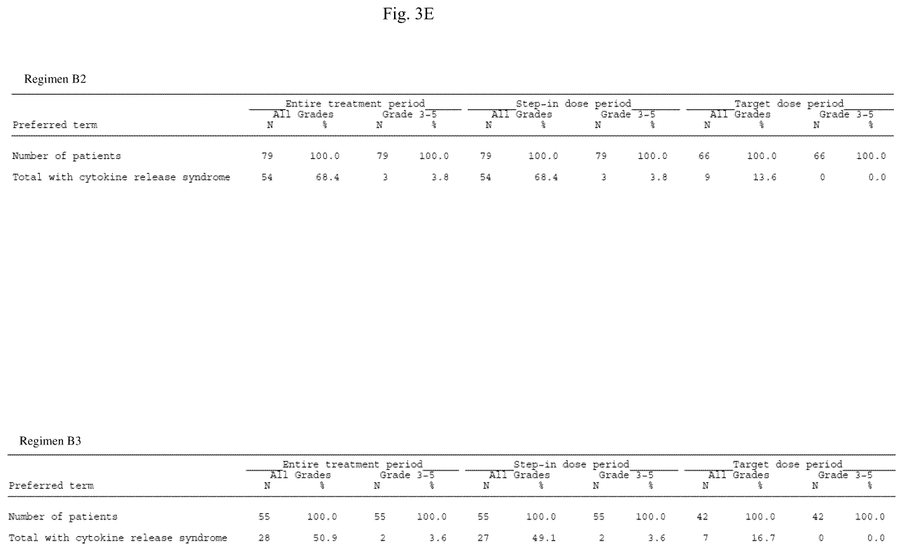

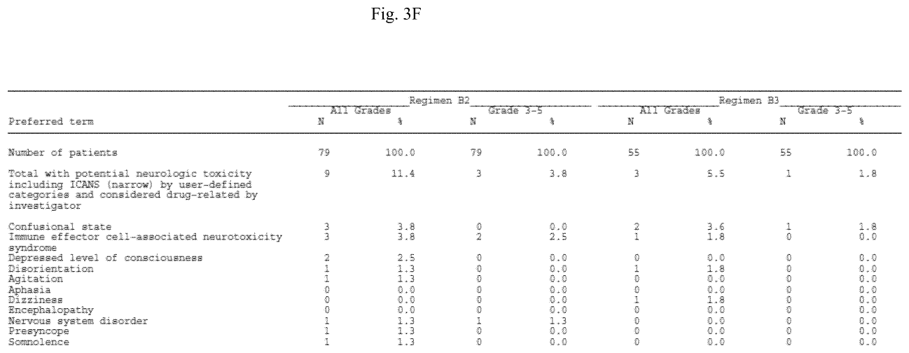

Step-in strategy in Regimen B3: safety data for Regimen B2 (most CRS cases were observed at the step-in doses with the majority at the initial 30 μg/kg step-in dose, FIG. 3C1) indicated that the initial step-in dose is critical for the overall tolerability of BI 764532 and further modifications of step-in dosing might improve the safety. With the aim to further reduce the frequency and severity of adverse events, especially CRS and potentially as well ICANS, during the step-in phase, a lower initial step-in dose of 10 μg/kg was added to the step-in regimen. Accordingly, patients in Regimen B3 received a first step in dose (10 μg/kg) in step-in cycle on day 1, a second step-in dose (30 μg/kg) in step-in cycle on day 8, and a third step-in dose (90 μg/kg) in step-in cycle on day 15. Administration of target dose started one week after the 3^{rd} step-in dose, i.e. at treatment cycle 1 day 1. In case Grade 2 or 3 drug related AEs occurred at step-in doses the same step-in dose could be given at the next administration or next drug administration could be delayed to extend the step in period. For individual patients the target dose might be reached at a later time.

Target doses in Regimen B3:_based on the observation in Regimen B2 (tolerability of target doses 90-1530 μg/kg) target doses of BI 764532 were escalated in Regimen B3 from 90 μg/kg to 1080 μg/kg over a total of 6 dose levels. In treatment cycle 1 target doses were administered weekly (i.e. treatment cycle 1 on day 1, treatment cycle 1 on day 8, and treatment cycle 1 on day 15). From treatment cycle 2 onwards target dose was administered once per cycle (cycle x day 1), i.e. once every 3 weeks (q3w) until progression or toxicity, maximum of 36 months. The rationale for “hybrid-dosing”, i.e. switching from q1w to q3w administration from treatment cycle 1 to treatment cycle 2 is that administration regimen q3w may be more convenient for patients.

Regimens B4a, B4b, B4c and B4d are further included in the Phase I study described in the present example, build on clinical results generated with Regimen B3 and aim to further optimize tolerability and time clinically active doses by a modified step-in strategy. Furthermore, Regimens B4a, B4b, B4c and B4d will start with body-weight adapted dosing with a switch to fixed doses (i.e. doses without adaptation to body weight) at subsequent administration—based on the rationale described in Example 4 below). Regimens B4a, B4b, B4c and B4d are “hybrid regimen” starting with q1w dosing and continuing in treatment cycles 2 onwards with q3w administration.

B4a: Treatment cycles of 3 week (21 day) duration. BI 764532 monotherapy. Dosing regimen comprising i.v. administration of a first step in dose (10 μg/kg) in step-in cycle on day 1, a second step-in dose (90 μg/kg) in step-in cycle on day 8, followed by administration of the body weight-independent target dose of 10 mg (Week 3). The target dose of 10 mg will further be administered weekly in treatment cycle 1 (i.e. on day 1, 8 and 15 of the 21 day cycle) followed by once every three week (q3w) administration of the target dose in repeated treatment cycles 2 (i.e. on day 1).

In case Grade 2 or 3 drug related AEs occur in step-in cycle the investigator may decide to repeat at the next administration the same step-in dose or delay next drug administration to extend the step in period (as a result for individual patients the target dose might be reached at a later time). Based on safety findings at the target dose individual target dose reduction will be allowed as needed.

Regimen B4a:
one cycle = 3 weeks duration
Step-in cycle: initial 3 doses on weekly intervals as “step-in”
Treatment cycle 1: Target dose 10 mg administered weekly
Treatment cycles 2 (repeated): Target dose 10 mg administered once every
3 weeks (q3w) until progression or toxicity, maximum of 36 months

| | | | | | | | Treatment |
|---|---|---|---|---|---|---|---|
| | | | | | | | cycles 2 |
| | Step-in cycle | | | Treatment cycle 1 | | | (repeated) |
| | d 1 | d 8 | d 15 | d 1 | d 8 | d 15 | d 1 |
| dose | 10 | 90 | 10 | Target | Target | Target | Target |
| | μg/kg | μg/kg | mg | dose: | dose: | dose: | dose: |
| | | | | 10 mg | 10 mg | 10 mg | 10 mg |

Regimen B4b:
one cycle = 3 weeks duration
Step-in cycle: initial 3 doses on weekly intervals as “step-in”
Treatment cycle 1: Target dose 60 mg administered weekly
Treatment cycles 2 (repeated): Target dose 60 mg administered once every
3 weeks (q3w) until progression or toxicity, maximum of 36 months

| | | | | | | | Treatment |
|---|---|---|---|---|---|---|---|
| | | | | | | | cycles 2 |
| | Step-in cycle | | | Treatment cycle 1 | | | (repeated) |
| | d 1 | d 8 | d 15 | d 1 | d 8 | d 15 | d 1 |
| dose | 10 | 10 | 60 | Target | Target | Target | Target |
| | μg/kg | mg | mg | dose: | dose: | dose: | dose: |
| | | | | 60 mg | 60 mg | 60 mg | 60 mg |

B4c: Treatment cycles of 3 week (21 day) duration. BI 764532 monotherapy. Dosing regimen comprising i.v. administration of a first step in dose (10 μg/kg) in step-in cycle on day 1, a second step-in dose (60 μg/kg) in step-in cycle on day 8, followed by administration of the body weight-independent target dose of 10 mg (Week 3). The target dose of 10 mg will further be administered weekly in treatment cycle 1 (i.e. on day 1, 8 and 15 of the 21 day cycle) followed by once every three week (q3w) administration of the target dose in repeated treatment cycles 2 (i.e. on day 1).

In case Grade 2 or 3 drug related AEs occur in step-in cycle the investigator may decide to repeat at the next administration the same step-in dose or delay next drug administration to extend the step in period (as a result for individual patients the target dose might be reached at a later time). Based on safety findings at the target dose individual target dose reduction will be allowed as needed.

Regimen B4c:
one cycle = 3 weeks duration
Step-in cycle: initial 3 doses on weekly intervals as “step-in”
Treatment cycle 1: Target dose 10 mg administered weekly
Treatment cycles 2 (repeated): Target dose 10 mg administered once every
3 weeks (q3w) until progression or toxicity, maximum of 36 months

| | | | | | | | Treatment |
|---|---|---|---|---|---|---|---|
| | | | | | | | cycles 2 |
| | Step-in cycle | | | Treatment cycle 1 | | | (repeated) |
| | d 1 | d 8 | d 15 | d 1 | d 8 | d 15 | d 1 |
| dose | 10 | 60 | 10 | Target | Target | Target | Target |
| | μg/kg | μg/kg | mg | dose: | dose: | dose: | dose: |
| | | | | 10 mg | 10 mg | 10 mg | 10 mg |

B4d: Treatment cycles of 3 week (21 day) duration. BI 764532 monotherapy. Dosing regimen comprising i.v. administration of a first step in dose (10 μg/kg) in step-in cycle on day 1, a second step-in dose (60 μg/kg) in step-in cycle on day 8, followed by administration of the body weight-independent target dose of 60 mg (Week 3). The target dose of 60 mg will further be administered weekly in treatment cycle 1 (i.e. on day 1, 8 and 15 of the 21 day cycle) followed by once every three week (q3w) administration of the target dose in repeated treatment cycles 2 (i.e. on day 1).

In case Grade 2 or 3 drug related AEs occur in step-in cycle the investigator may decide to repeat at the next administration the same step-in dose or delay next drug administration to extend the step in period (as a result for individual patients the target dose might be reached at a later time). Based on safety findings at the target dose individual target dose reduction will be allowed as needed.

Regimen B4d:
one cycle = 3 weeks duration
Step-in cycle: initial 3 doses on weekly intervals as “step-in”
Treatment cycle 1: Target dose 10 mg administered weekly
Treatment cycles 2 (repeated): Target dose 10 mg administered once every
3 weeks (q3w) until progression or toxicity, maximum of 36 months

| | | | | | | | Treatment |
|---|---|---|---|---|---|---|---|
| | | | | | | | cycles 2 |
| | Step-in cycle | | | Treatment cycle 1 | | | (repeated) |
| | d 1 | d 8 | d 15 | d 1 | d 8 | d 15 | d 1 |
| dose | 10 | 60 | 60 | Target | Target | Target | Target |
| | μg/kg | μg/kg | mg | dose: | dose: | dose: | dose: |
| | | | | 60 mg | 60 mg | 60 mg | 60 mg |

### Main Inclusion Criteria

Patients are adults (18 years) with locally advanced or metastatic tumors of the following histologies: SCLC; large cell neuroendocrine lung carcinoma; NEC or small cell carcinoma of any other origin. Patients have failed or are not eligible for available standard therapies and are previously received ≥1 line of chemotherapy (for SCLC platinum-based). Patients have an Eastern Cooperative Oncology Group performance status of 0 or 1, at least one evaluable lesion outside of the central nervous system (CNS), as defined by modified Response Evaluation Criteria In Solid Tumors (RECIST) version 1.1 criteria, adequate organ function, and have provided written informed consent. Patients are DLL3 positive (>0% DLL3 positivity).

### Study Endpoints

The primary endpoints of the Phase Ia period are: MTD in any studied regimen, defined as the highest dose with <25% risk of the true DLT rate being ≥33% during the MTD evaluation period, with separate MTDs determined in Regimen A and Regimen B; and the number of patients with DLTs in the MTD evaluation period.

The secondary endpoints of the Phase Ia period are objective response based on RECIST 1.1 criteria in patients with measurable disease, and the PK parameters maximum measured concentration of BI 764532 (Cmax) and the area under the concentration-time curve of the analyte over a uniform dosing interval τ (AUCτ). Further endpoints include DLL3 expression; tumor pathology and grade; immunogenic response; pharmacodynamic biomarker modulation in tumor tissue and blood; the number of patients with DLT during the entire treatment period; progression-free survival and duration of response; changes in soluble DLL3 in plasma over time; changes in circulating tumor cell counts over time (in SCLC patients only); and additional PK parameters.

### Statistical Analysis

### Determination of the MTD

For each dosing regimen, a separate BLRM employing the EWOC principle is used during the escalation phase for the selection of the dose levels and the estimation of the MTD, where applicable (Babb J, Rogatko A, Zacks S. Cancer phase I clinical trials: efficient dose escalation with overdose control. Stat Med 17(10), 1103-1120 (1998); Neuenschwander B, Branson M, Gsponer T. Critical aspects of the Bayesian approach to phase I cancer trials. Stat Med 27(13), 2420-2439 (2008)). The estimated probability of a DLT at each dose level from the model is summarized using the following intervals: under toxicity, 0.00-0.16; targeted toxicity, 0.16-0.33; and over toxicity, 0.33-1.00. Applying the EWOC criterion, it should be unlikely (less than 25% probability) that the DLT rate at that dose exceeds 0.33.

The MTD may be considered reached if one of the following criteria is fulfilled: the posterior probability of the true DLT rate in the target toxicity interval of the MTD is above 0.5, or at least 15 patients have been treated in the dose escalation phase of the trial, of which at least six have been treated at the MTD.

Safety Data from Phase I Study Across Regimens a, 81 and 82

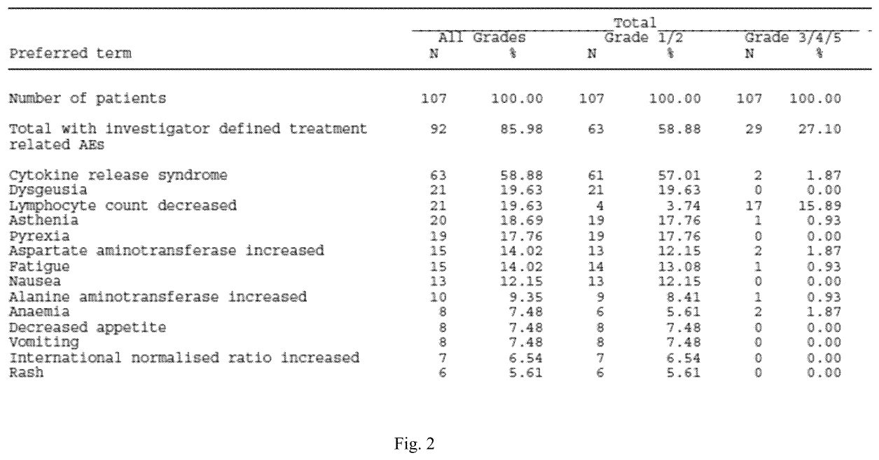

Safety data from the patients treated in the regimens A, B1 and B2 described above were collected using the CTCAE-version 5. The observed drug-related adverse events (AEs) are shown for these regimens in FIG. 2.

As expected from the BI 764532 Mode of Action (MoA) the most frequent drug-related AE was CRS which occurs in approximately 60% of the patients (all grades), most of the CRS events were low grade (CTCAE 1-2) and only approx. 2% of the patients experienced CRS grade 3 or higher.

Based on MoA and other published data for other TCE treatments as well neurological AEs, especially ICANS, could be expected as well.

### Results of Phase I Study

### A1

For Regimen A (see Table 3A above) the starting dose of BI 764532 was 0.03 μg/kg, based on a pharmacologically active dose, estimated from the 20% effect concentrations of the most sensitive in vitro pharmacology assay. For BI 764532 dose escalation the following provisional dose levels were planned per protocol: 0.1, 0.3, 0.9, 2.5, 5, 10, 20, 40 and 80 μg/kg. The trial protocol allowed for lower or higher doses to be investigated as long as they fulfil the escalation with overdose control (EWOC) principle. Application of the EWOC principle means that any dose tested should be unlikely (<25% posterior probability) to result in a DLT rate of ≥33%.

Based on reported safety per dose group the BI 764532 dose was escalated over a total of 9 dose levels (0.03 μg/kg, 0.1 μg/kg, 0.3 μg/kg; 0.9 μg/kg, 2.5 μg/kg, 5 μg/kg, 10 μg/kg, 30 ag/kg, 90 μg/kg). CRS (all grades) per dose level was reported in Regimen A at the following frequencies: 0% at 0.03 μg/kg, 0% at 0.1 μg/kg, 0% at 0.3 μg/kg; 33% at 0.9 μg/kg, 33% at 2.5 μg/kg, 33% at 5 μg/kg, 33% at 10 μg/kg, 33% 30 μg/kg, 100% at 90 μg/kg. These results indicate that frequency of CRS increases with dose (dose safety correlation). At the dose of 90 μg/kg all pts experienced CRS (6 out of 6, 100%, FIG. 3A3) in 2 cases the CRS was CTCAE grade 2 (33%). All pts treated at the dose of 90 μg/kg experienced CRS at the first BI 764532 administration; at the 2^{nd} administration 2 out of 6 pts experienced CRS and at subsequent administrations (≥3) only one patient was reported with CRS (data not shown). These results indicate that the CRS frequency decreases with repeated dosing. Furthermore, 3 pts (50% of pts) were reported with ICANS at BI 764532 90 μg/kg, ICANS was grade 2 in 2 cases and grade 3 in one case. Based on these safety findings it was concluded that 90 μg/kg in Regimen A was the upper limit where the dose can be safely escalated, and it was decided to stop dose escalation in Regimen A.

Tumor response was assessed in all patients enrolled. No response (CR or PR) by RECIST 1.1 was reported at BI 764532 dose levels of 0.03 μg/kg, 0.1 μg/kg, 0.3 μg/kg; 0.9 μg/kg, 2.5 μg/kg, 5 μg/kg, 10 μg/kg and 30 μg/kg. At 90 μg/kg a PR was reported in two patients. Accordingly, in Regimen A the 90 μg/kg dose was the only dose with a signal of clinical efficacy (data not shown).

B1 Dose escalation steps in Regimen B1 (see Table 3B): Based on reported safety per dose

group in Regimen A it was decided to escalate in Regimen B1 the BI 764532 dose from 0.9 ag/kg to 30 μg/kg over a total of 3 dose levels (0.9 μg/kg, 10 μg/kg and 30 μg/kg). CRS (all grades) per dose level was reported in Regimen B1 at the following frequencies: 0% at 0.9 ag/kg, 33% at 10 μg/kg, 50% at 30 μg/kg (FIG. 3B). Most of the CRS events occurred at the initial BI 764532 administration. The B1 data indicate that with q1w administration similar doses (per administration) are tolerable as with q3w dosing, i.e., the dose per administration rather than the cumulative dose per cycle seemed to determine tolerability. Dose escalation in Regimen B1 was stopped when the dose level 30 μg/kg was completed based on results from Regimen A indicating that further dose escalation in B1 to 90 μg/kg would not have been tolerable.

Tumor response was assessed in all patients enrolled. No response (CR or PR) by RECIST 1.1 was reported in Regimen B1 at any BI 764532 dose level. Accordingly, in Regimen B1 no signal of clinical efficacy was detected.

### B2

### Rationale for B2: In Regimens A and B1

dose escalation was stopped at 90 and 30 μg/kg, respectively, because of safety findings at the initial 90 μg/kg administration that did not allow further dose escalation despite premedication including steroids.
CRS frequency at the initial administration increased with dose (dose safety correlation)
CRS frequency decreased with repeated dosing

These results led to the conclusion that starting with a lower dose at the initial administration and increasing the dose at subsequent administrations (step-in dosing) should improve tolerability and might allow to reach target doses >90 μg/kg. Regimen B2 (step-in dosing, see Table 3C) was accordingly implemented aiming to reduce the frequency and severity of adverse events, such as CRS and ICANS and to reach higher therapeutic doses (target dose). For Regimen B2 the first administration (d1) was chosen to be 30 μg/kg and the second administration (d8) was chosen to be 90 μg/kg based on safety data generated in Regimens A and B1. The third administration (d15) in B2 was defined to be the target dose. During Regimen B2 the initial administration of 30 μg/kg and second administration of 90 μg/kg remained fixed and target dose was escalated.

Based on reported safety per dose group it was decided to escalate the BI 764532 target dose from 90 μg/kg to 1530 μg/kg over a total of 8 dose levels (90 μg/kg, 180 μg/kg, 270 μg/kg, 360 μg/kg, 540 μg/kg, 720 μg/kg, 1080 μg/kg, 1530 μg/kg). CRS (all grades) was reported in Regimen B2 (all dose levels) in 67% of patients; most of the reported CRS events were low grade (CTCAE grade 1-2), and only approx. 3% of patients were reported with CRS CTCAE grade 3 and higher. The vast majority of the CRS events occurred at the initial BI 764532 step-in administrations, i.e. at the step-in doses of 30 μg/kg (CRS all grade 63%, CRS grade ≥3 approx 1.5%) and 90 μg/kg (CRS all grade approx 30%, CRS grade ≥3 approx 1.5%, FIG. 3C1). Surprisingly, at target dose levels (after step-in) the CRS severity was low (max grade 2), CRS frequency was well controlled (max 20%) and no CRS treatment with steroids or tocilizumab was required (data not shown, FIG. 3C1-C3).

Extended step-in period—in case Grade 2 or 3 drug-related AEs occurred during step-in dosing, the investigator was allowed to continue with the same step-in dose at the next administration, or to delay the next trial drug administration. In both cases, the step-in period was extended. Thus, the target dose was reached later for individual patients. In case of additional step-in doses, additional hospitalizations were required (until target dose is reached).

Safety conclusion: while in Regimens without step-in dosing dose escalation had to be stopped at 90 μg/kg, with implementation of step-in dosing the BI 764532 target dose could be increased up to 1530 μg/kg; and even at this target dose level in Regimen B2 no MTD is defined yet.

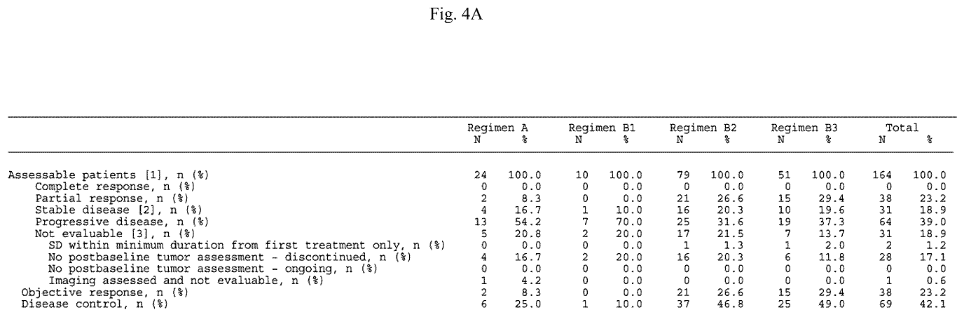

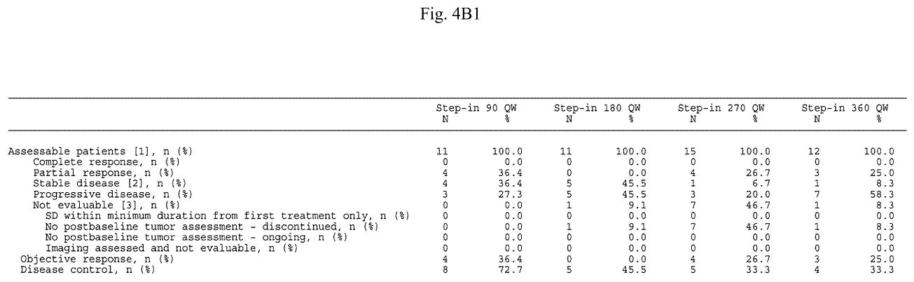

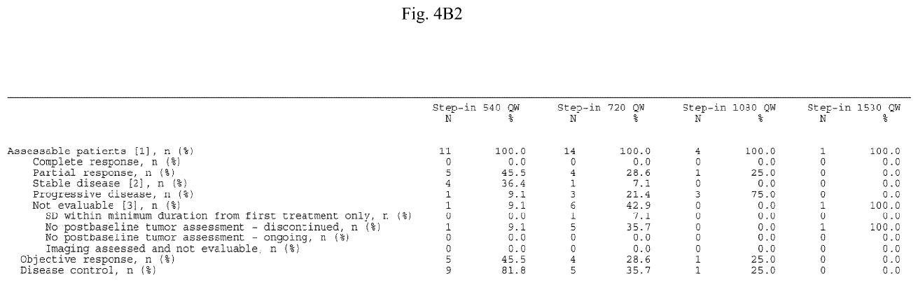

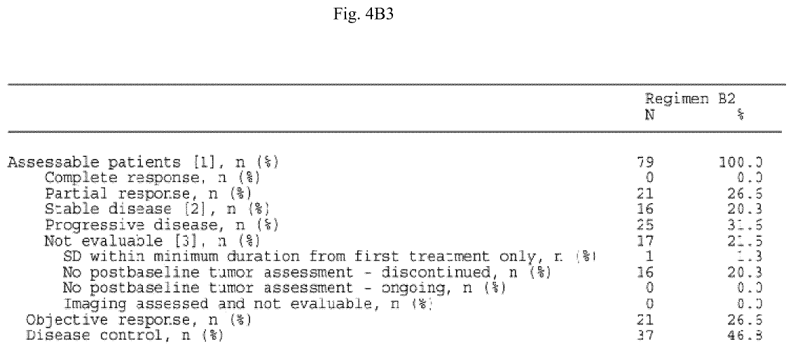

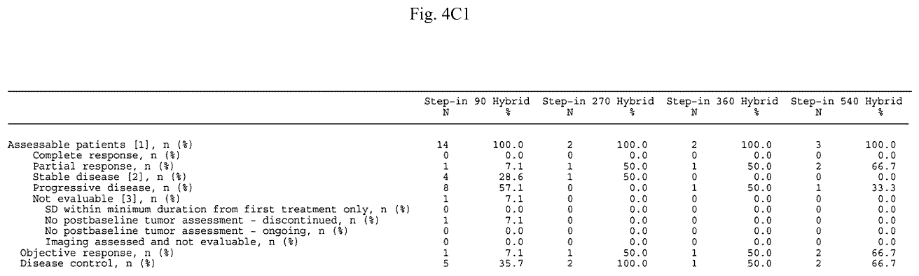

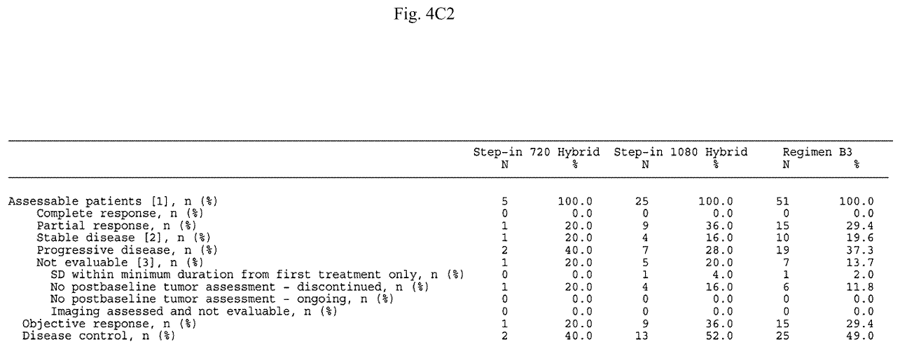

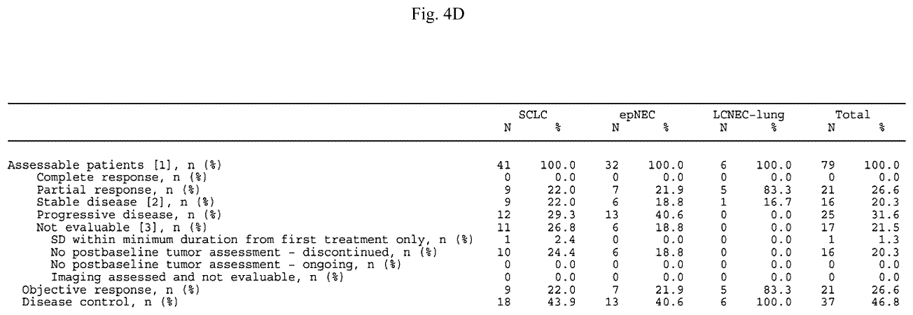

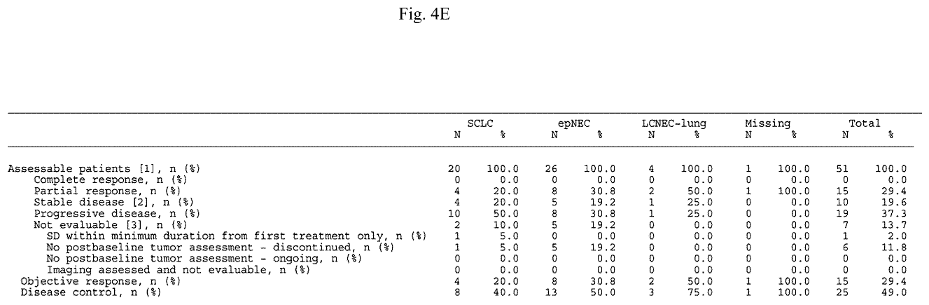

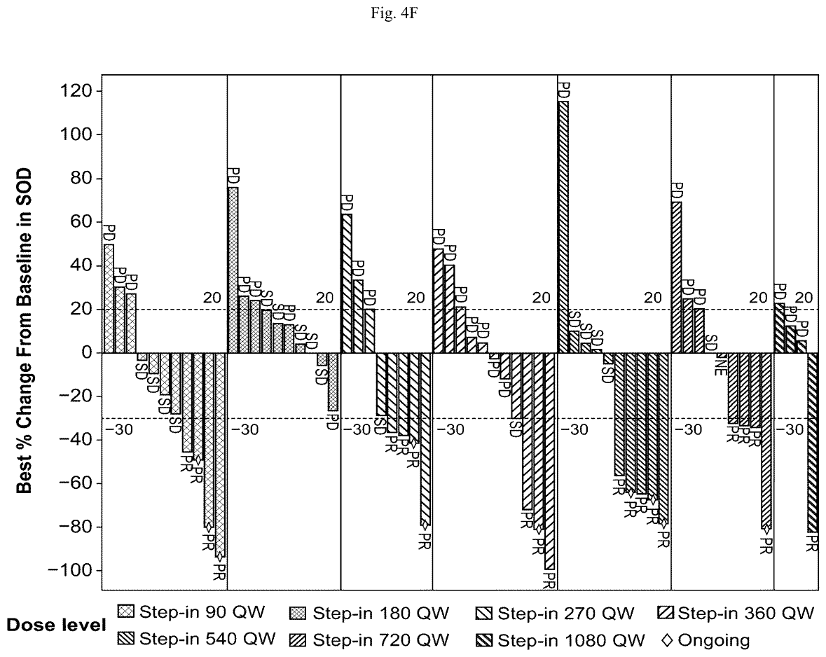

Tumor response was assessed in all patients enrolled in Regimen B2. Best overall tumor response PR by RECIST 1.1 was reported in 21 out of 79 evaluable patients (26.6%) across all BI 764532 dose levels in Regimen B2 (see FIG. 4B3).

### B3

Surprisingly, the overall CRS frequency (all CTCAE grades) in Regimen B3 was reduced as compared to Regimen B2 (50.9% vs 68.4%, FIGS. 3D1 and 3C1) and similarly CRS events of higher severity (CTCAE grade 2) were reported in B3 at a lower frequency (9.1% (5.5% grade 2, 3.6% grade 3) as compared to B2 (19% (15.2% grade 2, 2.5% grade 3, 1.3% grade 4)) (FIGS. 3D1 and 3C1). Especially during the step-in cycle the reduced frequency of CRS was notable comparing Regimens B2 (CRS, all grades reported in 68.4% of patients, FIG. 3E) and B3 (CRS, all grades reported in 49.1% of patients, FIG. 3E) while the frequency of CRS was low at target dose levels (treatment cycles) and comparable in both regimens (13.6% in B2 and 16.7% in B3) (FIG. 3E). At target dose levels (after step-in) no higher grade CRS (CTCAE grade 3) was reported and CRS treatment with steroids or tocilizumab was required only in a few cases (see FIGS. 3D1-D3 and 3C1-C3).

The overall ICANS frequency (all CTCAE grades) in Regimen B3 was surprisingly reduced as compared to Regimen B2 (5.5% vs 11.4%, FIG. 3F) and similarly ICANS events of higher severity (CTCAE grade 3) were reported in B3 at a lower frequency (1.8%) as compared to B2 (3.8%) (FIG. 3F).

Surprisingly, the clinical efficacy of treatment as measured by best overall tumor response and disease control was similar for regimens B2 (objective response 26.6%, disease control 46.8%) and B3 (objective response 29.4%, disease control 49%) (FIGS. 4A and 4C2).

Conclusion: the step-in Regimen in B3 improved tolerability of BI 764532 treatment as compared to the step-in Regimen in B2, by reducing the frequency of CRS occurring at step-in doses as well as reducing the frequency of ICANS while allowing to reach similar target doses in both regimens (B2 and B3) and achieving comparable clinical efficacy. Notably, clinical activity of BI 764532 treatment was reported in all indications (SCLC, epNEC, LCNEC) investigated in the Phase I study (FIGS. 4D, 4E)

### Example 2: Interferon-Gamma Levels at the Target Dose can be Reduced by Introduction of the Step-In Dosing

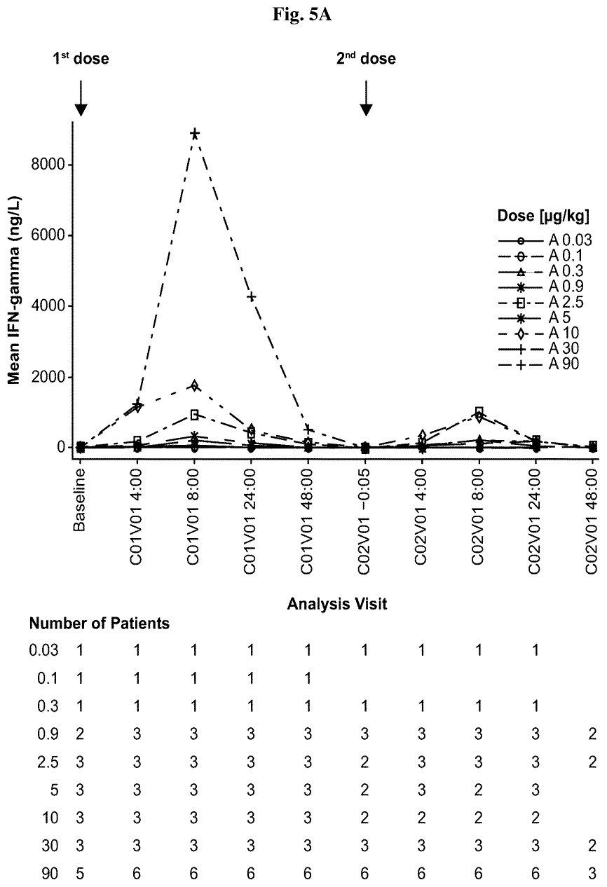

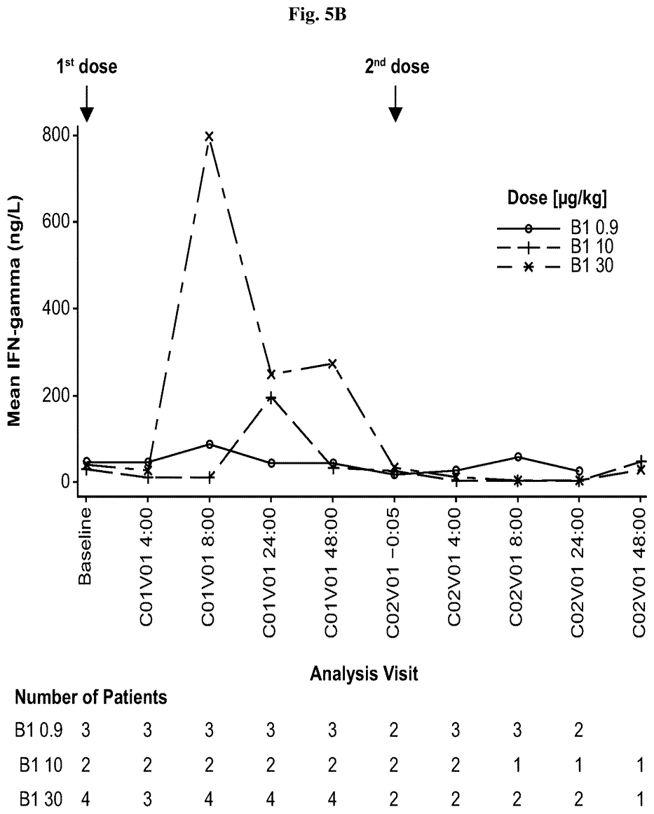

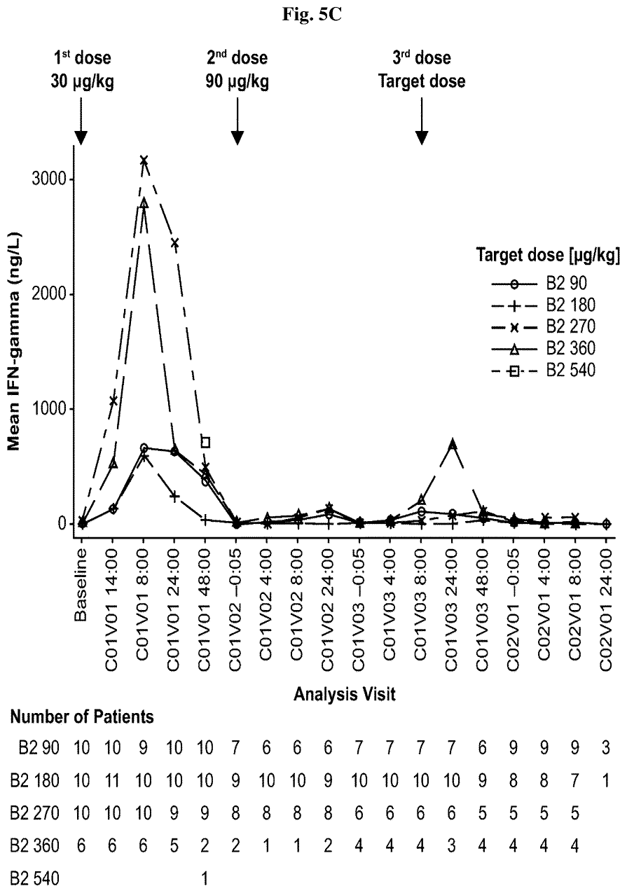

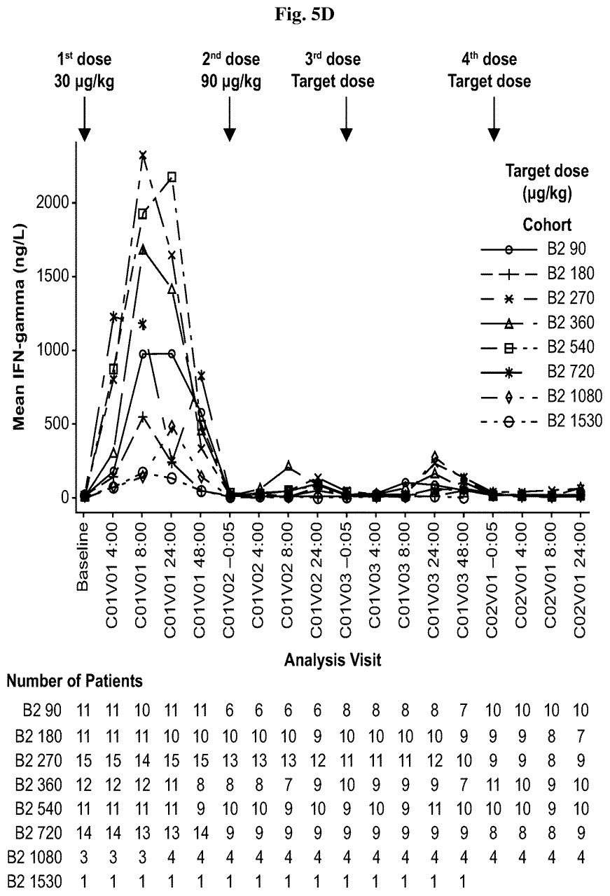

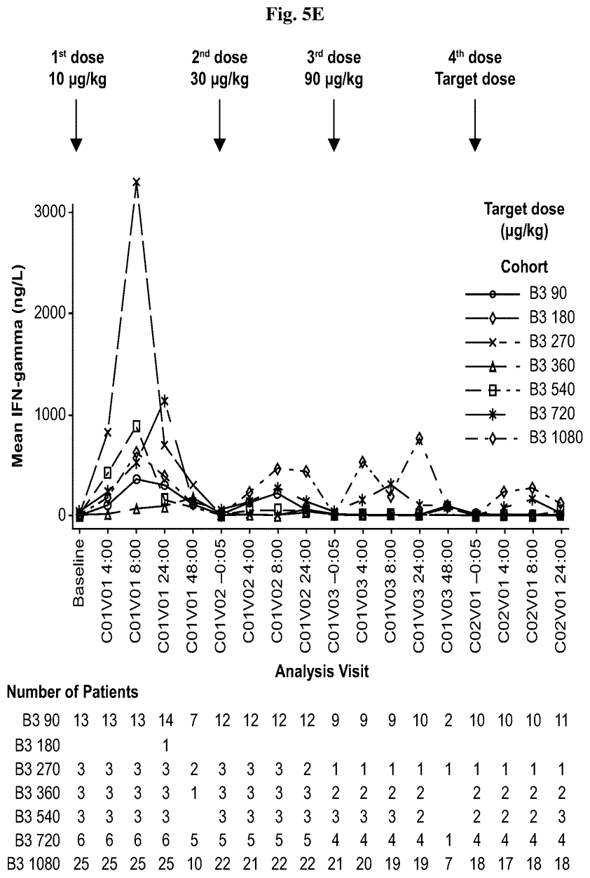

Serum levels of Interferon-gamma were quantified at various time points after administration of BI 764532. Initial peak of Interferon-gamma serum levels was observed 8 hours after administration of the first dose of BI 764532. After receiving the second dose of BI 764532 peak of Interferon-gamma levels were reduced when administered 3 weeks after the first dose (FIG. 5A, Regimen A) as well as when administered one week after the first dose (FIG. 5B, Regimen B1). When patients received a first dose of 30 μg/kg followed by a second dose of 90 μg/kg one week later, or a first dose of 10 μg/kg followed by a second dose of 30 μg/kg one week later, followed by a third dose of 90 μg/kg one week later, Interferon-gamma levels after the 3^{rd} or 4^{th} dose of 90, 180, 270, 360,540, 720, 1080, or 1530 μg/kg BI 764532 (target dose) were reduced compared to when patients received a first dose of 90 μg/kg (FIG. 5C, FIG. 5D, FIG. 5E, Regimen B2, Regimen B3).

### Example 3. Dareon™-5 an Open-Label, Multi-Center Phase II Dose Selection Trial of Intravenous BI 764532, a DLL3-Targeting T Cell Engager, in Patients with Relapsed/Refractory Extensive-Stage Small Cell Lung Cancer and in Patients with Other Relapsed/Refractory Neuroendocrine Carcinomas

### Dosing Regimen B2F and B3F

### Dosing Regimen B2F “B2 Flat”

Cycles of 3 week (21 day) duration. BI 764532 monotherapy. Dosing regimen comprising intravenous (i.v.) administration of a first step in dose (x1 μg/kg) in step-in cycle on day 1, a second step-in dose (x2 μg/kg) in step-in cycle on day 8, and a target dose (x3 mg) in step-in cycle on day 15, followed by weekly i.v. administration of the target (x mg) dose in treatment cycle 1 (i.e. on day 1, 8 and 15 of each 21 day cycle), followed by once every three week (q3w) administration of the target dose in repeated treatment cycles 2 (i.e. on day 1). A step in dose of 30 μg/kg, 90 μg/kg can be fairly assumed to have the same positive effects for a flat target dose of 10 mg, 30 mg or 60 mg in terms of the implementation of flat dosing scheme B2F for phase II in view of the safety conclusions of B2.

### Dosing Regimen B3F “B3 Flat”

Treatment cycles of 3 week (21 day) duration. BI 764532 monotherapy. Dosing regimen comprising a first step in dose (x1 μg/kg) in step-in cycle on day 1, a second step-in dose (x2 μg/kg) in step-in cycle on day 8, a third step-in dose (x3 μg/kg) in step-in cycle on day 15, followed by weekly administration of the target dose (fixed dose x mg) in treatment cycle 1 (i.e. on day 1, 8 and 15), followed by once every three week (q3w) administration of the target dose in repeated treatment cycles 2 (i.e. on day 1).

Following patients are included in this study: patients with SCLC who have had progression or recurrence following at least two prior lines of therapy, including at least one platinum-based regimen (in countries where standard of care in first line therapy includes PD-L1 inhibitor treatment patients should have received the combination of platinum-based regimen plus PD-L1 inhibitor unless they have been unable to receive checkpoint inhibitor treatment) and in patients with histologically or cytologically confirmed epNEC or LCNEC of the lung, who have progression or recurrence following at least one platinum-based regimen. In addition, the DLL3 positivity of those patients is ≥0%.

Regimen B2F and B3F
1 cycle = 3 weeks duration
Step-in cycle: initial 2 or 3 doses (×μg/kg) on weekly intervals
as “step-in”, optionally one target dose on day 15 if only 2
weight-based doses
Treatment cycle 1: Target doses (×mg) administered weekly
Treatment cycles 2 (repeated): Target dose (×mg) administered once every
3 weeks (q3w) until progression or toxicity, maximum of 36 months
(please, note that target doses are NOT weight based):

| | | | | | | | Treatment |
|---|---|---|---|---|---|---|---|
| | | | | | | | cycles 2 |
| | Step-in cycle | | | Treatment cycle 1 | | | (repeated) |
| | d 1 | d 8 | d 15 | d 1 | d 8 | d 15 | d 1 |
| Dose | 30 | 90 | Target | Target | Target | Target | Target |
| (B2F) | μg/kg | μg/kg | dose: | dose: | dose: | dose: | dose: |
| | | | From | From | From | From | From |
| | | | 5 mg to | 5 mg to | 5 mg to | 5 mg to | 5 mg to |
| | | | 60 mg | 60 mg | 60 mg | 60 mg | 60 mg |
| | | | | (i.e. 10, | (i.e. 10, | (i.e. 10, | (i.e. 10, |
| | | | | 30 or | 30 or | 30 or | 30 or |
| | | | | 60 mg) | 60 mg) | 60 mg) | 60 mg) |
| Dose | 10 | 30 | 90 | Target | Target | Target | Target |
| (B3F) | μg/kg | μg/kg | μg/kg | dose: | dose: | dose: | dose: |
| | | | | From | From | From | From |
| | | | | 5 mg to | 5 mg to | 5 mg to | 5 mg to |
| | | | | 60 mg | 60 mg | 60 mg | 60 mg |
| | | | | (i.e. 10, | (i.e. 10, | (i.e. 10, | (i.e. 10, |
| | | | | 30 or | 30 or | 30 or | 30 or |
| | | | | 60 mg) | 60 mg) | 60 mg) | 60 mg) |

Step-in strategy in B2F and B3F (step-in dosing with ≥2 or 3 steps and weight based dosing at least in d1 and d8 (10 μg/kg, 30 μg/kg, 90 μg/kg, or 30 μg/kg, 90 ag/kg, target dose (10, 30 or 60 mg)).
Target doses in Regimen B2F and B3F: at the target doses the doses will be fixed without adaptation to body weight. The rationale for fixed target doses is the following:
1) Tolerability of a wide range of target doses in Regimen B2 and B3:
target doses between 90 μg/kg and 1530 μg/kg were tolerated in Regimen B2 and B3 without results pointing to a dose-safety correlation at the target dose (i.e. no increased AE frequency in B2 or B3 at higher target doses). This indicates that adaptions of target dose to body weight are not needed for safety reasons. Accordingly, target doses of 10 mg and 60 mg (corresponding to 125 μg/kg and 750 μg/kg for a 80 kg patient) will be tested in Regimen B2F and B3F.
2) convenience of fixed doses: fixed dosing reduces complexity of drug preparation as well as drug wasting.

In contrast to the fixed target doses applied during treatment cycles in Regimen B2F and B3F at least two of the step-in doses in B3F and B2F will be adapted to body weight. The rationale for weight-adapted step in dosing is as follows:
dosing/exposure is critical for tolerability at the initial administrations (during step-in)
risk of CRS is considered to correlate with body weight.
weight-based step-in dosing was determined in Regimes B2 and B3 to result in manageable safety

### Example 4. a Phase I, Non-Randomized, Open-Label, Multi-Center Dose Escalation Trial of BI 764532 Combined with Ezabenlimab in Patients with Small Cell Lung Carcinoma and Other Neuroendocrine Neoplasms Expressing DLL3

### Dosing Regimen C

Treatment cycles of 3 week (21 day) duration. BI 764532 combined with a checkpoint inhibitor (ezabenlimab).
Dosing regimen comprising a first step in BI 764532 dose (x1 μg/kg) in step-in cycle on day 1, a second step-in dose (x2 μg/kg) in step-in cycle on day 8, a third step-in dose (x3 μg/kg) in step-in cycle on day 15, followed by weekly administration of the target dose (fixed dose x mg) in treatment cycle 1 (i.e. on day 1, 8 and 15), followed by once every three week (q3w) administration of the target dose in repeated treatment cycles 2 (i.e. on day 1).
The checkpoint-inhibitor (ezabenlimab) will be administered on day 1 of each treatment cycle.
Patients were DLL3 positive (>0% DLL3 positivity).

Combination of BI 764532 and ezabenlimab
1 cycle = 3 weeks duration
BI 764532 dosing (dose escalation in subsequent dose cohorts):
Step-in cycle: initial 3 doses (×μg/kg) on weekly intervals as “step-in”
Treatment cycle 1: Target doses (×mg) administered weekly
Treatment cycles 2 (repeated): Target dose (×mg) administered once every
3 weeks (q3w) until progression or toxicity, maximum of 36 months
Ezabenlimab (PD-1 inhibitor) dosing (fixed dose, no dose escalation):
240 mg i.v. infusion on day 1 in all cycles

| | | | | | | | Treatment |
|---|---|---|---|---|---|---|---|
| | | | | | | | cycles 2 |
| | Step-in cycle | | | Treatment cycle 1 | | | (repeated) |
| | d 1 | d 8 | d 15 | d 1 | d 8 | d 15 | d 1 |
| BI | 5-10 | 15-30 | 30-90 | Target | Target | Target | Target |
| 764532 | μg/kg | μg/kg | μg/kg | dose: | dose: | dose: | dose: |
| dose | | | | From | From | From | From |
| | | | | 5 mg to | 5 mg to | 5 mg to | 5 mg to |
| | | | | 60 mg | 60 mg | 60 mg | 60 mg |
| Eza- | 240 | | | 240 | | | 240 |
| benlimab | mg | | | mg | | | mg |
| dose | | | | | | | |

Step-in strategy in Regimen C: step-in dosing will start at reduced doses compared to B2, B3, B2F and B3F: initial step-in dosing with ≥3 steps and weight-based dosing (5 μg/kg, 15 μg/kg, 30 μg/kg). If the reduced step-in in combination with ezabenlimab is tolerated well the step-in doses might be increased in subsequent dosing cohorts
Target doses in Regimen C: the fixed target doses (i.e. without adaptation to body weight) will be escalated in subsequent dosing cohorts to define the MTD/RDE.
The rationale for body weight adapted step-in doses and fixed target doses is the same as for Regimen B2F and B3F
Depending on tolerability of the combination the BI 764532 dosing days might be shifted (e.g. to avoid BI 764532 dosing on days with ezabenlimab administration).

### Example 5A: Dareon™-9 Combination of BI 764532 with Topotecan, Lurbinectedin or Amrubicin in Patients with SCLC

### Dosing Regimen D

Treatment cycles of 3 week (21 day) duration. BI 764532 combined with a Topotecan, lurbinectedin or amrubicin.
Dosing regimen comprising a first step in BI 764532 dose (x1 μg/kg) in step-in cycle on day 1, a second step-in dose (x2 μg/kg) in step-in cycle on day 8, a third step-in dose (x3 μg/kg) in step-in cycle on day 15, followed by weekly administration of the target dose (fixed dose x mg) in treatment cycle 1 (i.e. on day 1, 8 and 15), followed by once every three week (q3w) administration of the target dose in repeated treatment cycles 2 (i.e. on day 1).
Topotecan will be administered either with a dose of 2.3 mg/m² orally (p.o.) or 1.5 mg/m² by intravenous infusion daily on Day 1 to Day 5 during each cycle every 21 days.
Patients were enrolled irrespective of DLL3 expression (≥0% DLL3 positivity).

Combination of BI 764532 with topotecan 1 cycle = 3 weeks duration
BI 764532 dosing (dose escalation in subsequent dose cohorts):
Step-in cycle: initial 3 doses (×μg/kg) on weekly intervals as “step-in”
Treatment cycle 1: Target doses (×mg) administered weekly
Treatment cycles 2: Target dose (×mg) administered once every 3
weeks (q3w) until progression or toxicity, maximum of 36 months
Topotecan: 2.3 mg/m² orally (p.o.) OR 1.5 mg/m² by intravenous
infusion daily on Day 1 to Day 5 during each cycle every 21 days.
If topotecan treatment is stopped (e.g. because planned treatment duration is reached
or for tolerability reasons) BI 764532 treatment will continue as monotherapy

| | | | | | | | Treatment |
|---|---|---|---|---|---|---|---|
| | | | | | | | cycle 2 |
| | Step-in cycle | | | Treatment cycle 1 | | | (repeated) |
| | d 1 | d 8 | d 15 | d 1 | d 8 | d 15 | d 1 |
| BI | 10 | 30 | 90 | Target | Target | Target | Target |
| 764532 | μg /kg | μg /kg | μg /kg | dose: | dose: | dose: | dose: |
| dose | | | | From | From | From | From |
| | | | | 5 mg to | 5 mg to | 5 mg to | 5 mg to |
| | | | | 60 mg | 60 mg | 60 mg | 60 mg |
| | d 1-5 | | | d 1-5 | | | d 1-5 |
| Topotecan | 2.3 | | | 2.3 | | | 2.3 |
| | mg/m²/d | | | mg/m²/d | | | mg/m²/d |
| | p.o. | | | p.o. | | | p.o. |
| | OR 1.5 | | | OR 1.5 | | | OR 1.5 |
| | mg/m²/d | | | mg/m²/d | | | mg/m²/d |
| | i.v. | | | i.v. | | | i.v. |

Combination of BI 764532 with lurbinectedin 1 cycle = 3 weeks duration
BI 764532 dosing (dose escalation in subsequent dose cohorts):
Step-in cycle: initial 3 doses (×μg/kg) on weekly intervals as “step-in”
Treatment cycle 1: Target doses (×mg) administered weekly
Treatment cycles 2: Target dose (×mg) administered once every 3
weeks (q3w) until progression or toxicity, maximum of 36 months
Lurbinectedin: 3.2 mg/m² as intravenous infusion (IV)
daily on Day 1 during each cycle every 21 days.
If topotecan treatment is stopped (e.g. because planned treatment duration is
reached or for tolerability reasons) BI 764532 treatment will continue as monotherapy

| | | | | | | | Treatment |
|---|---|---|---|---|---|---|---|
| | | | | | | | cycle 2 |
| | Step-in cycle | | | Treatment cycle 1 | | | (repeated) |
| | d 1 | d 8 | d 15 | d 1 | d 8 | d 15 | d 1 |
| BI | 10 | 30 | 90 | Target | Target | Target | Target |
| 764532 | μg /kg | μg /kg | μg/kg | dose: | dose: | dose: | dose: |
| dose | | | | From | From | From | From |
| | | | | 5 mg to | 5 mg to | 5 mg to | 5 mg to |
| | | | | 60 mg | 60 mg, | 60 mg, | 60 mg |
| | d 1 | | | d 1 | | | d 1 |
| Lurbinectedin | 3.2 | | | 3.2 | | | 3.2 |
| | mg/m²/d | | | mg/m²/d | | | mg/m²/d |
| | IV | | | IV | | | IV |

Combination of BI 764532 with amrubicin 1 cycle = 3 weeks duration
BI 764532 dosing (dose escalation in subsequent dose cohorts):
Step-in cycle: initial 3 doses (×μg/kg) on weekly intervals as “step-in”
Treatment cycle 1: Target doses (×mg) administered weekly
Treatment cycles 2: Target dose (×mg) administered once every 3
weeks (q3w) until progression or toxicity, maximum of 36 months
Amrubicin: 40 mg/m² as intravenous infusion (IV)
daily on Day 1 to Day 5 during each cycle every 21 days.
If topotecan treatment is stopped (e.g. because planned treatment duration is
reached or for tolerability reasons) BI 764532 treatment will continue as monotherapy

| | | | | | | | Treatment |
|---|---|---|---|---|---|---|---|
| | | | | | | | cycle 2 |
| | Step-in cycle | | | Treatment cycle 1 | | | (repeated) |
| | d 1 | d 8 | d 15 | d 1 | d 8 | d 15 | d 1 |
| BI | 10 | 30 | 90 | Target | Target | Target | Target |
| 764532 | μg/kg | μg/kg | μg/kg | dose: | dose: | dose: | dose: |
| dose | | | | From | From | From | From |
| | | | | 5 mg to | 5 mg to | 5 mg to | 5 mg to |
| | | | | 60 mg | 60 mg | 60 mg | 60 mg |
| | d 1-3 | | | d 1-3 | | | d 1-3 |
| Amrubicin | 40 | | | 40 | | | 40 |
| | mg/m²/d | | | mg/m²/d | | | mg/m²/d |
| | IV | | | IV | | | IV |

Step-in strategy in Regimen D: step-in dosing will apply weight based dosing based on Regimen B3 results
Target doses in Regimen D: the fixed target doses (i.e. without adaptation to body weight) will be escalated in subsequent dosing cohorts (e.g. 10 mg, 30 mg, 60 mg) to define the MTD/RDE.
The rationale for body weight adapted step-in doses and fixed target doses is the same as for Dareon™_5
Following patients will be included in this study: Patients with ES-SCLC that progressed or recurred following at least one line of platinum-based treatment as per local standard of care (SOC), including anti-PD-1 or PD-L1, as applicable.

### Example 5B: Dareon™-9 Results of Combination of BI 764532 with Topotecan, in Patients with SCLC

### Patients Treated:

As of 22 May 2025 (cutoff date for described results), 30 pts had received 1 cycle of treatment. 10 patients at each obrixtamig target dose level (10 mg, 30 mg, 60 mg). 15 patients were ongoing on study treatment at the cutoff date. The median number of cycles for obrixtamig/topotecan was 8 (range: 1-21)/6 (range: 1-20). Median age was 65 years (range: 38-81); (Eastern Cooperative Oncology Group Performance Status (ECOG PS) was 0/1 in 47%/53% of pts. Patients were enrolled irrespective of DLL3 expression (≥0% DLL3 positivity).

### Determination of the MTD

A BLRM employing the EWOC principle was used during the escalation phase for the selection of the dose levels and the estimation of the MTD, where applicable (as described in detail above).

Safety Data from Dareon™-9 of Combination of BI 764532 with Topotecan

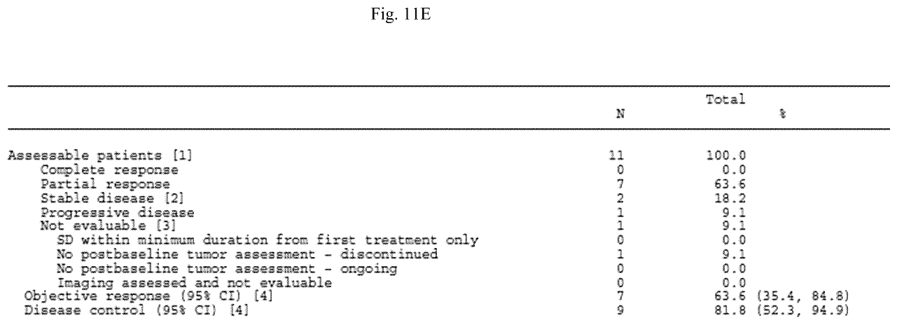

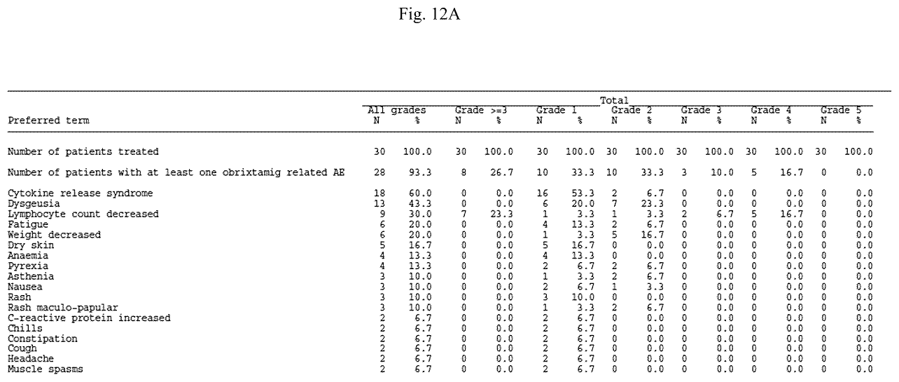

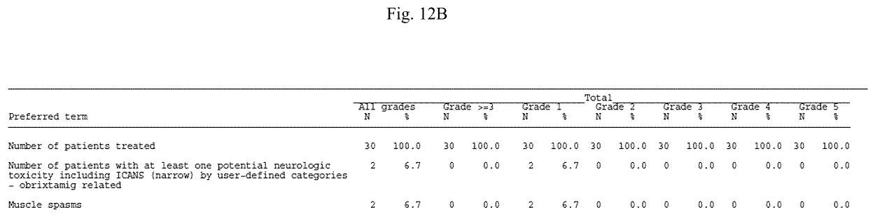

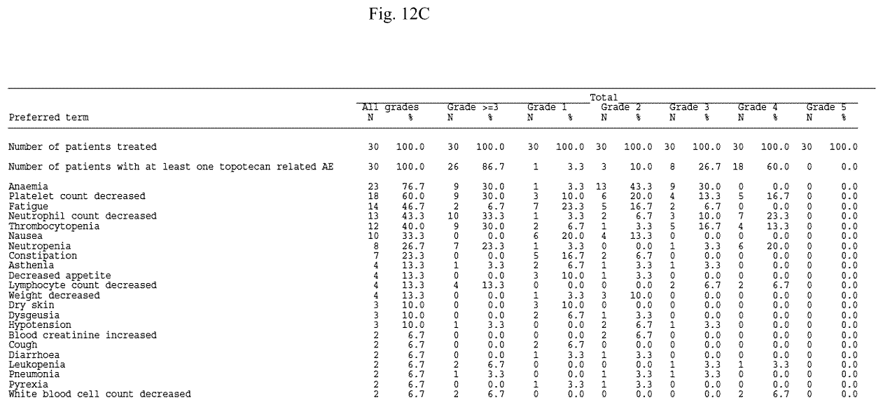

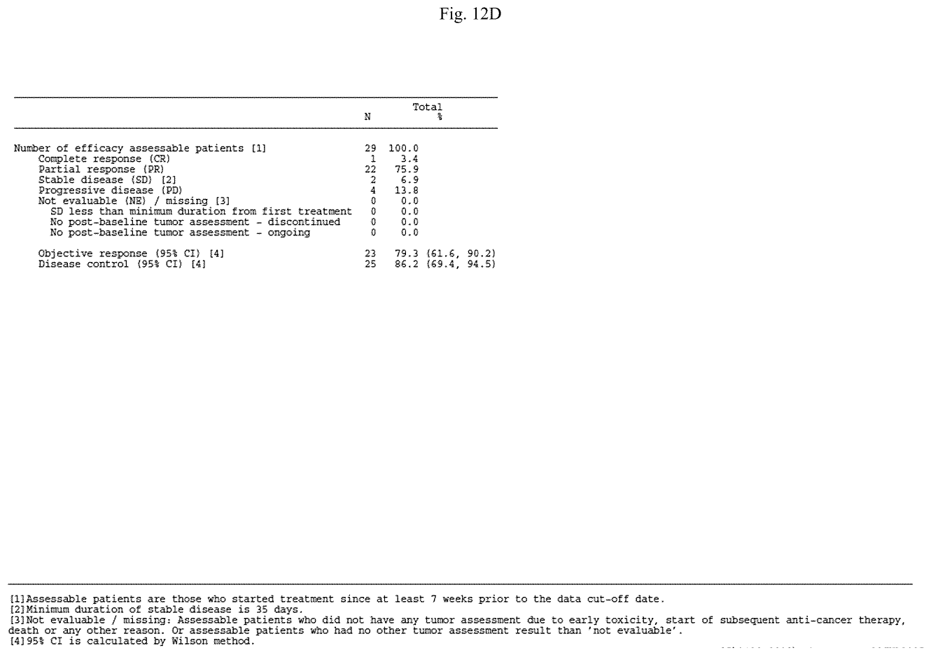

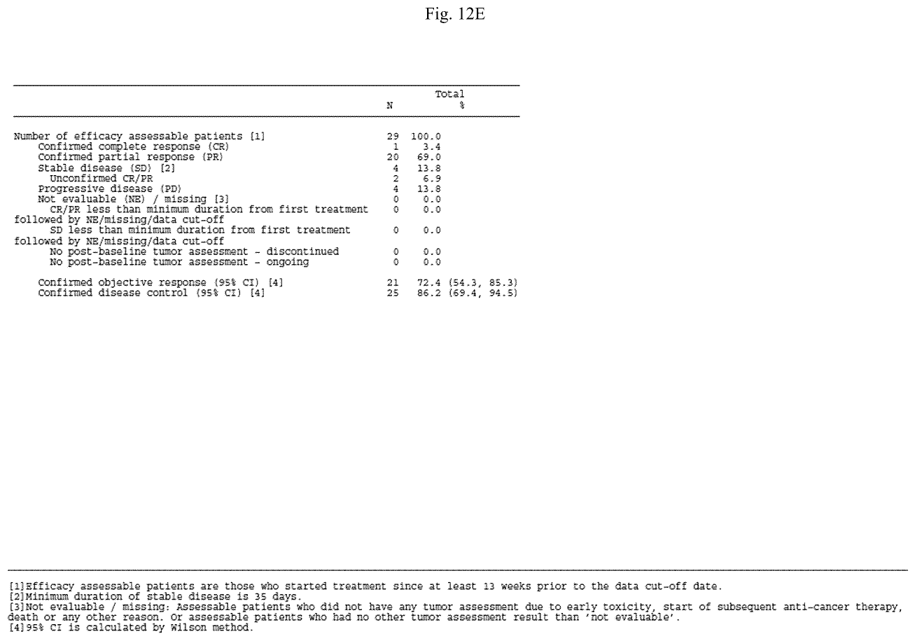

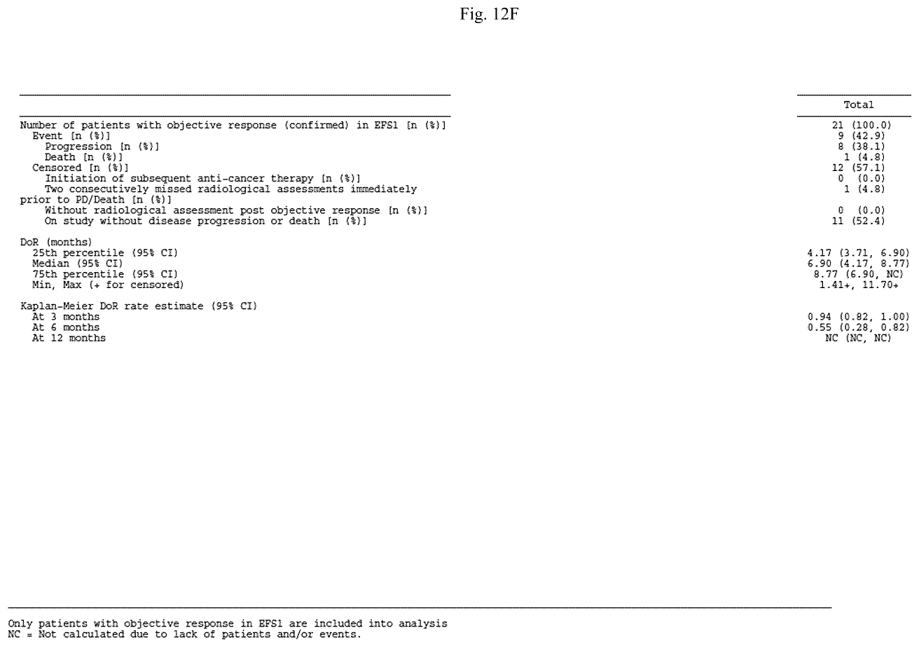

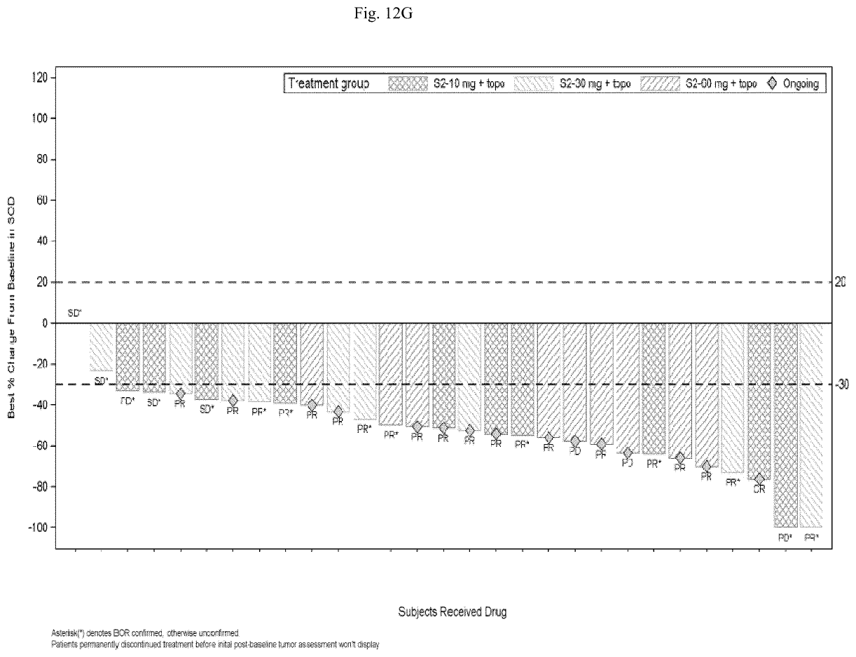

Safety data from the patients treated in Dareon™-9 combination of BI 764532 with topotecan, as described above, were collected using the CTCAE-version 5. The observed most frequent (>5%) obrixtamig-related adverse events (AEs) are shown in FIG. 12A. As expected from the BI 764532 Mode of Action (MoA) the most frequent obrixtamig-related AE was CRS which occurs in approximately 60% of the patients (all grades), all of the CRS events were low grade (CTCAE 1-2) and no patients experienced CRS grade 3 or higher. Surprisingly, the combination with topotecan seems to impact neither the frequency nor the severity of BI 764532 related adverse events as compared to BI 764532 monotherapy (see example 1 above).

Based on the BI 764532 MoA and published data for other TCE treatments neurological AEs, especially ICANS, could be expected as well. The overall frequency of potential neurologic toxicity including ICANS by user-defined categories (all CTCAE grades, BI 764532 related) in Dareon™-9 combination of BI 764532 with topotecan was 6.7% (FIG. 12B) with CTCAE grade 1 for all ICANS events. The combination with topotecan seems to impact neither the frequency nor the severity of BI 764532 related ICANS events as compared to BI 764542 monotherapy (see example 1 above and FIG. 3F).

As expected from the topotecan MoA among the most frequent topotecan-related AEs were predominantly cytopenias (anaemia, thrombocytopenia/decreased platelet counts, neutropenia/decreased neutrophil counts) which occurred in the majority of patients. The observed most frequent (>5%) topotecan-related adverse events (AEs) are shown in FIG. 12C. The combination with BI 764532 worsened neither the frequency nor the severity of topotecan related adverse events as compared to published data (FDA Prescribing Information for TOPOTECAN INJECTION, for intravenous use, https://www.accessdata.fda.gov/drugsatfda_docs/label/2014/022453s002lbl.pdf accessed 18 Nov. 2025).

Safety conclusion: frequency and severity of adverse events (AEs) observed with combination treatment of obrixtamig+topotecan were consistent with expected safety findings for obrixtamig or topotecan as monotherapy. Surprisingly, the combination treatment with obrixtamig+topotecan (both at full monotherapy doses) was tolerable with no indicators of overlapping toxicity.

### Tumor Response was Assessed in all Patients Enrolled.

Unconfirmed ORR was 79.3% (95% CI 61.6, 90.2) (FIG. 12D). The confirmed ORR was 72.4% (95% CI 54.3, 85.3) and the disease control rate was 86.2 (95% CI 69.4, 94.5) (FIG. 12E); the median duration of confirmed response (DoR) was 6.90 months (95% CI 4.17, 8.77) (FIG. 12F). Tumor shrinkage in target lesions was observed in 28 out of 29 pts (97%) (FIG. 12G)

Efficacy conclusion: efficacy data for the combination of obrixtamig+topotecan show synergistic efficacy in 2L+SCLC as compared to the reported monotherapy activity for obrixtamig (see data of Example 1) and topotecan (published FDA data on topotecan monotherapy, where overall response rate is 24%, see https://www.accessdata.fda.gov/drugsatfda_docs/label/2014/022453s002lbl.pdf accessed 18 Nov. 2025), respectively.

### Example 6A: Dareon™-7 Combination of BI 764532 with Standard of Care (Platinum and Etoposide) in First-Line Treatment of Patients with EPNEC, Pulmonary LCNEC and NEC of Unknown Primary Site

### Dosing Regimen E

Treatment cycles of 3 week (21 day) duration. BI 764532 combined with a platin+etoposide.
Patients were DLL3 positive (>0% DLL3 positivity).
Treatment with BI 764532 will start after initial cycles (e.g. 1-2 cycles) of platin+etoposide is completed (i.e. the step-in cycle of the combination BI 764532+platin+etoposide (described as step-in cycle below) is the 2^{nd} cycle of platinum+etoposide in case of one initial cycle of chemotherapy)
BI 764532 dosing regimen comprising a first step-in dose (x1 μg/kg) in step-in cycle on day 1, a second step-in dose (x2 μg/kg) in step-in cycle on day 8, a third step-in dose (x3 μg/kg) in step-in cycle on day 15, followed by weekly administration of the target dose (fixed dose x mg) in combination treatment cycle 1 (i.e. on day 1, 8 and 15), followed by once every three week (q3w) administration of the target dose in treatment cycles 2 (i.e. on day 1).
Platin will be administered on Day 1 of each cycle, etoposide will be administered on days 1-3 of each cycle.
A total of 3-5 cycles of BI 764532+platin+etoposide will be administered. After completion of BI 764532+platin+etoposide, monotherapy with BI 764532 will continue until progression or toxicity for a maximum of 36 months

Combination of BI 764532 with platin + etoposide (SoC
for 1L epNEC, LC NEC lung, NEC of unknown primary site)
1 cycle = 3 weeks duration
BI 764532 + platin + etoposide will start after at least one initial cycle of
platin + eptoposide is completed (i.e. the step-in cycle of the combination BI
764532 + platin + etoposide (described as step-in cylce below) is at least the
2nd cycle of platinum + etoposide)
BI 764532 dosing (dose escalation in subsequent dose cohorts):
Step-in cycle: initial 3 doses (×μg/kg) on weekly intervals as “step-in”
Treatment cycle 1: Target doses (×mg) administered weekly
Treatment cycle 2 (repeated): Target dose (×mg) administered once every 3
weeks (q3w) until progression or toxicity, maximum of 36 months
Platin: d 1 of each cycle
Etoposide: d 1-3 of each cycle

| | | | | | | | Treatment cycles 2 | Treatment |
|---|---|---|---|---|---|---|---|---|
| | | | | | | | (combi) administered | cycles 2 |
| | Step-in cycle | | | Treatment cycle 1 | | | 1 up to 3 times | (mono) |
| | d 1 | d 8 | d 15 | d 1 | d 8 | d 15 | d 1 | D 1 |
| BI | 10 | 30 | 90 | Target | Target | Target | Target | Target |
| 764532 | μg/kg | μg/kg | μg/kg | dose: | dose: | dose: | dose: | dose: |
| dose | | | | From | From | From | From | From |
| | | | | 5 mg to | 5 mg to | 5 mg to | 5 mg to | 5 mg to |
| | | | | 60 mg | 60 mg | 60 mg | 60 mg | 60 mg |
| Platin | Carboplatin | | | Carboplatin | | | Carboplatin | |
| | AUC 5 | | | AUC 5 | | | AUC 5 | |
| | mg/mL | | | mg/mL | | | mg/mL | |
| | min | | | min | | | min | |
| | OR | | | OR | | | OR | |
| | Cisplatin | | | Cisplatin | | | Cisplatin | |
| | 75 | | | 75 | | | 75 | |
| | mg/m2 | | | mg/m2 | | | mg/m2 | |
| | d 1-3 | | | d 1-3 | | | d 1-3 | |
| Etopopside | 100 | | | 100 | | | 100 | |
| | mg/m2 | | | mg/m2 | | | mg/m2 | |

Step-in strategy in Regimen E: step-in dosing will apply weight-based dosing based on Regimen B3 results
Target doses in Regimen E: the fixed target doses (i.e. without adaptation to body weight) will be escalated in subsequent dosing cohorts (e.g. 10 mg, 30 mg, 60 mg) to define the MTD/RDE.
The rationale for body weight adapted step-in doses and fixed target doses is the same as for Dareon™_5

### Example 6B: Dareon™-7 Combination of BI 764532 with Standard of Care (Platinum and Etoposide) in First-Line Treatment of Patients with EPNEC, Pulmonary LCNEC and NEC of Unknown Primary Site

### Patients Treated:

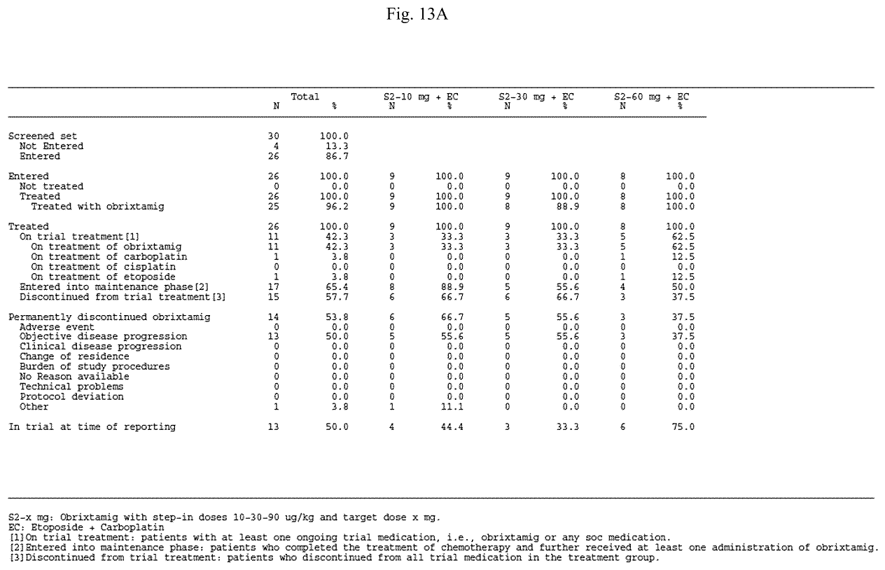

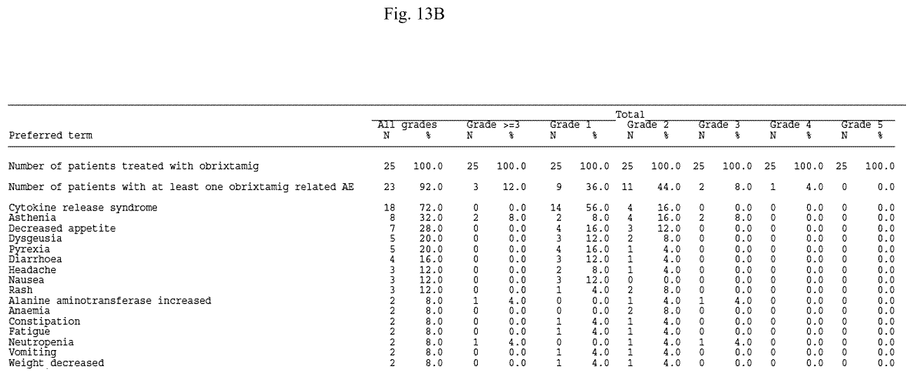

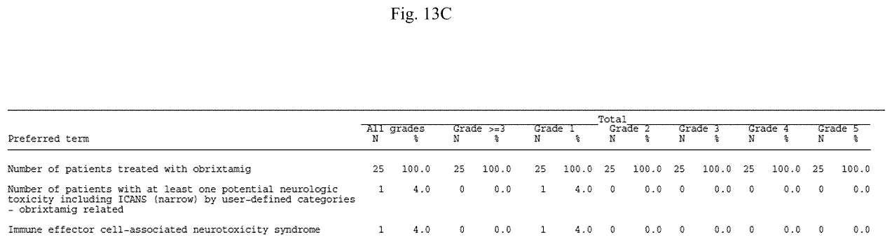

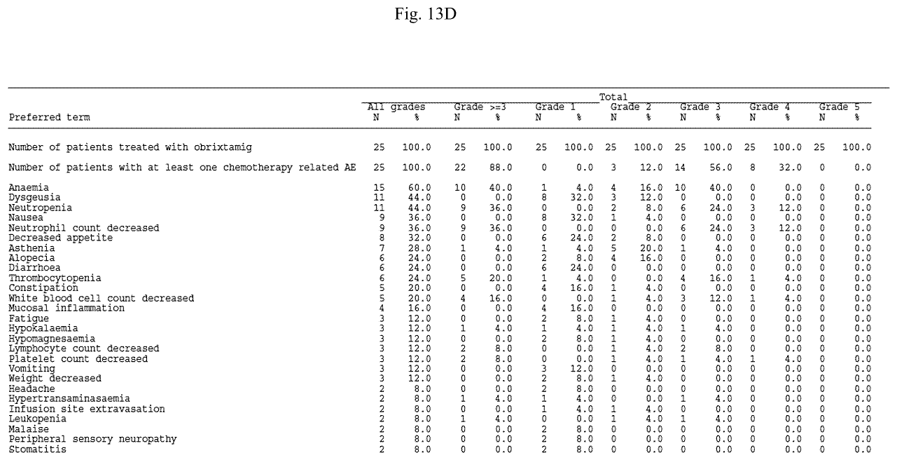

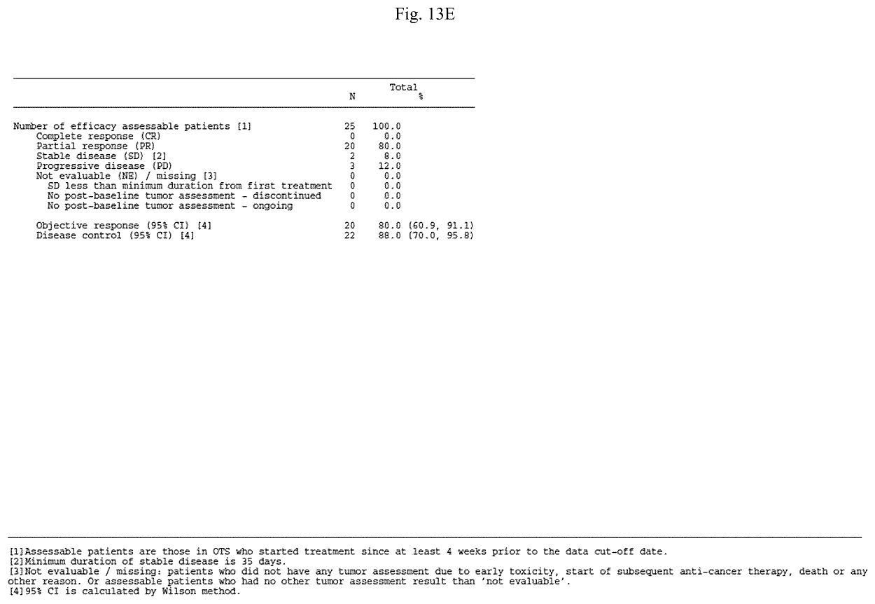

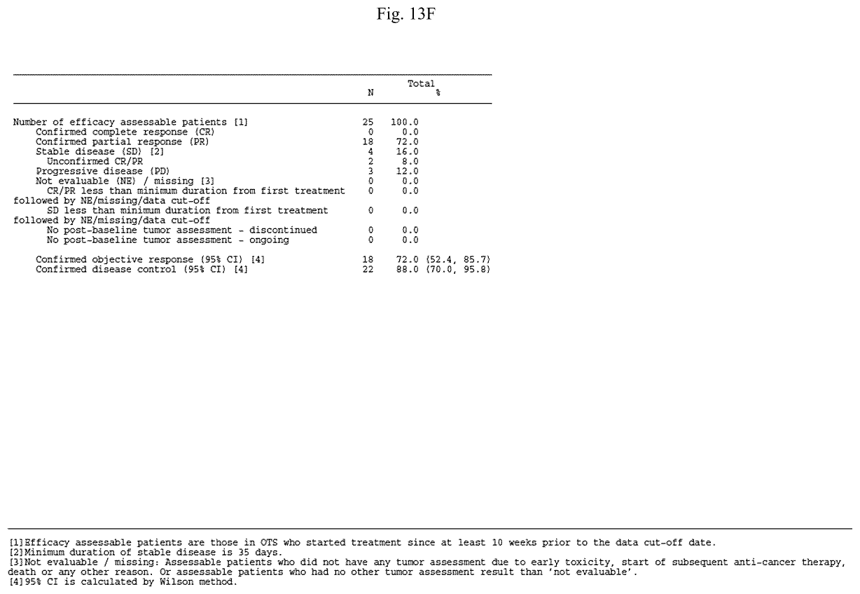

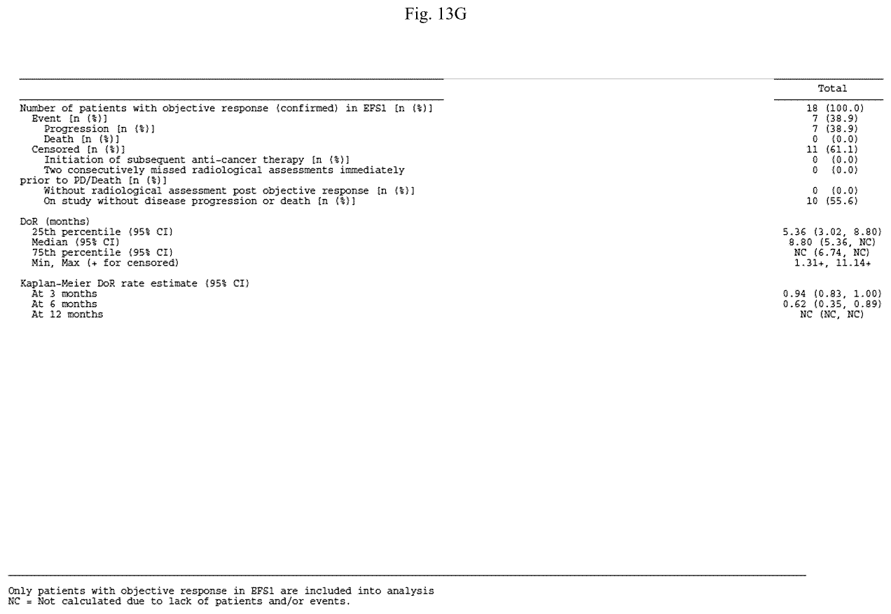

As of 29 Aug. 2025 (cutoff date for described results), 25 pts had received 8 764532 with platinum and etoposide. 9, 9 and 8 patients were treated at the different 8 764532 target dose levels (10 mg, 30 mg, 60 mg). 11 patients were ongoing on study treatment at the cutoff date (see FIG. 13A). Median age was 67 years (range: 42-79); ECOG PS was 0/1 in 76%/24% of pts. DLL3 positive (>0% DLL3 positivity) was an enrolment criterion.

### Determination of the MTD

A BLRM employing the EWOC principle was used during the escalation phase for the selection of the dose levels and the estimation of the MTD, where applicable (as described in detail above).

Safety data from Dareon™-7 of combination of BI 764532 with standard of care (platinum+etoposide) in first-line treatment of patients with epNEC, pulmonary LCNEC and NEC of unknown primary site.

Safety data from the patients treated in Dareon™-7 of combination of BI 764532 with standard of care (platinum+etoposide), as described above, were collected using the CTCAE-version 5. The observed most frequent (>5%) obrixtamig-related adverse events (AEs) are shown in FIG. 13B. As expected from the BI 764532 Mode of Action (MoA) the most frequent obrixtamig-related AE was CRS which occurs in 72% of the patients (all grades), all of the CRS events were low grade (CTCAE grade 156%, CTCAE grade 2 16%) and no patients experienced CRS grade 3 or higher. Surprisingly, the combination with standard of care chemotherapy (platinum+etoposide) seems to impact neither the frequency nor the severity of BI 764532 related adverse events as compared to BI 764532 monotherapy (see example 1 above).

Based on the BI 764532 MoA and other published data for TCE treatments as well neurological AEs, especially ICANS, could be expected. The overall frequency of potential neurologic toxicity including ICANS by user-defined categories (all CTCAE grades, BI 764532 related) in Dareon™-7 combination of BI 764532 with standard of care chemotherapy (platinum+etoposide) was 4% (FIG. 13C) with CTCAE grade 1 for the reported ICANS event. The combination with standard of care chemotherapy (platinum+etoposide) seems to negatively impact neither the frequency nor the severity of BI 764532 related ICANS events as compared to BI 764532 monotherapy (see example 1 above and FIG. 3F).

As expected from the standard of care chemotherapy (platinum+etoposide) MoA among the most frequent chemotherapy-related AEs were predominantly cytopenias (anaemia, thrombocytopenia/decreased platelet counts, neutropenia/decreased neutrophil counts) and other AEs known for cytotoxic treatments. The observed most frequent (>5%) chemotherapy (platinum+etoposide)—related adverse events (AEs) are shown in FIG. 13D. The combination with BI 764532 seems not worsen either the frequency or the severity of chemotherapy related adverse events as compared to chemotherapy (platinum+etoposide) alone.

Safety conclusion: BI 764532 plus chemotherapy was tolerable with no unexpected toxicities. The reported frequency/severity of AEs was consistent with the expected safety profile of the individual drugs used in the combination treatment. Surprisingly, the combination treatment of BI 764532+chemotherapy (platinum etoposide) allowed administration of obrixtamig at full monotherapy dose and chemotherapeutic drugs at established full doses with no results indicating additional or overlapping toxicities.

Tumor response was assessed in all patients enrolled.

The unconfirmed ORR was 80% (95% CI 60.9, 91.1) (FIG. 13E). The confirmed ORR was 72% (95% CI 52.4, 85.7) and the disease control rate was 88% (95% CI 70.0, 95.8) (FIG. 13F); the median duration of confirmed response (DoR) was 8.80 months (95% CI 5.36, NC) (FIG. 13G).

Efficacy conclusion: efficacy data for the combination of BI 764532 plus chemotherapy were encouraging.

### Example 7A: Dareon™-8 Combination of BI 764532 with Platin+Etoposide+Anti-PD-L1 (Soc for 1L SCLC) in Patients with Extensive-Stage Small Cell Lung Carcinoma

### Dosing Regimen F

Treatment cycles of 3 week (21 day) duration. BI 764532 combined with a platin+eptoposide+aPD-L1 (e.g. atezolizumab, durvalumab).
Treatment with BI 764532 will start after at least one initial cycle of platin+etoposide+aPD-L1 is completed (i.e. the step-in cycle of the combination BI 764532+platin+etoposide+aPD-L1 (described as step-in cycle below) is at least the 2^{nd} cycle of platinum+etoposide+aPD-L1)
BI 764532 dosing regimen comprising a first step-in dose (x1 μg/kg) in step-in cycle on day 1, a second step-in dose (x2 μg/kg) in step-in cycle on day 8, a third step-in dose (x3 μg/kg) in step-in cycle on day 15, followed by weekly administration of the target dose (fixed dose x mg) in treatment cycle 1 (i.e. on day 1, 8 and 15), followed by once every three week (q3w) administration of the target dose in repeated treatment cycles 2 (i.e. on day 1).
Platin will be administered on Day 1 of each cycle, etoposide will be administered on days 1-3 of each cycle, aPD-L1 will be administered on day 1 of each cycle.
A total of 3-5 cycles of BI 764532+platin+etoposide+aPD-L1 will be administered. After completion of BI 764532+platin+etoposide+aPD-L1, maintenance treatment (aPD-L1+BI 764532 or monotherapy with BI 764532) will continue until progression or toxicity for a maximum of 36 months.
Patients were enrolled irrespective of DLL3 positivity (≥0% DLL3 positivity)

Combination of BI 764532 with platin + etoposide + anti-PD-L1 (SoC for 1 L SCLC)
1 cycle = 3 weeks duration
BI 764532 + platin + etoposide + aPD-L1 will start after at least one initial cycle of
platin + etoposide + aPD-L1 is completed (i.e. the first cycle of the combination BI
764532 + platin + etoposide + aPD-L1 (described as step-in cycle below) is at least the
2nd cycle of platinum + etoposide + aPD-L1)
BI 764532 dosing (dose escalation in subsequent dose cohorts):
Step-in cycle: initial 3 doses (×μg/kg) on weekly intervals as “step-in”
Treatment cycle 1: Target doses (×mg) administered weekly
Treatment cycles 2 (repeated): Target dose (×mg) administered once every 3 weeks
(q3w) until progression or toxicity, maximum of 36 months
Platin: d 1 of each cycle
Etoposide: d 1-3 of each cycle

| | | | | | | | Treatment | Treatment |
|---|---|---|---|---|---|---|---|---|
| | | | | | | | cycles 2 | cycles 2 |
| | Step-in cycle | | | Treatment cycle 1 | | | (combi) | (mono) |
| | d 1 | d 8 | d 15 | d 1 | d 8 | d 15 | d 1 | d 1 |
| BI | 10 | 30 | 90 | Target | Target | Target | Target | Target |
| 764532 | μg/kg | μg/kg | μg/kg | dose: | dose: | dose: | dose: | dose: |
| dose | | | | From 5 | From 5 | From 5 | From 5 | From 5 |
| | | | | mg to | mg to | mg to | mg to | mg to |
| | | | | 60 mg | 60 mg | 60 mg | 60 mg | 60 mg |
| SoC | Carboplatin | | | Carboplatin | | | Carboplatin | |
| | AUC 5 | | | AUC 5 | | | AUC 5 | |
| | mg/mL | | | mg/mL | | | mg/mL | |
| | min | | | min | | | min | |
| | OR | | | OR | | | OR | |
| | Cisplatin | | | Cisplatin | | | Cisplatin | |
| | 75 | | | 75 | | | 75 | |
| | mg/m2 | | | mg/m2 | | | mg/m2 | |
| aPD-L1 | Atezolizumab | | | Atezolizumab | | | Atezolizumab | Atezolizumab |
| | OR | | | OR | | | OR | OR |
| | Durvalumab | | | Durvalumab | | | Durvalumab | Durvalumab |
| | | | | | | | | (will be |
| | | | | | | | | dose q4w) |
| | d 1-3 | | | d 1-3 | | | d 1-3 | |
| Etopopside | 100 | | | 100 | | | 100 | |
| | mg/m2 | | | mg/m2 | | | mg/m2 | |

Step-in strategy in Regimen F: step-in dosing will apply weight based dosing based on Regimen B3 results
Target doses in Regimen F: the fixed target doses (i.e. without adaptation to body weight) will be escalated in subsequent dosing cohorts (e.g. 10 mg, 30 mg, 60 mg) to define the MTD/RDE.
The rationale for body weight adapted step-in doses and fixed target doses is the same as for Dareon™_5

### Example 7B: Dareon™-8 Combination of BI 764532 with Platin+Etoposide+Anti-PD-L1 (Soc for 1L SCLC) in Patients with Extensive-Stage Small Cell Lung Carcinoma

### Patients Treated:

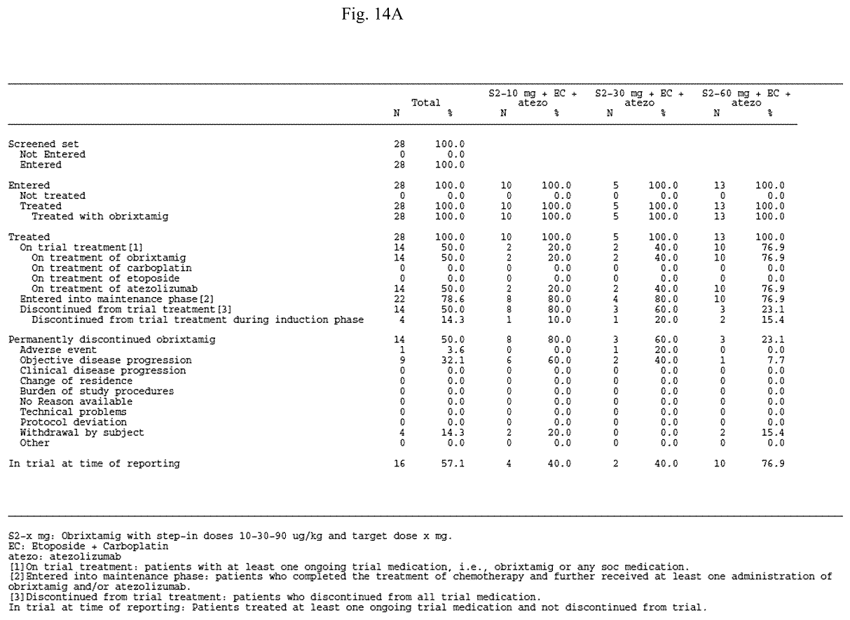

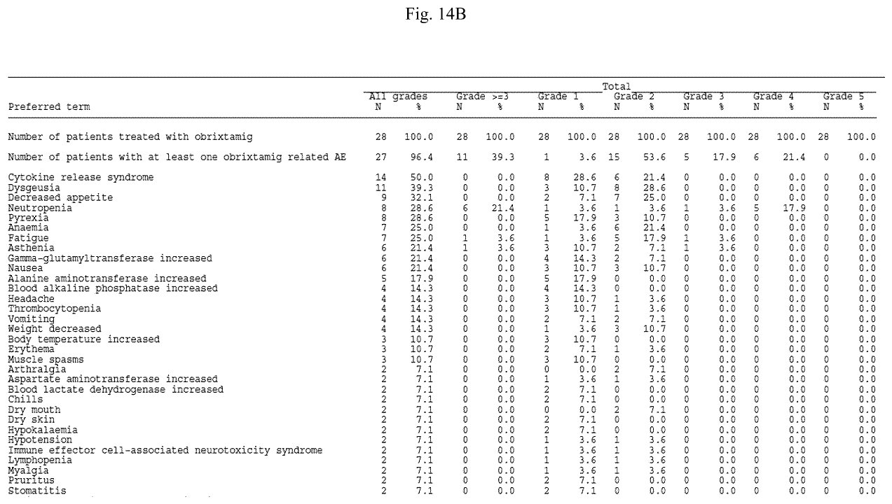

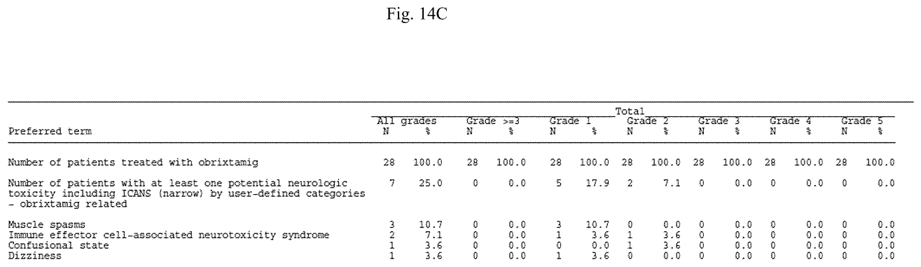

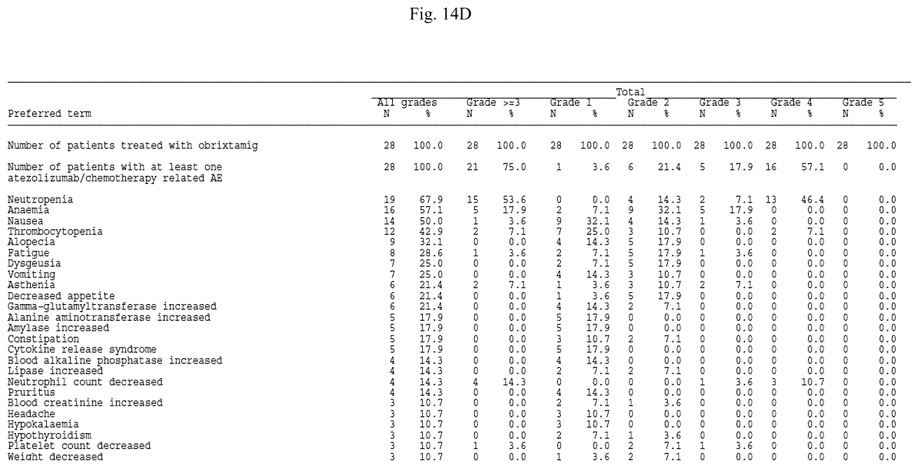

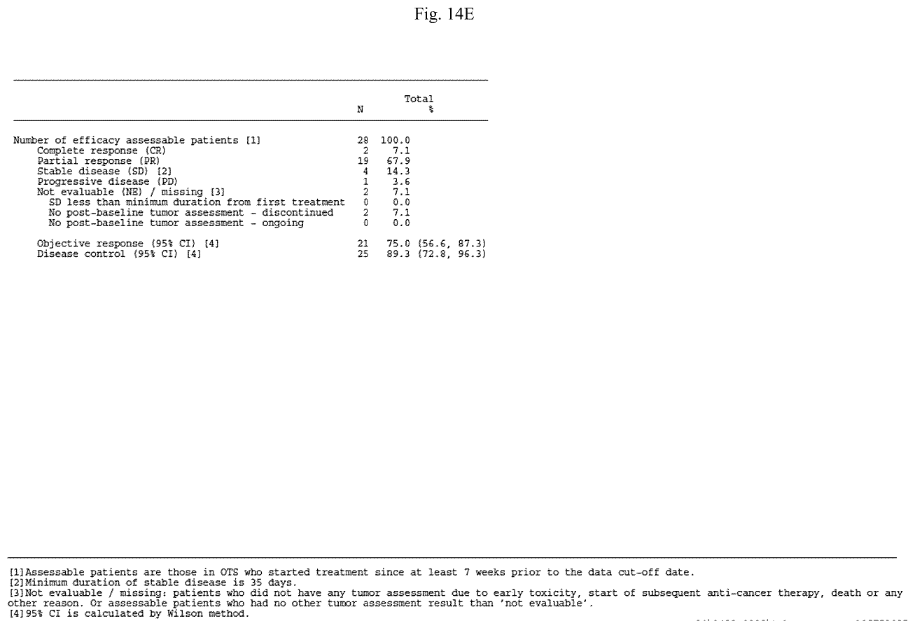

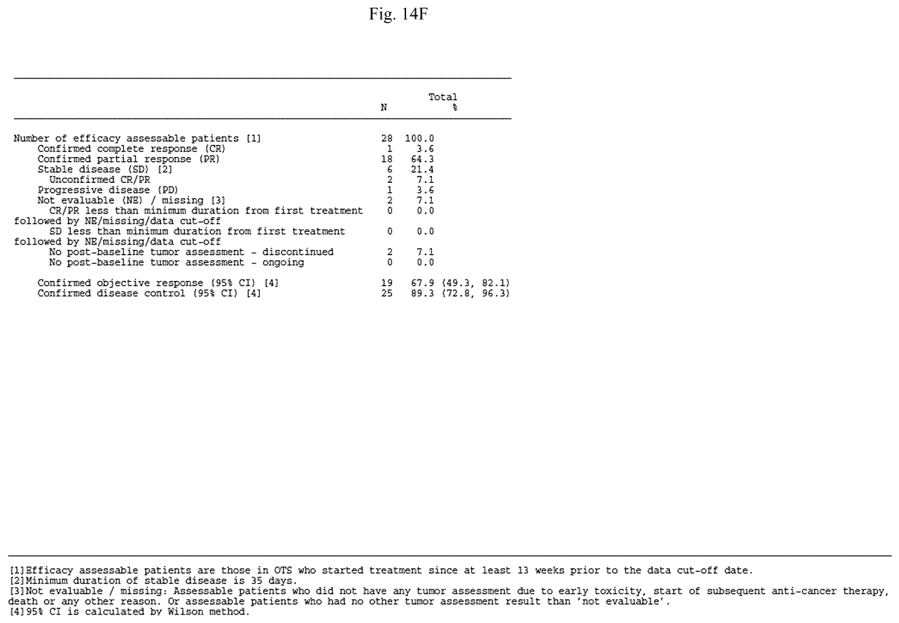

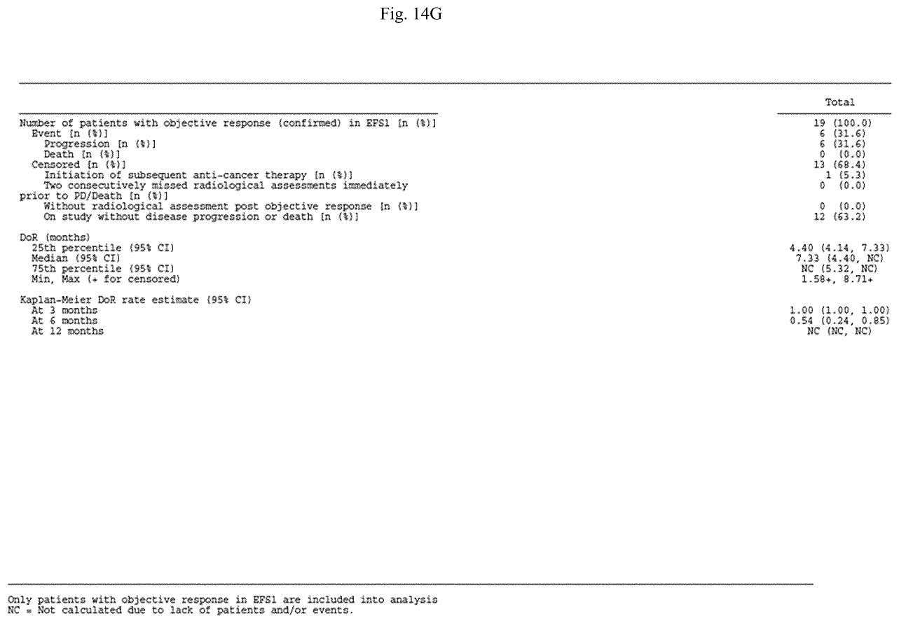

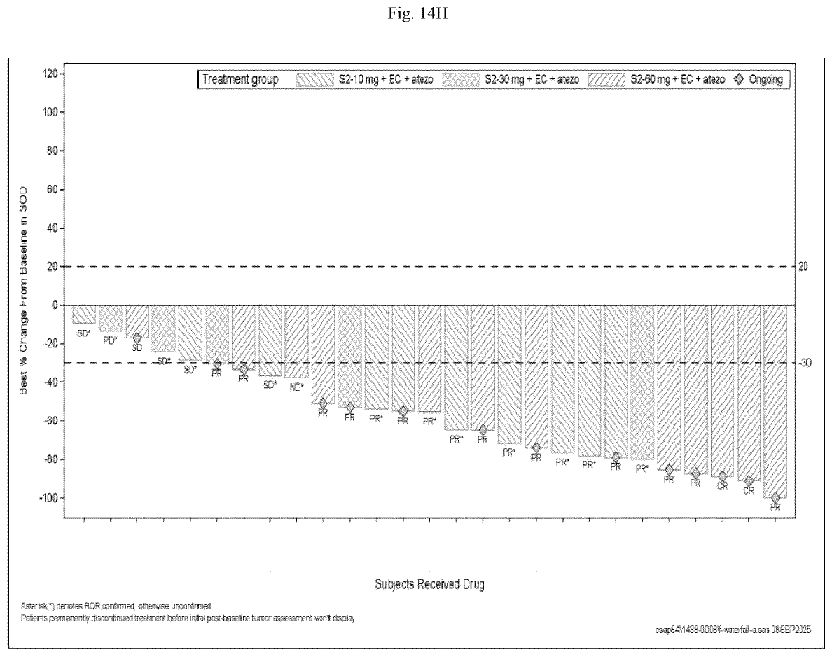

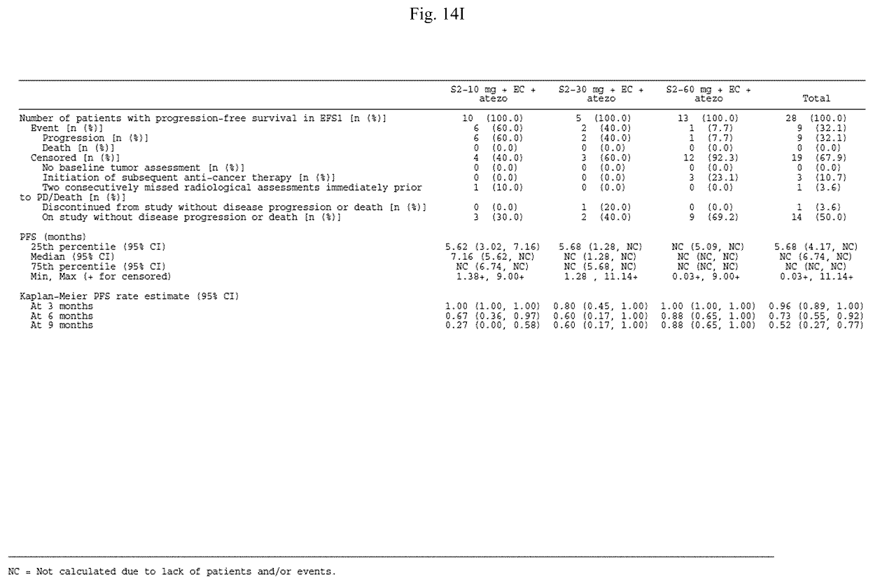

As of 21 Aug. 2025 (cutoff date for described results), 28 pts had received 8 764532 with platinum, etoposide and atezolizumab. 10, 5 and 13 patients were treated at each obrixtamig target dose level (10 mg, 30 mg, 60 mg). 14 patients were ongoing on study treatment at the cutoff date (see FIG. 14A).

Median age was 67.5 years (range: 44-75); ECOG PS was 0/1 in 25%/75% of pts. Patients were enrolled irrespectively of DLL3 expression (≥0% DLL3 positivity).

### Determination of the MTD

A BLRM employing the EWOC principle was used during the escalation phase for the selection of the dose levels and the estimation of the MTD, where applicable (as described in detail above).

Safety data from Dareon™-8 exploring the combination of BI 764532 with standard of care (platinum+etoposide+anti-PD-L1 (atezolizumab)) in first-line treatment of patients with extensive stage SCLC.

Safety data from the patients treated in Dareon™-8 (combination of BI 764532 with standard of care (platinum+etoposide+anti-PD-L1 (atezolizumab))), as described above, were collected using the CTCAE-version 5. The observed most frequent (>5%) obrixtamig-related adverse events (AEs) are shown in FIG. 14B. As expected from the BI 764532 Mode of Action (MoA) the most frequent obrixtamig-related AE was CRS which occurred in 50% of the patients (all grades), all of the CRS events were low grade (CTCAE grade 1 28.6%, CTCAE grade 2 21.4%) and no patients experienced CRS grade 3 or higher. Based on the BI 764532 MoA and other published data for TCE treatments as well neurological AEs, especially ICANS, could be expected. The overall frequency of potential neurologic toxicity including ICANS by user-defined categories (all CTCAE grades, BI 764532 related) in Dareon™-8 (combination of BI 764532 with standard of care (platinum+etoposide+atezolizumab)) was 25% (FIG. 14C) with CTCAE grade 1 or 2 for all reported potential neurologic toxicity events and no AEs grade 3 or higher; the reported frequency of Immune effector cell-associated neurotoxicity syndrome (ICANS) was 7% (CTCAE grades 1 and 2).

Surprisingly, the combination of BI 764532 with standard of care therapy (platinum+etoposide+atezolizumab) did neither impact the frequency nor the severity of BI 764532 related adverse events, especially regarding the AEs of special interest CRS and ICANS, as compared to BI 764542 monotherapy (see example 1 above).

As expected from the MoAs of standard of care therapy (platinum+etoposide+atezolizumab) among the most frequent SoC-related AEs were predominantly cytopenias (anaemia, thrombocytopenia/decreased platelet counts, neutropenia/decreased neutrophil counts) and other AEs previously described for this SoC treatment. The observed most frequent (>10%) SoC (platinum+etoposide+atezolizumab)—related adverse events (AEs) are shown in FIG. 14D. The combination with BI 764532 seems neither to worsen the frequency nor the severity of SoC related adverse events as compared to SoC (platinum+etoposide+atezolizumab) alone.

Safety conclusion: BI 764532 plus SoC was tolerable with no unexpected toxicities. The combination surprisingly demonstrated a safety profile consistent with individual treatments, reinforcing its favorable tolerability and allowing administration of obrixtamig at full monotherapy dose in combination with SoC drugs at established full doses with no results indicating additional or overlapping toxicities. There were no grade ≥3 CRS/ICANS reported.

Tumor response was assessed in all patients enrolled.

The unconfirmed ORR was 75% (95% CI 56.6, 87.3) (FIG. 14E). The confirmed ORR was 67.9% (95% CI 49.3, 82.1) and the disease control rate was 89.3% (95% CI 72.8, 96.3) (FIG. 14F); the median duration of confirmed response (DoR) was 7.33 months (95% CI 4.40, NC) (FIG. 14G). Tumor shrinkage in target lesions was observed in all evaluable pts (FIG. 14H).

Obrixtamig in combination with SoC as induction and maintenance treatment shows encouraging clinical outcomes, including PFS rates of 73% and 52% at 6 and 9 months, respectively (see FIG. 14I); the median PFS was not yet reached at the data cut-off for the presented analysis (see FIG. 14J)

Efficacy conclusion: efficacy data for the combination of BI 764532 plus chemotherapy were encouraging.

### Example 8: Phase I Study of BI 764532 in Glioma

The study described herein is a first-in-human, Phase I dose escalation study of BI 764532 monotherapy in patients with glioma expressing DLL3 (NCT05916313).

The objectives of this study (Part A) are to characterize the dose-tolerability curve for BI 764532 in patients with diffuse glioma expressing DLL3 by assessing multiple increasing doses. The main objectives are to characterise safety of BI 764532 by assessing multiple doses with overdose control and to determine the maximum tolerated dose (MTD) and the recommended dose for expansion (RDE) in patients with diffuse glioma expressing DLL3. The main objective of this study (Part B—Expansion) is to confirm safety at the RDE.

### Methods and Analysis

### Study Design

This Phase I study is a multicenter, open-label, non-randomized, dose-escalation study. In the Part A BI 764532 is administered multiple increasing doses. In Part B (Expansion) BI 764532 is administered at dose(s) determined RDE in Part A to confirm safety

### Dosing Regimen

Intravenous (i.v.) administration. BI 764532 will be administered in a step-in dosing regimen. That is, the initial 3 administrations will be composed of a fraction of the target dose. Thereafter, the full dose (=target dose) will be administered weekly for 3 weeks, and then every 3 weeks.

Glioma Ph1 dosing regimen:
one cycle = 3 weeks duration
Step-in cycle: initial 3 doses on weekly intervals as “step-in”
Treatment cycle 1: provisional target doses (90 μg/kg, 180 μg/kg,
360 μg/kg, or 720 μg/kg) administered weekly
Treatment cycles 2 (repeated): provisional target doses 90 μg/kg, 180
μg/kg, 360 μg/kg, or 720 μg/kg) administered once every 3 weeks
(q3w) until progression or toxicity, maximum of 36 months.
Further doses (step-in and target doses; doses between and above the
depicted dose levels) may be investigated based on safety results

| | | | | | | | Treatment |
|---|---|---|---|---|---|---|---|
| | | | | | | | cycles 2 |
| | Step-in cycle | | | Treatment cycle 1 | | | (repeated) |
| | d 1 | d 8 | d 15 | d 1 | d 8 | d 15 | d 1 |
| dose | 10 | 10-30 | 30-90 | Target | Target | Target | Target |
| | μg/kg | μg/kg | μg/kg | dose: | dose: | dose: | dose: |
| | | | | 90-720 | 90-720 | 90-720 | 90-720 |
| | | | | μg/kg | μg/kg | μg/kg | μg/kg |

### Main Inclusion Criteria

Patients with histologically confirmed primary progressive diffuse glioma who have failed standard of care therapies.
Eligible tumour histologies include:
astrocytoma IDH mutant, CNS WHO Grade 2-4
oligodendroglioma IDH mutant and 1p19q co-deleted, CNS WHO Grade 2 and 3
glioblastoma (IDH wild type) according to the WHO 2021 brain tumour classification
Tumours must be positive for DLL3 expression

### Study Endpoints

### Part A—Dose Escalation:

Primary endpoint: Occurrence of dose-limiting toxicity (DLT) during the MTD evaluation period
Secondary endpoint: Occurrence of DLT during the entire treatment period Part B—Expansion:
Primary endpoint: Occurrence of DLT during the entire treatment period
Secondary endpoints: None

### Statistical Analysis, Determination of the MTD

Dose escalation is guided by Bayesian logistic regression model (BLRM) with overdose control that will be fitted to binary toxicity outcomes. The estimate of parameters will be updated as data are accumulated using the BLRM. At the end of the dose escalation, the toxicity probability at each dose level will be calculated to determine an estimate of the MTD and/or the RDE. Descriptive analysis (summary statistics) will be used to describe other safety and efficacy endpoints.

### Example 9: Scoring of DLL3 Expression in Samples from Individuals Treated with BI 764532 (Obrixtamig)

The DLL3 expression status in tumor specimens was evaluated using an anti-DLL3-specific antibody kit (VENTANA® DLL3 (SP347) Assay) intended for detection of DLL3 in sections of human formalin-fixed and paraffin-embedded (FFPE) tissues by immunohistochemistry (IHC). In brief, 4-5-μm thick tissue sections were prepared from FFPE tissue blocks and stained on the BenchMark ULTRA automated slide staining platform (Roche Diagnostics) using the OptiView DAB IHC Detection Kit (Roche Diagnostics) for visualization. A negative control (Rabbit Monoclonal Negative Control, Roche Diagnostics) was included in each staining run and was evaluated for acceptable background and signal/noise staining. Hematoxylin and Eosin/H&E) staining was performed to evaluate the tissue sections for tumor content and assessment of tissue quality.

To quantify DLL3 positivity in tumor samples, a DLL3 scoring system was developed to measure DLL3-specific signal in tumor cells. Tumor cell staining is expressed as the percent of all tumor cells showing membranous and/or cytoplasmic staining at moderate to strong intensity in evaluable tumor cells.

$$DLL 3 positivity [ \% ] = \frac{DLL 3 positive tumor cells}{( DLL 3 positive tumors cells ) + ( DLL 3 negative tumor cells )}$$

### DLL3 Status Criteria

| DLL3 Status | Staining Criteria |
|---|---|
| Negative | Absence of staining or only weak membranous and/or |
| | cytoplasmic staining in evaluable tumor cells |
| Positive | >0% of tumor cells with moderate to strong membranous |
| | and/or cytoplasmic staining in evaluable tumor cells |
| | ≥25% of tumor cells with moderate to strong membranous |
| | and/or cytoplasmic staining in evaluable tumor cells |
| | ≥50% of tumor cells with moderate to strong membranous |
| | and/or cytoplasmic staining in evaluable tumor cells |
| | ≥75% of tumor cells with moderate to strong membranous |
| | and/or cytoplasmic staining in evaluable tumor cells |

DLL3 expression and H&E analysis and interpretation of results was performed by a qualified pathologist. Only samples with a minimum of 100 evaluable tumor cells were evaluated for DLL3 expression status. DLL3 staining was evaluated at any magnification. DLL3-specific tumor cell staining was expressed as the percent of all tumor cells showing membranous and/or cytoplasmic staining at moderate to strong intensity in evaluable tumor cells. Strong membranous and/or cytoplasmic staining of tumor cells was scored as “3” or “3+,”, moderate membranous and/or cytoplasmic staining as “2” or “2+,” and weak membranous and/or cytoplasmic staining as “1” or “1+.” Absence of staining was given a score of “0.” DLL3 positivity of the tumor samples was defined as greater than 0% DLL3 positive tumor cells at moderate to strong intensity.

### Example 10: Baseline DLL3 Expression

DLL3 expression status in tumor tissue of 134 patients treated with BI 764532/obrixtamig in study 1438-0001, i.e. the study described in Example 1 (cut-off date: 21 Jun. 2024) was analyzed with the Method described in Example 9. The analysis included 134 patients, 63 patients with extensive stage small cell lung cancer (ES-SCLC), 60 patients with extrapulmonary neuroendocrine carcinoma (epNEC), and 11 patients with large cell neuroendocrine carcinoma of the lung (LCNEC-Lung).

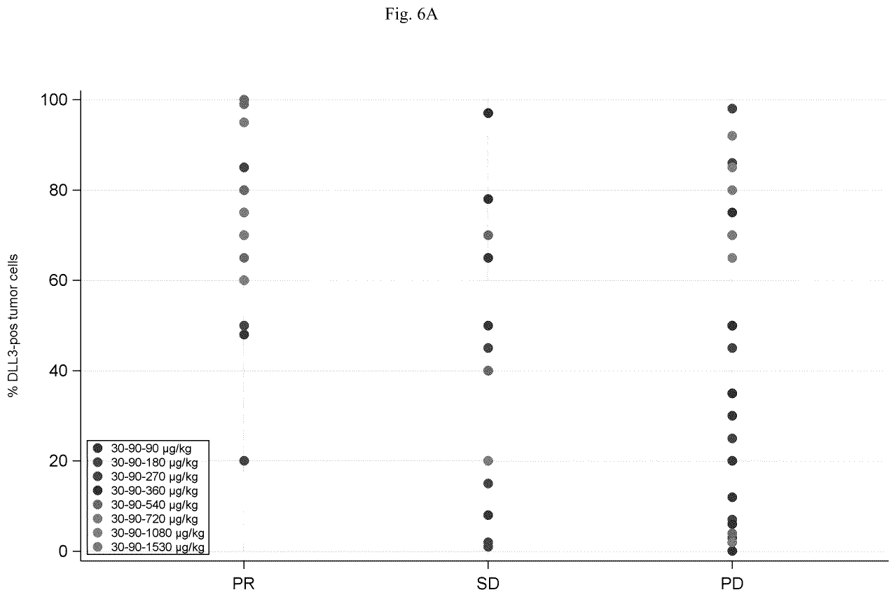

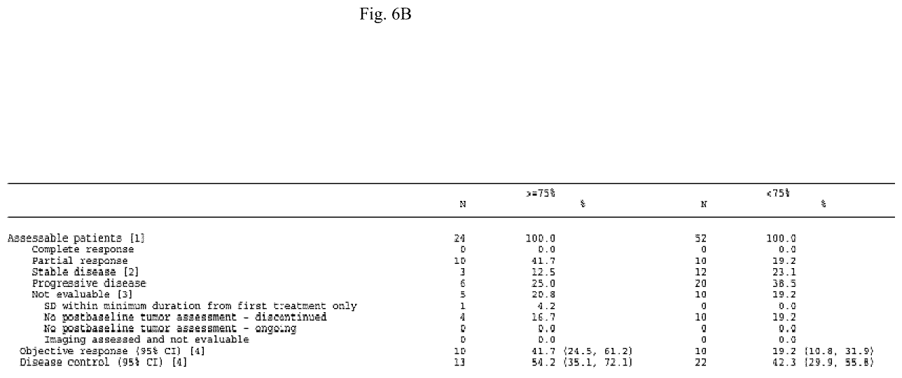

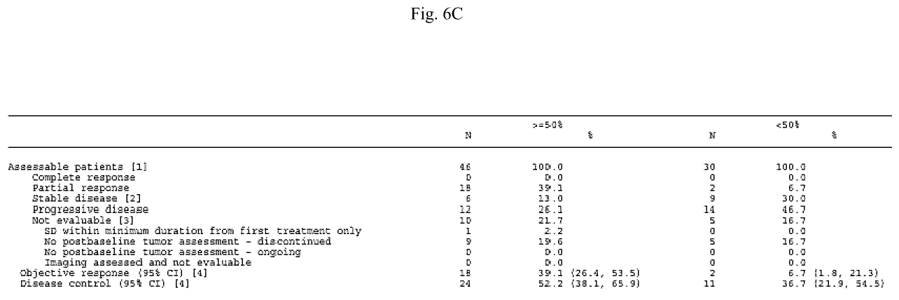

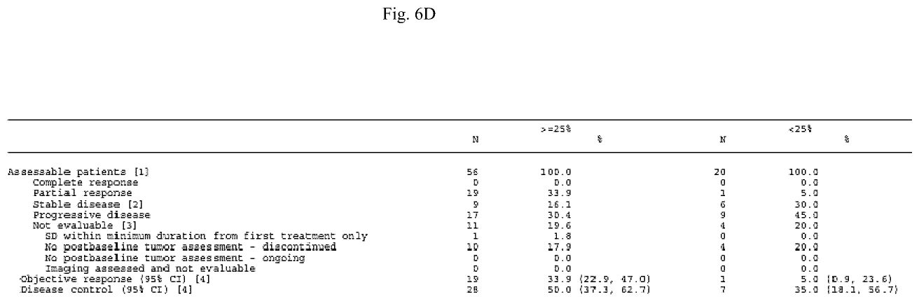

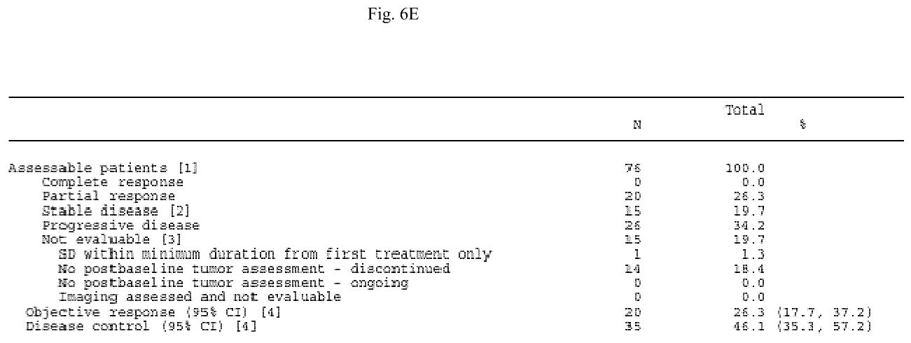

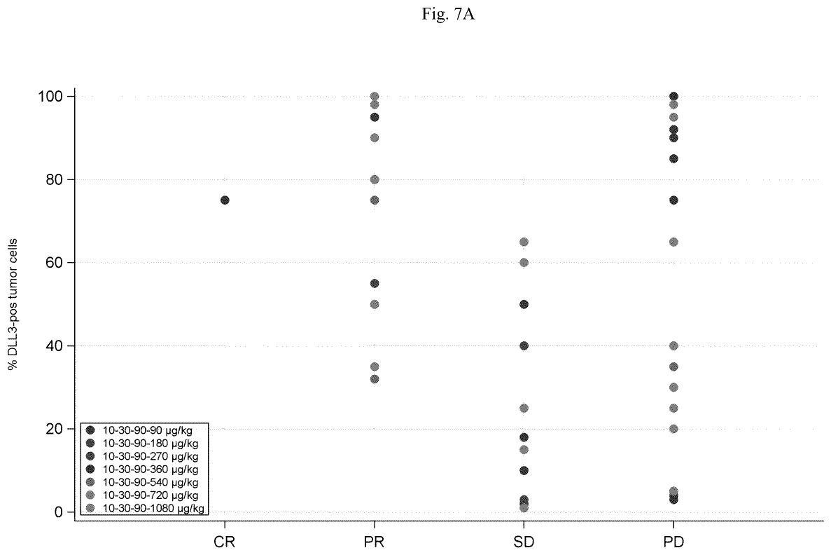

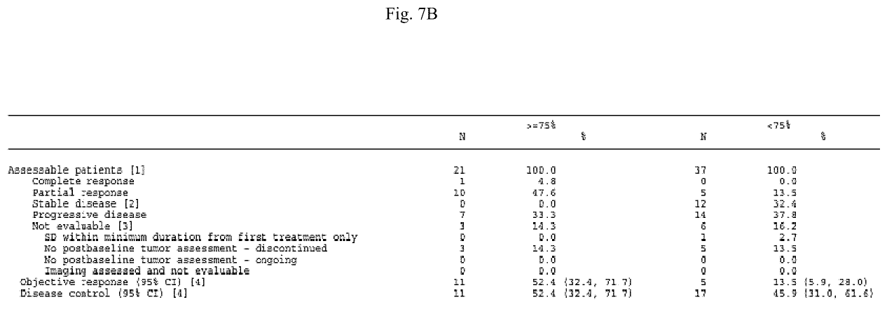

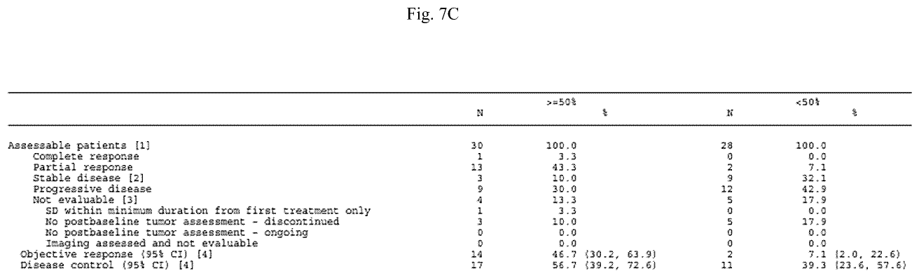

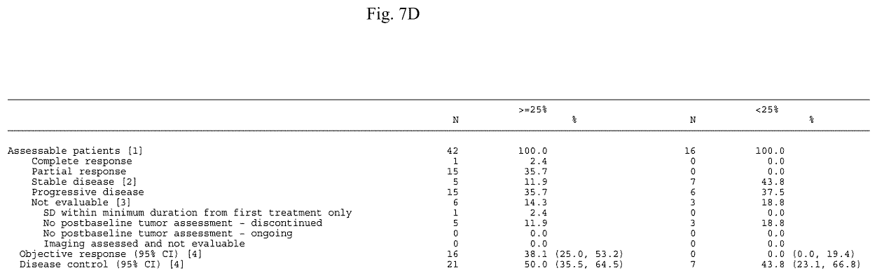

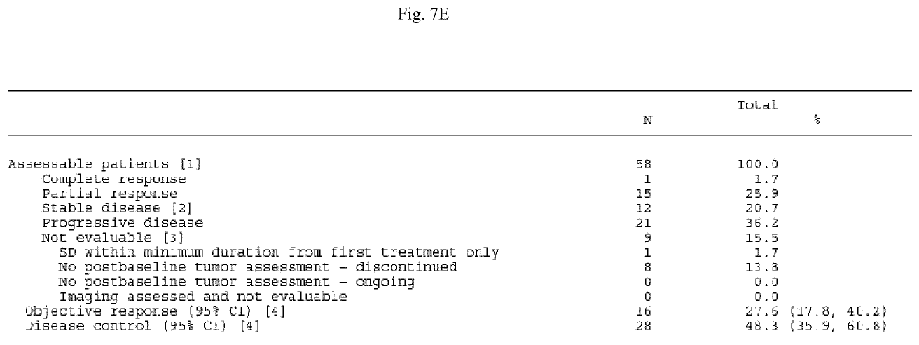

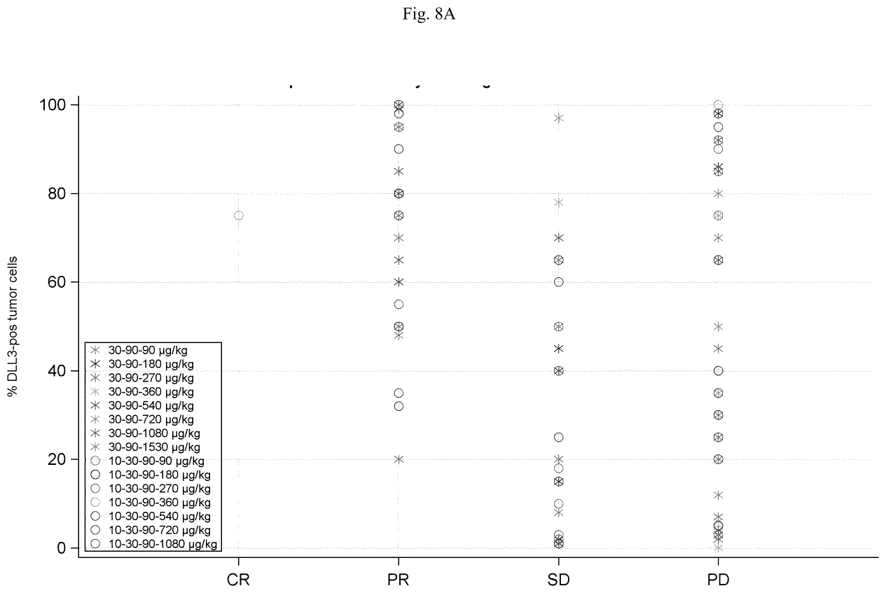

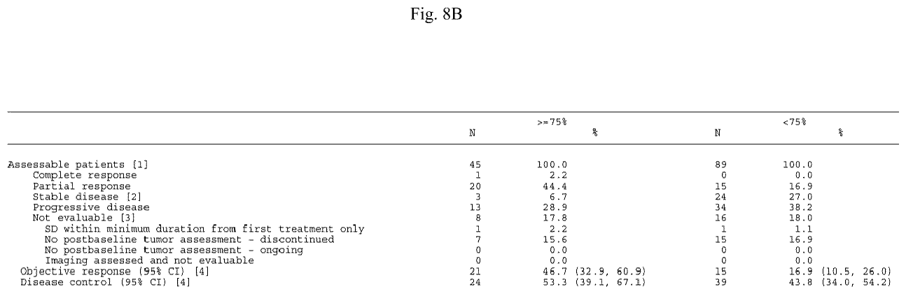

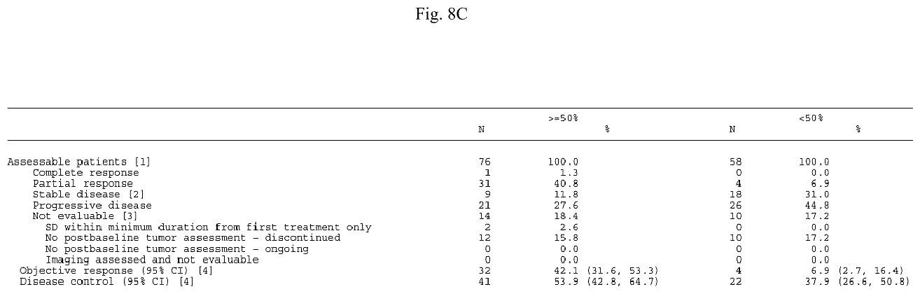

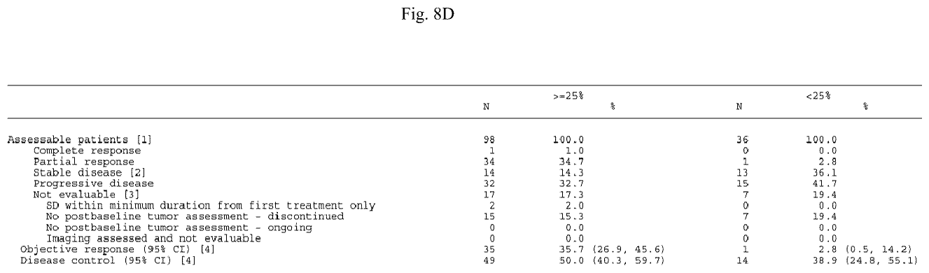

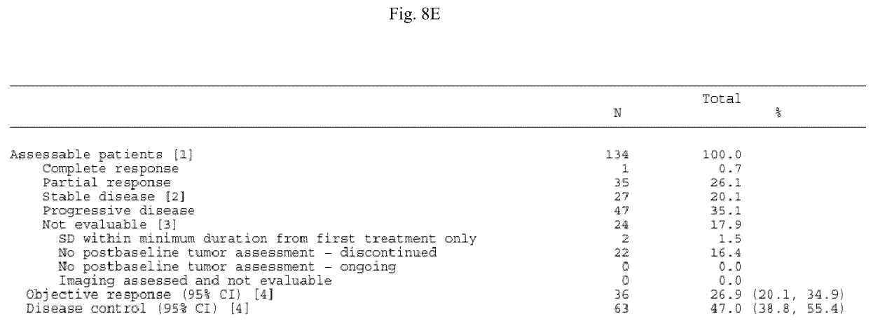

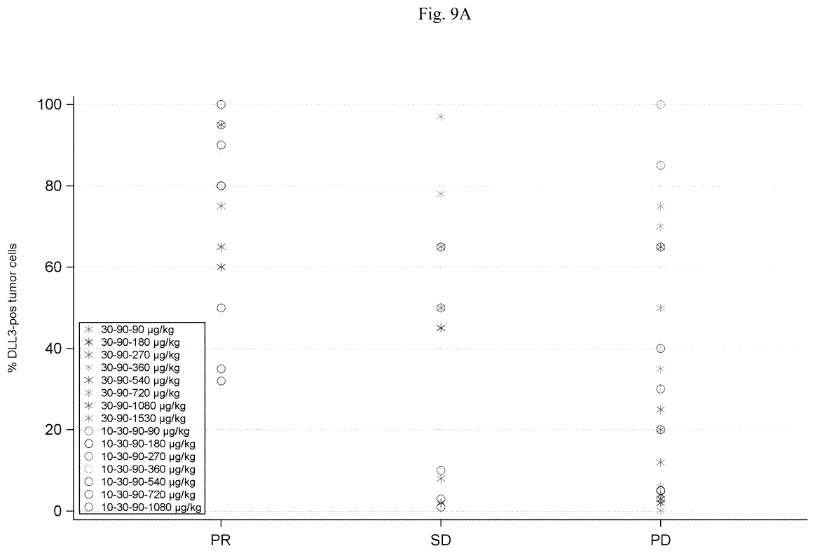

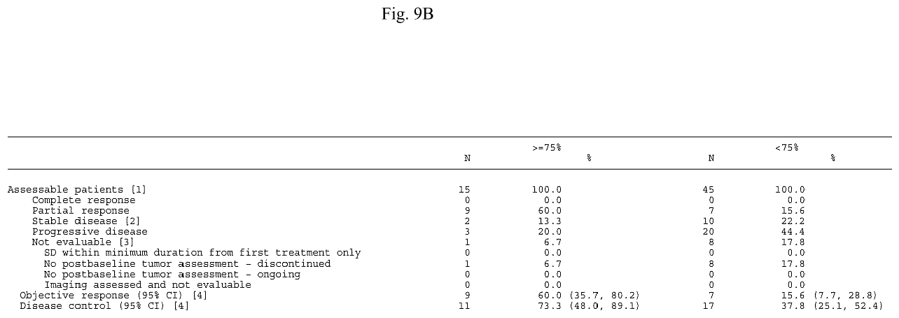

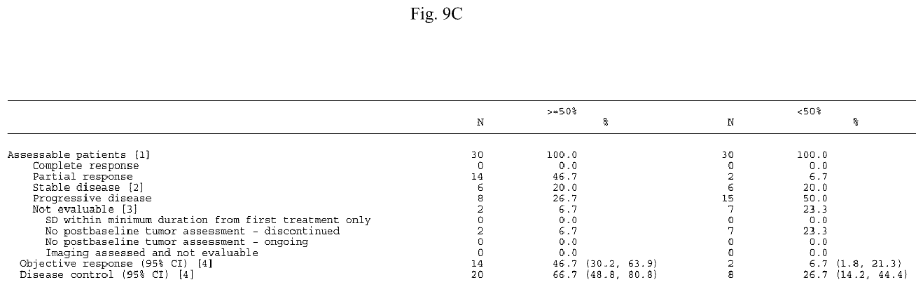

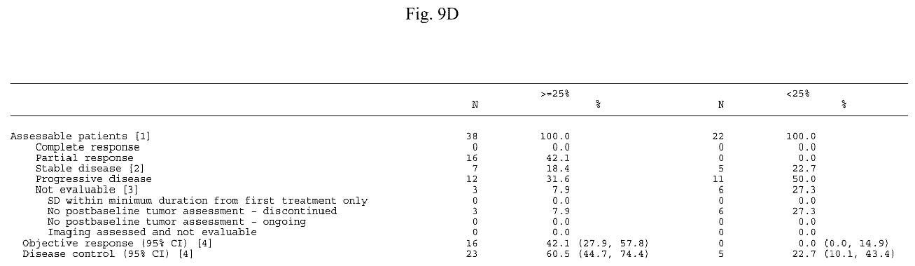

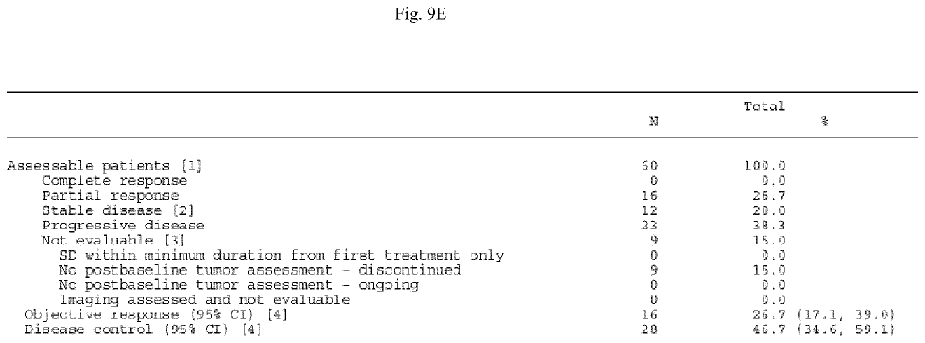

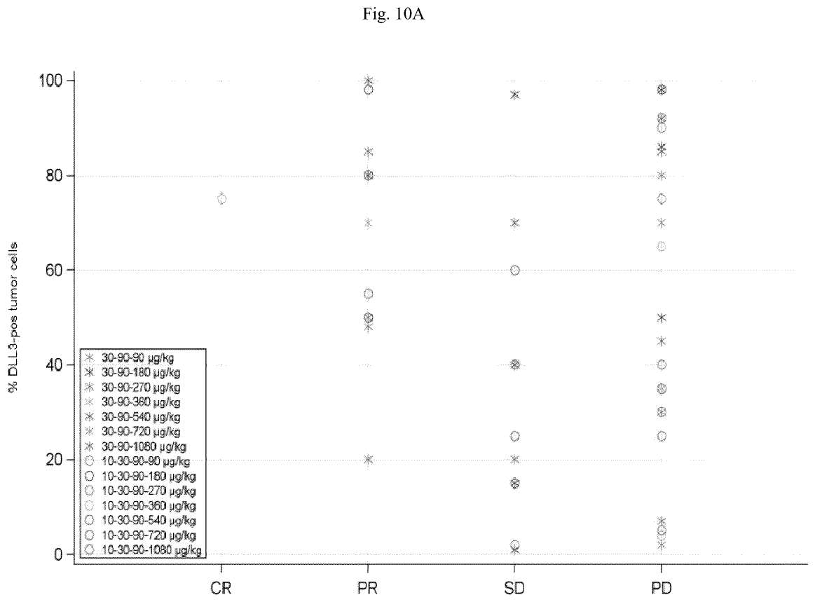

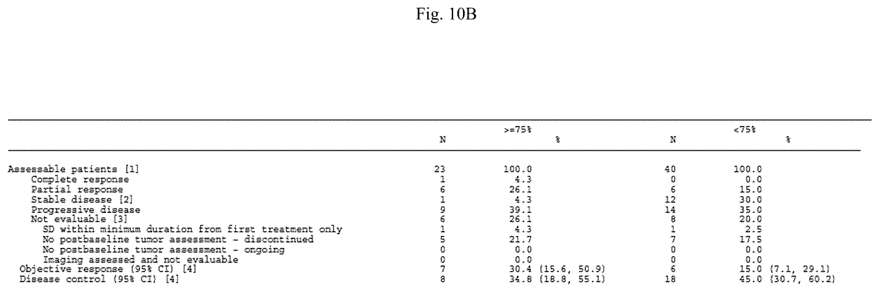

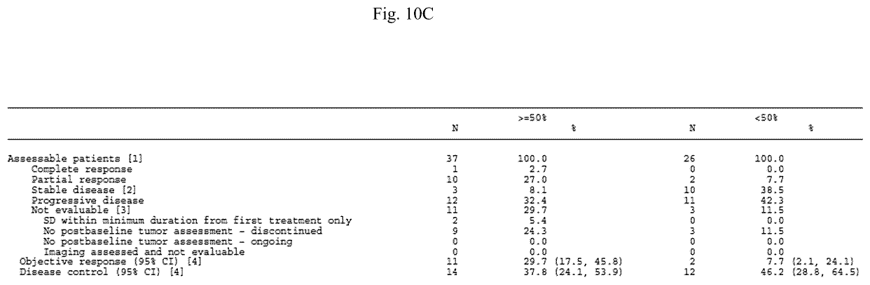

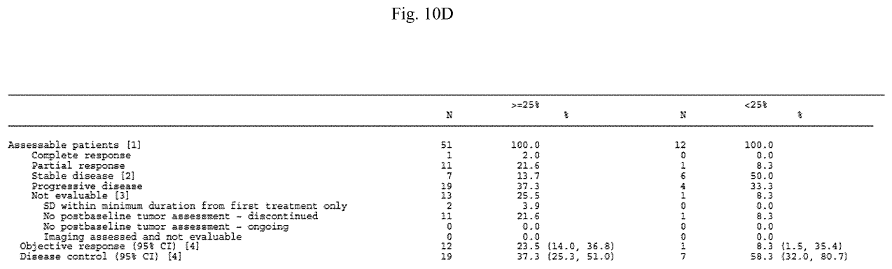

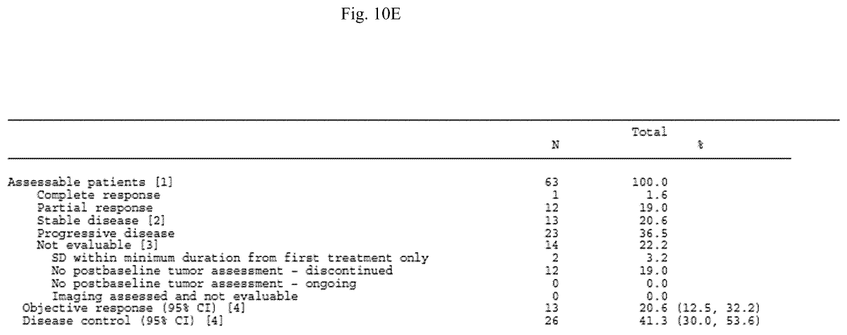

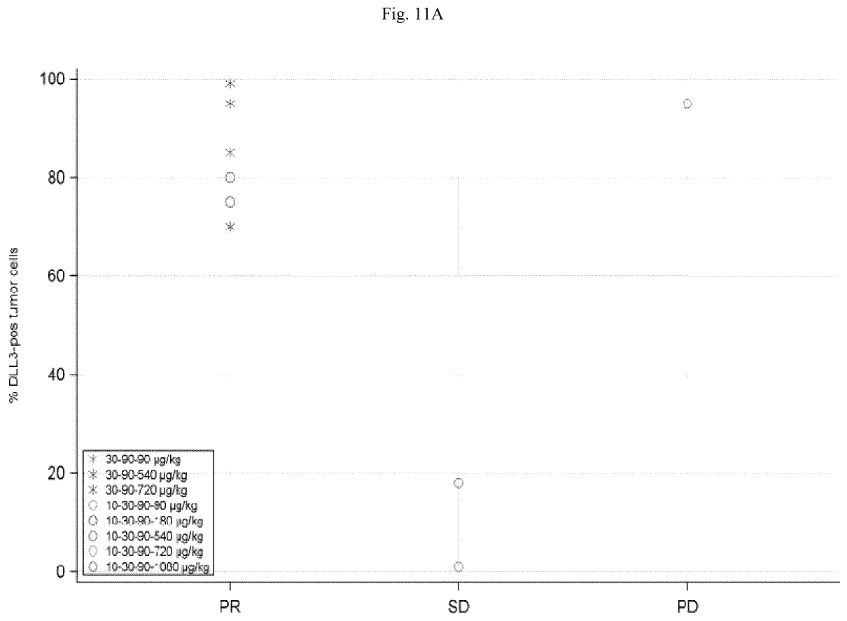

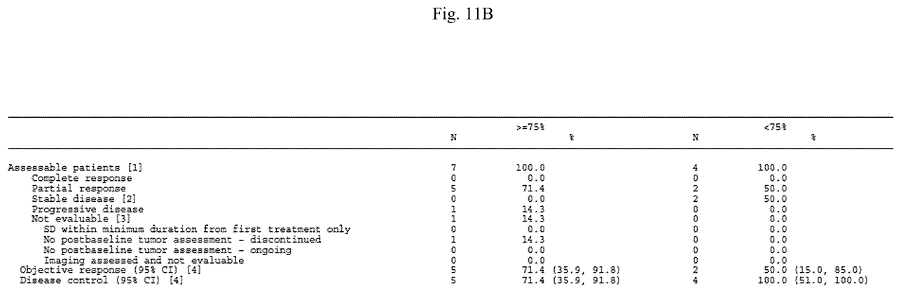

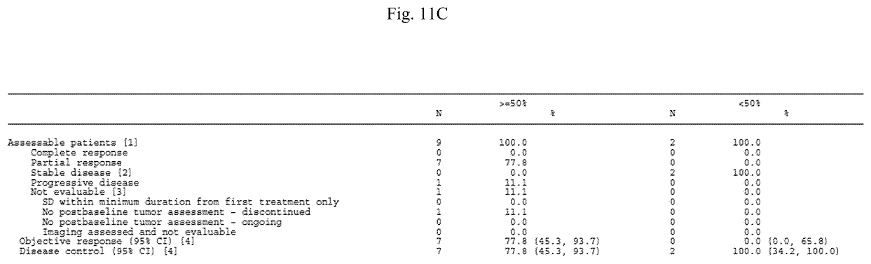

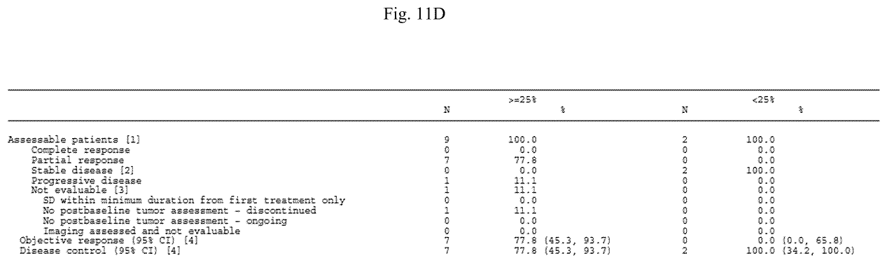

76 patients were treated in regimen B2 and 58 patients were treated in regimen B3.
Of 134 patients with DLL3-positive tumor tissues treated in Regimen B2 and B3, 98 patients (73%) had tumors with ≥25% DLL3-positive tumor cells, 76 patients (57%) of patients had tumors with ≥50% DLL3-positive tumor cells, and 45 patients (34%) of patients had tumors with ≥75% DLL3-positive tumor cells (FIG. 8B-D).
Of 63 SCLC patients with DLL3-positive tumor tissues treated in Regimen B2 and B3, 51 SCLC patients (81%) had tumors with ≥25% DLL3-positive tumor cells, 37 SCLC patients (59%) of patients had tumors with ≥50% DLL3-positive tumor cells, and 23 SCLC patients (37%) of patients had tumors with ≥75% DLL3-positive tumor cells (FIG. 10B-D).
Of 60 epNEC patients with DLL3-positive tumor tissues treated in Regimen B2 and B3, 38 epNEC patients (63%) had tumors with ≥25% DLL3-positive tumor cells, epNEC patients (50%) of patients had tumors with ≥50% DLL3-positive tumor cells, and 15 epNEC patients (25%) of patients had tumors with ≥75% DLL3-positive tumor cells (FIG. 9B-D).
Of 11 LCNEC patients with DLL3-positive tumor tissues treated in Regimen B2 and B3, 9 LCNEC patients (82%) had tumors with ≥25% DLL3-positive tumor cells, 9 LCNEC patients (82%) of patients had tumors with ≥50% DLL3-positive tumor cells, and 7 LCNEC patients (64%) of patients had tumors with ≥75% DLL3-positive tumor cells (FIG. 11B-D).
Of 76 patients with DLL3-positive tumor tissues treated in Regimen B2, 56 patients (74%) had tumors with ≥25% DLL3-positive tumor cells, 46 patients (57%) of patients had tumors with ≥50% DLL3-positive tumor cells, and 24 patients (32%) of patients had tumors with ≥75% DLL3-positive tumor cells (FIG. 6B-D).
Of 58 patients with DLL3-positive tumor tissues treated in Regimen B3, 42 patients (72%) had tumors with ≥25% DLL3-positive tumor cells, 30 patients (71%) of patients had tumors with ≥50% DLL3-positive tumor cells, and 21 patients (36%) of patients had tumors with ≥75% DLL3-positive tumor cells (FIG. 7B-D).

### Example 11: Correlation of Baseline DLL3 Expression with Objective Response to BI 764532 (Obrixtamig) Treatment

DLL3 expression status in tumor tissue and efficacy analysis were performed in 134 patients as described in Example 10 treated with BI 764532/obrixtamig in the study of Example 1 (regimens B2 and B3/cut-off date: 21 Jun. 2024) was analyzed with the Method described in Example 9. Objective response (OR) is defined as the best overall response of confirmed complete response (CR) or confirmed partial response (PR) according to RECIST v1.1 by investigator assessment from the date of treatment start until the earliest date of disease progression, death, or last evaluable tumor assessment before start of subsequent anticancer therapy, loss to follow-up, or withdrawal of consent. Best overall tumor response and disease control according to RECIST v1.1 (by investigator assessment) in DLL3 subgroups are shown.
Of the 134 patients, 1 patient (0.7%) had confirmed CR, 35 patients (26.1%) had confirmed PR, 27 patients (20.1%) had confirmed SD. The confirmed OR rate (ORR) was 26.9%. The disease control rate was 47%. (FIG. 8A+E)
In the group of patients with ≥25% of tumor cells with moderate to strong membranous and/or cytoplasmic staining in evaluable tumor cells, 1 patient (1%) had confirmed CR, 34 patients (34.7%) had confirmed PR, 14 patients (14.3%) had confirmed SD. The confirmed OR rate (ORR) was 35.7%. The disease control rate was 50%. (FIG. 8D)
In the group of patients with ≥50% of tumor cells with moderate to strong membranous and/or cytoplasmic staining in evaluable tumor cells, 1 patients (1.3%) had confirmed CR, 31 patients (40.8%) had confirmed PR, 9 patients (11.8%) had confirmed SD. The confirmed OR rate (ORR) was 42.1%. The disease control rate was 53.9%. (FIG. 8C)
In the group of patients with ≥75% of tumor cells with moderate to strong membranous and/or cytoplasmic staining in evaluable tumor cells, 1 patients (2.2%) had confirmed CR, 20 patients (44.4%) had confirmed PR, 3 patients (6.7%) had confirmed SD. The confirmed OR rate (ORR) was 46.7%. The disease control rate was 53.3%. (FIG. 8B,)
Of the 63 SCLC patients, 1 SCLC patients (1.6%) had confirmed CR, 12 SCLC patients (19%) had confirmed PR, 13 SCLC patients (20.6%) had confirmed SD. The confirmed OR rate (ORR) was 20.6%. The disease control rate was 41.3%. (FIG. 10A+E)
In the group of SCLC patients with ≥25% of tumor cells with moderate to strong membranous and/or cytoplasmic staining in evaluable tumor cells, 1 SCLC patients (2%) had confirmed CR, 11 SCLC patients (21.6%) had confirmed PR, 7 SCLC patients (13.7%) had confirmed SD. The confirmed OR rate (ORR) was 23.5%. The disease control rate was 37.3%. (FIG. 10D)
In the group of SCLC patients with ≥50% of tumor cells with moderate to strong membranous and/or cytoplasmic staining in evaluable tumor cells, 1 SCLC patients (2.7%) had confirmed CR, 10 SCLC patients (27%) had confirmed PR, 3 SCLC patients (8.1%) had confirmed SD. The confirmed OR rate (ORR) was 29.7%. The disease control rate was 37.8%. (FIG. 10C)
In the group of SCLC patients with ≥75% of tumor cells with moderate to strong membranous and/or cytoplasmic staining in evaluable tumor cells, 1 SCLC patients (4.3%) had confirmed CR, 6 SCLC patients (26.1%) had confirmed PR, 1 SCLC patients (4.3%) had confirmed SD. The confirmed OR rate (ORR) was 30.4%. The disease control rate was 34.8%. (FIG. 10B)
Of the 60 epNEC patients, 16 epNEC patients (26.7%) had confirmed PR, 12 epNEC patients (20%) had confirmed SD. The confirmed OR rate (ORR) was 26.7%. The disease control rate was 46.7%. (FIG. 9A+E)
In the group of epNEC patients with ≥25% of tumor cells with moderate to strong membranous and/or cytoplasmic staining in evaluable tumor cells, 16 epNEC patients (42.1%) had confirmed PR, 7 epNEC patients (18.4%) had confirmed SD. The confirmed OR rate (ORR) was 42.1%. The disease control rate was 60.5%. (FIG. 9D)
In the group of epNEC patients with ≥50% of tumor cells with moderate to strong membranous and/or cytoplasmic staining in evaluable tumor cells, 14 epNEC patients (46.7%) had confirmed PR, 6 epNEC patients (20%) had confirmed SD. The confirmed OR rate (ORR) was 46.7%. The disease control rate was 66.7%. (FIG. 9C,)
In the group of epNEC patients with ≥75% of tumor cells with moderate to strong membranous and/or cytoplasmic staining in evaluable tumor cells, 9 epNEC patients (60%) had confirmed PR, 2 epNEC patients (13.3%) had confirmed SD. The confirmed OR rate (ORR) was 60%. The disease control rate was 73.3%. (FIG. 9B)
Of the 11 LCNEC patients, 7 LCNEC patients (63.6%) had confirmed PR, 2 LCNEC patients (18.2%) had confirmed SD. The confirmed OR rate (ORR) was 63.6%. The disease control rate was 81.8%. (FIG. 11A+E)
In the group of LCNEC patients with ≥25% of tumor cells with moderate to strong membranous and/or cytoplasmic staining in evaluable tumor cells, 7 LCNEC patients (77.8%) had confirmed PR. The confirmed OR rate (ORR) was 77.8%. (FIG. 11D)
In the group of LCNEC patients with ≥50% of tumor cells with moderate to strong membranous and/or cytoplasmic staining in evaluable tumor cells, 7 LCNEC patients (77.8%) had confirmed PR. The confirmed OR rate (ORR) was 77.8%. (FIG. 11C)
In the group of LCNEC patients with ≥75% of tumor cells with moderate to strong membranous and/or cytoplasmic staining in evaluable tumor cells, 5 LCNEC patients (71.4%) had confirmed PR. The confirmed OR rate (ORR) was 71.4%. (FIG. 11B)

### Example 12: Correlation of Baseline DLL3 Expression with Objective Response to BI 764532 (Obrixtamig) Treatment

Efficacy analyses were performed in 76 patients as described in Example 10 treated with BI 764532/obrixtamig in the study described in Example 1, dosing regimens B2/cut-off date: 21 Jun. 2024).

Objective response (OR) is defined as the best overall response of confirmed complete response (CR) or confirmed partial response (PR) according to RECIST v1.1 by investigator assessment from the date of treatment start until the earliest date of disease progression, death, or last evaluable tumor assessment before start of subsequent anticancer therapy, loss to follow-up, or withdrawal of consent.

Best overall tumor response and disease control according to RECIST v1.1 (by investigator assessment) in 76 patients in DLL3 subgroups are shown.
Of the 76 patients, 20 patients (26.3%) had confirmed PR, 15 patients (19.7%) had confirmed SD. The confirmed OR rate (ORR) was 26.3%. The disease control rate was 46.1%. (FIG. 6A+E)
In the group of patients with ≥25% of tumor cells with moderate to strong membranous and/or cytoplasmic staining in evaluable tumor cells, 19 patients (33.9%) had confirmed PR, 9 patients (16.1%) had confirmed SD. The confirmed OR rate (ORR) was 33.9%. The disease control rate was 50%. (FIG. 6D)
In the group of patients with ≥50% of tumor cells with moderate to strong membranous and/or cytoplasmic staining in evaluable tumor cells, 18 patients (39.1%) had confirmed PR, 6 patients (13%) had confirmed SD. The confirmed OR rate (ORR) was 39.1%. The disease control rate was 52.2%. (FIG. 6C)
In the group of patients with ≥75% of tumor cells with moderate to strong membranous and/or cytoplasmic staining in evaluable tumor cells, 10 patients (41.7%) had confirmed PR, 3 patients (12.5%) had confirmed SD. The confirmed OR rate (ORR) was 41.7%. The disease control rate was 45.2%. (FIG. 6B)

### Example 13: Correlation of Baseline DLL3 Expression with Objective Response to BI 764532 (Obrixtamig) Treatment

Efficacy analyses were performed in 58 patients as described in Example 10 treated with BI 764532/obrixtamig in the study described in Example 1, dosing regimens B3 combined/cut-off date: 21 Jun. 2024).

Objective response (OR) is defined as the best overall response of confirmed complete response (CR) or confirmed partial response (PR) according to RECIST v1.1 by investigator assessment from the date of treatment start until the earliest date of disease progression, death, or last evaluable tumor assessment before start of subsequent anticancer therapy, loss to follow-up, or withdrawal of consent.

Best overall tumor response and disease control according to RECIST v1.1 (by investigator assessment) in 58 patients in DLL3 subgroups are shown.
Of the 58 patients, 1 patients (1.7%) had confirmed CR, 15 patients (25.9%) had confirmed PR, 12 patients (20.7%) had confirmed SD. The confirmed OR rate (ORR) was 27.6%. The disease control rate was 48.3%. (FIG. 7A+E)
In the group of patients with ≥25% of tumor cells with moderate to strong membranous and/or cytoplasmic staining in evaluable tumor cells, 1 patient (2.4%) had confirmed CR, 15 patients (35.7%) had confirmed PR, 5 patients (11.9%) had confirmed SD. The confirmed OR rate (ORR) was 38.1%. The disease control rate was 50%. (FIG. 7D)
In the group of patients with ≥50% of tumor cells with moderate to strong membranous and/or cytoplasmic staining in evaluable tumor cells, 1 patient (3.3%) had confirmed CR, 13 patients (43.3%) had confirmed PR, 3 patients (10%) had confirmed SD. The confirmed OR rate (ORR) was 46.7%. The disease control rate was 56.7%. (FIG. 7C,)
In the group of patients with ≥75% of tumor cells with moderate to strong membranous and/or cytoplasmic staining in evaluable tumor cells, 1 patient (4.8%) had confirmed CR, 10 patients (47.6%) had confirmed PR. The confirmed OR rate (ORR) was 52.4%. The disease control rate was 52.4% (FIG. 7B)

### Example 14: Integrated Efficacy by DLL3 Subgroups Analysis in epNEC Patients from Monotherapy 1438-0001, Dareon-5 (Part-I) Trials

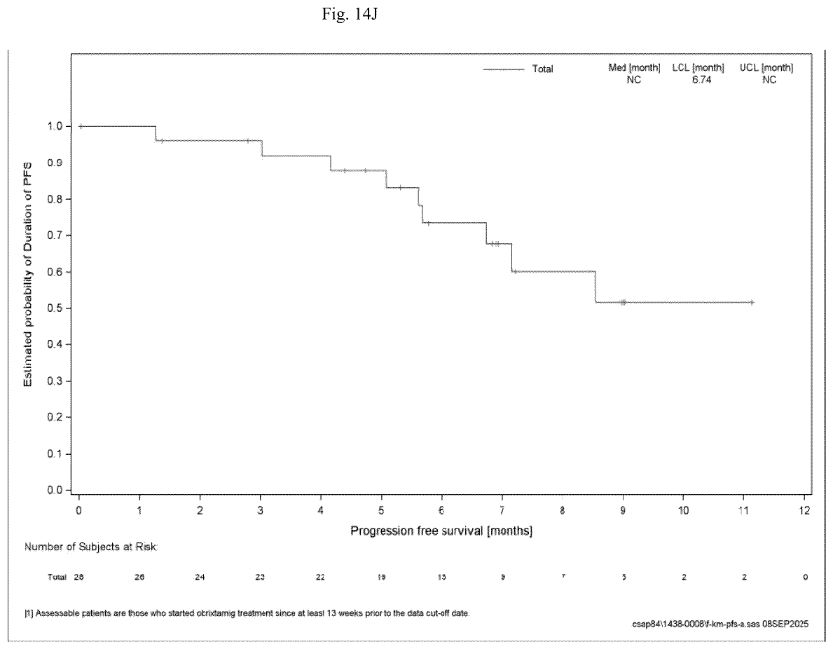

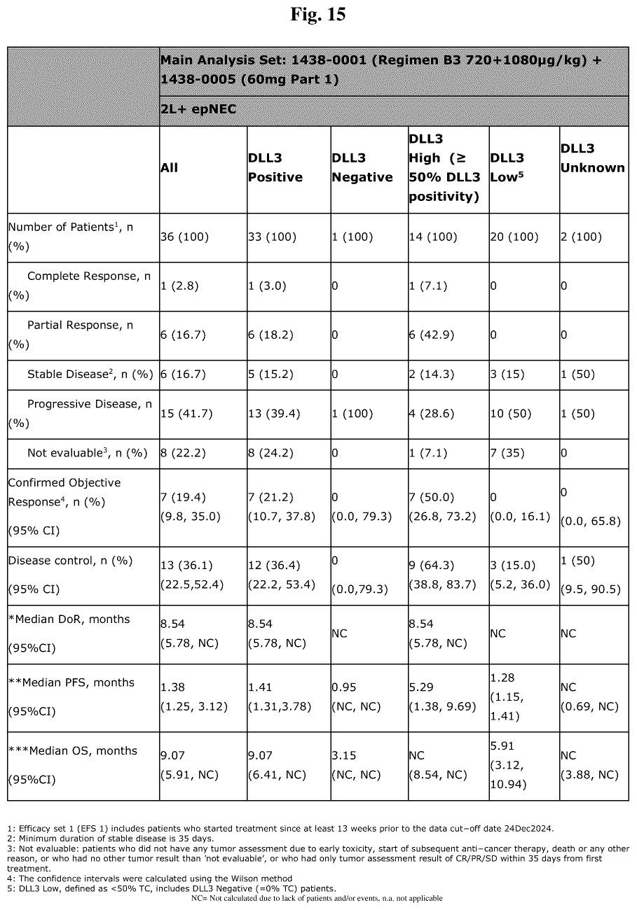

FIG. 15 includes a summary of an integrated efficacy analysis by DLL3 subgroups across patients with previously treated epNEC from the obrixtamig monotherapy trials, 1438-0001 (see Example 1) and DAREON®-5 (Dareon-5 Part-I, see Example 3). Data from epNEC patients who received the 3-dose step-up regimen with a “high target dose” of obrixtamig (720 μg/kg and 1080 μg/kg¹) from Regimen B3 of 1438-0001 and 60 mg from Dareon-5 (Part-1) were pooled for the following efficacy analysis.

As of Dec. 24, 2024, 36 patients with epNEC from the main analysis set have been followed up for median of 9.56 months. Among these patients, 38.9% ( 14/36) patients had tumors with ≥50% of tumor cells with moderate to strong membranous and/or cytoplasmic staining in evaluable tumor cells and 55.6% ( 20/36) with <50% of tumor cells with DLL3 staining and 5.6% ( 2/36) had unknown DLL3 status.
Of the 36 epNEC patients, regardless of DLL3 expression, 1 patient showed complete response (2.8%), 6 epNEC patients (16.7%) had confirmed PR, 6 epNEC patients (16.7%) had confirmed SD. The confirmed OR rate (ORR) was 19.4%. The disease control rate was 36.1%. (FIG. 15, column “All”), column “All”)
In the group of 33 epNEC patients with >0% of tumor cells with moderate to strong membranous and/or cytoplasmic staining in evaluable tumor cells, 6 epNEC patients (18.2%) had confirmed PR, 5 epNEC patients (15.2%) had confirmed SD. The confirmed OR rate (ORR) was 21.2%. The disease control rate was 36.4%. (FIG. 15, column “DLL3 Positive”)
In the group of 14 epNEC patients with ≥50% of tumor cells with moderate to strong membranous and/or cytoplasmic staining in evaluable tumor cells, 1 patient (7.1%) showed confirmed complete response, 6 epNEC patients (42.9%) had confirmed PR, 2 epNEC patients (14.3%) had confirmed SD. The confirmed OR rate (ORR) was 50.0%. The disease control rate was 64.3%. (FIG. 15, column “DLL3 High”)

In the main analysis set, of 14 patients with epNEC who had DLL3 with ≥50% of tumor cells expressing DLL3, 7 patients (50.0%; 95% CI 26.8, 73.2) had confirmed objective response, including 1 complete response (CR). Disease control was observed in 9 patients (64.3%; 95% CI 38.8, 83.7). The responses have been durable: the duration of response (DoR) rate at 6 months and at 9 months are 67% and 44%, respectively.

### Example 15: Efficacy by DLL3 Subgroups Analysis in EPNEC Patients from Dareon-7 (1438-0007) Trial

Data from DAREON®-7 (1438-0007, see Examples 6A and 6B) trial, from patients with previously untreated epNEC who received the 3-dose step-up regimen with all 3 dose levels were analyzed for the efficacy in regard to DLL3 expression levels.

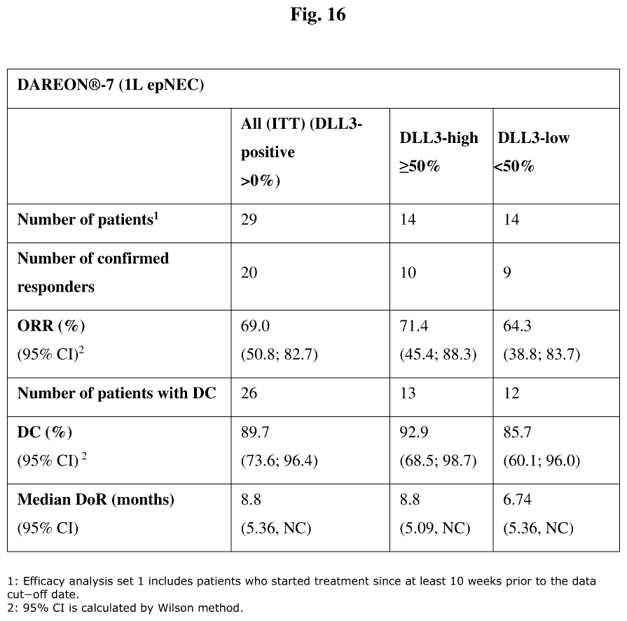

As of Aug. 28, 2025, data from 29 patients who started treatment since at least 10 weeks prior to the data cut-off date were available.
Of the 29 epNEC patients, with >0% of tumor cells with moderate to strong membranous and/or cytoplasmic staining in evaluable tumor cells 20 epNEC patients (69.0%) had confirmed PR, 26 epNEC patients (89.7%) had confirmed DC. The confirmed OR rate (ORR) was 69.0%. The disease control rate was 89.7%. (FIG. 16, column “All (ITT) (DLL3-positive >0%). The classification of one of the patients among the 29 patients is unclear so the patient was not classified as DLL3-high or low (see FIG. 16).
In the group of 14 epNEC patients with ≥50% of tumor cells with moderate to strong membranous and/or cytoplasmic staining in evaluable tumor cells, 10 epNEC patients (71.4%) had confirmed PR, 13 epNEC patients (92.9%) had confirmed DC. The confirmed OR rate (ORR) was 71.4%. The disease control rate was 92.9%. (FIG. 16, column “DLL3-high ≥50%”)

### Example 16: Integrated Efficacy by DLL3 Subgroups Analysis in ES-SCLC Patients from Monotherapy 1438-0001, Dareon-5 (Part-I) Trials

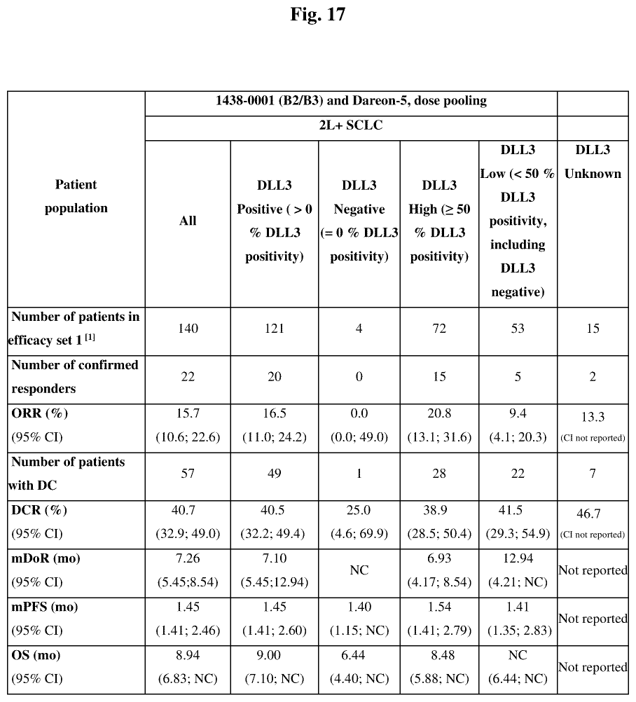

FIG. 17 includes a summary of an integrated efficacy analysis by DLL3 subgroups across patients with previously treated ES-SCLC from the obrixtamig monotherapy trials, 1438-0001 and DAREON®-5 (Dareon-5 Part-I) (see Examples 1 and 3). Data from 140 ES-SCLC patients, who were DLL3 positive and received the B2/B3 regimens across all doses of obrixtamig (from 1438-0001 trial) and patients regardless of DLL3 expression, who received B3F regimen of obrixtamig with either 10 mg or 60 mg target dose (from 1438-0005 trial) were pooled for the following efficacy analysis (cut-off date: 24 Dec. 2024).
Of the 140 ES-SCLC patients, 22 ES-SCLC patients (15.7%) had confirmed PR, 57 ES-SCLC patients (40.7%) had confirmed SD. The confirmed OR rate (ORR) was 15.7%. The disease control rate was 40.7%. (FIG. 17, column “All”)
In the group of 121 ES-SCLC patients with >0% of tumor cells with moderate to strong membranous and/or cytoplasmic staining in evaluable tumor cells, 20 ES-SCLC patients (16.5%) had confirmed PR, 49 ES-SCLC patients (40.5%) had confirmed DC. The confirmed OR rate (ORR) was 16.5%. The disease control rate was 40.5%. (FIG. 17, column “DLL3 Positive”)
In the group of 72 ES-SCLC patients with ≥50% of tumor cells with moderate to strong membranous and/or cytoplasmic staining in evaluable tumor cells, 15 ES-SCLC patients (20.8%) had confirmed PR, 28 ES-SCLC patients (38.9%) had confirmed DC. The confirmed OR rate (ORR) was 20.8%. The disease control rate was 38.9%. (FIG. 17, column “DLL3 High”)

### Example 17: Efficacy by DLL3 Subgroups Analysis in ES-SCLC Patients from Dareon-8 (1438-0008) Trial

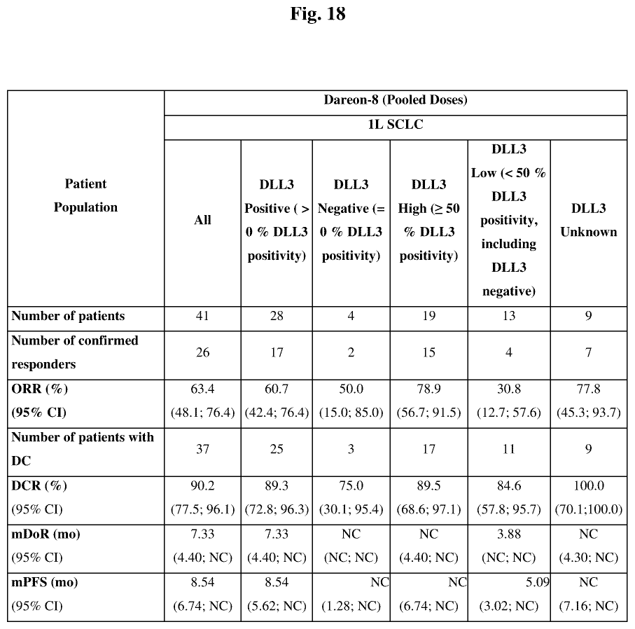

FIG. 18 includes a summary of the efficacy by DLL3 subgroups from the Dareon-8 trial (see Examples 7A and 7B), in which patients with previously untreated ES-SCLC were treated with obrixtamig in combination with standard of care (platinum, etoposide, and atezolizumab).

As of Aug. 21, 2025, data from 41 patients who started treatment since at least 13 weeks prior to the data cut-off date were available.
Of the 41 ES-SCLC patients, regardless of DLL3 expression, 26 ES-SCLC patients (63.4%) had confirmed PR, 37 ES-SCLC patients (90.2%) had confirmed DC. The confirmed OR rate (ORR) was 63.4%. The disease control rate was 90.2%. (FIG. 18, column “All”)
Of the 28 ES-SCLC patients, with >0% of tumor cells with moderate to strong membranous and/or cytoplasmic staining in evaluable tumor cells 17 ES-SCLC patients (60.7%) had confirmed PR, 25 ES-SCLC patients (89.3%) had confirmed DC. The confirmed OR rate (ORR) was 60.7%. The disease control rate was 89.3%. (FIG. 18, column “DLL3 Positive”)
In the group of 19 ES-SCLC patients with ≥50% of tumor cells with moderate to strong membranous and/or cytoplasmic staining in evaluable tumor cells, 15 ES-SCLC patients (78.9%) had confirmed PR, 17 ES-SCLC patients (89.5%) had confirmed DC. The confirmed OR rate (ORR) was 78.9%. The disease control rate was 89.5%. (FIG. 18, column “DLL3 High”)

### Example 18: Post-Medication (in BI 764532 Monotherapy Treatment Regimen)

Dareon-5 Part 2 is an indication-specific expansion cohort including epNEC patients with DLL3 high expressing tumours (for DLL3 high definition see Example 14). BI 764532 is administered with a schedule B3F (see Example 3), including step-in doses of 10, 30, 90 μg/kg and a target dose of 60 mg.

The premedication in Dareon-5 before BI 764532 administration includes:
Acetaminophen/paracetamol 1000 mg p.o. or i.v., or equivalent.
Antihistamine (diphenhydramine 50 mg p.o. or i.v., or equivalent) and/or
Steroids (dexamethasone 8 mg p.o. or i.v., or equivalent intermediate-acting corticosteroid).

In addition, in Dareon-5 Part 2 post-medication with steroids is also administered at least after all step-in doses of obrixtamig and after the first obrixtamig target dose administration:
Dexamethasone 8 mg p.o. or i.v. or equivalent intermediate-acting corticosteroid is administered 8 (±2) hours following the start of administration of BI 764532. Depending on the duration of monitoring, patients may be discharged with an oral steroid and advised to take them at home.

If the individual patient has tolerated BI 764532 with steroid premedication and post-medication without >Grade 1 IRR or CRS for the step-in doses, steroids premedication and post-medication in this patient may be reduced to 50% (e.g. steroids i.v., dexamethasone 4 mg) with subsequent administrations. If one BI 764532 target dose with reduced steroid premedication and post-medication is tolerated without >Grade 1 IRR or CRS in an individual patient, steroid premedication and post-medication should be skipped in repeated treatment cycles for the respective patient.

Pre- and post-medication for obrixtamig

| | Acetaminophen/Paracetamol & | Dexamethasone |
|---|---|---|
| Obrixtamig Dose | Antihistamine pre-medication | (or equivalent steroid) |
| 1^{st} | Yes | 8 mg prior to obrixtamig administration |
| 10 μg/kg (step-in) | | and |
| | | 8 mg post-obrixtamig administration |
| 2^{nd} | Yes | 8 mg prior to obrixtamig administration |
| 30 μg/kg (step-in) | | and |
| | | 8 mg post-obrixtamig administration |
| 3^{rd} | Yes | 8 mg prior to obrixtamig administration |
| 90 μg/kg (step-in) | | and |
| | | 8 mg post-obrixtamig administration |
| 4^{th} dose | Yes | 4 mg prior to obrixtamig administration |
| 60 mg (target) | | and |
| | | 4 mg post-obrixtamig administration* |
| 5^{th} dose | Yes | None if previously well tolerated* |
| 60 mg (target) | | |
| 6^{th} dose | Yes | None if previously well tolerated* |
| 60 mg (target) | | |
| 7^{th} dose | Yes | None if previously well tolerated* |
| 60 mg (target) | | |
| (1^{st} dose after 3-week break) | | |
| ≥8^{th} dose | Yes | None if previously well tolerated* |
| 60 mg (target) | | |

*Dexamethasone (or equivalent) to be reduced only if no CRS/IRR ≥ 2 and/or ICANS (any grade) occurred following the prior dose. If CRS/IRR/ICANS occur the dexamethasone dose at the next drug administration must remain the same as the current visit and must not be reduced. Dexamethasone reduction may be considered for subsequent administrations.

### Example 19: Post-Medication (in BI 764532 Combination Treatment Regimens)

DAREON®-Lung-1 is a Phase III multi-center, open-label, randomised trial of intravenous obrixtamig in combination with atezolizumab, carboplatin, and etoposide vs. atezolizumab, carboplatin, and etoposide as first-line treatment in patients with extensive-stage small cell lung cancer.

DAREON®-NEC-1 is a Phase III, multi-center, open-label, randomised, controlled trial of intravenous obrixtamig in combination with carboplatin and etoposide vs carboplatin and etoposide as first-line therapy in DLL3 positive patients with unresectable locally advanced or metastatic extrapulmonary neuroendocrine carcinomas.

For the combination treatments in DAREON®-Lung-1 and DAREON®-NEC-1 pre- and post-medication is administered at least before and/or after all step-in dose administrations and the first and second target dose administration of obrixtamig to prevent or reduce severity of potential CRS, infusion-related reaction (IRR), or neurotoxicity. It is recommended that the premedication is completed 30-60 min before administration of obrixtamig.

The pre- and post-medication for each obrixtamig administration includes:
Acetaminophen/paracetamol 1000 mg p.o. or i.v., or equivalent prior to obrixtamig
Antihistamine (diphenhydramine 50 mg p.o. or i.v., or equivalent) prior to obrixtamig; and/or
Steroids (dexamethasone p.o. or i.v. or equivalent intermediate-acting corticosteroid):
   For the first two step-up doses (10 μg/kg and 30 μg/kg): 8 mg prior to +8 mg post-obrixtamig administration (8 h±2 h after the end of infusion) of dexamethasone p.o. or i.v.
   If no CRS/IRR/ICANS occurs, dexamethasone reduction may be considered for subsequent administrations as per Table below. If CRS/IRR/ICANS occurs the dexamethasone dose at the next drug administration must remain the same as the current visit and must not be reduced.

Pre- and post-medication for obrixtamig

| | Acetaminophen/Paracetamol & | Dexamethasone |
|---|---|---|
| Obrixtamig Dose | Antihistamine pre-medication | (or equivalent steroid) |
| 1^{st} | Yes | 8 mg prior to obrixtamig administration |
| 10 μg/kg (step-in) | | and |
| | | 8 mg post-obrixtamig administration |
| 2^{nd} | Yes | 8 mg prior to obrixtamig administration |
| 30 μg/kg (step-in) | | and |
| | | 8 mg post-obrixtamig administration |
| 3^{rd} | Yes | 8 mg prior to obrixtamig administration* |
| 90 μg/kg (step-in) | | |
| 4^{th} dose | Yes | 8 mg premedication* |
| 60 mg (target) | | |
| 5^{th} dose | Yes | 4 mg premedication* |
| 60 mg (target) | | |
| 6^{th} dose | Yes | None if previously well tolerated* |
| 60 mg (target) | | |
| 7^{th} dose | Yes | None if previously well tolerated* |
| 60 mg (target) | | |
| (1^{st} dose after 3-week break) | | |
| ≥8^{th} dose | Yes | None if previously well tolerated* |
| 60 mg (target) | | |

*Dexamethasone (or equivalent) to be reduced only if no CRS ≥ 2 and/or ICANS (any grade) occurred following the prior dose. If CRS/IRR/ICANS occur the dexamethasone dose at the next drug administration must remain the same as the current visit and must not be reduced. Dexamethasone reduction may be considered for subsequent administrations.

1. A T-cell engaging protein comprising a first antigen binding unit specifically binding to DLL3 and a second antigen binding unit specifically binding to CD3 for use in a method for treating a DLL3-positive cancer or a cancer selected from a Neuroendocrine Neo-plasm (NEN), Merkel cell carcinoma (MCC), medullary thyroidal carcinoma and glioma in a subject exhibiting at least a threshold of DLL3-positivity, the method comprising administering to the subject a dose of said T-cell engaging protein, wherein
said T-cell engaging protein comprises a first antigen binding unit specifically binding to DLL3, comprising light chain CDRs comprising the amino acid sequences of SEQ ID NO:13 (CDR1), SEQ ID NO:14 (CDR2) and SEQ ID NO:15 (CDR3) and heavy chain CDRs comprising the amino acid sequences of SEQ ID NO:16 (CDR1), SEQ ID NO:17 (CDR2) and SEQ ID NO:18 (CDR3) and a second antigen binding unit specifically binding to CD3 comprising light chain CDRs comprising the amino acid sequences of SEQ ID NO:55 (CDR1), SEQ ID NO:56 (CDR2) and SEQ ID NO:57 (CDR3) and heavy chain CDRs comprising the amino acid sequences of SEQ ID NO:58 (CDR1), SEQ ID NO:59 (CDR2) and SEQ ID NO:60 (CDR3).

2. The T-cell engaging protein for use according to claim 1, wherein the subject has been determined to exhibit at least a threshold of DLL3-positivity.

3. The T-cell engaging protein for use according to any of the preceding claims, comprising the step of determining or having determined that the subject exhibits at least a threshold of DLL3-positivity.

4. The T-cell engaging protein for use according to any of the preceding claims, wherein the threshold is at least about 10%, at least about 20% at least about 25%, at least about 30%, at least about 40%, at least about 50% or at least about 75%.

5. The T-cell engaging protein for use according to any of the preceding claims, wherein the threshold is at least about 25%, at least about 50%, or at least about 75%.

6. The T-cell engaging protein for use according any one of the preceding claims, wherein the DLL3 positivity in a sample is calculated according to the following formula:
$$DLL 3 - positivity [ \% ] = \frac{DLL 3 - positive tumor cells in the sample}{\begin{matrix}( DLL 3 - positive tumors cells in the sample ) + \\ ( DLL 3 - negative tumor cells in the sample )\end{matrix}} .$$

7. The T-cell engaging protein for use according to claim 6, wherein the DLL3-positive tumor cell exhibits moderate to strong membranous and/or cytoplasmic staining.

8. The T-cell engaging protein for use according to any one of the proceeding claims, wherein DLL3 positivity is determined using an immunohistochemical (IHC) assay.

9. The T-cell engaging protein for use according to any one of the proceeding claims, wherein DLL3 positivity is determined in a tissue sample from the subject, preferably in a formalin fixed paraffin embedded (FFPE) tissue sample.

10. The T-cell engaging protein for use according to any one of the preceding claims, wherein said Neuroendocrine Neoplasm (NEN) is a Neuroendocrine Carcinoma (NEC), preferably selected from the group consisting of Lung-NEC such as LCNEC of the lung, Extrapulmonary (ep) NEC including NEC with unknown primary site; small cell lung cancer (SCLC) including extensive-stage (ES)-SCLC, and limited-stage (LS) SCLC; or said NEN is a Neuroendocrine Tumor (NET) such as grade 3 NET.

11. The T-cell engaging protein for use according to any one of the preceding claims, wherein at least one further anti-cancer agent is to be administered, said further anti-cancer agent preferably being a chemotherapeutic agent and/or an immune-checkpoint inhibitor such as an anti-PD-1 antibody or an anti-PD-L1 antibody.

12. A method for selecting a subject suffering from a DLL3-positive cancer or a cancer selected from a Neuroendocrine Neoplasm (NEN), Merkel cell carcinoma (MCC), medullary thyroidal carcinoma and glioma for treatment with a T-cell engaging protein comprising a first antigen binding unit specifically binding to DLL3 and a second antigen binding unit specifically binding to CD3, the method comprising:
(a) determining or having determined in a sample that the subject exhibits DLL3 positivity, wherein the subject is selected for treatment with said T-cell engaging protein when the DLL3-positivity exhibits at least a threshold, and
wherein said T-cell engaging protein comprises a first antigen binding unit specifically binding to DLL3, comprising light chain CDRs comprising the amino acid sequences of SEQ ID NO:13 (CDR1), SEQ ID NO:14 (CDR2) and SEQ ID NO:15 (CDR3) and heavy chain CDRs comprising the amino acid sequences of SEQ ID NO:16 (CDR1), SEQ ID NO:17 (CDR2) and SEQ ID NO:18 (CDR3) and a second antigen binding unit specifically binding to CD3 comprising light chain CDRs comprising the amino acid sequences of SEQ ID NO:55 (CDR1), SEQ ID NO:56 (CDR2) and SEQ ID NO:57 (CDR3) and heavy chain CDRs comprising the amino acid sequences of SEQ ID NO:58 (CDR1), SEQ ID NO:59 (CDR2) and SEQ ID NO:60 (CDR3).

13. The method according to claim 12, wherein the DLL-3 positivity is calculated according to the following formula:
$$DLL 3 - positivity [ \% ] = \frac{DLL 3 - positive tumor cells in the sample}{\begin{matrix}( DLL 3 - positive tumors cells in the sample ) + \\ ( DLL 3 - negative tumor cells in the sample )\end{matrix}} .$$

14. The method according to claim 12 or 13, wherein the threshold is at least 10%, at least about 20% at least about 25%, at least about 30%, at least about 40%, at least about 50% or at least about 75%.

15. The method according to any one of claims 12 to 14, wherein the threshold is at least about 25%, at least about 50%, or at least about 75%.

16. The method according to any one of claims 12 to 15, wherein the number of DLL3-positive tumor cells in the sample is determined by counting immunostained tumor cells in the sample.


SEQUENCE LISTING (90 sequences)

SEQ ID NO: 1 - protein, 12 aa, synthetic construct
RASQSVSSNFLV

SEQ ID NO: 2 - protein, 7 aa, synthetic construct
GASTRAS

SEQ ID NO: 3 - protein, 9 aa, synthetic construct
QQYGDSPYT

SEQ ID NO: 4 - protein, 10 aa, synthetic construct
GNTFTNYYMH

SEQ ID NO: 5 - protein, 17 aa, synthetic construct
IIDPSVGSKSYAQKFLG

SEQ ID NO: 6 - protein, 12 aa, synthetic construct
AGKRFGESYFDY

SEQ ID NO: 7 - protein, 11 aa, synthetic construct
RASQGISNYLA

SEQ ID NO: 8 - protein, 7 aa, synthetic construct
AASSLQS

SEQ ID NO: 9 - protein, 9 aa, synthetic construct
LQHNSSPYT

SEQ ID NO: 10 - protein, 10 aa, synthetic construct
GYTFTSYYMH

SEQ ID NO: 11 - protein, 17 aa, synthetic construct
IINPSGGSTSYAQKFQG

SEQ ID NO: 12 - protein, 15 aa, synthetic construct
GEAVGGNYYYYGMDV

SEQ ID NO: 13 - protein, 11 aa, synthetic construct
RASQGISNYLV

SEQ ID NO: 14 - protein, 7 aa, synthetic construct
AVSSLYS

SEQ ID NO: 15 - protein, 9 aa, synthetic construct
LQHDSYPYT

SEQ ID NO: 16 - protein, 10 aa, synthetic construct
GYTFTSYYVH

SEQ ID NO: 17 - protein, 17 aa, synthetic construct
IINPGGGTTSYAQKFLG

SEQ ID NO: 18 - protein, 15 aa, synthetic construct
GEAVTGNYFYYGMDV

SEQ ID NO: 19-36 - skipped (18 entries)

SEQ ID NO: 37 - protein, 108 aa, synthetic construct
EIVLTQSPGTLSLSPGERATLSCRASQSVSSNFLVWYQQKPGQAPRPLIYGASTRASGIPDRFSGSGSGADFTLTISRLEPEDFALYYCQQYGDSPYTFGQGTTLEIK

SEQ ID NO: 38 - protein, 121 aa, synthetic construct
QVQLVQSGAEVKKPGASVKVSCKASGNTFTNYYMHWVRQAPGPGLEWMGIIDPSVGSKSYAQKFLGRVTIARDTSTSTVFLDLYSLRSEDTAVYFCARAGKRFGESYFDYWGQGTLVTVSS

SEQ ID NO: 39 - protein, 107 aa, synthetic construct
DIQMTQSPSAMSASVGDRVTITCRASQGISNYLAWFQQKPGKVPEPLIYAASSLQSGVPSRFSGSGSVTEFTLTISSLQPEDFATYYCLQHNSSPYTFGQGTKLEIK

SEQ ID NO: 40 - protein, 124 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGEAVGGNYYYYGMDVWGQGTTVTVSS

SEQ ID NO: 41 - protein, 107 aa, synthetic construct
DIQMTQSPSAMSASVGDRVTITCRASQGISNYLVWFQQKPGKAPKRLIYAVSSLYSGVPSRFSGSGSGTEFTLTISSLQPEDFATYYCLQHDSYPYTFGQGTKLEIK

SEQ ID NO: 42 - protein, 124 aa, synthetic construct
QVQLVQSGAEVKKPGASVKVSCKASGYTFTSYYVHWVRQAPGQGLEWMVIINPGGGTTSYAQKFLGRVTMTRDTSTNTVYMELKSLRSEDTAVYYCARGEAVTGNYFYYGMDVWGQGTTVTVSS

SEQ ID NO: 43-48 - skipped (6 entries)

SEQ ID NO: 49 - protein, 477 aa, synthetic construct
EIVLTQSPGTLSLSPGERATLSCRASQSVSSNFLVWYQQKPGQAPRPLIYGASTRASGIPDRFSGSGSGADFTLTISRLEPEDFALYYCQQYGDSPYTFGQGTTLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEGKSSGSGSESKSTEGKSSGSGSESKSTGGGGSQVQLVQSGAEVKKPGASVKVSCKASGNTFTNYYMHWVRQAPGPGLEWMGIIDPSVGSKSYAQKFLGRVTIARDTSTSTVFLDLYSLRSEDTAVYFCARAGKRFGESYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 50 - protein, 479 aa, synthetic construct
DIQMTQSPSAMSASVGDRVTITCRASQGISNYLAWFQQKPGKVPEPLIYAASSLQSGVPSRFSGSGSVTEFTLTISSLQPEDFATYYCLQHNSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEGKSSGSGSESKSTEGKSSGSGSESKSTGGGGSQVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGEAVGGNYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 51 - protein, 479 aa, synthetic construct
DIQMTQSPSAMSASVGDRVTITCRASQGISNYLVWFQQKPGKAPKRLIYAVSSLYSGVPSRFSGSGSGTEFTLTISSLQPEDFATYYCLQHDSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEGKSSGSGSESKSTEGKSSGSGSESKSTGGGGSQVQLVQSGAEVKKPGASVKVSCKASGYTFTSYYVHWVRQAPGQGLEWMVIINPGGGTTSYAQKFLGRVTMTRDTSTNTVYMELKSLRSEDTAVYYCARGEAVTGNYFYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 52-54 - skipped (3 entries)

SEQ ID NO: 55 - protein, 14 aa, synthetic construct
RSSTGAVTTSNYAN

SEQ ID NO: 56 - protein, 7 aa, synthetic construct
GTNKRAP

SEQ ID NO: 57 - protein, 9 aa, synthetic construct
ALWYSNLWV

SEQ ID NO: 58 - protein, 10 aa, synthetic construct
GFTFNTYAMN

SEQ ID NO: 59 - protein, 19 aa, synthetic construct
RIRSKYNNYATYYADSVKD

SEQ ID NO: 60 - protein, 14 aa, synthetic construct
HGNFGNSYVSWFAY

SEQ ID NO: 61 - protein, 14 aa, synthetic construct
RSSTGAVTTSNYAN

SEQ ID NO: 62 - protein, 7 aa, synthetic construct
GTNKRAP

SEQ ID NO: 63 - protein, 9 aa, synthetic construct
ALWYSNLWV

SEQ ID NO: 64 - protein, 10 aa, synthetic construct
GFTFNTYAMN

SEQ ID NO: 65 - protein, 19 aa, synthetic construct
RIRSKYINYATYYADSVKD

SEQ ID NO: 66 - protein, 14 aa, synthetic construct
HGNFGNSYVSWFAY

SEQ ID NO: 67 - protein, 109 aa, synthetic construct
EAVVTQEPSLTVSPGGTVTLTCRSSTGAVTTSNYANWVQEKPGQLPRGLIGGTNKRAPWVPARFSGSLLGGKAALTLSGAQPEDEAEYFCALWYSNLWVFGGGTKLTVL

SEQ ID NO: 68 - protein, 125 aa, synthetic construct
EVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSA

SEQ ID NO: 69 - protein, 109 aa, synthetic construct; identical to SEQ ID NO: 67

SEQ ID NO: 70 - protein, 125 aa, synthetic construct
EVQLVESGGGLVQPGGSLKLSCAASGFTFNTYAMNWVRQAPGKGLEWVARIRSKYINYATYYADSVKDRFTISRDDSKNTAYLQMNNLKTEDTAVYYCVRHGNFGNSYVSWFAYWGQGTLVTVSA

SEQ ID NO: 71 - protein, 481 aa, synthetic construct
EAVVTQEPSLTVSPGGTVTLTCRSSTGAVTTSNYANWVQEKPGQLPRGLIGGTNKRAPWVPARFSGSLLGGKAALTLSGAQPEDEAEYFCALWYSNLWVFGGGTKLTVLGQPKAAPSVTLFPPSSEELQANKATLVCLISDFYPGAVKVAWKADGSPVNTGVETTTPSKQSNNKYAASSYLSLTPEQWKSHRSYSCQVTHEGSTVEKTVAPAECSGGGGSEGKSSGSGSESKSTEGKSSGSGSESKSTGGGGSEVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 72 - protein, 481 aa, synthetic construct
EAVVTQEPSLTVSPGGTVTLTCRSSTGAVTTSNYANWVQEKPGQLPRGLIGGTNKRAPWVPARFSGSLLGGKAALTLSGAQPEDEAEYFCALWYSNLWVFGGGTKLTVLGQPKAAPSVTLFPPSSEELQANKATLVCLISDFYPGAVKVAWKADGSPVNTGVETTTPSKQSNNKYAASSYLSLTPEQWKSHRSYSCQVTHEGSTVEKTVAPAECSGGGGSEGKSSGSGSESKSTEGKSSGSGSESKSTGGGGSEVQLVESGGGLVQPGGSLKLSCAASGFTFNTYAMNWVRQAPGKGLEWVARIRSKYINYATYYADSVKDRFTISRDDSKNTAYLQMNNLKTEDTAVYYCVRHGNFGNSYVSWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 73 - protein, 703 aa, synthetic construct
EIVLTQSPGTLSLSPGERATLSCRASQSVSSNFLVWYQQKPGQAPRPLIYGASTRASGIPDRFSGSGSGADFTLTISRLEPEDFALYYCQQYGDSPYTFGQGTTLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEGKSSGSGSESKSTEGKSSGSGSESKSTGGGGSQVQLVQSGAEVKKPGASVKVSCKASGNTFTNYYMHWVRQAPGPGLEWMGIIDPSVGSKSYAQKFLGRVTIARDTSTSTVFLDLYSLRSEDTAVYFCARAGKRFGESYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 74 - protein, 705 aa, synthetic construct
DIQMTQSPSAMSASVGDRVTITCRASQGISNYLAWFQQKPGKVPEPLIYAASSLQSGVPSRFSGSGSVTEFTLTISSLQPEDFATYYCLQHNSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEGKSSGSGSESKSTEGKSSGSGSESKSTGGGGSQVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGEAVGGNYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 75 - protein, 705 aa, synthetic construct
DIQMTQSPSAMSASVGDRVTITCRASQGISNYLVWFQQKPGKAPKRLIYAVSSLYSGVPSRFSGSGSGTEFTLTISSLQPEDFATYYCLQHDSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEGKSSGSGSESKSTEGKSSGSGSESKSTGGGGSQVQLVQSGAEVKKPGASVKVSCKASGYTFTSYYVHWVRQAPGQGLEWMVIINPGGGTTSYAQKFLGRVTMTRDTSTNTVYMELKSLRSEDTAVYYCARGEAVTGNYFYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 76-78 - skipped (3 entries)

SEQ ID NO: 79 - protein, 707 aa, synthetic construct
EAVVTQEPSLTVSPGGTVTLTCRSSTGAVTTSNYANWVQEKPGQLPRGLIGGTNKRAPWVPARFSGSLLGGKAALTLSGAQPEDEAEYFCALWYSNLWVFGGGTKLTVLGQPKAAPSVTLFPPSSEELQANKATLVCLISDFYPGAVKVAWKADGSPVNTGVETTTPSKQSNNKYAASSYLSLTPEQWKSHRSYSCQVTHEGSTVEKTVAPAECSGGGGSEGKSSGSGSESKSTEGKSSGSGSESKSTGGGGSEVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 80 - protein, 707 aa, synthetic construct
EAVVTQEPSLTVSPGGTVTLTCRSSTGAVTTSNYANWVQEKPGQLPRGLIGGTNKRAPWVPARFSGSLLGGKAALTLSGAQPEDEAEYFCALWYSNLWVFGGGTKLTVLGQPKAAPSVTLFPPSSEELQANKATLVCLISDFYPGAVKVAWKADGSPVNTGVETTTPSKQSNNKYAASSYLSLTPEQWKSHRSYSCQVTHEGSTVEKTVAPAECSGGGGSEGKSSGSGSESKSTEGKSSGSGSESKSTGGGGSEVQLVESGGGLVQPGGSLKLSCAASGFTFNTYAMNWVRQAPGKGLEWVARIRSKYINYATYYADSVKDRFTISRDDSKNTAYLQMNNLKTEDTAVYYCVRHGNFGNSYVSWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 81 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 82 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 83 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPG

SEQ ID NO: 84 - protein, 228 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 85 - protein, 228 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLG

SEQ ID NO: 86 - protein, 228 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNRFTQKSLSLSLG

SEQ ID NO: 87 - protein, 107 aa, synthetic construct [sequence omitted]

SEQ ID NO: 88 - protein, 106 aa, synthetic construct
GQPKAAPSVTLFPPSSEELQANKATLVCLISDFYPGAVKVAWKADGSPVNTGVETTTPSKQSNNKYAASSYLSLTPEQWKSHRSYSCQVTHEGSTVEKTVAPAECS

SEQ ID NO: 89 - protein, 38 aa, synthetic construct
GGGGSEGKSSGSGSESKSTEGKSSGSGSESKSTGGGGS

SEQ ID NO: 90 - protein, 103 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC